(19) United States
(12) Patent Application Publication
(10) Pub. No.: US 2003/0103992 A1
(43) Pub. Date: Jun. 5, 2003

(54) Clasp membrane proteins

(75) Inventors: Peter S. Lu, Mountain View, CA (US); Jonathan David Garman, San Jose, CA (US); Albert F. Candia III, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ARBOR VITA CORPORATION, SUNNYVALE, CA

(21) Appl. No.: 09/978,244

(22) Filed: Oct. 15, 2001

Related U.S. Application Data
(63) Continuation-in-part of application No. 09/737,246, filed on Dec. 13, 2000.
(63) Continuation-in-part of application No. 09/547,276, filed on Apr. 11, 2000.
(63) Continuation-in-part of application No. 09/736,969, filed on May 7, 2001.
(63) Continuation-in-part of application No. 09/547,276, filed on Apr. 11, 2000.
(63) Continuation-in-part of application No. 09/736,968, filed on Dec. 13, 2000.
(63) Continuation-in-part of application No. 09/547,276, filed on Apr. 11, 2000.
(63) Continuation-in-part of application No. 09/736,960, filed on Dec. 13, 2000.
(63) Continuation-in-part of application No. 09/547,276, filed on Apr. 11, 2000.
(60) Provisional application No. 60/310,028, filed on Aug. 3, 2001.
(60) Provisional application No. 60/240,545, filed on Oct. 13, 2000.
(60) Provisional application No. 60/240,508, filed on Oct. 13, 2000.
(60) Provisional application No. 60/240,503, filed on Oct. 13, 2000.
(60) Provisional application No. 60/240,539, filed on Oct. 13, 2000.
(60) Provisional application No. 60/240,543, filed on Oct. 13, 2000.
(60) Provisional application No. 60/196,267, filed on Apr. 11, 2000.
(60) Provisional application No. 60/196,527, filed on Apr. 11, 2000.
(60) Provisional application No. 60/196,528, filed on Apr. 11, 2000.
(60) Provisional application No. 60/196,460, filed on Apr. 11, 2000.
(60) Provisional application No. 60/160,860, filed on Oct. 21, 1999.
(60) Provisional application No. 60/162,498, filed on Oct. 29, 1999.
(60) Provisional application No. 60/170,453, filed on Dec. 13, 1999.
(60) Provisional application No. 60/176,195, filed on Jan. 14, 2000.
(60) Provisional application No. 60/182,296, filed on Feb. 14, 2000.
(60) Provisional application No. 60/240,545, filed on Oct. 13, 2000.
(60) Provisional application No. 60/240,508, filed on Oct. 13, 2000.
(60) Provisional application No. 60/240,503, filed on Oct. 13, 2000.
(60) Provisional application No. 60/240,539, filed on Oct. 13, 2000.
(60) Provisional application No. 60/240,543, filed on Oct. 13, 2000.
(60) Provisional application No. 60/240,545, filed on Oct. 13, 2000.
(60) Provisional application No. 60/240,508, filed on Oct. 13, 2000.
(60) Provisional application No. 60/240,503, filed on Oct. 13, 2000.
(60) Provisional application No. 60/240,539, filed on Oct. 13, 2000.
(60) Provisional application No. 60/240,543, filed on Oct. 13, 2000.
(60) Provisional application No. 60/240,545, filed on Oct. 13, 2000.
(60) Provisional application No. 60/240,508, filed on Oct. 13, 2000.
(60) Provisional application No. 60/240,503, filed on Oct. 13, 2000.
(60) Provisional application No. 60/240,539, filed on Oct. 13, 2000.
(60) Provisional application No. 60/240,543, filed on Oct. 13, 2000.

(51) Int. Cl. C07H 21/04; A61K 39/00; C12N 9/64; C12P 21/02; C12N 5/06

(52) U.S. Cl. 424/185.1; 435/226; 536/23.2; 435/69.1; 435/320.1; 435/325

(57) ABSTRACT

The present invention relates to cell surface molecules, designated cadherin-like asymmetry proteins (“CLASPs”). In particular, it relates to CLASP polynucleotides, polypeptides, fusion proteins, and antibodies. The invention also relates to methods of modulating an immune response by interfering with CLASP function.

### 0.0 CROSS REFERENCE TO RELATED APPLICATIONS

This application claims priority to U.S. Provisional Application No. 60/310,028 (filed Aug. 3, 2001), U.S. application Ser. Nos. 09/737,246, 09/736,969, 09/736,960, 09/736,968 (all filed Dec. 13, 2000), 60/240,545, 60/240,508, 60/240,503, 60/240,539, 60/240,543 (all filed Oct. 13, 2000); 09/547,276, 60/196,267, 60/196,527, 60/196,528, 60/196,460 (all filed Apr. 11, 2000); 60/182,296 (filed Feb. 14, 2000), 60/176,195 (filed Jan. 14, 2000), 60/170,453 (filed Dec. 13, 1999), 60/162,498 (filed Oct. 29, 1999), 60/160,860 (filed Oct. 21, 1999).

### 1.0 FIELD OF THE INVENTION

The present invention relates to molecules expressed in cells of the immune system. In particular, the invention relates to a membrane protein that contains certain classical cadherin characteristics.

### 2.0 BACKGROUND OF THE INVENTION

The generation of an immune response against an antigen is carried out by a number of distinct immune cell types that work in concert within the context of a particular antigen. The helper T cell (T_{H}) plays a pivotal role to coordinate two types of antigen-specific immune response; i.e., cellular and humoral immune response. Recognition of antigen by T cells requires the formation of a specialized junction between the T cell and the antigen-presenting cell (APC) called the “immunological synapse” (Dustin, et al., 1998, Cell 94: 667-677). The immune synapse orchestrates recruitment and exclusion of specific proteins from the contact area by an unknown mechanism and is thought to be initiated by T-cell antigen receptor (TCR) recognition of peptides bound to MHC molecules (antigen) (Monk, et al. 1998, Nature 395: 82). However, the low affinity of the TCR for antigen as well as limited number of ligands makes it unlikely that TCR: antigen interaction alone is sufficient to drive the formation of the immunological synapse (Matsui et al., 1994, Proc. Natl. Acad. Sci. U.S.A. 91: 12861-12866).

Costimulatory molecules such as CD4, ICAM-1, LFA-1, CD28, CD2 have been proposed to stabilize the cell-cell contact (Dustin, et al., 1999, Science 283: 649). However, since these molecules are recruited to the synapse after activation they cannot account for the high specificity and avidity during the early phases of T-cell antigen recognition. Recent work demonstrated that a portion of the T cell surface at the leading edge is specialized to mediate the early phases of synapse formation (Negulescu, et al., 1996, Immunity 4: 421-430). Such a specialization must be a pre-formed structure containing cell surface adhesion proteins (ectodomains) to augment TCR engagement and corresponding cytoplasmic portions (endodomains) to transduce signals and bind cytoskeleton to maintain structural/functional polarity.

The ectodomain of the pre-formed synapse or “immune gateway” was recently discovered and is created in part by CLASP-1 (U.S. Ser. No. 09/411,328, filed Oct. 1, 1999; PCT/US99/22996). In addition to cadherin motifs, CLASP-1 also contains a CRK-SH3 binding domain, tyrosine phosphorylation sites, and coiled/coil domains suggesting direct interaction with cytoskeleton and regulation by adaptor molecules such as CRK. The CLASP-1 transcript is present in lymphoid organs and neural tissue, and the protein is expressed by T and B lymphocytes and macrophages in the MOMA-1 subregion of the marginal zone of the spleen, an area known to be important in T:B cell interaction. CLASP-1 staining of individual T and B cells exhibits a preactivation structural polarity, being organized as a “ball” or “cap” structure in B cells, and forming a “ring”, “ball” or “cap” structure in T cells. The placement of these structures is adjacent to the microtubule-organizing center (“MTOC”). CLASP-1 antibody staining indicates that CLASP-1 is at the interface of T-B cell conjugates that are fully committed to differentiation. Antibodies to the extracellular domain of CLASP-1 also block T-B cell conjugate formation and T cell activation.

### 3.0 SUMMARY OF THE INVENTION

The present invention relates to several related cell surface molecules, designated cadherin-like asymmetry protein(s) (“CLASP(s)”). In particular, it relates to a polynucleotide comprising a coding sequence for CLASP-1, -2, -3, -4, -5, or -7, a polynucleotide that selectively hybridizes to the complement of a CLASP coding sequence, expression vectors containing such polynucleotides, genetically-engineered host cells containing such polynucleotides, CLASP polypeptides, CLASP fusion proteins, therapeutic compositions, CLASP domain mutants, antibodies specific for CLASP polypeptides, methods for detecting the expression of CLASPs, and methods of inhibiting an immune response by interfering with CLASP function. A wide variety of uses are encompassed by the invention, including but not limited to, treatment of autoimmune diseases and hypersensitivities, prevention of transplantation rejection responses, and augmentation of immune responsiveness in immunodeficiency states.

In one aspect, the invention provides an isolated or recombinant polynucleotide comprising a nucleotide sequence that encodes any one of a CLASP-1. -2, -3, -4, -5, or -7, or a variant or fragment thereof, or the complementary strand of such a polynucleotide. In various embodiments, the polynucleotide has a sequence as shown in FIG. 1, FIG. 3, FIG. 6A or encodes a polypeptide having a sequence shown in FIG. 1, FIG. 3, FIG. 6A or one or more domains thereof.

In a further aspect, the invention provides an isolated CLASP polynucleotide that is: (a) a polynucleotide that has the sequence as shown in FIG. 1, FIG. 3, FIG. 6A (b) a polynucleotide that hybridizes under stringent hybridization conditions to (a) and encodes a polypeptide having the sequence as shown in FIG. 1, FIG. 3, FIG. 6A or an allelic variant or homologue of a polypeptide having the sequence of as shown in FIG. 1, FIG. 3, FIG. 6A; or (c) a polynucleotide that hybridizes under stringent hybridization conditions to (a) and encodes a polypeptide with at least 25 contiguous residues of the polypeptide of as shown in FIG. 1, FIG. 3, FIG. 6A; or (d) a polynucleotide that hybridizes under stringent hybridization conditions to (a) and has at least 12 contiguous bases identical to or exactly complementary to as shown in FIG. 1, FIG. 3, FIG. 6A.

In a related aspect, the invention provides a CLASP polynucleotide that encodes a polypeptide having the full-length sequence as shown in FIG. 1, FIG. 3, FIG. 6A or the cDNA sequence encoded by the inserts of ATCC Deposit No. ______.

In another aspect, the invention further provides an isolated CLASP polynucleotide comprising a nucleotide sequence that has at least 90% percent identity to the sequence as shown in FIG. 1, FIG. 3, FIG. 6A as calculated using FASTA wherein said sequences are aligned so that highest order match between said sequences is obtained.

The invention further provides an isolated CLASP polypeptide comprising a nucleotide sequence that has at least 90% sequence identity to the sequence as shown in FIG. 1, FIG. 3, FIG. 6A and is immunologically crossreactive with the sequence as shown in FIG. 1, FIG. 3, FIG. 6A or shares a biological function with the native CLASP polypeptide.

The invention also provides vectors, such as expression vectors, comprising a polynucleotide sequence of the invention In other embodiments, the invention provides host cells or progeny of the host cells comprising a vector of the invention. In certain embodiments, the host cell is a eukaryote. In other embodiments, the expression vector comprises a CLASP polynucleotide in which the nucleotide sequence of the polynucleotide is operatively linked with a regulatory sequence that controls expression of the polynucleotide in a host cell. In certain embodiments, the invention provides a host cell comprising a CLASP polynucleotide, wherein the nucleotide sequence of the polynucleotide is operatively linked with a regulatory sequence that controls expression of the polynucleotide in a host cell, or progeny of the cell.

In another aspect, the invention further provides a CLASP polynucleotide that is an antisense polynucleotide. In a preferred embodiment, the antisense polynucleotide is less than about 200 bases in length. In other embodiments, the invention provides an antisense oligonucleotide complementary to a messenger RNA comprising the sequence as shown in FIG. 1, FIG. 3, FIG. 6A and encoding the CLASP polynucleotide, wherein the oligonucleotide inhibits the expression of the particular CLASP polynucleotide.

In another aspect, the invention provides an isolated DNA that encodes a CLASP protein as shown in FIG. 1, FIG. 3, FIG. 6A. In certain embodiments, the CLASP polynucleotide is RNA.

The invention provides a method for producing a polypeptide comprising: (a) culturing the host cell containing a CLASP polynucleotide under conditions such that the polypeptide is expressed; and (b) recovering the polypeptide from the cultured host cell or its cultured medium.

The invention further provides an isolated CLASP polypeptide encoded by a CLASP polynucleotide. In some embodiments, the CLASP polypeptide has the amino acid sequence s shown in FIG. 1, FIG. 3, FIG. 6A, or a fragment thereof. In some embodiments, the isolated CLASP polypeptide is cell-membrane associated. In other embodiments, the isolated CLASP polypeptide is soluble. In other embodiments, the soluble CLASP polypeptide is fused with a heterologous polypeptide.

The invention further provides an isolated CLASP protein having the sequence as shown in FIG. 1, FIG. 3, FIG. 6A. In some embodiments, the invention provides a CLASP protein comprising the sequence as shown in FIG. 1, FIG. 3, FIG. 6A and variants thereof that are at least 95% identical to the sequence as shown in FIG. 1, FIG. 3, FIG. 6A and specifically binds a cytoskeletal protein. In certain embodiments the cytoskeletal protein is spectrin.

The invention further provides an isolated antibody that specifically binds to a polypeptide having the amino acid sequence as shown in as shown in FIG. 1, FIG. 3, FIG. 6A, or a binding fragment thereof. In some embodiments the antibody is monoclonal. In other embodiments, the invention provides a hybridoma capable of secreting the antibody.

The invention further provides a method of identifying a compound or agent that binds a CLASP polypeptide comprising: i) contacting a CLASP polypeptide with the compound or agent under conditions which allow binding of the compound to the CLASP polypeptide to form a complex and ii) detecting the presence of the complex.

The invention further provides a method of detecting a CLASP polypeptide in a sample, comprising: (a) contacting the sample with a CLASP antibody or binding fragment and (b) determining whether a complex has been formed between the antibody and with CLASP polypeptide.

The invention further provides a method of detecting a CLASP polypeptide in a sample, comprising: (a) contacting the sample with a CLASP polynucleotide or a polynucleotide that comprises a sequence of at least 12 nucleotides and is complementary to a contiguous sequence of the CLASP-1 polynucleotide and (b) determining whether a hybridization complex has been formed.

The invention further provides a method of detecting a CLASP nucleotide in a sample, comprising: (a) using a polynucleotide that comprises a sequence of at least 12 nucleotides and is complementary to a contiguous sequence of a CLASP polynucleotide in an amplification process; and (b) determining whether a specific amplification product has been formed.

The invention further provides pharmaceutical compositions comprising a CLASP polynucleotide, a CLASP polypeptide, or a CLASP antibody and a pharmaceutically acceptable carrier.

In one aspect, the invention provides a method of inhibiting an immune response in a cell comprising: (a) interfering with the expression of a CLASP gene in the cell; (b) interfering with the ability of a CLASP protein to mediate cell-cell interaction (e.g., interfering with a heterotypic and/or homotypic interaction) between CLASP and an extracellular protein; (c) interfering with the ability of a CLASP protein to bind to another protein. In some such methods, the cell is a T cell, a B cell, a macrophage, or a dendritic cell. Some such methods comprise contacting the cell with an effective amount of a polypeptide which comprises the amino acid sequence as shown in FIG. 1, FIG. 3, FIG. 6A or a fragment thereof.

In another aspect, the invention provides a method of inhibiting an immune response in a subject, comprising administering to the subject a therapeutically effective amount of an antibody which specifically binds a CLASP polypeptide, e.g., a CLASP extracellular region, to inhibit or stimulate the immune response. The invention also provides a method of treating an autoimmune disease in a subject, comprising administering to the subject a therapeutically effective amount of a polypeptide which comprises the amino acid sequence of a CLASP polypeptide disclosed herein.

In another aspect, the invention provides a method of preventing or treating a CLASP-mediated disease comprising administering to a subject in need thereof a therapeutically effective amount of a CLASP pharmaceutical composition. In some such methods, the CLASP-mediated disease is an autoimmune disease.

The invention further provides a method of treating an autoimmune disease in a subject caused or exacerbated by increased activity of T_{H}1 cells consisting of administering a therapeutically effective amount of a CLASP pharmaceutical composition to the subject.

In one aspect, the invention provides methods for modulating (inhibiting or enhancing) an immune response by changing CLASP expression or activity. Thus, in one embodiment, an immune response is inhibited or stimulated by interfering with CLASP expression (e.g., using antisense, ribozyme, or transcription or translation inhibitors). In a different embodiment, CLASP activity is inhibited by blocking the interaction of CLASP with an associated protein (e.g., a PTK protein, a cytoskeletal protein, a PDZ-domain containing protein, and the like), e.g., using a CLASP peptide, a small molecule antagonist of the interaction, and the like.

The invention further provides methods for in vitro or ex vivo enrichment or selection of cells by contacting hematopoietic cells, e.g., PBCs or bone marrow cells, with a CLASP polypeptide of the invention or, alternatively, an anti-CLASP antibody of the invention.

### BRIEF DESCRIPTION OF THE DRAWINGS

FIG. 1. Nucleotide and predicted amino acid sequence of full length and partial mouse CLASP cDNAs A) Full length cDNA sequence and predicted amino acid translation of the mouse CLASP-1 cDNA. Predicted initiator methionine (bolded and underlined) starts at nucleotide +1 . In-frame stop codon (underlined) upstream of the initiator methionine supports the fact that this sequence represents a full length coding region of mouse CLASP-1. Nucleotides 2261 and greater of FIG. 1A correspond to nucleotides 319 and greater of PCT/US99/22996. B) Partial cDNA sequence and predicted amino acid translation of the mouse CLASP-2 cDNA. Predicted initiator methionine (underlined and bolded) starts at nucleotide 67. In-frame stop codons upstream of the initiator methionine are not present, which is indicative of the partial nature of this cDNA sequence as well as the potential for the initiator methionine to be upstream of the presented sequence. However, the methionine at nucleotide 67 initiates the coding sequence in a similar position when compared to human CLASP-2 (see comparison in FIG. 3B). C) Partial cDNA sequence and predicted amino acid translation of the mouse CLASP-3 cDNA. D) Full length cDNA sequence and predicted amino acid translation of the mouse CLASP-4 cDNA. Predicted initiator methionine (bolded and underlined) starts at nucleotide +1. An in-frame stop codon (underlined) upstream of the initiator methionine supports the fact that this sequence represents a full length coding region of mouse CLASP-4. E) Full length cDNA sequence and predicted amino acid translation of the mouse CLASP-5 cDNA. Predicted initiator methionine (underlined and bolded) starts at nucleotide +1. In-frame stop codons upstream of the initiator methionine (underlined) support the fact that this sequence represents the full length coding region of mouse CLASP-5. F) Partial cDNA sequences and predicted amino acid translation of the mouse CLASP-7 cDNA.

FIG. 2. Allelic variations, alterations and deletions between the mouse CLASP cDNA isoforms. Sequencing multiple, independent cDNA products revealed nucleotide differences, which may indicate single nucleotide, allelic variations between CLASP cDNA isoforms. A) mouse CLASP-1 B) mouse CLASP-2 C) mouse CLASP-3 D) mouse CLASP-4 E) mouse CLASP-5

FIG. 3. Amino acid alignment and comparison between the human and mouse CLASPs. Amino acid sequences were aligned using ClustalW. Single letter amino acid abbreviations are used. Asterisks under the sequence indicate complete identity, while colons and periods indicate sequence similarity between the two sequences. Dashes within the sequence indicate gaps inserted by ClustalW to facilitate alignment. A) mouse and human CLASP-1. The mouse and human CLASP-1 protein sequences are 92.2% identical and 97.6% similar as determined by FASTA analysis (not shown). B) mouse and human CLASP-2. The mouse and human CLASP-2 protein sequences are 92.1% identical and 98.8% similar as determined by FASTA analysis (not shown). C) mouse and human CLASP-3. The mouse and human CLASP-3 protein sequences are 96.2% identical and 99% similar as determined by FASTA analysis (not shown). D) mouse and human CLASP-4. The mouse and human CLASP-4 protein sequences are 96.4% identical and 99.4% similar as determined by FASTA analysis (not shown). E) mouse and human CLASP-5. The mouse and human CLASP-5 protein sequences are 91% identical and 97.5% similar as determined by FASTA analysis (not shown). E) mouse and human CLASP-7. The presented mouse and human CLASP-7 protein sequences are 89% identical with respect to the mouse CLASP-7 sequence presented as determined by FASTA analysis.

FIG. 4. Expression profile of mouse CLASPs in multiple tissues. A) Mouse CLASP-1 expression by Northern analysis with RNA from multiple tissues. Northern analysis was performed to determine mouse CLASP-1 expression in various mouse tissues. A portion of the mouse CLASP-1 cDNA was used as a probe and revealed expression of an approximately 7.5 kb specific transcript in various tissues. Signal intensities correlate to the relative abundance of mouse CLASP-1-specific RNA. B) Mouse CLASP-2 expression profile as determined by Northern blot analysis. Northern analysis was performed to determine mouse CLASP-2 gene expression in various mouse tissues. A portion of the mouse CLASP-2 cDNA was used as probe and revealed expression of an approximately 7.5 kb and 5.5 kb mouse CLASP-2-specific transcripts in various tissues. Signal intensities correlate to the relative abundance of mouse CLASP-2-specific RNA. C) Mouse CLASP-3 expression profile as determined by Northern blot analysis. Northern analysis was performed to determine mouse CLASP-3 gene expression in various mouse tissues. A portion of the mouse CLASP-3 cDNA was used as probe and revealed expression of an approximately 7.5 kb and 5.5 kb mouse CLASP-3-specific transcripts in various tissues. Signal intensities correlate to the relative abundance of mouse CLASP-3-specific RNA. D) Mouse CLASP-4 expression profile as determined by Northern blot analysis. Northern analysis was performed to determine mouse CLASP-4 gene expression in various mouse tissues. A portion of the mouse CLASP-4 cDNA was used as probe and revealed expression of an approximately 7.4 kb mouse CLASP-4-specific transcript in various tissues. Signal intensities correlate to the relative abundance of mouse CLASP-4-specific RNA. E) mouse CLASP-5 expression profile as determined by Northern blot analysis. Northern analysis was performed to determine mouse CLASP-5 gene expression in various mouse tissues. A portion of the mouse CLASP-5 cDNA was used as probe and revealed expression of multiple transcripts of 7.5, 6.5, 4.5 and 3.5 kb. Signal intensities correlate to the relative abundance of mouse CLASP-5-specific RNA. F) Mouse CLASP-7 expression profile as determined by Northern blot analysis. Northern analysis was performed to determine mouse CLASP-7 gene expression in various mouse tissues. A portion of the mouse CLASP-7 cDNA was used as probe and revealed expression of an approximately 7.5 kb and 5.5 kb mouse CLASP-7-specific transcripts in various tissues. Signal intensities correlate to the relative abundance of mouse CLASP-7-specific RNA.

FIG. 5. Multiple alignment of mouse CLASP amino acid sequences. Alignment of mouse CLASP amino acid sequences was accomplished using ClustalW. Alignment shows that the mouse CLASPs are highly related to each other and comprise a family.

FIG. 6. A. Full length cDNA sequence and predicted amino acid translation of the human CLASP-1 gene. Predicted initiator methionine starts at nucleotide +1. An in-frame stop codon upstream of the initiator methionine (−42 to −40) supports the fact that this sequence represents the full length coding region of CLASP-1. Nucleotides 1522 and greater of FIG. 6 correspond to nucleotides 72 and greater of (U.S. Ser. No. 09/411,328, filed Oct. 1, 1999; PCT/US99/22996). B. Nucleotide polymorphisms between the human CLASP-1 cDNA isoforms. Sequencing multiple, independent cDNA products revealed nucleotide differences, which may indicate single nucleotide, allelic variations between CLASP-1 cDNA isoforms.

FIG. 7. Sequence of human CLASP-1 exons and introns, and promoter. A Sequence of human CLASP-1 exons and intron borders. Stretches of noncontigous genomic sequence from the Human Genome Project (Genbank entry gi8705162) were aligned using the human CLASP-1 cDNA as a template and Sequencher sequence analysis software (Gene Codes Corp). Due to the incompleteness of the Human Genome Project, only partial genomic sequence from human CLASP-1 was obtained. Six exons representing approximately the 5′ 10% of the human CLASP-1 cDNA sequence are presented in predicted 5′ to 3′ order. Exon sequences are underlined and are flanked by intron sequence. This exon/intron map could only have been produced having the isolated human CLASP-1 cDNA. Nucleotide numbers in parentheses refer to the exon sequence within the uniquely-generated, ordered gi8705162 sequence, which is presented in FIG. 7B. B. Ordered stretch of human genomic DNA at the CLASP-1 locus aligned from noncontiguous, shotgun sequencing from the Human Genome Project using the human CLASP-1 sequence from FIG. 6A to determine genomic DNA fragment order and orientation. C) Putative promoter sequence for human CLASP-1. The 5′ end of the cDNA is underlined. This sequence represents the reverse complement (i.e., antiparallel) of nucleotides 50451 to 52500 of Genbank entry gi8705162.

FIG. 8. Amino acid alignment and comparison between the human (h) CLASP family members. Amino acid sequences were aligned using ClustalW. The alignment is presented in order of their greatest pairwise similarity scores. Single letter amino acid abbreviations are used. Astericks indicate complete identity, while colons and periods indicate sequence similarity among CLASP family members. Dashes indicate gaps inserted in the amino acid sequence to facilitate alignment. Labelled boxes are domains with similarity to known protein motifs; unlabelled boxes represent regions of similarity between all CLASPs and may represent CLASP-specific domains.

FIG. 9. Expression of human CLASP-1 upon T-cell activation as assayed by Northern analysis. Jurkat cells were activated using PMA, Ionomycin, and αCD28. RNA was prepared from cell culture aliquots at 0, 1, 2, 4, 8, 14 hours post activation and Northern analysis was performed FIG. 9B. Hybridization signals obtained with a CLASP-1-specific probe were quantified using a phosphor imager system. Relative signal intensities (refers to total signal of the specific probe used) are shown in the bar diagram shown in FIG. 9B. The ethidium staining of the Northern gel in FIG. 9A demonstrates even RNA loading.

### DETAILED DESCRIPTION

5.0 Definitions

Except when noted, the terms “patient” or “subject” are used interchangeably and refer to mammals such as human patients and non-human primates, as well as experimental animals such as rabbits, rats, and mice, and other animals.

The term “biological sample” as used herein is a sample of biological tissue, fluid, or cells that contain CLASP or nucleic acids encoding CLASP proteins. Such samples include, but are not limited to, tissue isolated from humans. Biological samples may also include sections of tissues such as frozen sections taken for histologic purposes. A biological sample is typically obtained from a eukaryotic organism, preferably eukaryotes such as fungi, plants, insects, protozoa, birds, fish, reptiles, and preferably a mammal such as rat, mice, cow, dog, guinea pig, or rabbit, and most preferably a primate such as chimpanzees or humans.

The term “treating” includes the administration of the compounds or agents of the present invention to prevent or delay the onset of the symptoms, complications, or biochemical indicia of a disease, alleviating the symptoms or arresting or inhibiting further development of the disease, condition, or disorder (e.g., autoimmune disease). Treatment may be prophylactic (to prevent or delay the onset of the disease, or to prevent the manifestation of clinical or subclinical symptoms thereof) or therapeutic suppression or alleviation of symptoms after the manifestation of the disease.

The term “lymphocyte” as used herein has the normal meaning in the art, and refers to any of the mononuclear, nonphagocytic leukocytes, found in the blood, lymph, and lymphoid tissues, i.e., B and T lymphocytes.

The terms “isolated”, or “purified”, refer to material that is substantially free from components that normally accompany it as found in its native state (e.g., recombinantly produced or purified away from other cell components with which it is naturally associated). Purity and homogeneity are typically determined using analytical chemistry techniques such as polyacrylamide gel electrophoresis or high performance liquid chromatography. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “nucleic acid” and “polynucleotide” are used interchangeably and refer to DNA, RNA and nucleic acid polymers containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The terms “polypeptide,” “peptide” and “protein” are used interchangeably herein to refer to a polymer of amino acid residues. The amino acids may be natural amino acids, or include an artificial chemical mimetic of a corresponding naturally occurring amino acid.

As used herein a “nucleic acid probe” is defined as a nucleic acid capable of specifically binding to a target nucleic acid of complementary sequence (e.g., through complementary base pairing). As used herein, a probe may include natural (i.e., A, G, C, or T) or modified bases (7-deazaguanosine, inosine, and the like). In addition, the bases in a probe may be joined by a linkage other than a phosphodiester bond, so long as it does not interfere with hybridization (e.g., probes may be peptide nucleic acids). The probes can be directly labeled as with isotopes, chromophores, lumiphores, chromogens, or indirectly labeled such as with biotin to which a streptavidin complex may later bind.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or, in the case of cells, to progeny of a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “sequence identity” refers to a measure of similarity between amino acid or nucleotide sequences, and can be measured using methods known in the art, such as those described below:

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region (see, e.g., SEQ ID NO: ______), when compared and aligned for maximum correspondence over a comparison window, or designated region as measured using one of the following sequence comparison algorithms or by manual alignment and visual inspection.

The phrase “substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the following sequence comparison algorithms or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred embodiment, the sequences are substantially identical over the entire length of the coding regions.

The phrase “sequence similarity” in the context of two nucleic acids or polypeptides, refers to two or more sequences that are identitical or in the case of amino acids, have homologous amino acid substitutions at either 50%, often at least 60%, often at least 70%, preferably at least 80%, most preferably at least 90% or 95% of the indicated amino acid positions.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to CLASP nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2: 482), by the homology alignment algorithm of Needleman & Wunsch, 1970, J. Mol. Biol. 48: 443, by the search for similarity method of Pearson & Lipman, 1988, Proc. Natl. Acad. Sci. U.S.A. 85: 2444, by computerized implementations of these algorithms (FASTDB (Intelligenetics), BLAST (National Center for Biomedical Information), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., 1987 (1999 Suppl.), Current Protocols in Molecular Biology, Greene Publishing Associates and Wiley Interscience, N.Y.)

A preferred example of an algorithm that is suitable for determining percent sequence identity and sequence similarity is the FASTA algorithm, which is described in Pearson, W. R. & Lipman, D. J., 1988, Proc. Natl. Acad. Sci. U.S.A. 85: 2444. See also W. R. Pearson, 1996, Methods Enzymol. 266: 227-258. Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another preferred example of algorithm that is suitable for determining percent sequence identity and sequence similarity are the BLAST and BLAST 2.0 algorithms, which are described in Altschul et al., 1977, Nuc. Acids Res. 25: 3389-3402 and Altschul et al., 1990, J. Mol. Biol. 215: 403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. U.S.A. 89: 10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Natl. Acad. Sci. U.S.A. 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, 1987, J. Mol. Evol. 35: 351-360. The method used is similar to the method described by Higgins & Sharp, 1989, CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., 1984, Nuc. Acids Res. 12: 387-395.

Another preferred example of an algorithm that is suitable for multiple DNA and amino acid sequence alignments is the CLUSTALW program (Thompson, J. D. et al., 1994, Nucl. Acids. Res. 22: 4673-4680). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, 1992, Proc. Natl. Acad. Sci. U.S.A. 89: 10915-10919).

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available (e.g., the polypeptide of SEQ ID NO: ______ can be made detectable, e.g., by incorporating a radiolabel into the peptide, and used to detect antibodies specifically reactive with the peptide).

The term “sorting” in the context of cells as used herein to refers to both physical sorting of the cells, as can be accomplished using, e.g., a fluorescence activated cell sorter, as well as to analysis of cells based on expression of cell surface markers, e.g., FACS analysis in the absence of sorting.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “specifically (or selectively) binds” to an antibody refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample.

The phrase “specifically bind(s)” or “bind(s) specifically” when referring to a peptide refers to a peptide molecule which has intermediate or high binding affinity, exclusively or predominately, to a target molecule. The phrases “specifically binds to” refers to a binding reaction which is determinative of the presence of a target protein in the presence of a heterogeneous population of proteins and other biologics. Thus, under designated assay conditions, the specified binding moieties bind preferentially to a particular target protein and do not bind in a significant amount to other components present in a test sample. Specific binding to a target protein under such conditions may require a binding moiety that is selected for its specificity for a particular target antigen. A variety of assay formats may be used to select ligands that are specifically reactive with a particular protein. For example, solid-phase ELISA immunoassays, immunoprecipitation, Biacore and Western blot are used to identify peptides that specifically react with PDZ domain-containing proteins. Typically a specific or selective reaction will be at least twice background signal or noise and more typically more than 10 times background. Specific binding between a monovalent peptide and a PDZ-containing protein means a binding affinity of at least 10⁴ M⁻¹, and preferably 10⁵ or 10⁶ M⁻¹.

The phrase “homotypic interaction” refers to the binding of a given protein to another molecule of the same protein (e.g., the binding of a particular CLASP to itself, CLASP-2 to CLASP-2). The phrase “heterotypic interaction” refers to the binding of a given protein to a different protein or other molecule (e.g., the binding of a CLASP protein to a PDZ domain-containing protein or the binding of a transcription factor to DNA).

The phrase “immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

The terms “B lymphocyte response” and “B lymphocyte activity” are used interchangeably to refer to the component of immune response carried out by B lymphocytes (i.e. the proliferation and maturation of B lymphocytes, the binding of antigen to cell surface immunogobulin, the internalization of antigen and presentation of that antigen via MHC molecules to T lymphocytes, and the synthesis and secretion of antibodies).

The terms “T lymphocyte response” and “T lymphocyte activity” are used here interchangeably to refer to the component of immune response dependent on T lymphocytes (i.e., the proliferation and/or differentiation of T lymphocytes into helper, cytotoxic killer, or suppressor T lymphocytes, the provision of signals by helper T lymphocytes to B lymphocytes that cause or prevent antibody production, the killing of specific target cells by cytotoxic T lymphocytes, and the release of soluble factors such as cytokines that modulate the function of other immune cells).

The term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

Components of an immune response may be detected in vitro by various methods that are well known to those of ordinary skill in the art. For example, (1) cytotoxic T lymphocytes can be incubated with radioactively labeled target cells and the lysis of these target cells detected by the release of radioactivity, (2) helper T lymphocytes can be incubated with antigens and antigen presenting cells and the synthesis and secretion of cytokines measured by standard methods (Windhagen A; et al., 1995, Immunity 2(4): 373-80), (3) antigen presenting cells can be incubated with whole protein antigen and the presentation of that antigen on MHC detected by either T lymphocyte activation assays or biophysical methods (Harding et al., 1989, Proc. Natl. Acad. Sci., 86: 4230-4), (4) mast cells can be incubated with reagents that cross-link their Fc-epsilon receptors and histamine release measured by enzyme immunoassay (Siraganian, et al., 1983, TIPS 4: 432-437).

Similarly, products of an immune response in either a model organism (e.g., mouse) or a human patient can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., 1988, Blood 72: 1310-5); (3) the proliferation of peripheral blood mononuclear cells in response to mitogens or mixed lymphocyte reaction can be measured using ³H-thymidine; (4) the phagocytic capacity of granulocytes, macrophages, and other phagocytes in PBMCs can be measured by placing PMBCs in wells together with labeled particles (Peters et al., 1988); and (5) the differentiation of immune system cells can be measured by labeling PBMCs with antibodies to CD molecules such as CD4 and CD8 and measuring the fraction of the PBMCs expressing these markers.

As used herein, the phrase “signal transduction pathway” or “signal transduction event” refers to at least one biochemical reaction, but more commonly a series of biochemical reactions, which result from interaction of a cell with a stimulatory compound or agent. Thus, the interaction of a stimulatory compound with a cell generates a “signal” that is transmitted through the signal transduction pathway, ultimately resulting in a cellular response, e.g., an immune response described above.

A signal transduction pathway refers to the biochemical relationship between a variety of signal transduction molecules that play a role in the transmission of a signal from one portion of a cell to another portion of a cell. Signal transduction molecules of the present invention include, for example, extracellular and intracellular domains of the CLASP proteins. As used herein, the phrase “cell surface receptor” includes molecules and complexes of molecules capable of receiving a signal and the transmission of such a signal across the plasma membrane of a cell. An example of a “cell surface receptor” of the present invention is the T cell receptor (TCR). As used herein, the phrase “intracellular signal transduction molecule” includes those molecules or complexes of molecules involved in transmitting a signal from the plasma membrane of a cell through the cytoplasm of the cell, and in some instances, into the cell's nucleus. In the present invention, the CLASPs can be referred to as a “intracellular signal transduction molecules”, but can also be referred to as “signal transduction molecules”.

A signal transduction pathway in a cell can be initiated by interaction of a cell with a stimulator that is inside or outside of the cell. If an exterior (i.e., outside of the cell) stimulator (e.g., an MHC-antigen complex on an antigen presenting cell) interacts with a cell surface receptor (e.g., a T cell receptor), a signal transduction pathway can transmit a signal across the cell's membrane, through the cytoplasm of the cell, and in some instances into the nucleus. If an interior (e.g., inside the cell) stimulator interacts with an intracellular signal transduction molecule, a signal transduction pathway can result in transmission of a signal through the cell's cytoplasm, and in some instances into the cell's nucleus.

Signal transduction can occur through, e.g., the phosphorylation of a molecule; non-covalent allosteric interactions; complexing of molecules; the conformational change of a molecule; calcium release; inositol phosphate production; proteolytic cleavage; cyclic nucleotide production and diacylglyceride production. Typically, signal transduction occurs through phosphorylating a signal transduction molecule. According to the present invention, a CLASP signal transduction pathway refers generally to a pathway in which a CLASP protein regulates a pathway that includes engaged-receptors, PKC-substrates, G proteins, inositol phosphates, kinases and/or other molecules.

5.1. Introduction

The present invention relates to a family of novel membrane proteins, the CLASPs, that contain an endodomain that display the appropriate properties to organize the cytoskeleton and signal transduction apparatus of the immune gateway.

CLASP-1 is a membrane associated protein that plays important roles in the immune response and in maintenance of the immune system. A 4912 residue CLASP-1 polypeptide was described in PCT/US99/22996, published as WO 00/20434 on Apr. 13, 2000. The aformentioned publication is appended hereto as an Appendix and is herein incorporated by reference in its entirety for all purposes. The present Applicants have discovered (surprisingly) that CLASP-1 exists in forms in addition to and different from that previously described. For example, certain CLASP-1 polypeptides are 7281 residues in length, which are approximately 1632 residues longer than that previously known. It will be apparent that these polypeptide sequences and the corresponding CLASP-1 cDNA sequences have a variety of uses as disclosed herein.

CLASPs function in cells of the immune system, e.g., T cells and B cells, as well as non-immune cells. The CLASP proteins function in a variety of cellular processes, particularly related to immune function, regulation of T cell and B cell interactions, T cell activation, and in the organization, establishment and maintenance of the “immunological synapse” (see Dustin et al., 1999, Science 283: 680-682; Paul et al., 1994, Cell 76: 241-251; Dustin et al., 1996, J. Immunol. 157: 2014; Dustin et al., 1998, Cell 94: 667), including signal transduction, cytoskeletal interactions, and membrane organization.

Without intending to be bound by a particular mechanism or limited in any way, the CLASP proteins are believed to be a component of the lymphocyte organelle called the “immune gateway” that creates a docking site or portal for cell-cell contact during antigen-presentation. It is believed the cytoplasmic domains of CLASP proteins organize it into a patch at the leading edge of T cells. The carboxy-terminus encoded sequences mediate interaction with PDZ domain proteins and with cytoskeletal proteins (e.g., spectrin or ankyrin) to connect CLASPs to the microtubule network and hold the receptors at a polarized configuration just above the microtubule-organizing center (“MTOC”). Thus, when T cells engages a B cell acting as an APC, the CLASP molecules engage one another to dock the two cells and organize the immune synapse.

Modulating the expression of the CLASP proteins, and interference with, or enhancement of, CLASP protein interactions with other proteins has a number of beneficial physiological effects, e.g., altered signaling in response to antigen, altered T and B cell response to antigen, and modulation of T cell activation. In one aspect, a CLASP extracellular domain is targeted (e.g., using anti-CLASP antibodies, soluble CLASP fragments, and the like) to regulate T cell activation (and thus regulate immune responses). Mouse and human disorders that can be treated by disrupting CLASP function, include without limitation, multiple sclerosis, juvenile diabetes, rheumatoid arthritis, pemphigus, pemphigoid, epidermolysis bullosa acquista, lupus, endometriosis, toxemia or pregnancy induced hypertension, pruritic urticarial papules and plaques of pregnancy (PUPPP), herpes gestationis, impetigo herpetiformis, pruritus gravidarum, placenta-related disorders, and Rh incompatibility.

In another aspect, the present invention provides methods and reagents for detection of CLASP expression and CLASP-expressing cells. Abnormal expression patterns or expression levels are diagnostic for immune and other disorders, and animal model systems. For example, diseases characterized by overproduction or depletion of lymphocytes in blood or other organs may be detected or monitored by monitoring the level of CLASP polypeptides or mRNAs in a biological sample (e.g., peripheral blood), e.g., the number or percentage of CLASP expressing cells. Diseases characterized by overproduction of T cells include, e.g., leukemia (both ALL and CLL), lymphoma (including non-Hodgkins lymphoma, Burkitt's lymphoma, mycosis fungoides, and sezary syndrome), EBV, CMV, toxoplasmosis, syphilis, typhoid, brucellosis, tuberculosis, influenza, hepatitis, serum sickness, and thyrotoxicosis. Diseases associated with the depletion of T cells include, e.g., HIV and myelodysplasia. Diseases associated with the overproduction of B cells include, e.g., leukemia (both ALL and CLL), non-Hodgkins lymphoma, Burkitt's lymphoma, myeloma, EBV, CMV, toxoplasmosis, syphilis, typhoid, brucellosis, tuberculosis, influenza, hepatitis, serum sickness, and thyrotoxicosis. Diseases associated with the depletion of B cells include, e.g., myelodysplasia.

5.2. CLASP cDNA and Polypeptide Structures

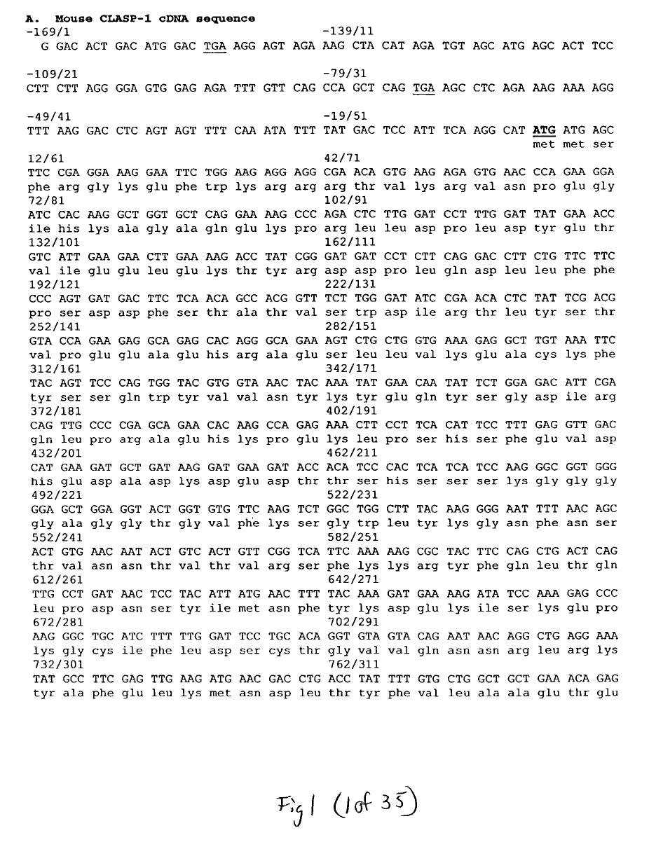

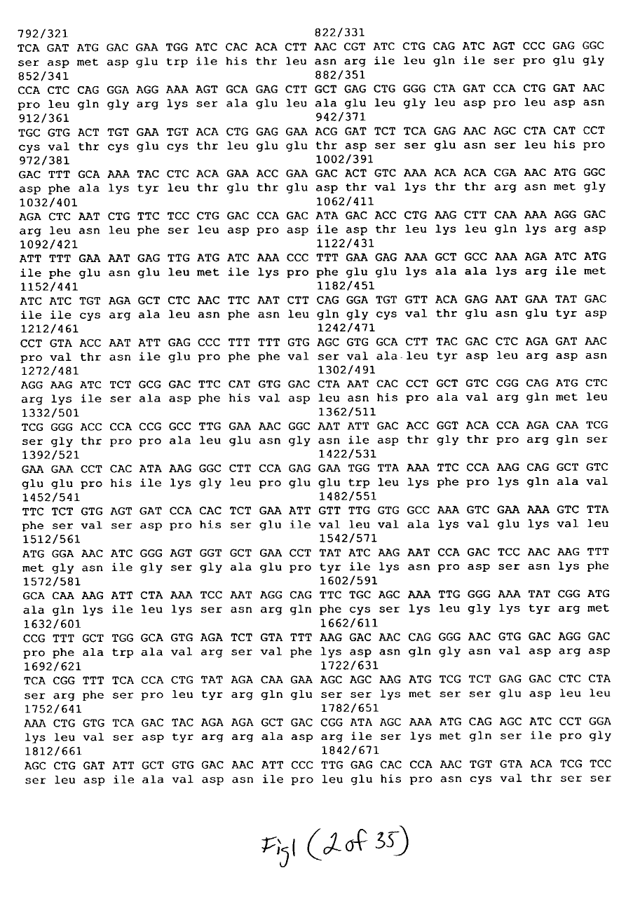

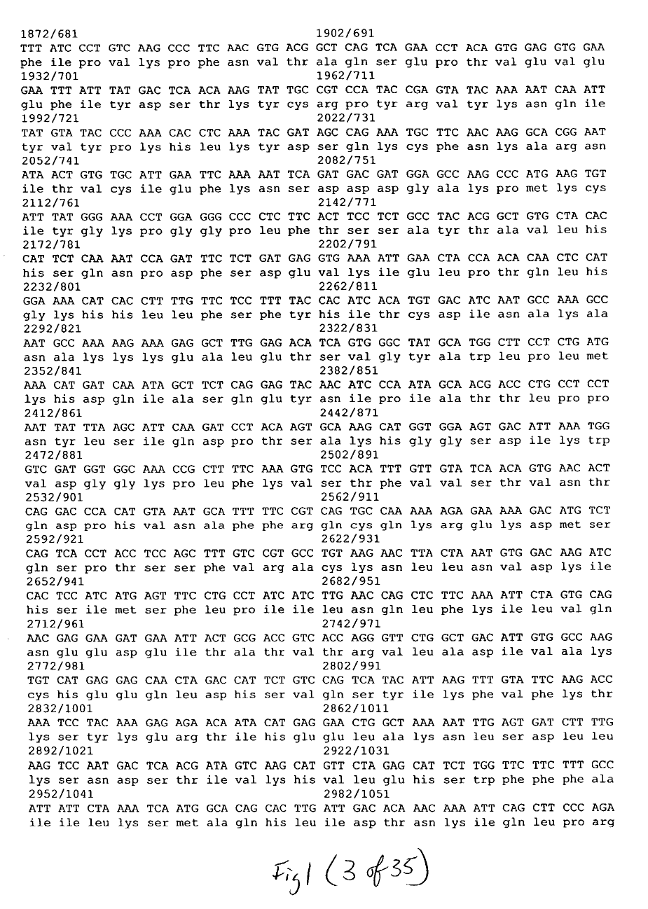

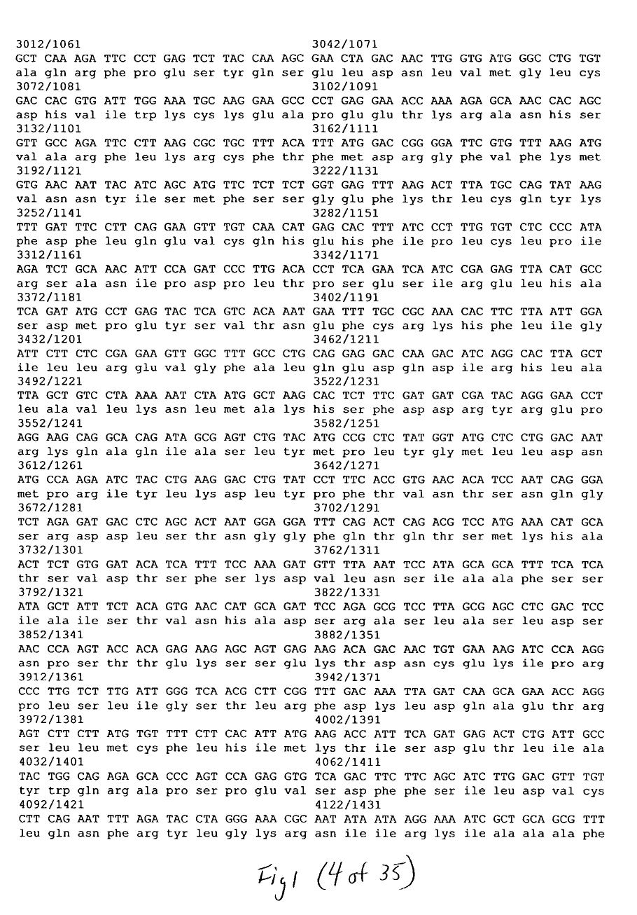

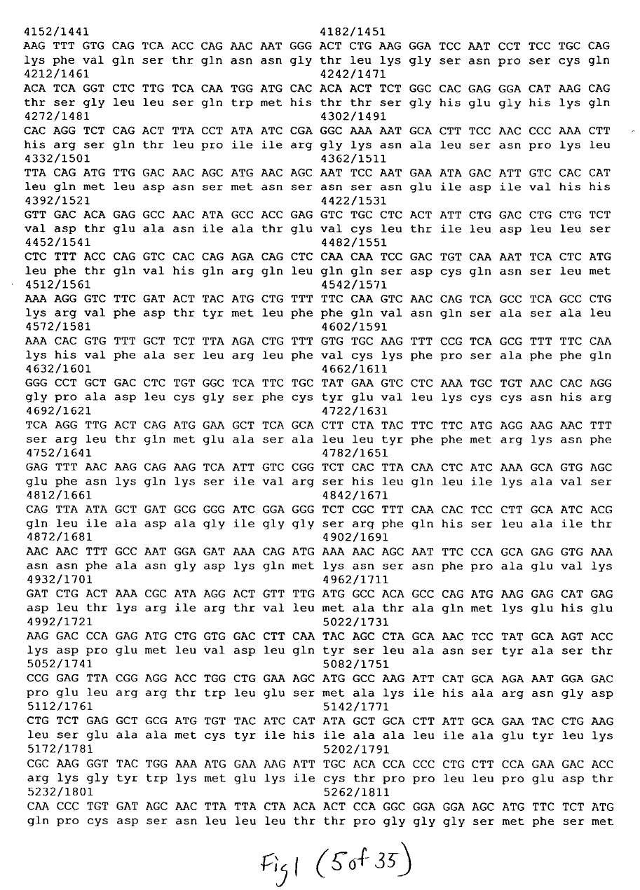

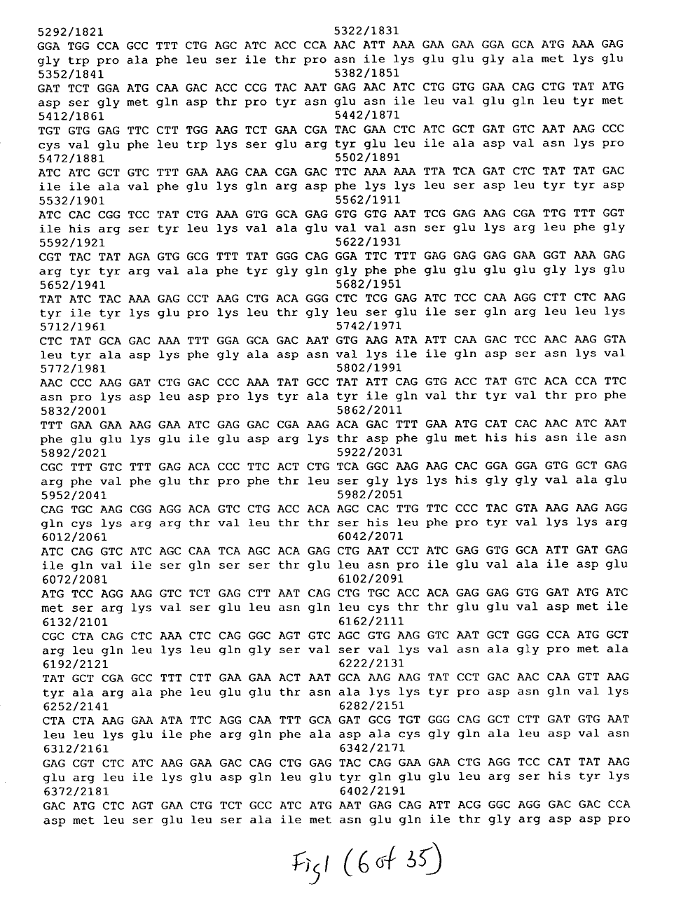

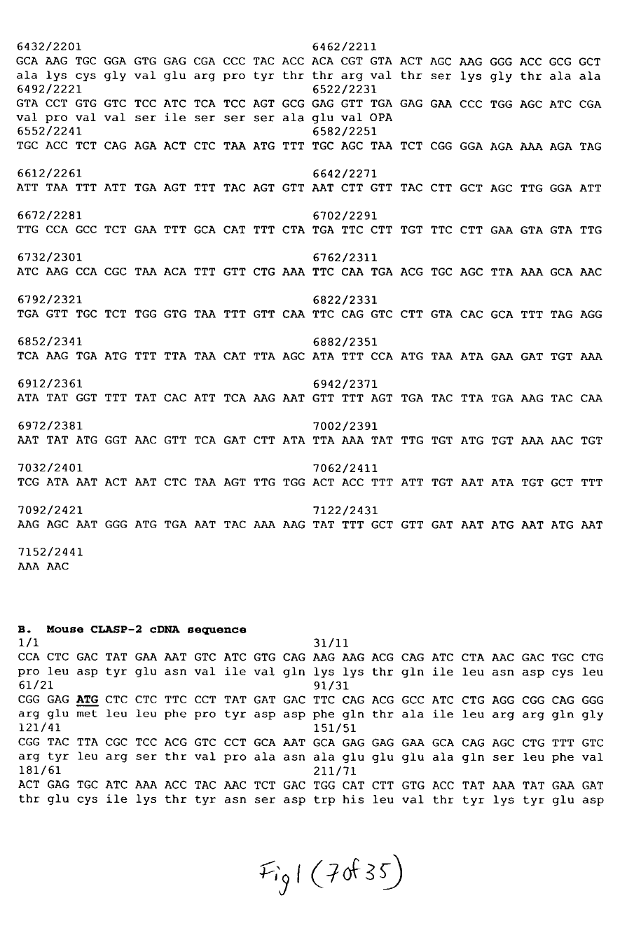

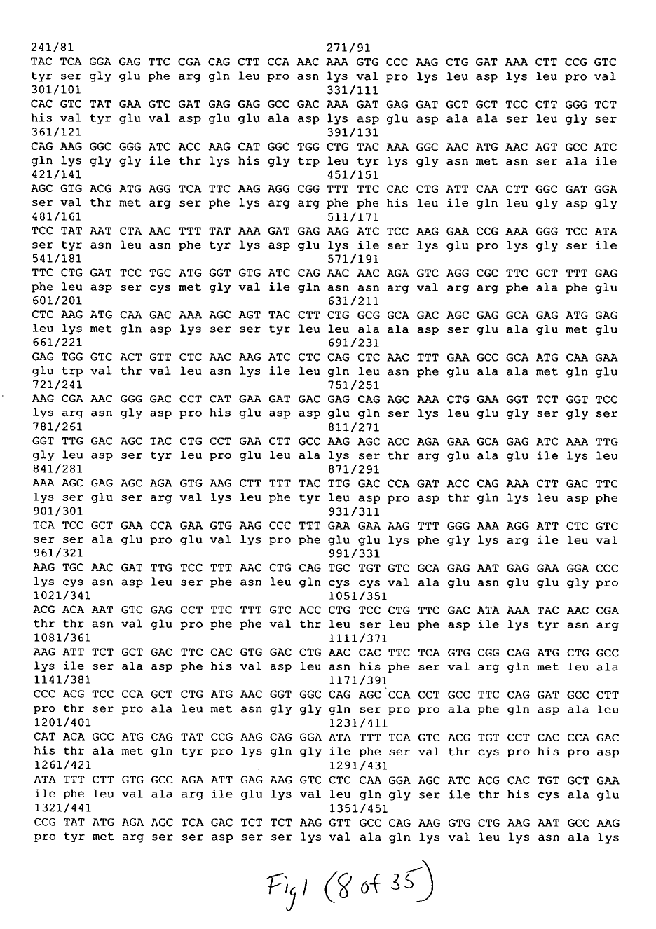

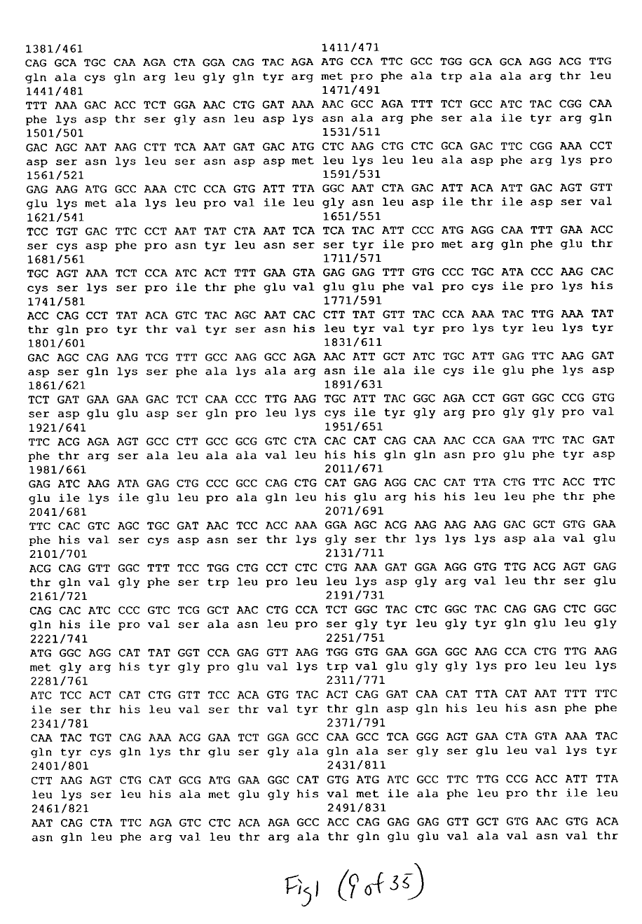

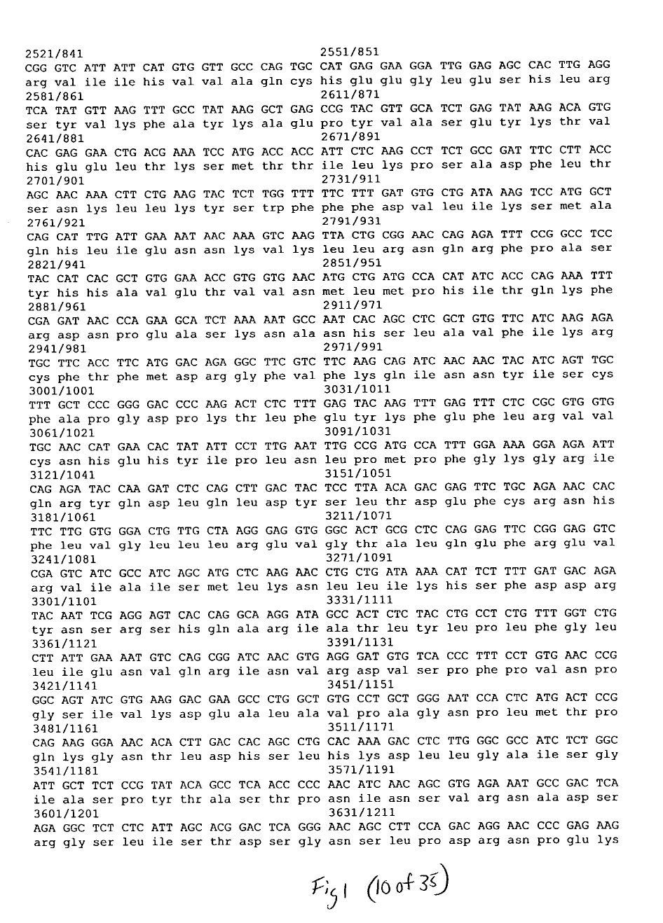

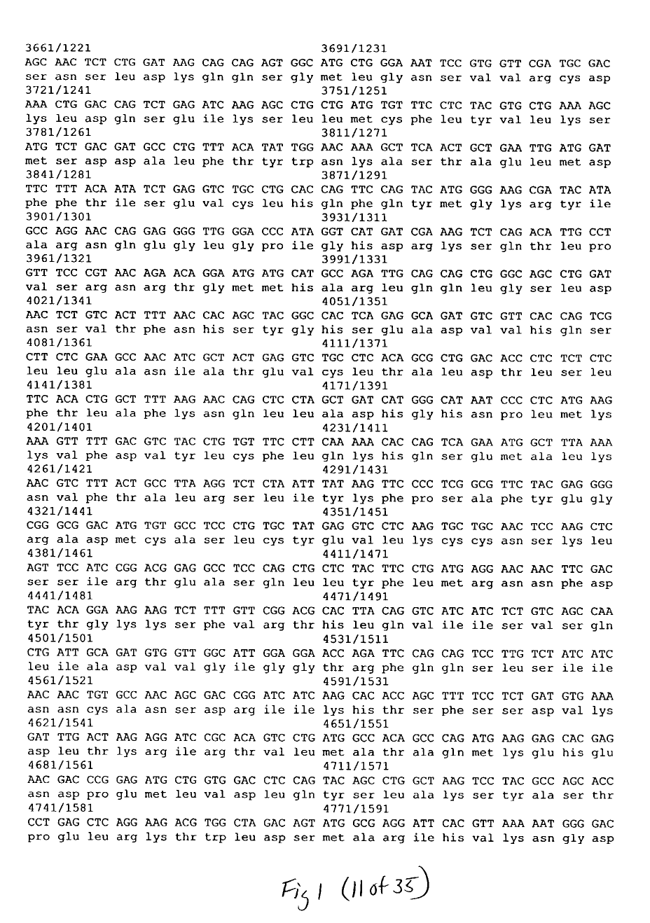

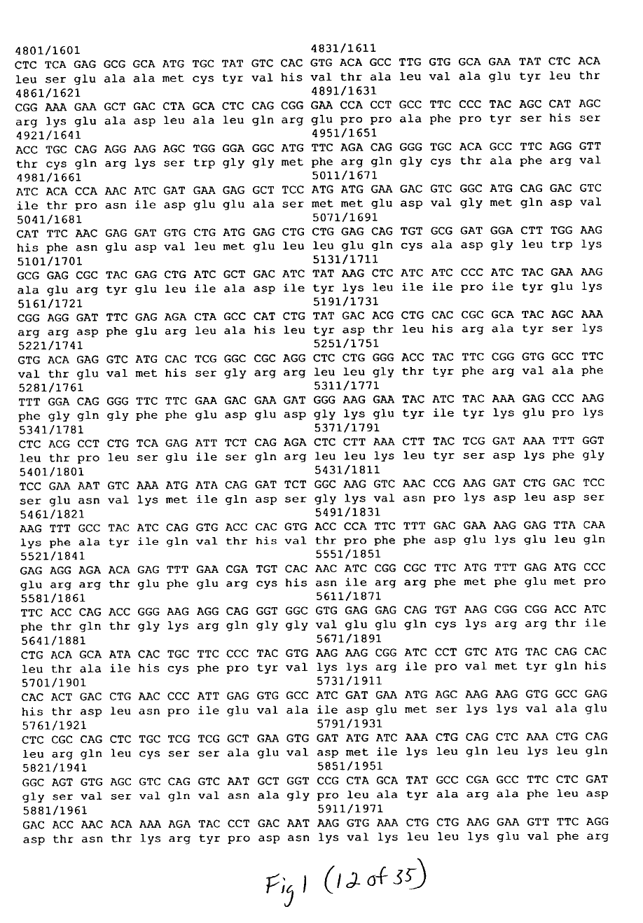

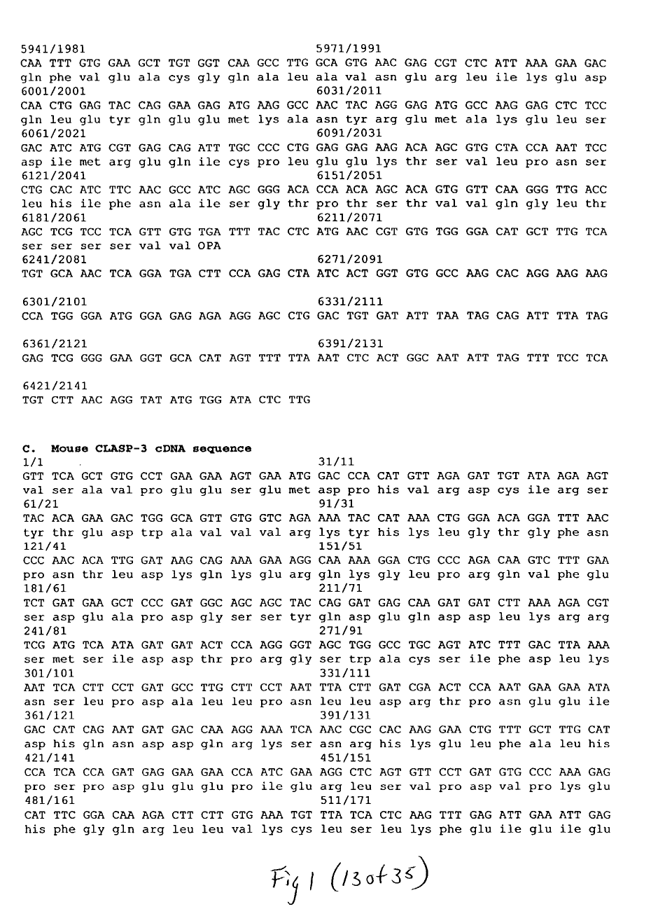

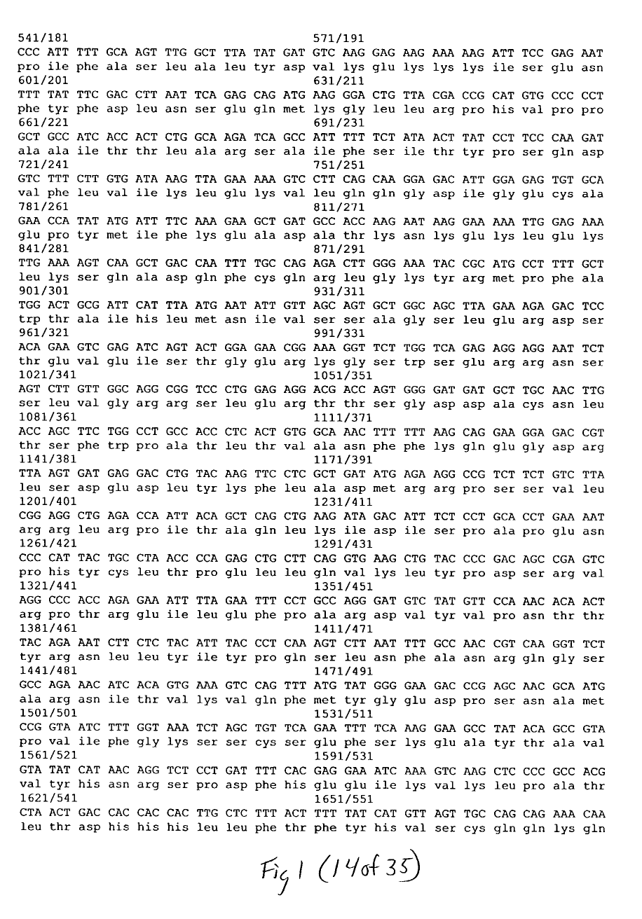

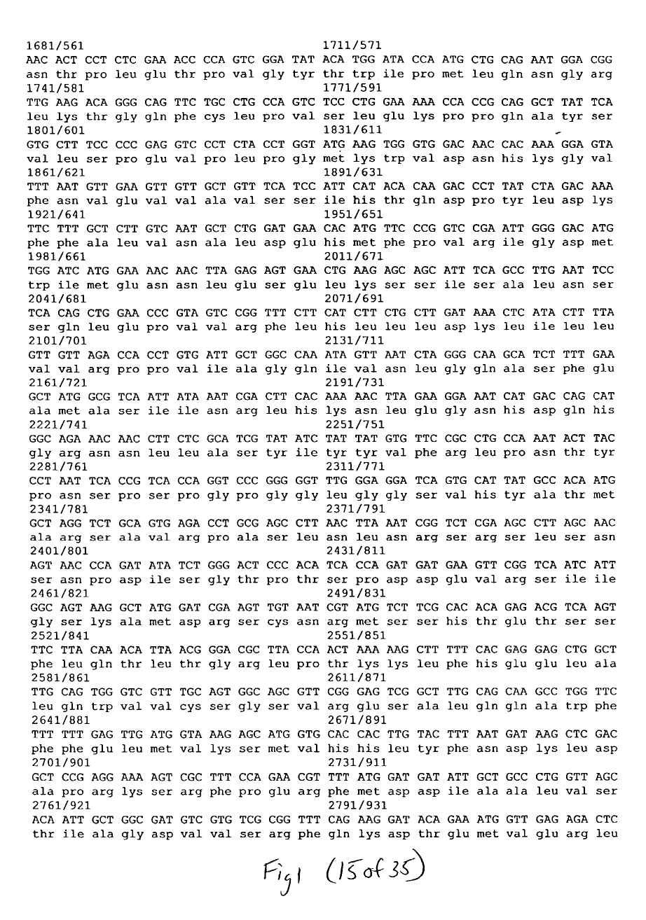

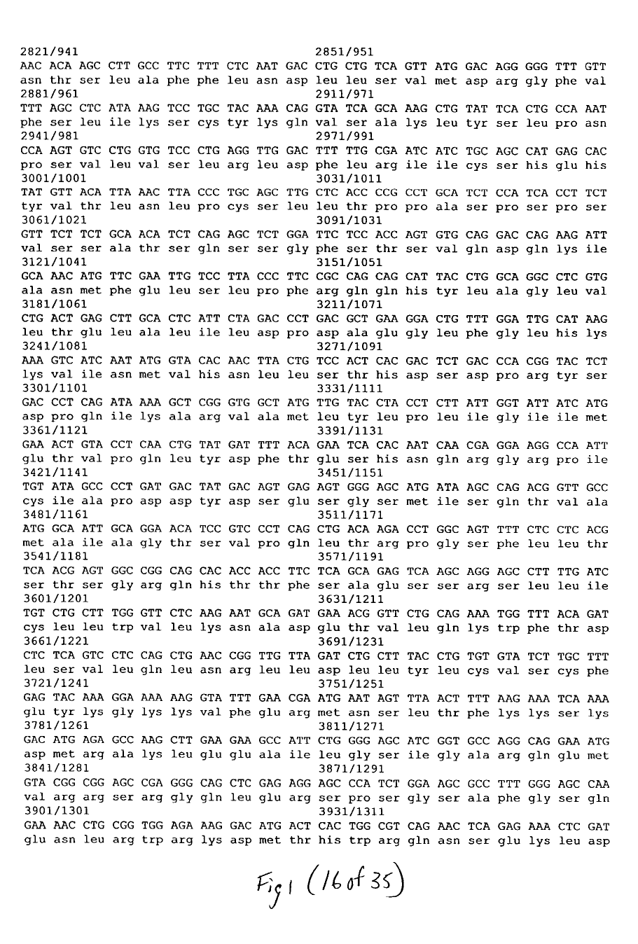

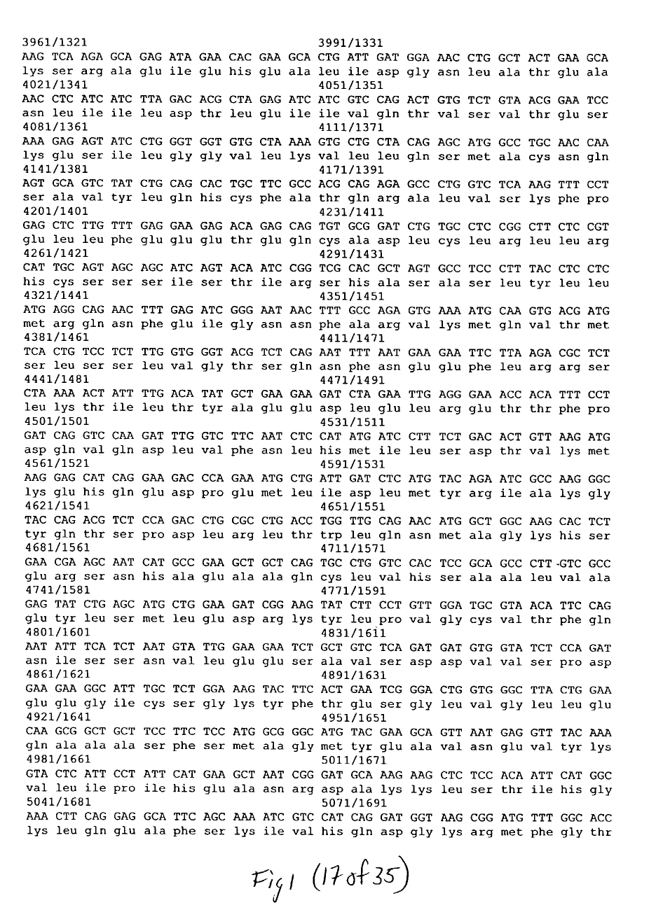

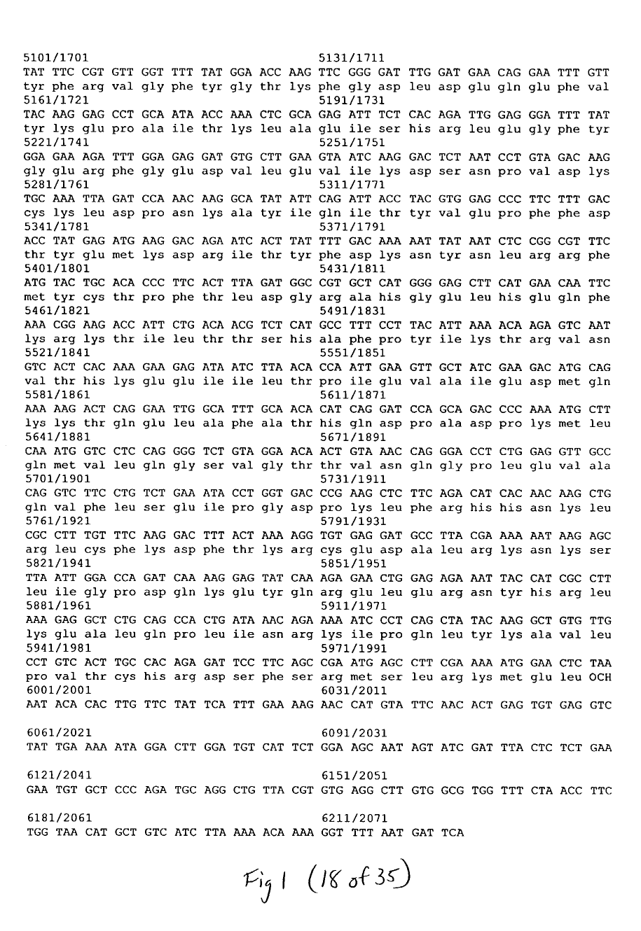

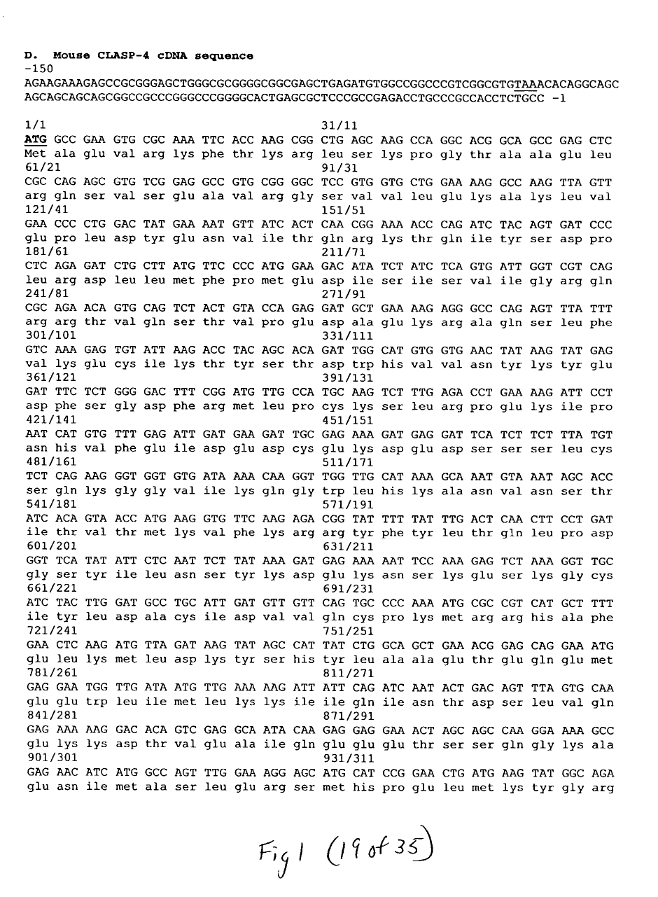

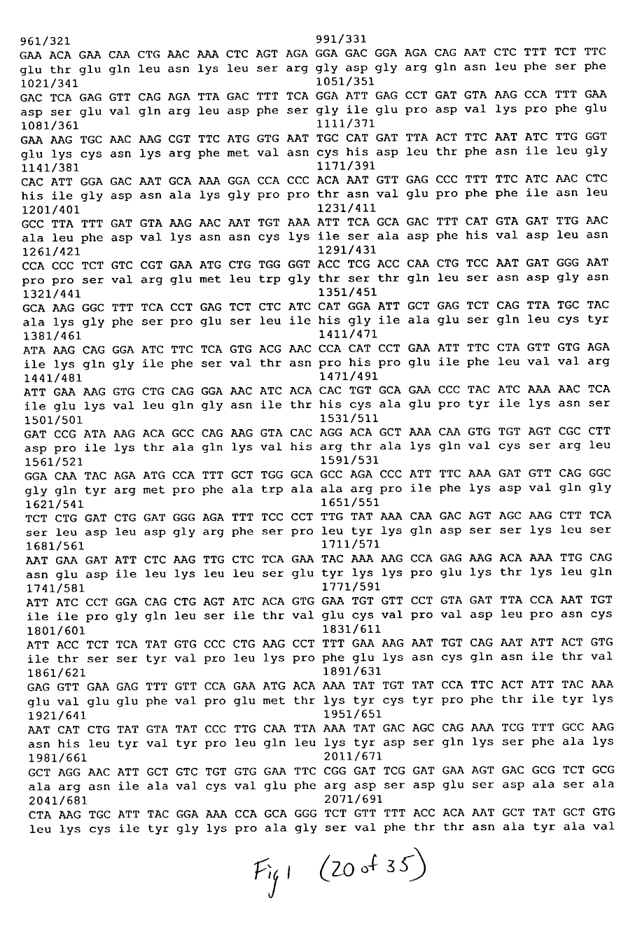

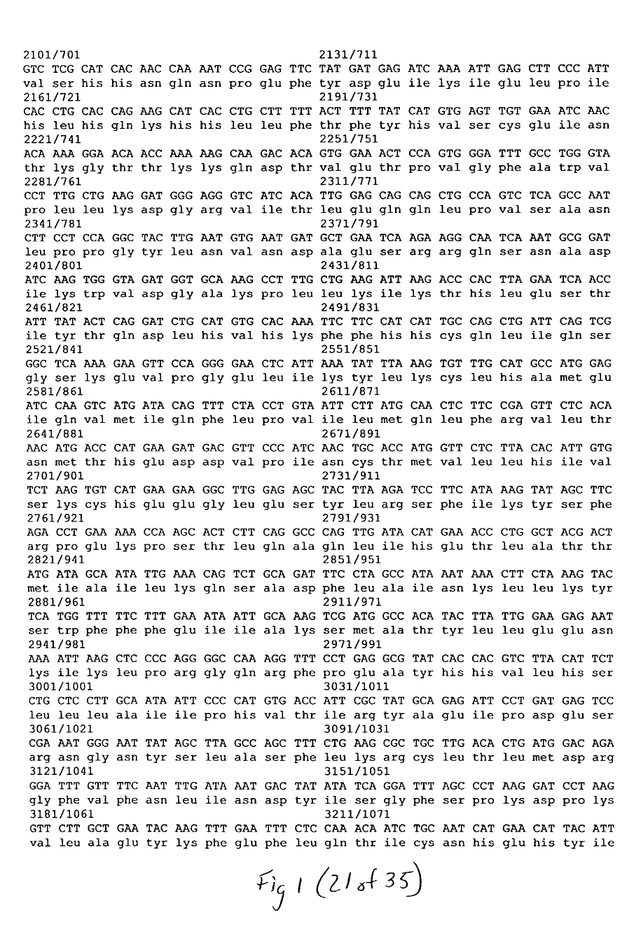

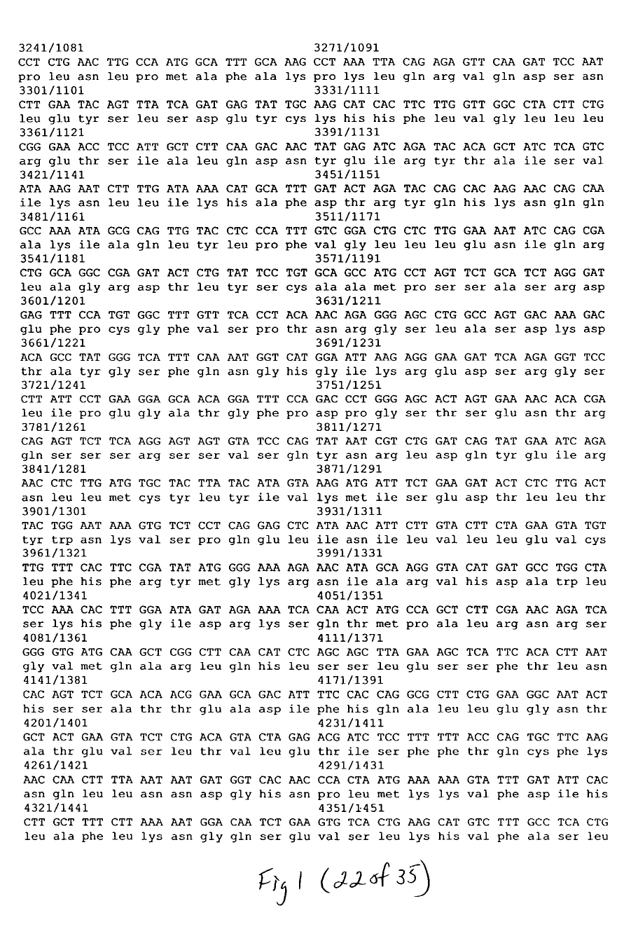

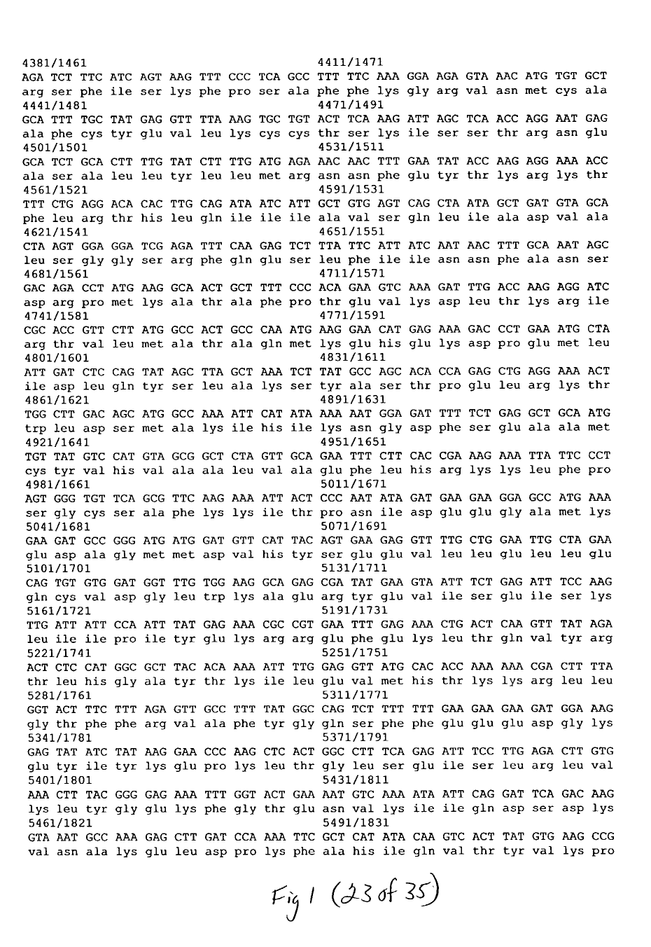

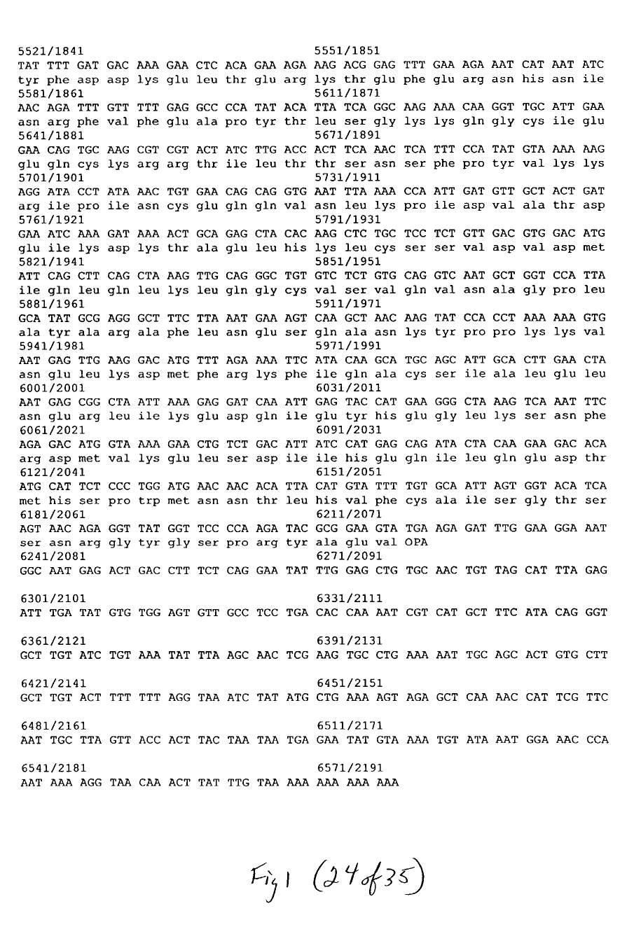

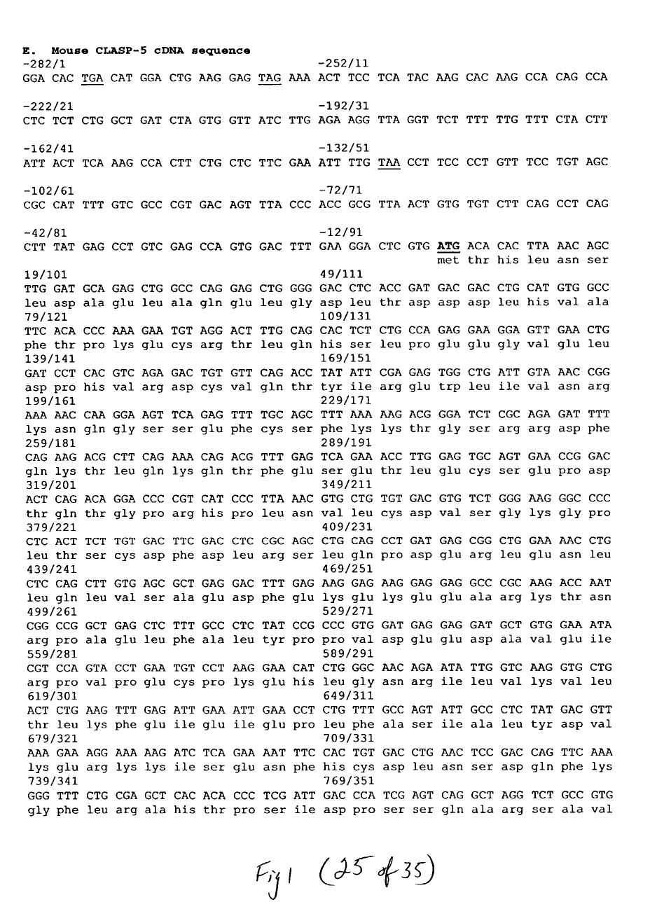

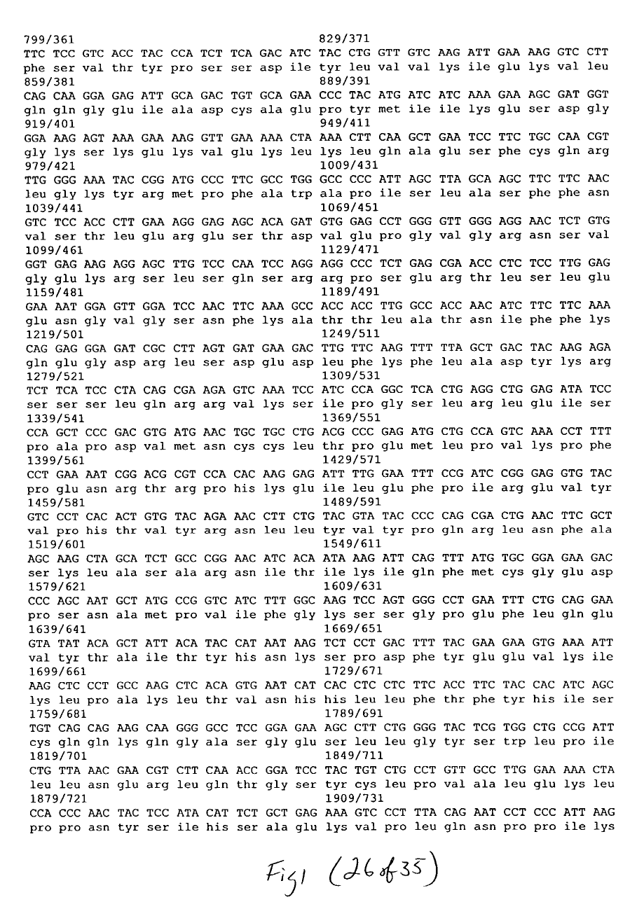

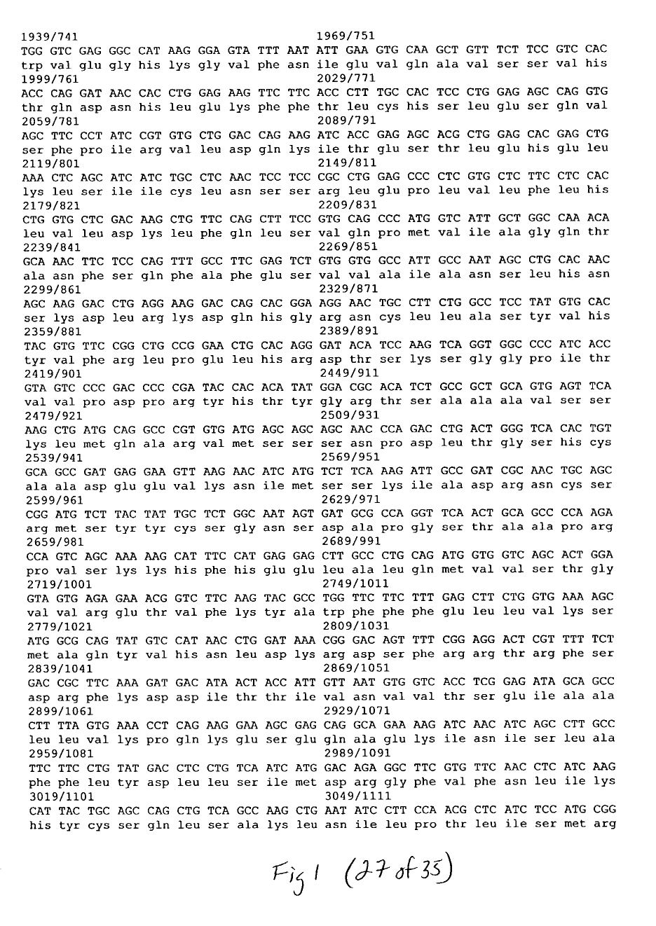

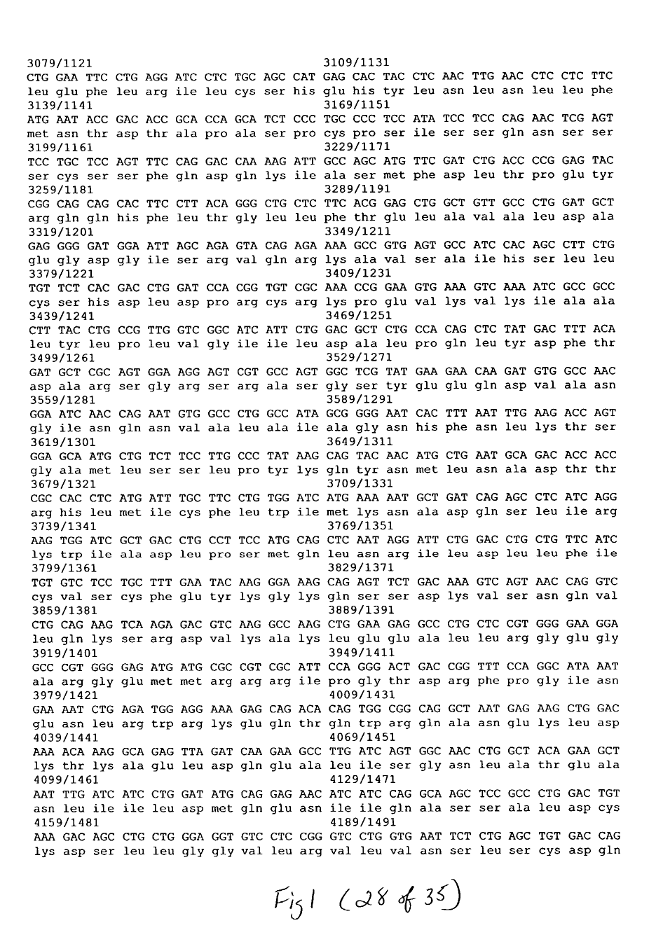

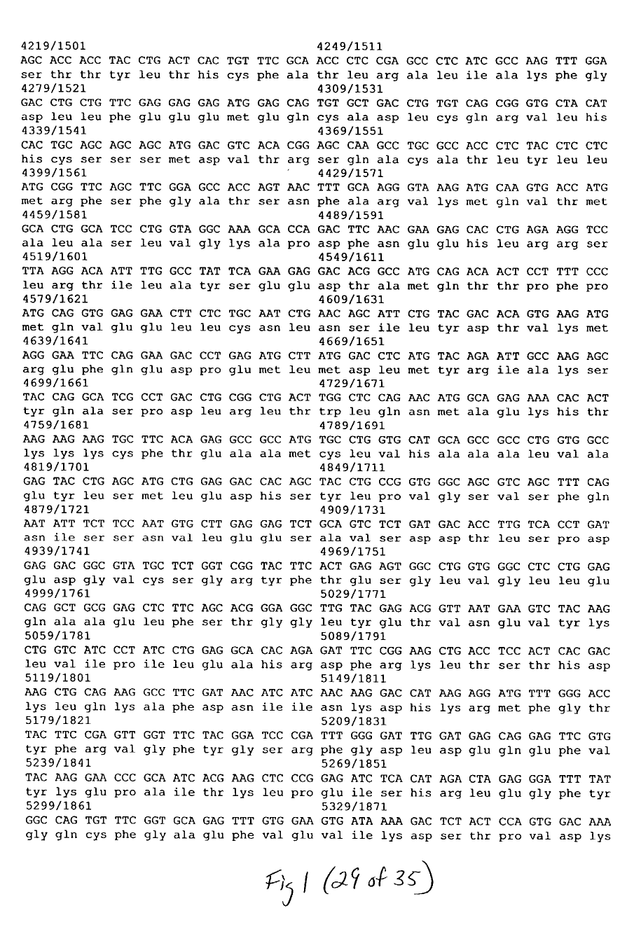

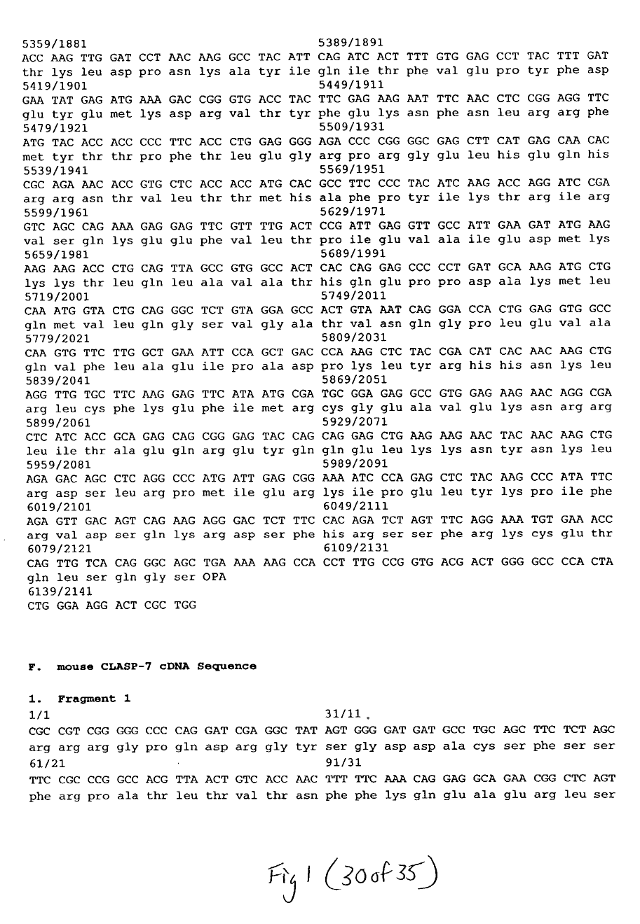

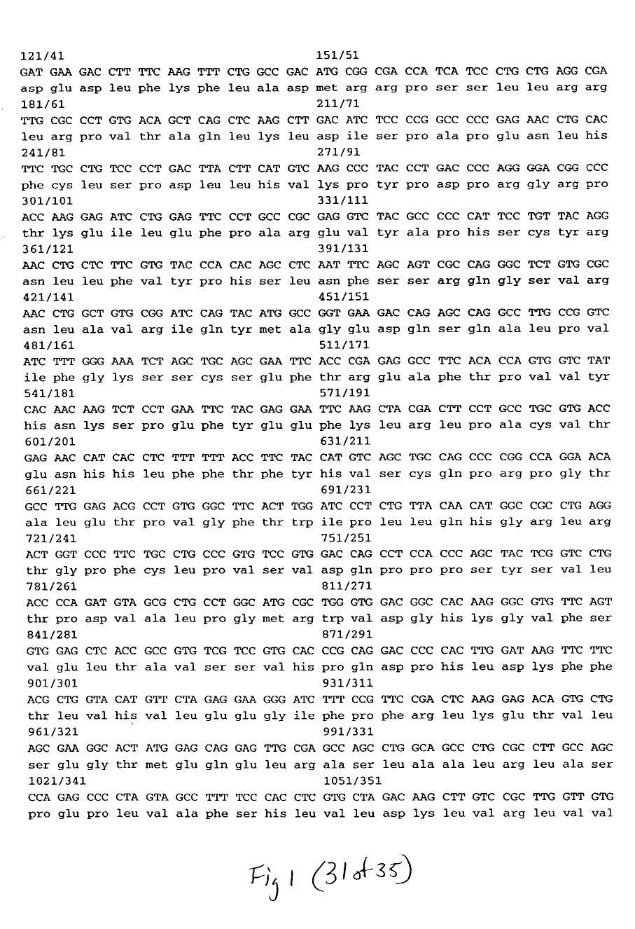

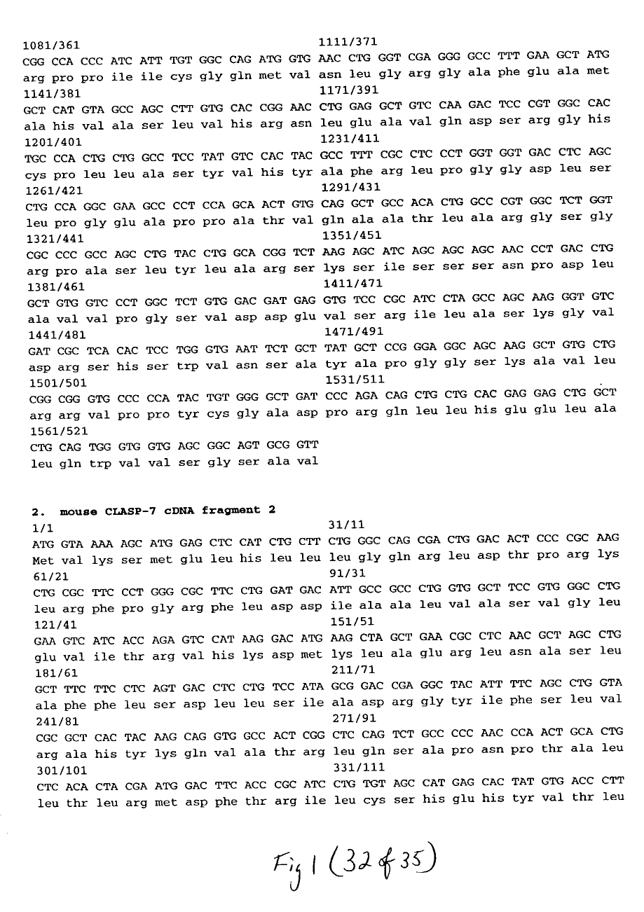

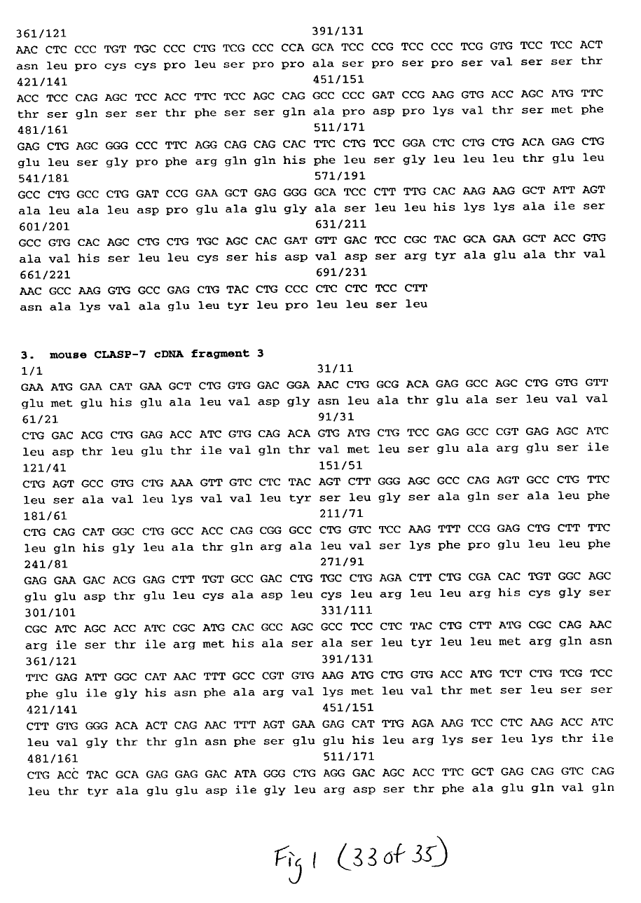

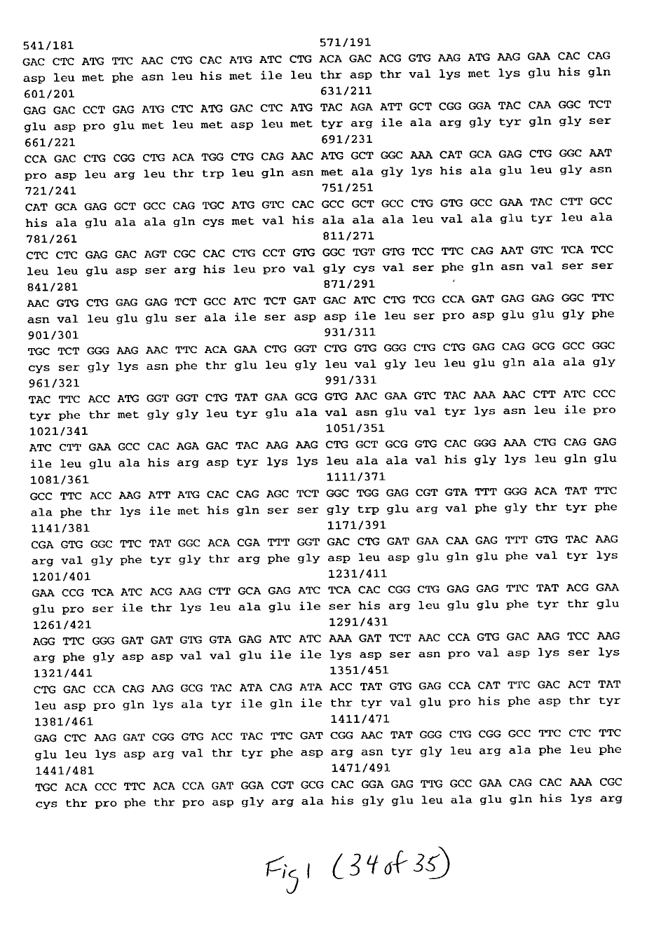

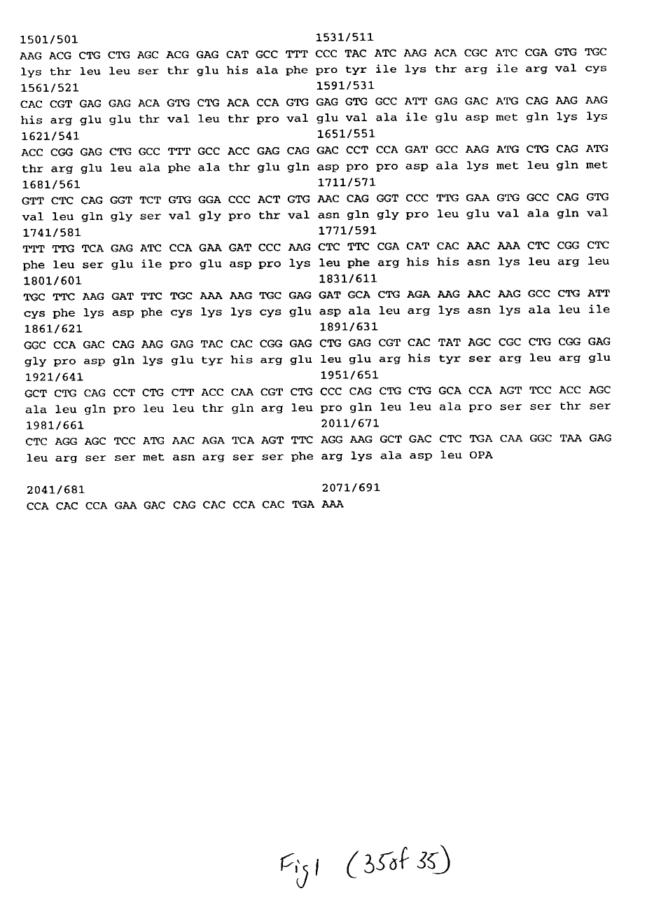

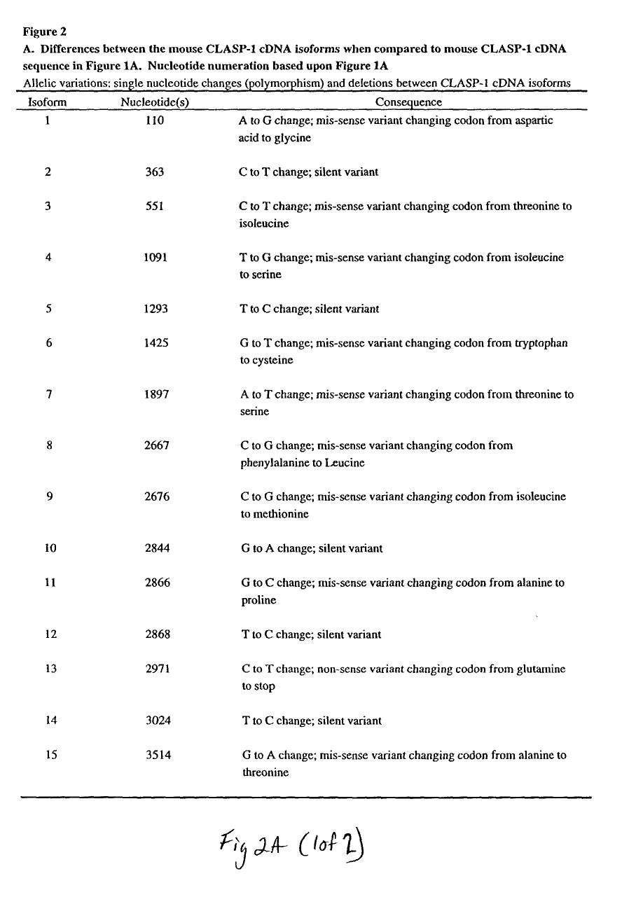

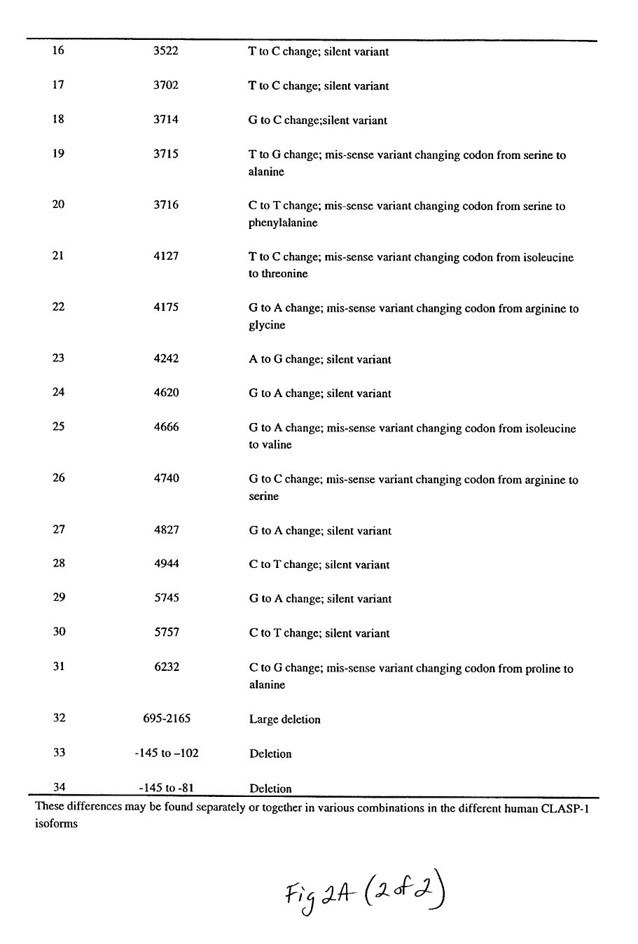

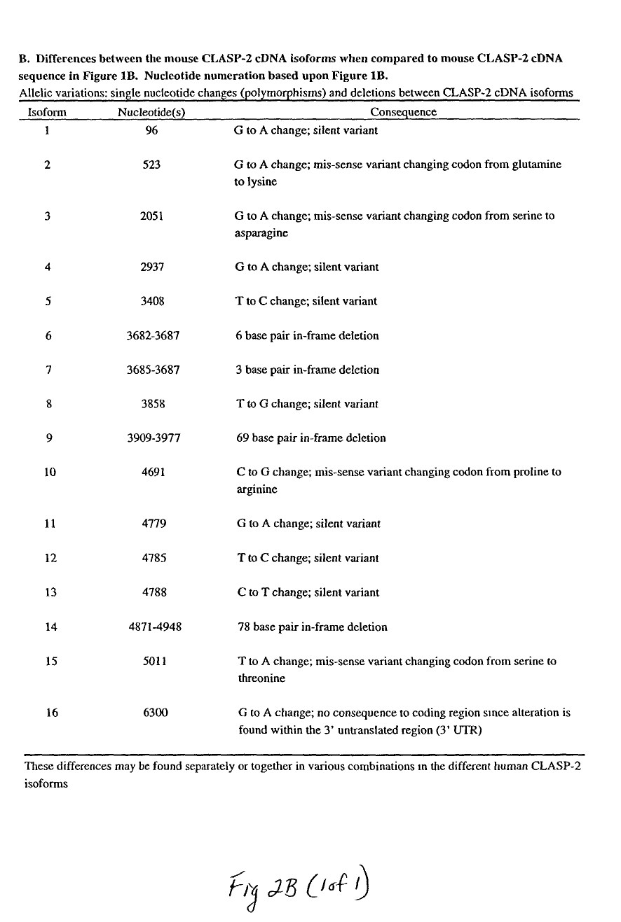

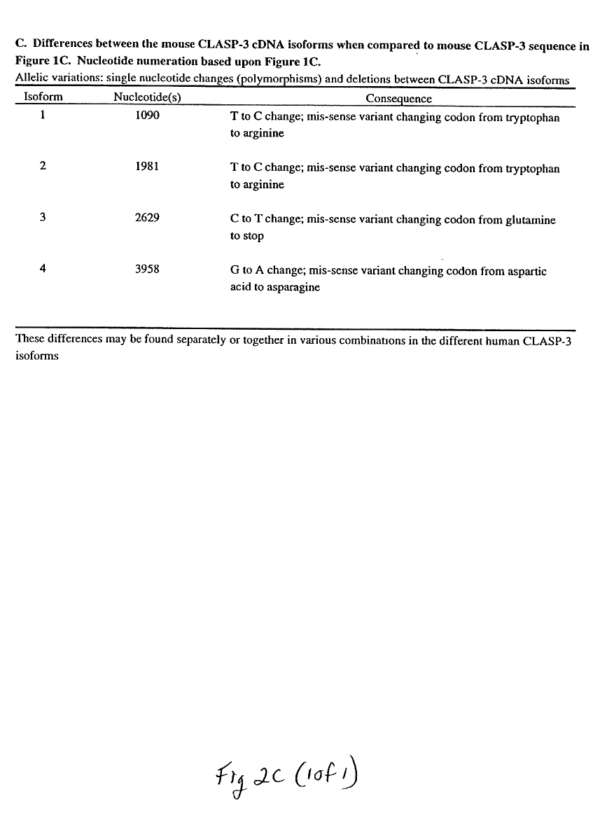

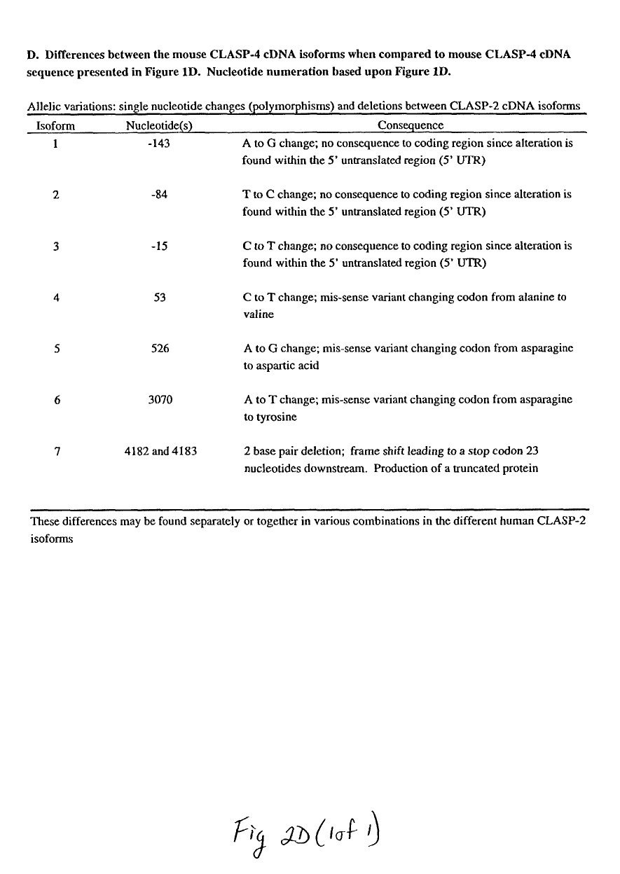

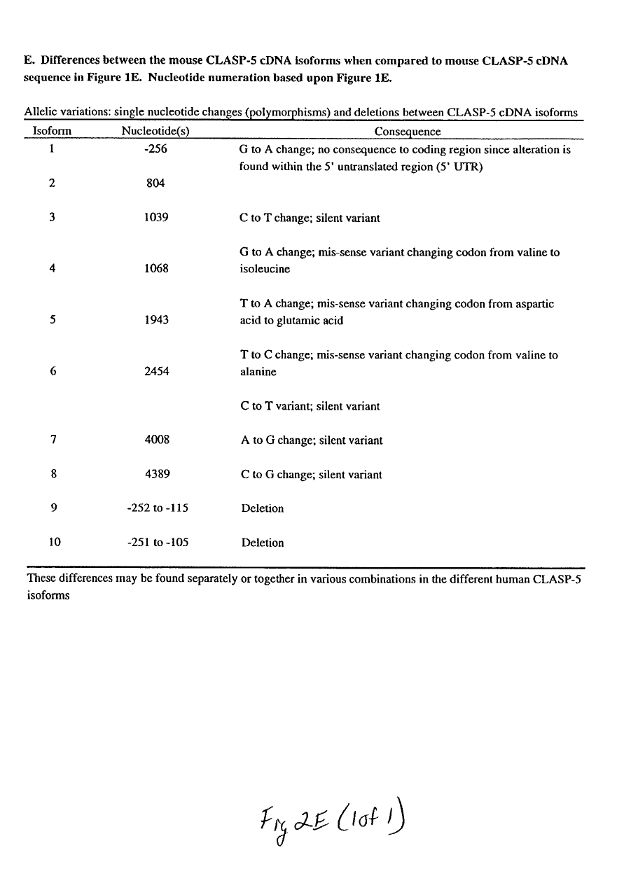

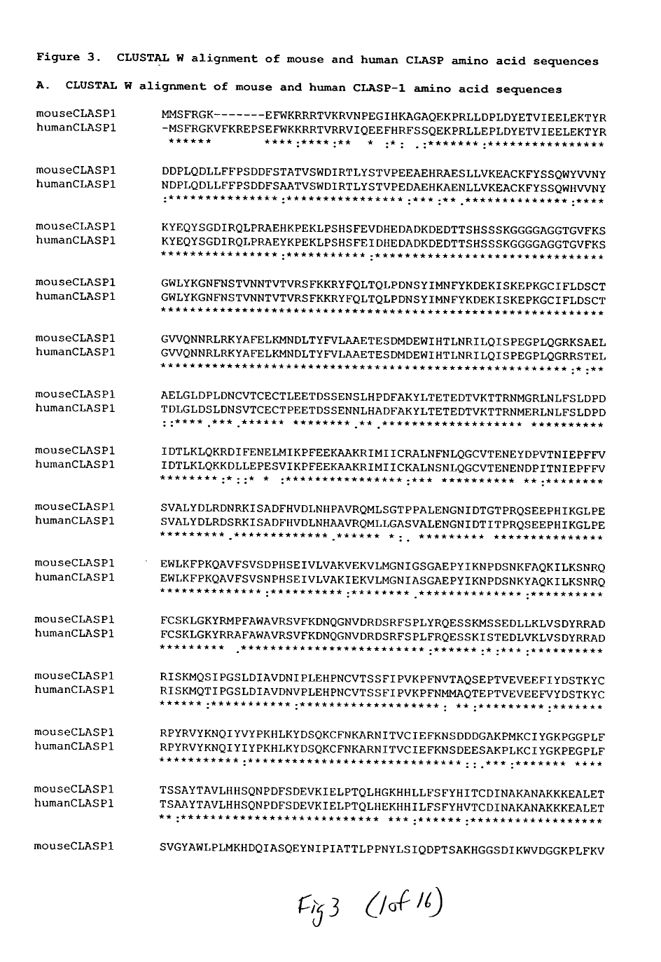

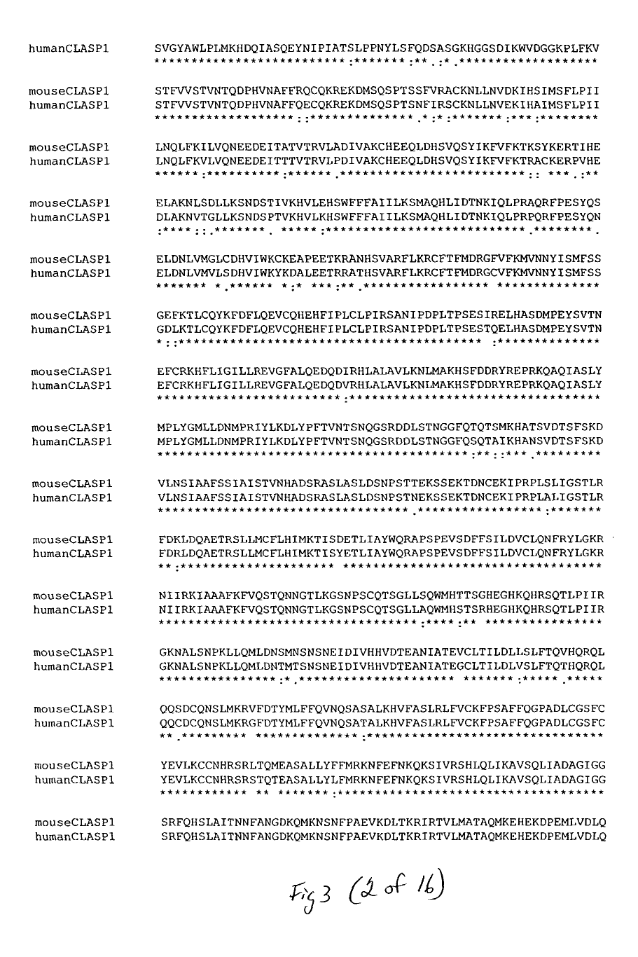

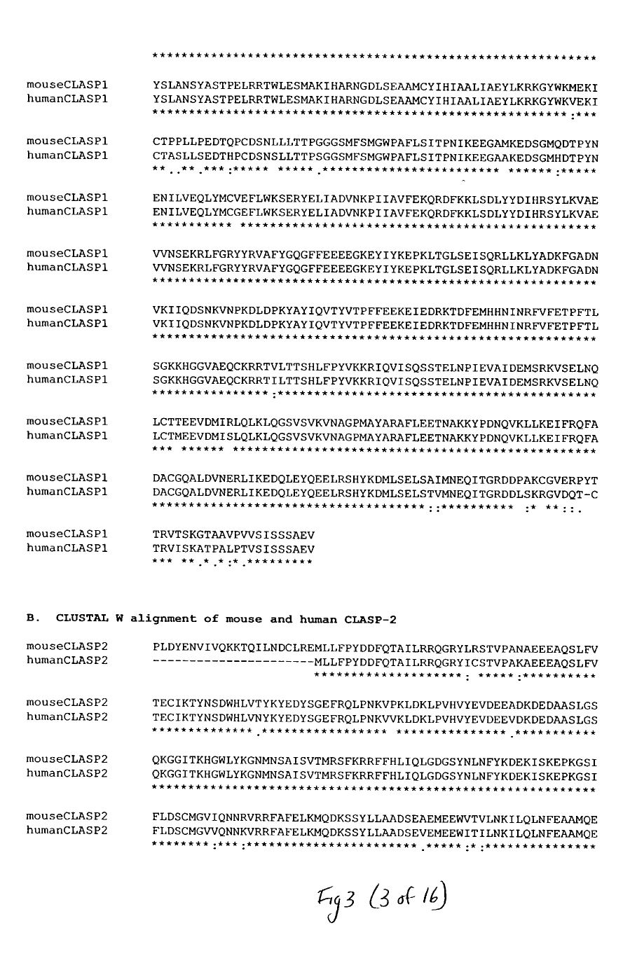

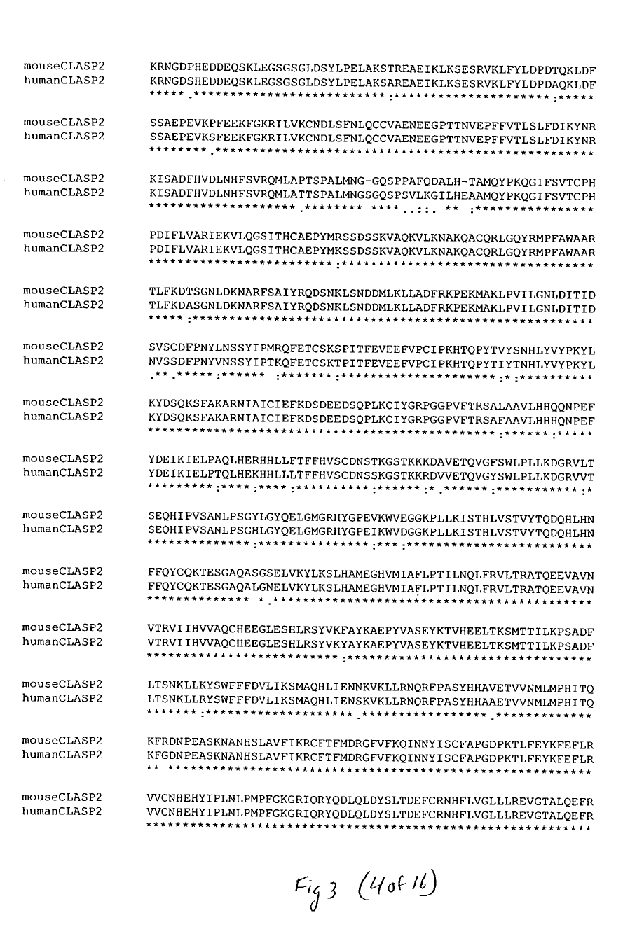

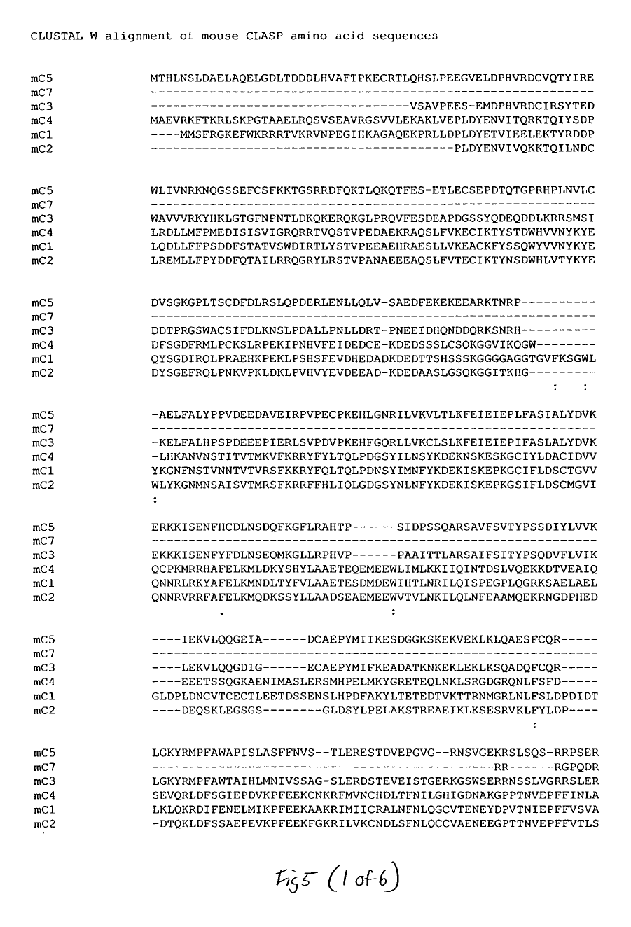

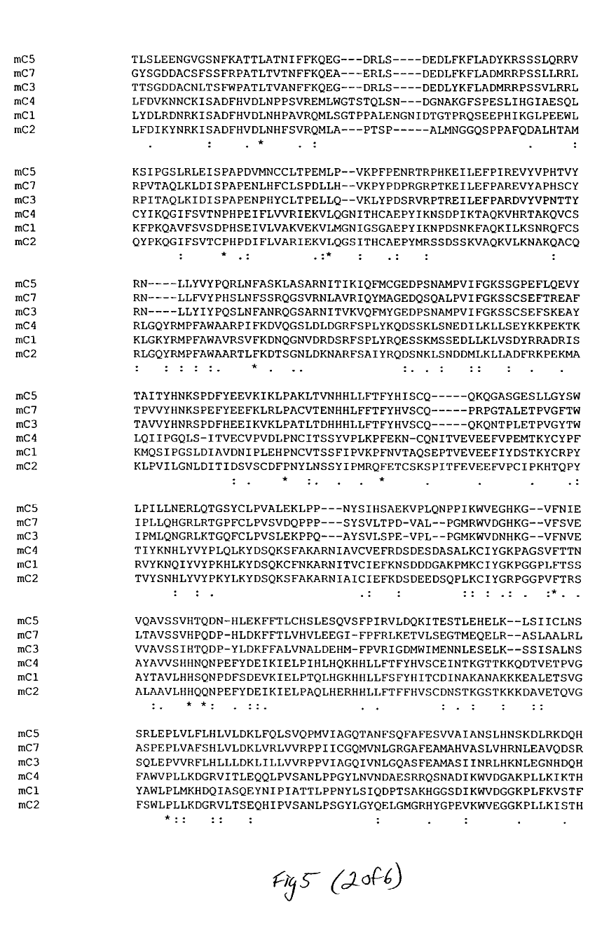

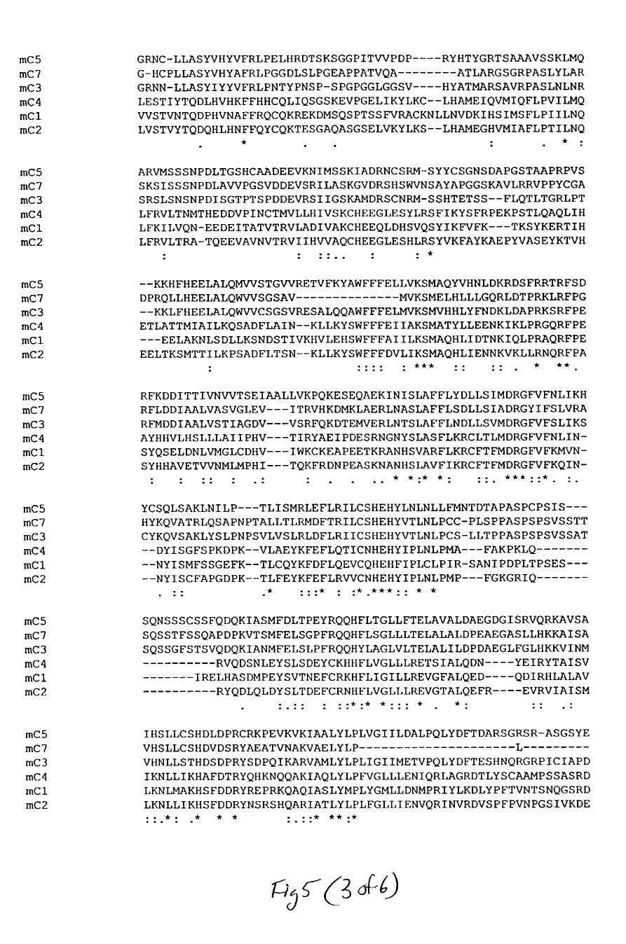

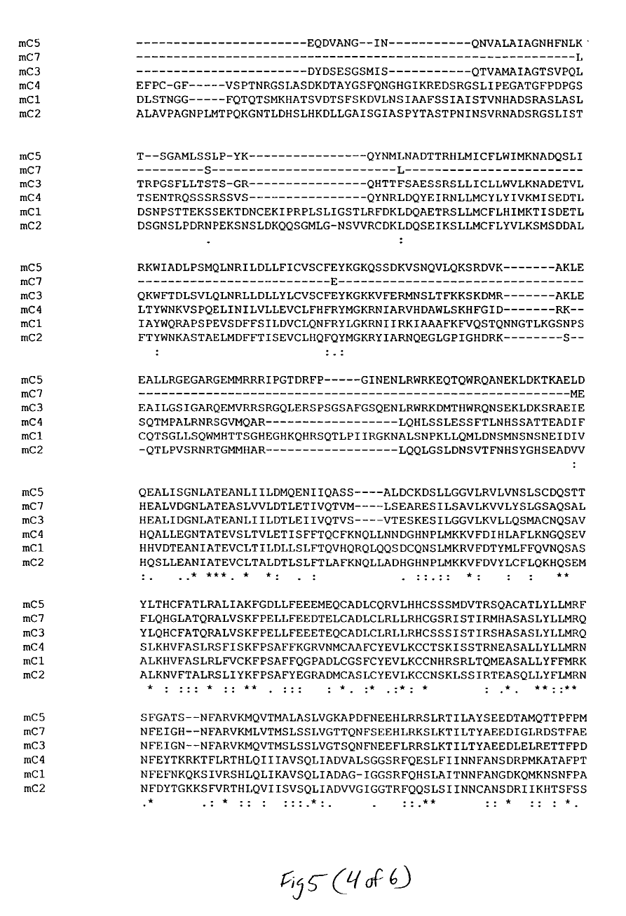

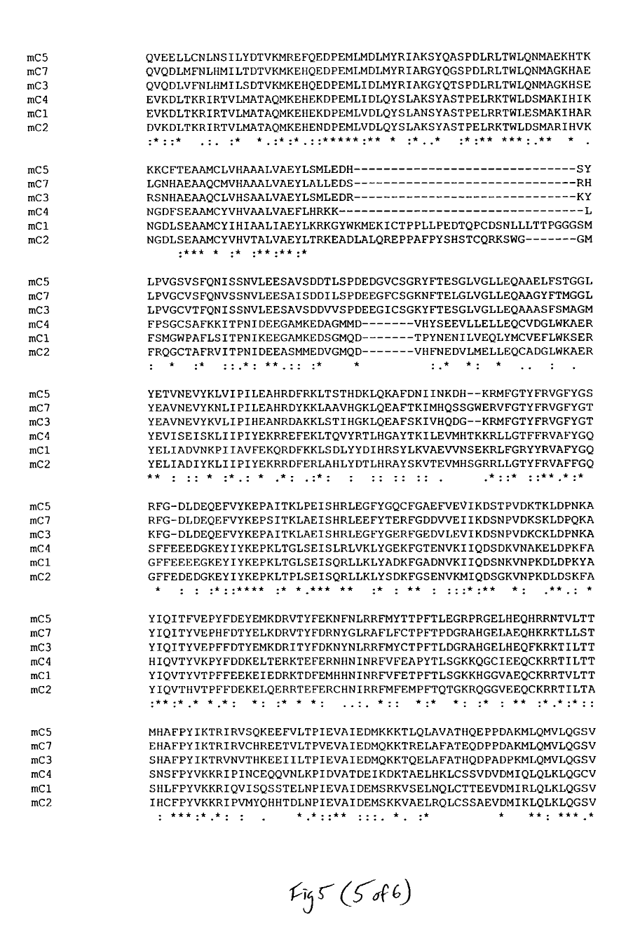

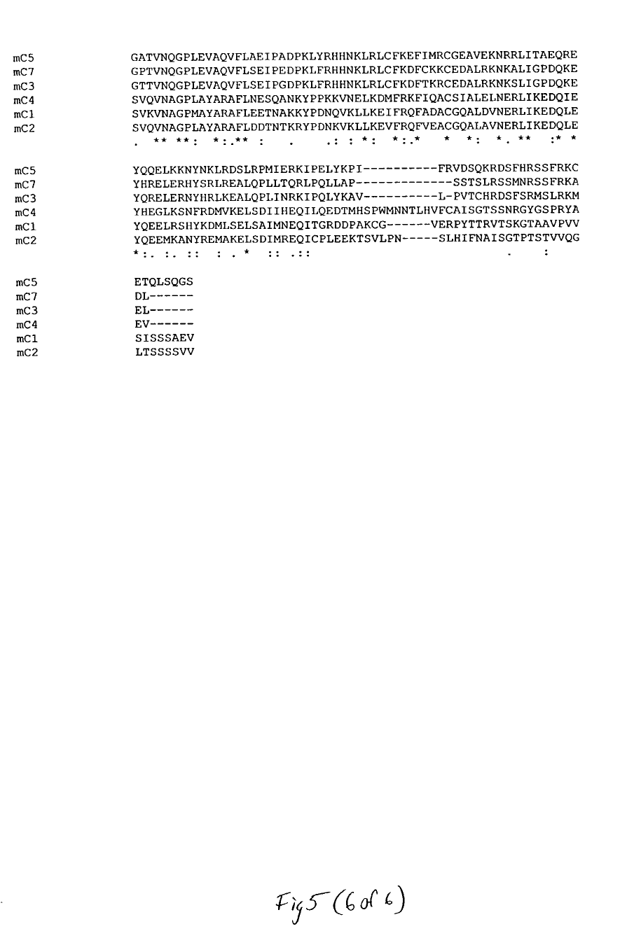

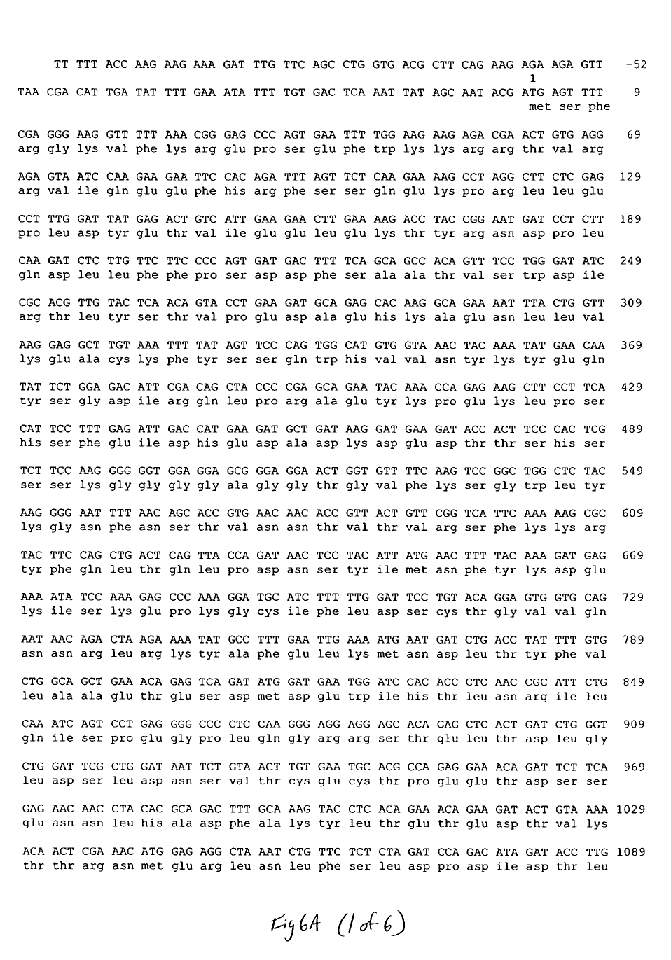

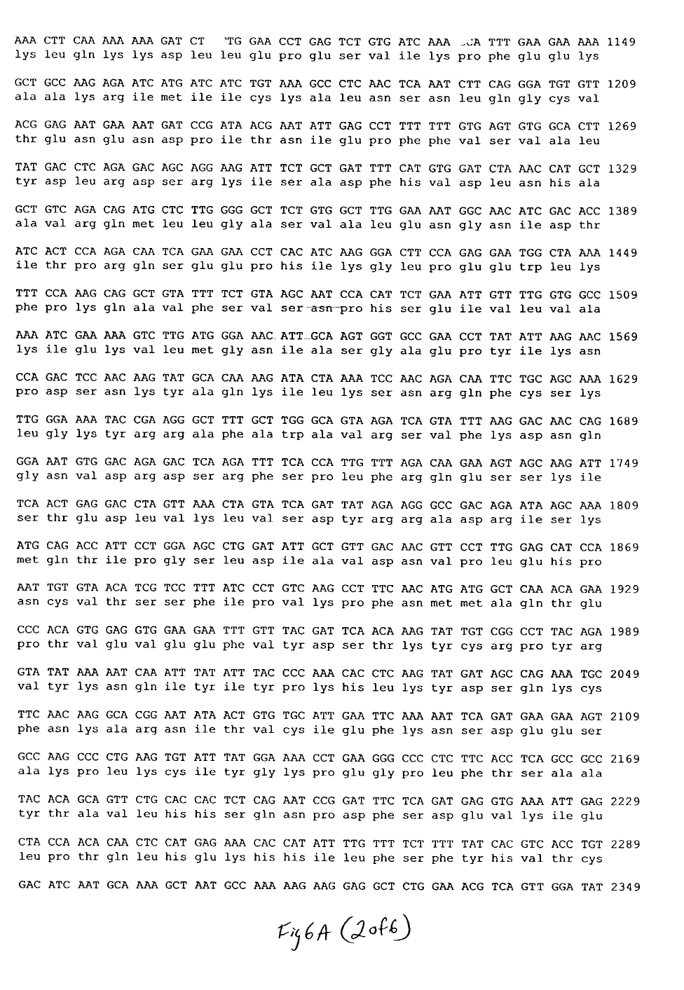

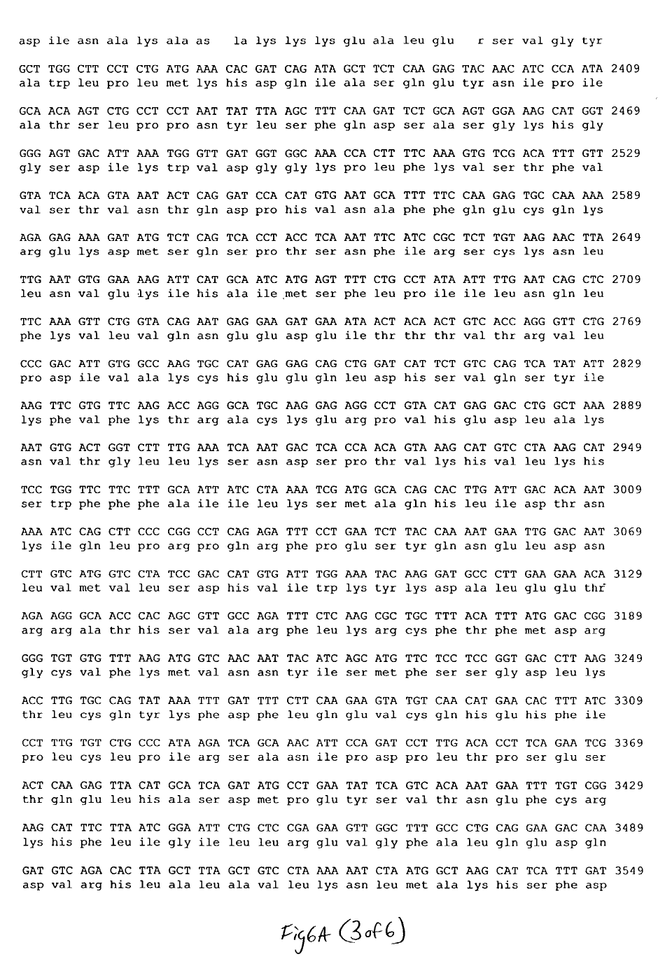

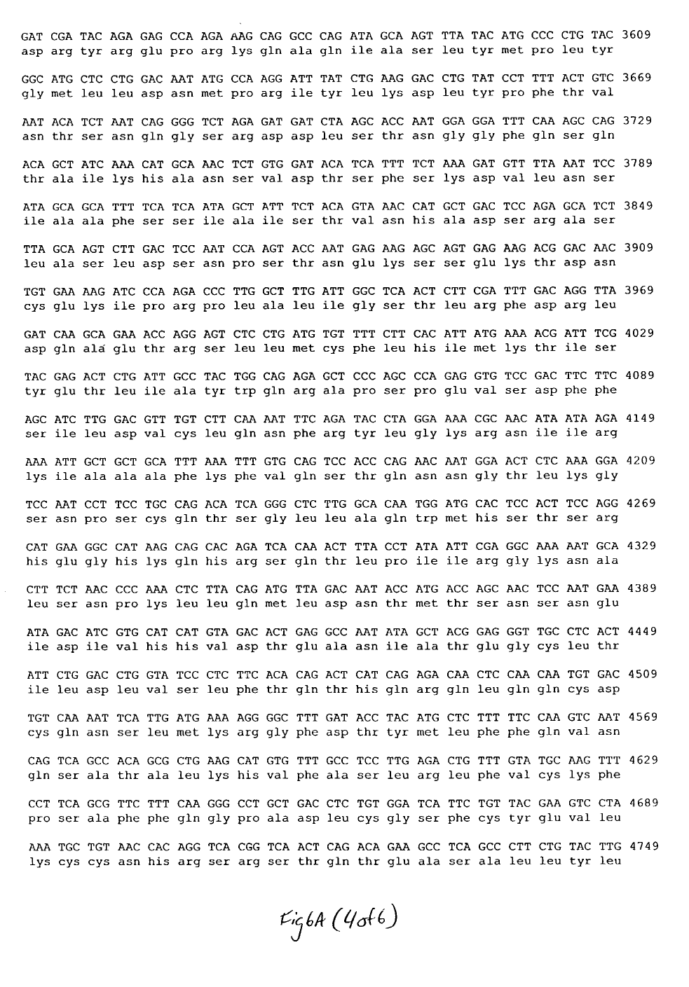

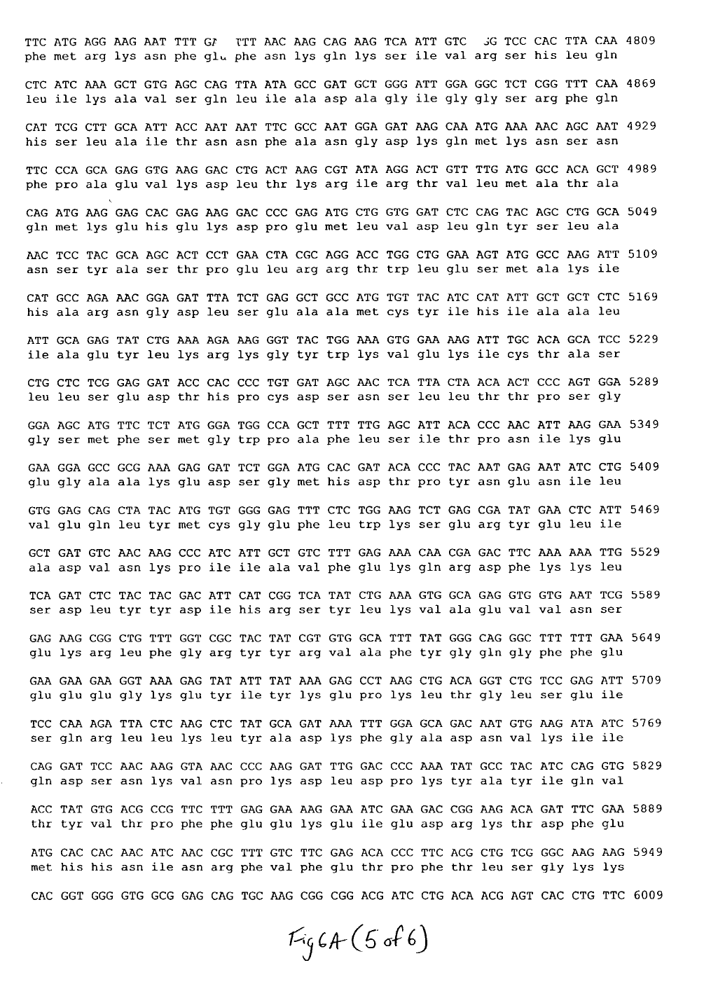

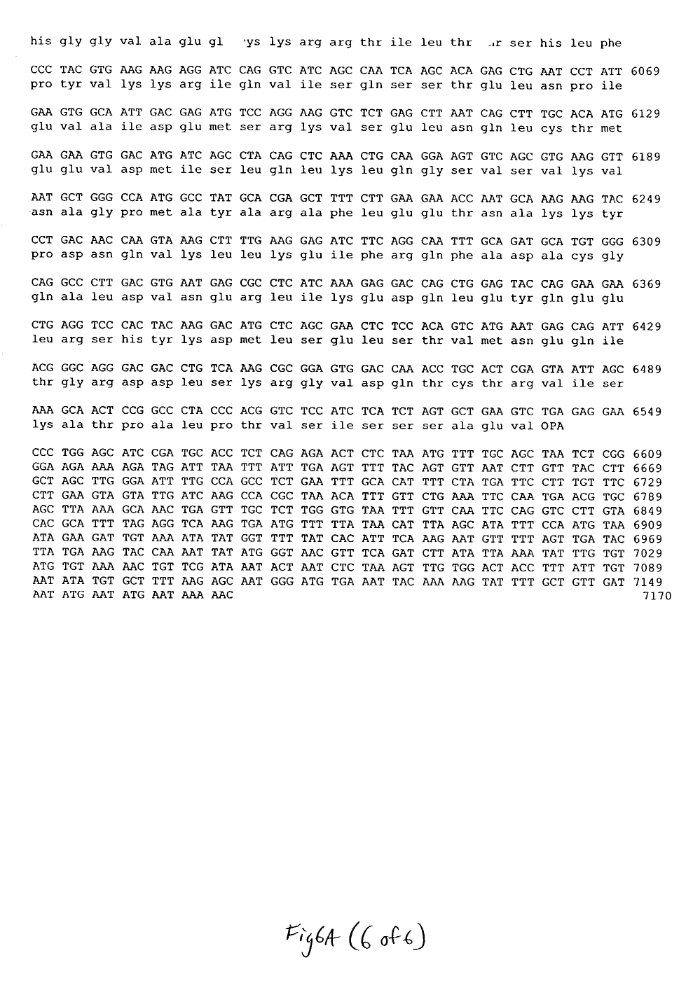

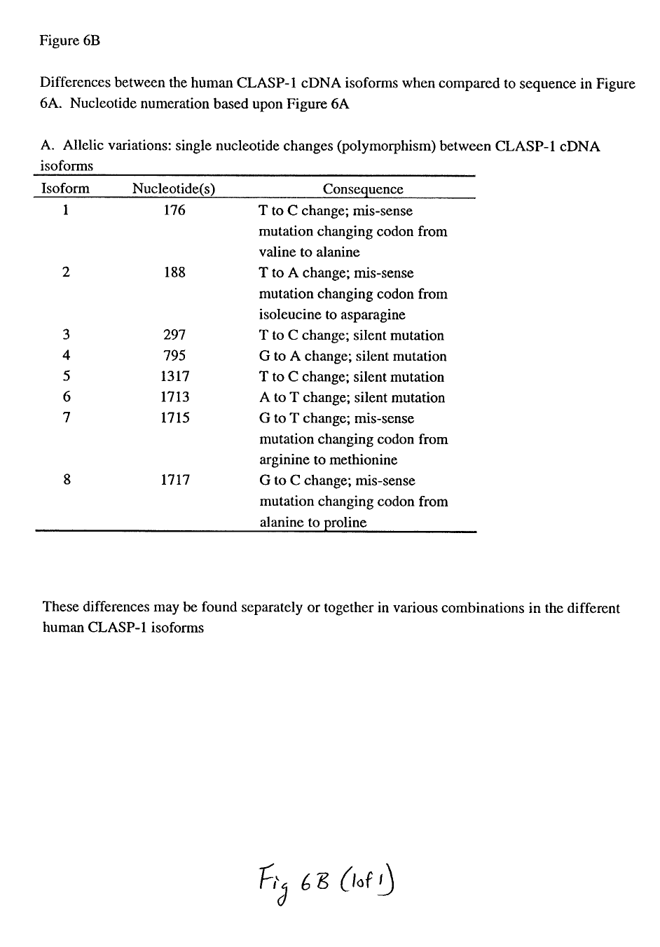

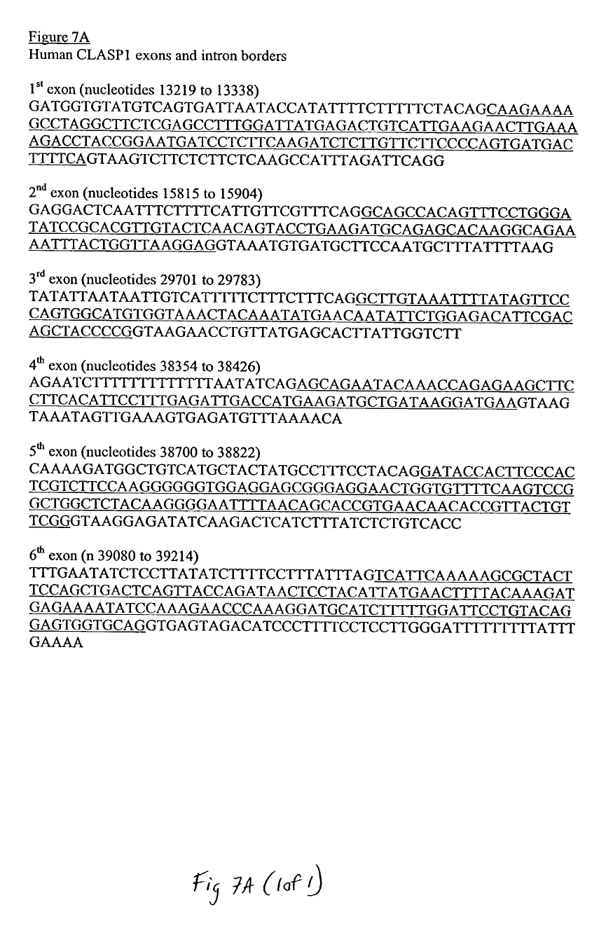

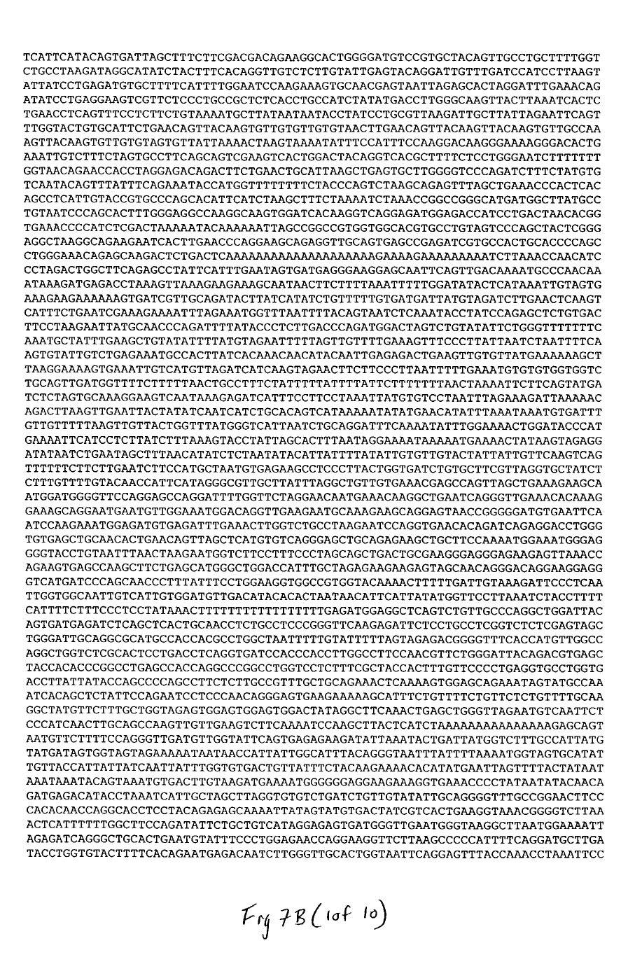

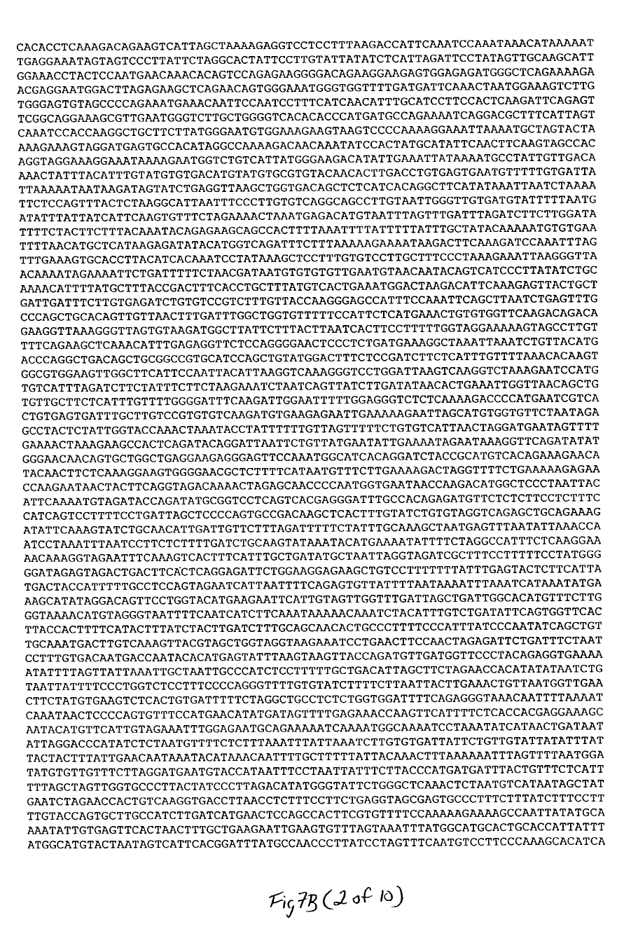

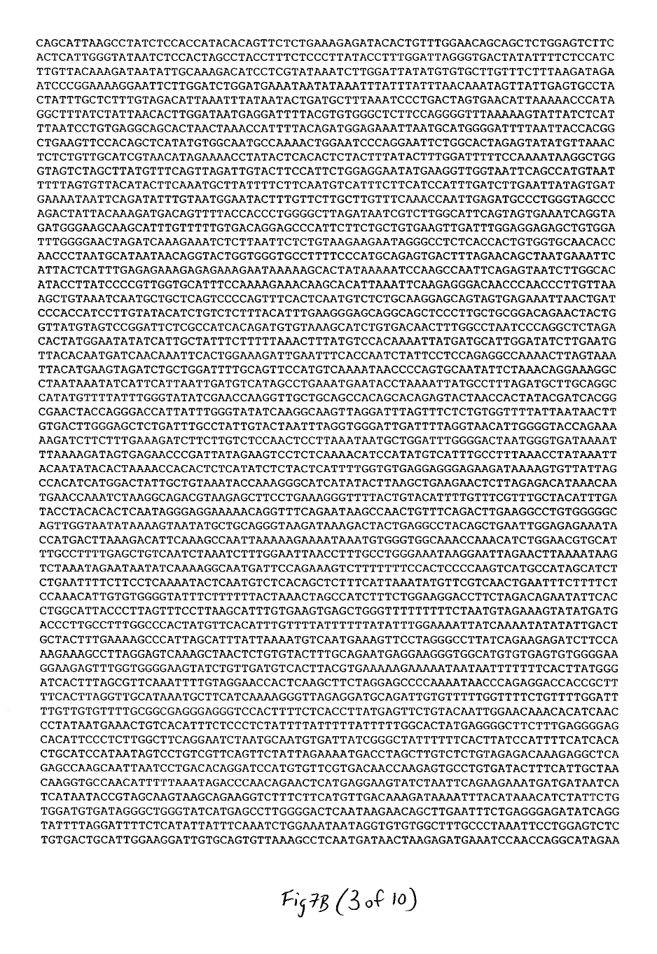

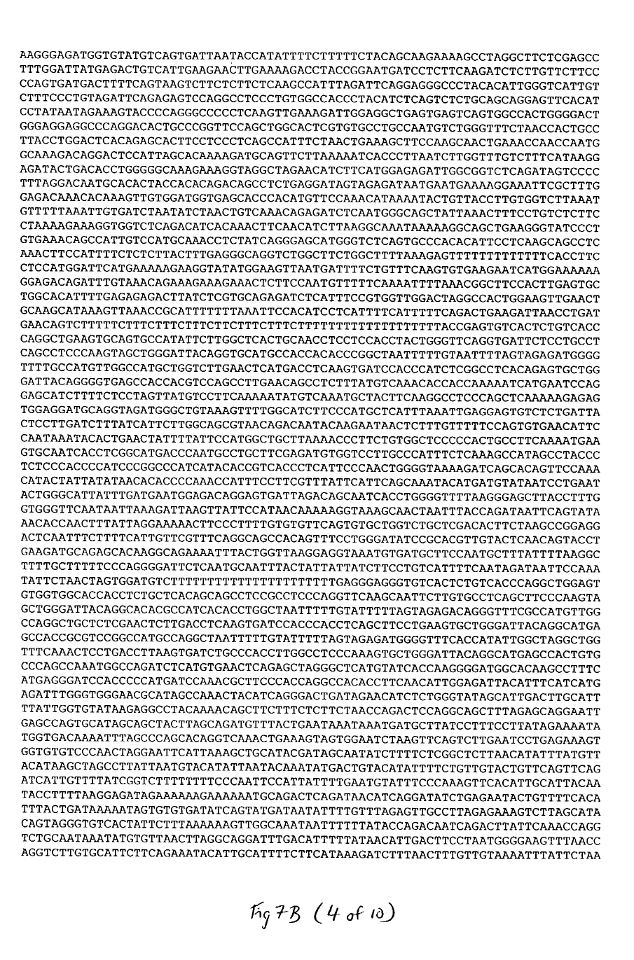

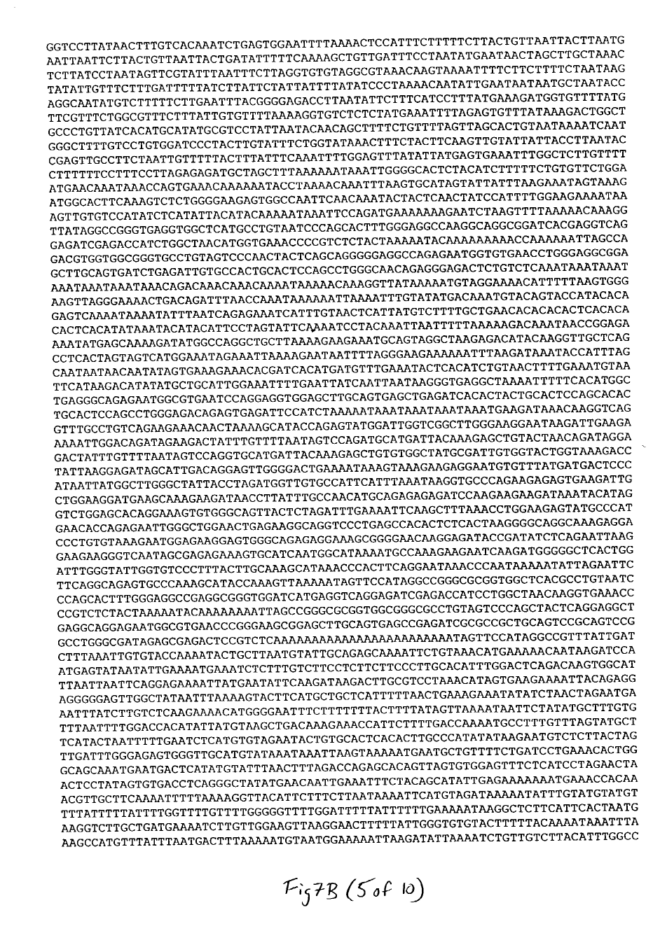

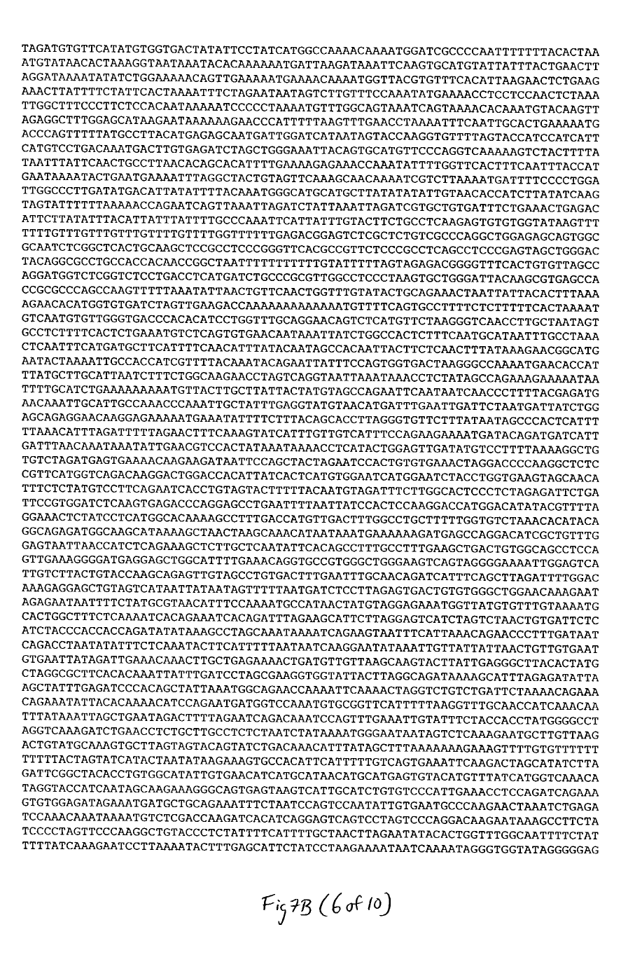

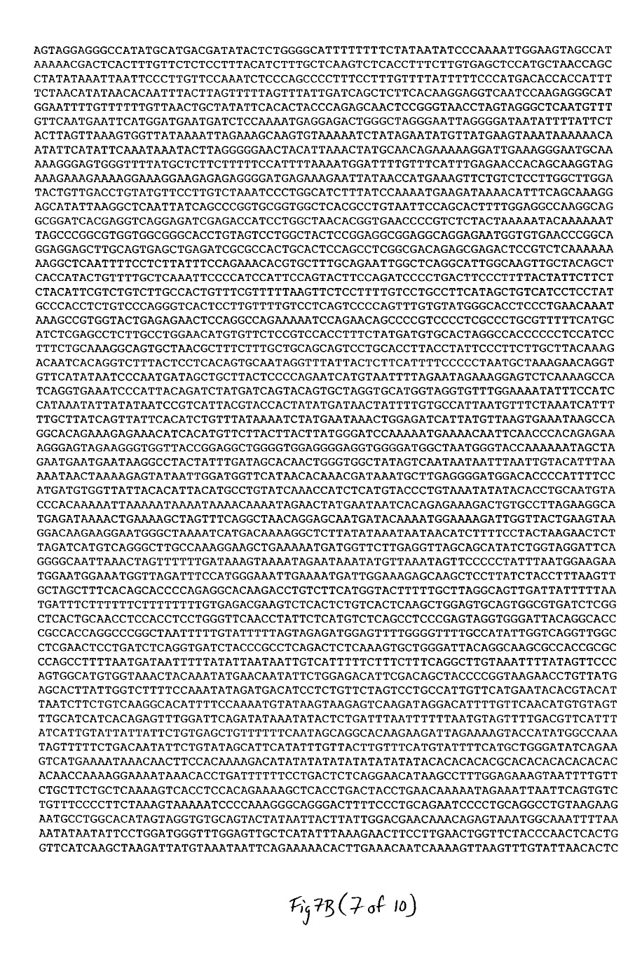

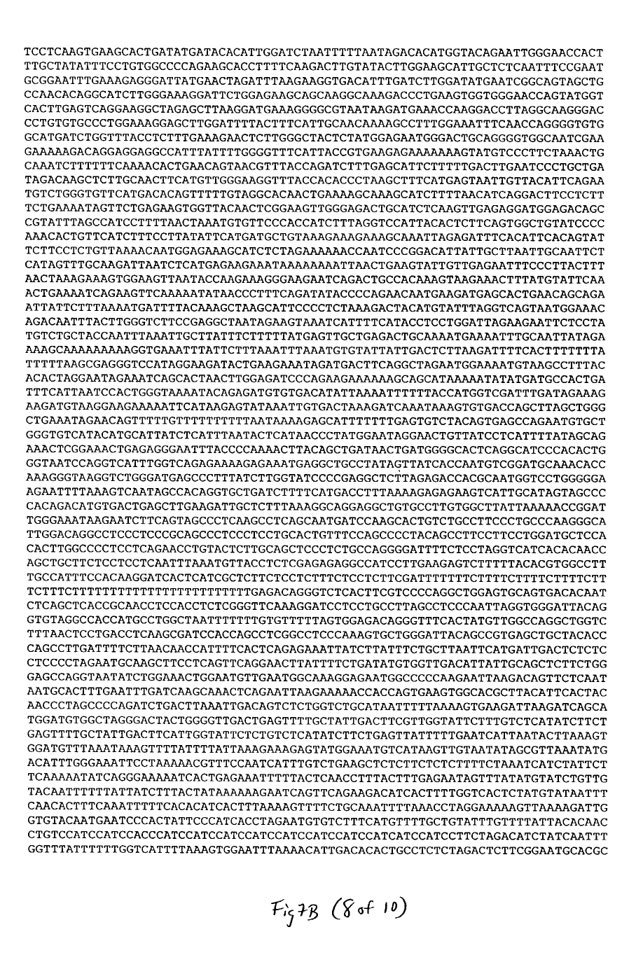

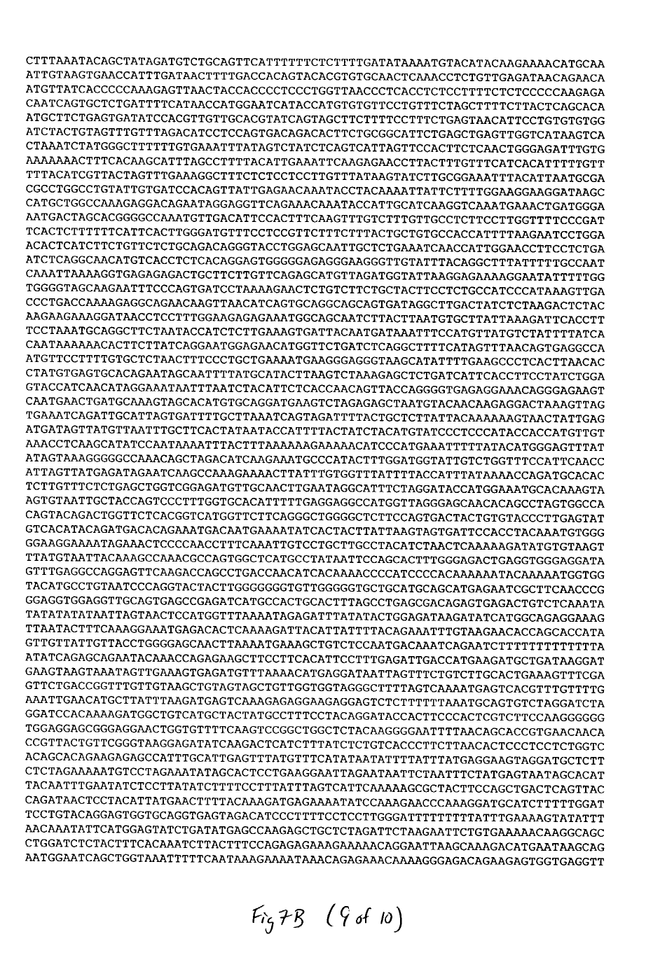

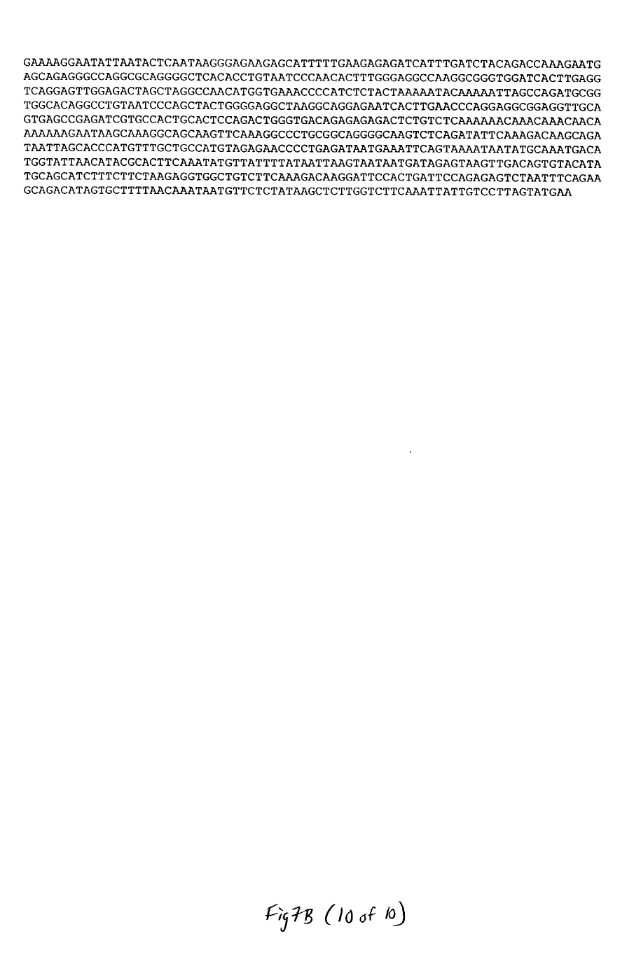

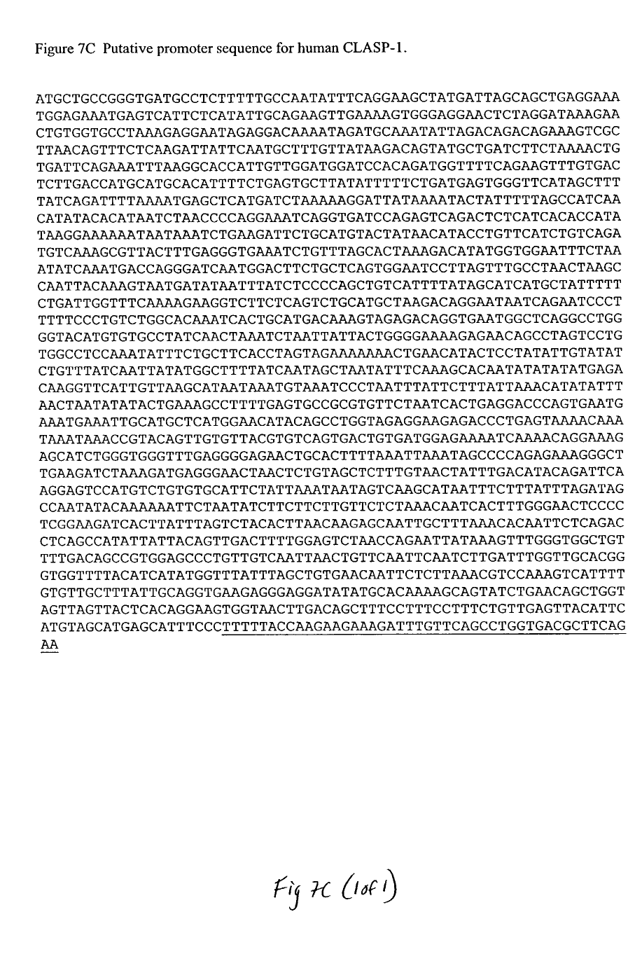

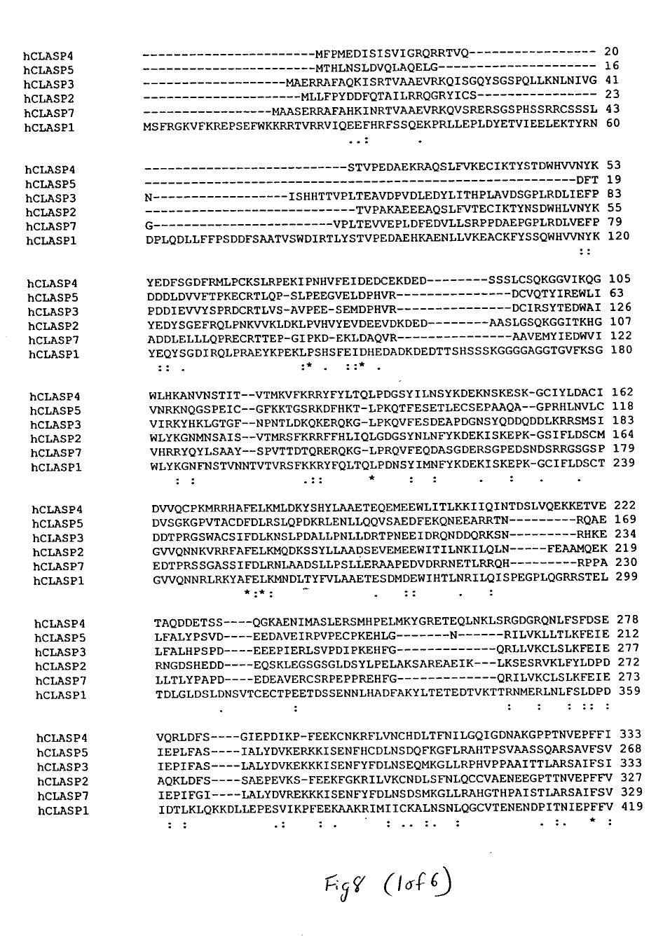

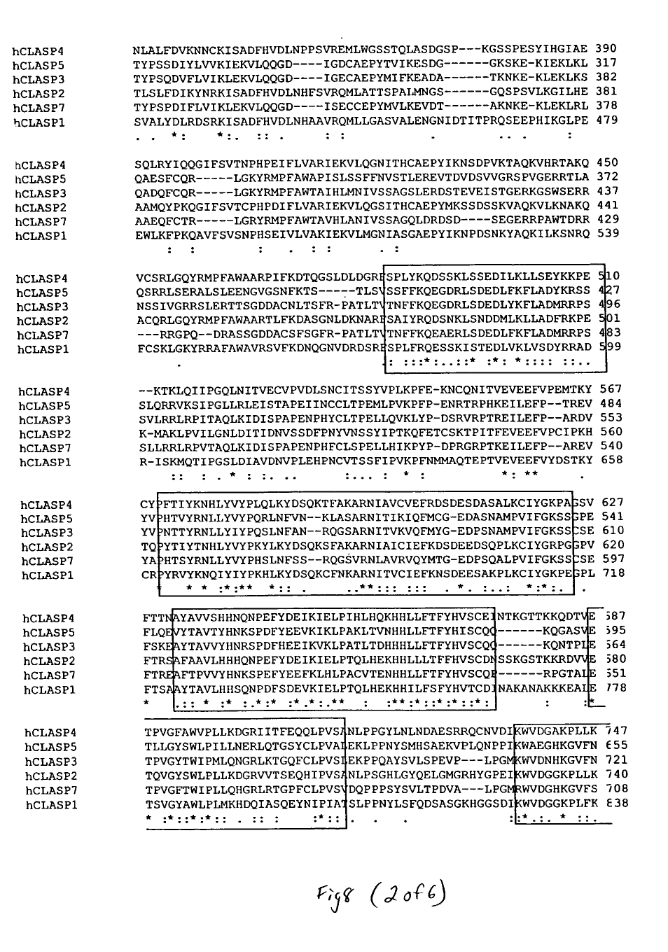

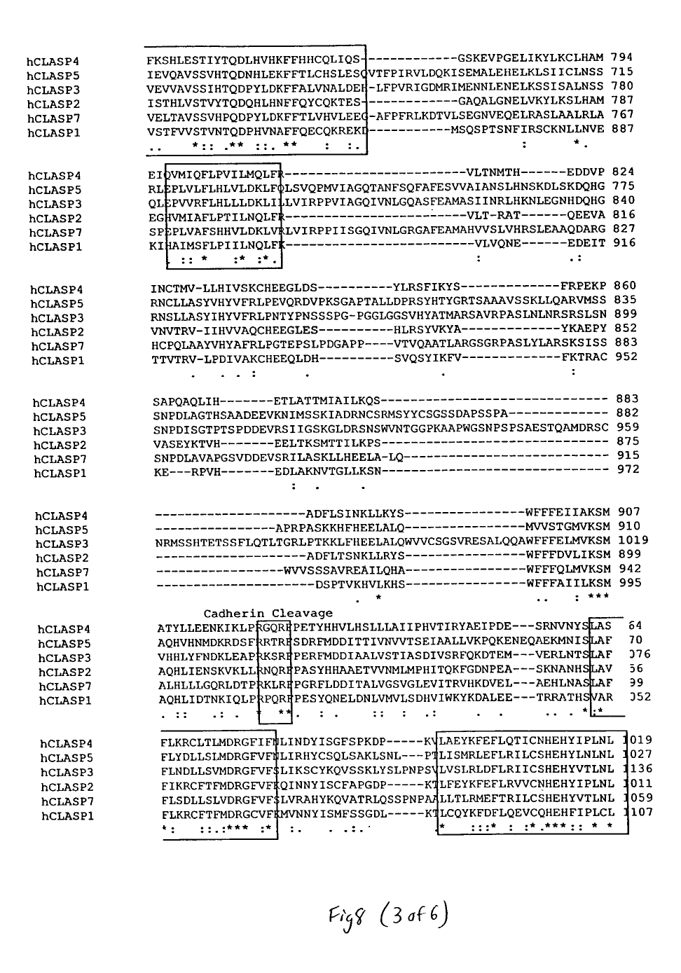

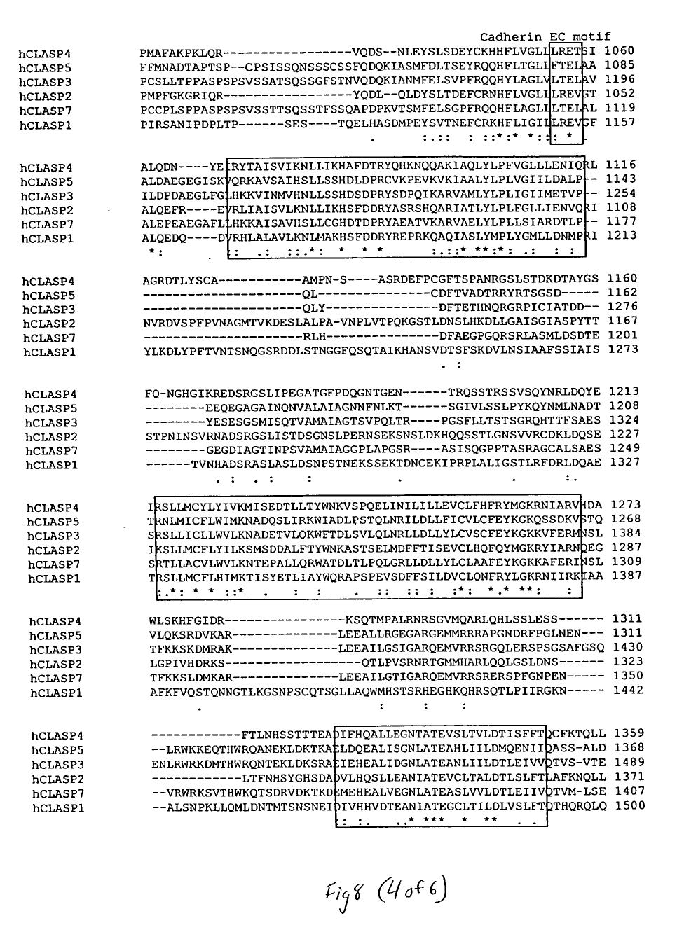

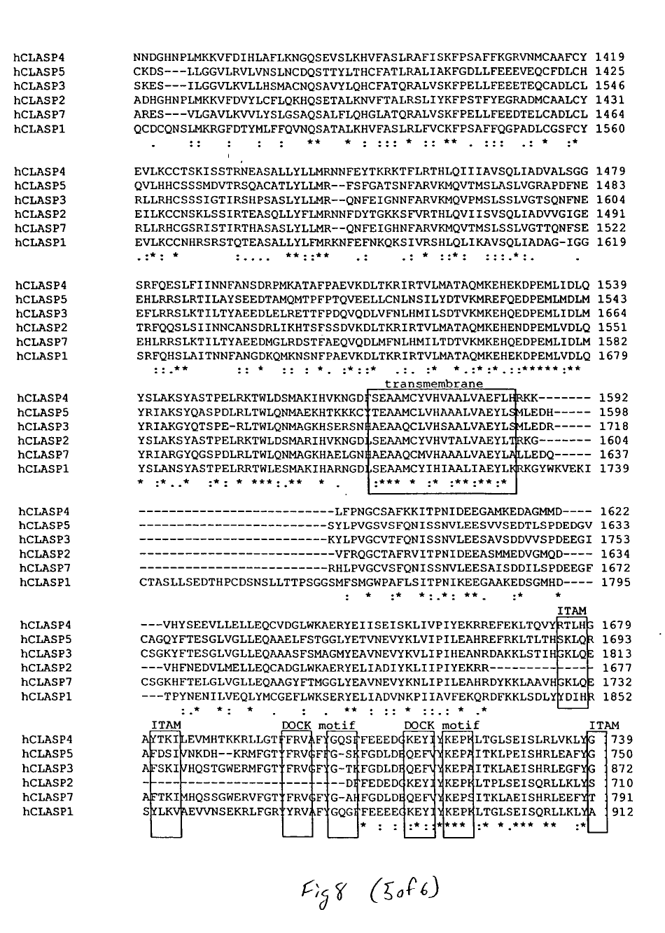

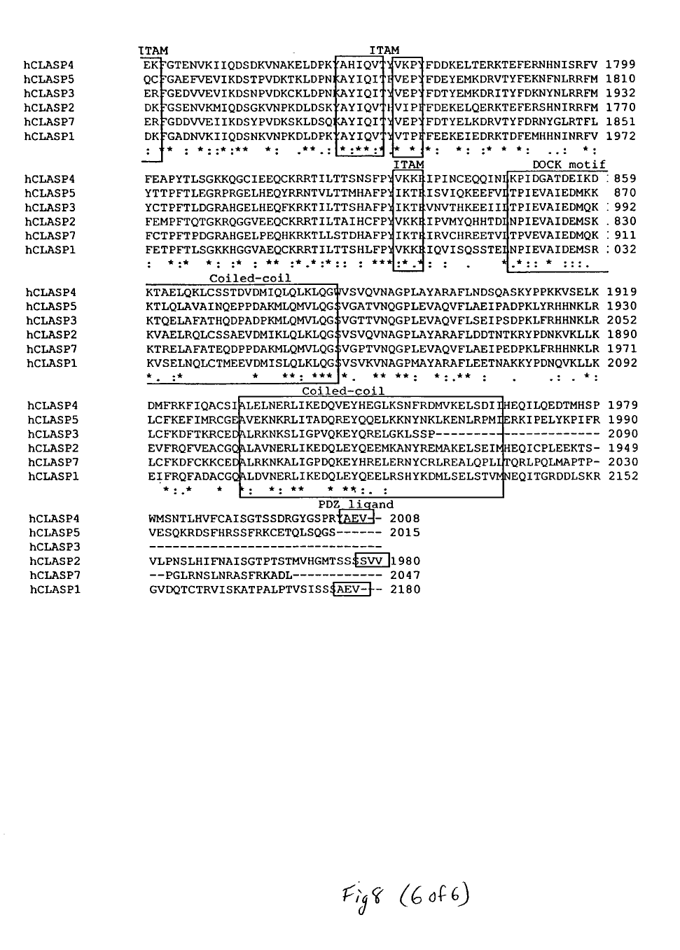

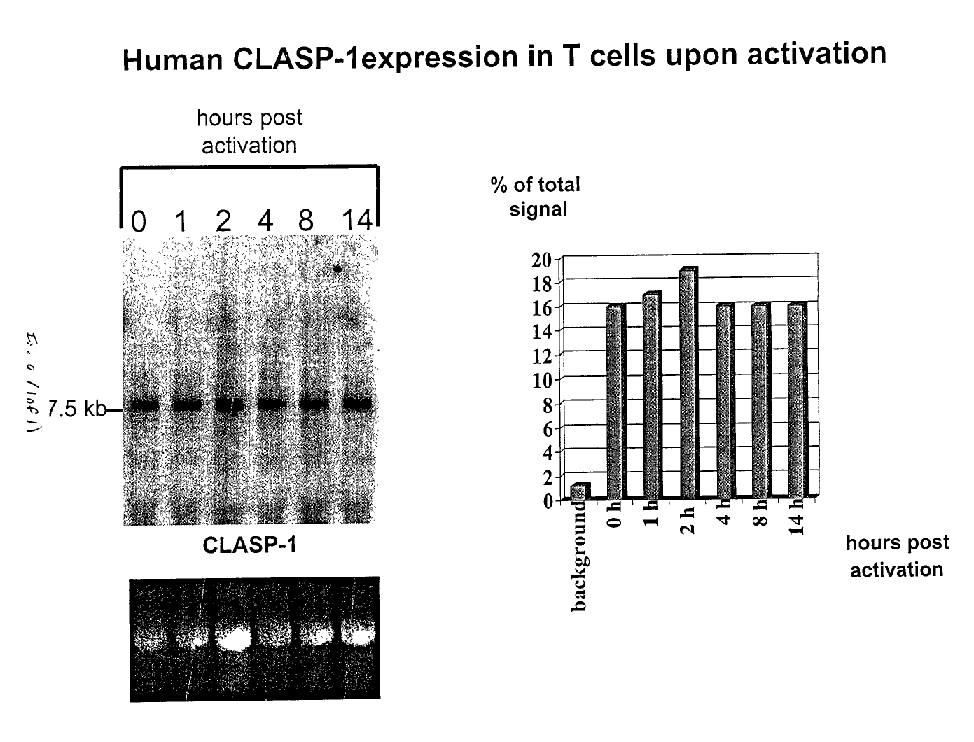

The CLASP proteins are a membrane associated or integrated proteins, characterized by multiple forms produced by alternative exon usage (i.e., production of splice variants). In one naturally occurring form, CLASPs have the conceptual translations shown in FIG. 1 and FIG. 6A. However, as discussed in detail infra, the CLASP genes encode a variety of gene product due to alternative splicing of mRNA. FIG. 2 shows the polymorphisms and alterations present in the various mouse CLASP cDNAs and their consequences in the resultant protein sequence (as shown in FIG. 1 and FIG. 3). In a similar manner, FIG. 6B details a number of nucleotide polymorphisms identified when compared to the polynucleotide sequence in FIG. 6A.

The present invention provides polynucleotides having the sequences as shown in FIG. 1, FIG. 3, FIG. 6A or fragments thereof, and polypeptides having the sequences as shown in FIG. 1, FIG. 3, FIG. 6A or fragments thereof. In addition, the invention provides polynucleotides comprising naturally occurring alleles of CLASPs, and CLASP variants as described herein, and methods for using CLASP polynucleotides, polypeptides, antibodies and other reagents.

5.2.1. CLASP Polypeptide Domains

One naturally occurring CLASP cDNA encodes a polypeptide characterized by several structural and functional domains and defined sequence motifs. To provide guidance to the practitioner, the structural features are described infra. However, it will be understood that the present invention is not limited to polypeptides that include all, or any particular one of these domains or motifs. For example, a CLASP fusion protein of the invention contains only the extracellular domain of CLASP-1.

It will be appreciated that the structurally (and functionally) different domains of CLASP polypeptides (and the corresponding region of the mRNA) are of interest, in part, because they may be separately targeted or modified (e.g., deleted or mutated) to affect the activity or expression of a CLASP gene product (in order to, for example, modulate an immune response). For example, the extracellular domain of a CLASP protein can be targeted (e.g., using an anti-CLASP monoclonal antibody to (a) block the interaction of a CLASP-expressing cell (e.g., a T cell) and a second cell (e.g., a B cell) displaying a protein that is bound by a CLASP (i.e., a CLASP ligand). Similarly, an intracellular domain can be targeted to interfere with signal transduction without interfering with extracellular ligand binding.

Generally, inhibiting CLASP expression or CLASP polypeptide function will result in modulation of immune function including, for example, changing the threshold for T cell activation by affecting formation of the immune synapse. Modulation of immune function can be screened and quantitated by a number of assays known in the art and described herein (see also §5.14).

5.2.1.2. Extracellular Domain

Through its extracellular domains, CLASPs may interact with ligands in a homotypic and/or heterotypic manner to establish the immunological synapse in conjunction with molecules such as TCR, MHC class I, MHC class II, CD3 complex and accessory molecules such as CD4, CD3, ICAM-1, LFA-1, and others.

Antibodies raised against the extracellular domain of a CLASP can be added to cells expressing CLASPs. These antibodies can either block the interaction of CLASPs with potential ligands or stabilize these interactions. Any immunoassay known in the art, e.g., listed and described herein, may be used to assess the modulation of immune function brought about by this approach.

Similarly, portions of the extracellular domain of CLASPs can be expressed as soluble protein. This soluble protein can then be added to cells expressing CLASPs. These proteins may interact with potential ligands to competitively inhibit their binding to endogenous CLASPs. This could modulate CLASPs function via the immunoassays described herein. Recombinant proteins could interfere in a positive or negative fashion with CLASPs interactions.

5.2.1.3. Intracellular Domains

The CLASP intracellular domains contain motifs corresponding to several types of protein domains. Depending on the specific CLASPs (i.e., specific family member or splice variant) all or only some of the domains may be present. Listed from amino terminus to carboxy terminus, the domains include: (1) a PH domain, (2) a newly discovered DOCK/CLASP motif, (3) a coiled-coil motif, and (4) a C-terminal PDZ binding motif (PBM) (also referred to as PDZ ligand or “PL”).

5.2.1.4. DOCK

CLASP polypeptides contain a new “DOCK” motif, not previously described in the scientific literature. The CLASP DOCK motif includes a series of five tyrosines surrounded by conserved sequences in regions A, B, C, D, and G (see FIG. 8). There are also two highly conserved non-tyrosine containing regions (E and G) separated by nine amino acids (P+EXAI+XM) and (LXMXL+GXVXXXVNXG) (where X is any amino acid).

The DOCK gene family includes three molecules that are the human homologues of the C. elegans CED proteins known to be involved in apoptosis. CED-5 (DOCK180), a major CRK-binding protein, alters cell morphology upon translocation to the membrane (mediates the membrane motion that scavenger cells exhibit as they surround and engulf dying cells; its function can be partially rescued by the human DOCK180 (Wu et al., 1998, Nature 392: 501-504). Myoblast City in Drosophila (MBC) is another member of the DOCK protein family and has been found to be involved in myoblast fusion (Erickson, et al., 1997, J. Cell Biol. 138: 589).

The DOCK family has been implicated in the control of cell shape. DOCK1, when transfected into spindle cells, can make them flattened and polygonal (Takai, et al., 1996, Genomics 35: 403-303). DOCK1 expression is ubiquitous except in hematopoietic cells. DOCK2 is expressed in hematopoietic cells and when transfected into spindle cells can make them round up (Nishihara, H., 1999, Hokkaido Igaku Zasshi 74: 157-66). DOCK2 is expressed in peripheral blood lymphocytes, thymus, spleen, and liver.

5.2.1.5. Coiled-coil

CLASPs have zero, one or two coiled-coil domains (Lupas et al., 1991, Science 252: 1162-64; Lupas, A., 1996, Meth. Enzymology 266: 513-525). Coiled-coil domains are known to interact directly with cytoskeleton, indicating that CLASP proteins interact directly with the cytoskeleton. Thus, it is believed that CLASPs bind cytoskeletal proteins, e.g., ankyrin, spectrin, hsp70, talin, ezrin, tropomyosin, myosin, plectin, syndecans, paralemmin, Band 3 protein, Cytoskeletal protein 4.1, Tyrosine phosphatase PTP36 and other molecules.

5.2.1.6. Pleckstrin Homology (PH) Domain

Both human and mouse CLASP-1 contain a single pleckstrin homology (PH) domain close to the amino terminus of the protein. Additionally, human and mouse CLASP-2 and CLASP-4 contain PH domains. The PH domain is a stretch of approximately 120 amino acids involved in intracellular signaling, localization of signal transduction molecules, and cytoskeletal rearrangement (Cantrell, 2001; Ma and Abrams, 1999; Toker and Cantley, 1997). The PH domain has been shown to bind several substrates including phosphoinositides, protein kinase C isoforms and heterotrimeric G proteins (Okoh and Vihinen et al). It has been the binding to phosphoinositides that has been shown to most directly relevant in vivo.

The presence of a PH domain in a protein results in its localization to the plasma membrane where phosphoinositdes are generated. This could indicate that CLASP protiens with PH domains would be localized to the plasma membrane to serve in intracellular signaling or cytoskeletal rearragnement.

PH domains are found in numerous proteins inlcuding pleckstrin, phosopholipase Cδ1, dynamin, SOS, Btk (Akt/PKB), Grp-1, cytohesin-1 and ARNO and β-adrenergic receptor kinase. The presence of PH domains in a wide variety of protiens, and a myriad of signal transduction pathways and cytoskeletal organizational processes lends support to the importance of PH domains.

Despite their presence in a wide variety of proteins, point mutations leading to single amino acid changes in the PH domain have specific detrimental effects leading to specific disease states. For example a single amino acid change in the PH domain of Btk leads to an immunodeficiency in mice (Thomas et al., 1993; Rawlings et al., 1993), and X-linked agammaglobulinemia in humans (Vihinen et al., 2001; Mattsson et al., 1996). In vitro it has been demonstrated that a single point mutation in Btk leads cells becoming unresponsive to specific growth factor-initiated signaling pathways (Kohn et al, 1996, Franke et al, 1997). Additionally, a point mutation in the PH domain of FGD1, another intracellular signaling protein, leads to Aarskog-Scott syndrome, a disease resulting in faciogeneital dysplasia. This demonstrates that while PH domains are conserved, they affect specific signaling pathways that can be associated with clear, distinct human diseases and phenotypes.

5.2.1.7. PDZ Ligand

CLASP proteins contain a PDZ-ligand motif (“PBM” or “PL”) at the C-terminus of the protein. This short (3-8 amino acid) motif mediates the binding of proteins terminating at their carboxyl terminus in the motif (most commonly S/T-X-V-free carboxyl-terminus) to other proteins containing one or more specific PDZ domains (See Songyang et al., 1997, Science 275: 72 and Doyle et al., 1996, Cell 85: 1067 for a discussion of PDZ-ligand structures).

PDZ domain-containing proteins are involved in the organization of ion channels and receptors at the neurological synapse and in establishing and maintaining polarity in epithelial cells via their binding to the C-termini of transmembrane receptors. It has been shown that PDZ-domain containing proteins can mediate protein-protein interactions in immune system cells (e.g., DLG1 binds to the lymphocyte potassium channel KV1.3 in human T lymphocytes, (Hanada et al., 1997, J. Biol. Chem. 272: 26899).

Biochemical evidence that human CLASP-2 interacts with the PDZ domains of three closely related proteins is has been described in WO 00/10158. These observations have been generalized to the CLASPs based on the sequence identity between the human and mouse CLASPs (see FIG. 3). Since the sequence motif used to bind PDZ domains are identical between human and mouse CLASPs it is reasonable to suggest that the mouse CLASPs can similarly bind PDZ domains.

5.2.1.7. Modulation of Immune Responses

CLASP proteins, as described above, modulate immune function in a variety of ways and through a variety of mechanisms (i.e., changing the threshold for T cell activation) by affecting formation of the immunological synapse. Establishment and maintenance of the immunological synapse can involve: (A) signal transduction, (B) cell-cell interactions, and (C) membrane organization.

(A) Signal Transduction

Several adaptor proteins including NCK, CBL (Bachmaier, K., 2000 Nature 403: 211-6), SHC, LNK, SLP-76, HS1, SIT, VAV, GrB2, and BRDG1, and two tyrosine phosphotases, EZRIN, SHP-1 and SHP-2 have been shown to interact with ITAM or SH3 domains. These proteins may also interact with CLASPs. These interactions can be defined by a number of different biochemical or cell biological methods including in vitro binding assays, co-immunoprecipitation assays, co-immunostaining (Harlow, E. and Lane, D., 1999, Using Antibodies: A laboratory Manual. Cold Spring Harbor Press) or genetic assays such as yeast the yeast two hybrid system, in which a CLASP protein or fragment can be used as “bait” (Zervos et al., 1993, Cell 72: 223-232; Madura et al., 1993, J. Biol. Chem 268: 12046-12054).

Other assays include in vitro binding assays, co-immunoprecipitation assays, co-immunostaining assays, and yeast two hybrid system screening assays in which a CLASP domain or fragment can be used as “bait” or “trap” protein (Zervos et al. (1993), Cell 72: 223-232; Madura et al. (1993) J. Biol. Chem. 268: 12046-12054).

In other embodiments, CLASP polypeptides are transfected into lymphocytes. After transfection, a variety of standard assays can be used to evaluate, for example, CLASP modulation of T cell activation. These assays include calcium influx assays, NF-AT nuclear translocation assays (e.g., Cell, 1998, 93: 851-61), NF-AT/luciferase reporter assays (e.g., MCB 1996 16: 7151-7160), tyrosine phosphorylation of early response proteins such as HS1, PLC-γ, ZAP-76, and Vav (e.g., J. Biol. Chem. 1997, 272: 14562-14570).

5.2.2. CLASP Alternative Splice Variants

Alternative splicing and RNA editing are mechanisms generate a variety of proteins from the same gene that are closely related to each other but that can differ in structure and thus can exert distinct functions. An example for how alternative splicing is used to generate thousands of different proteins from only a few genes is represented by the Neurexin gene family (for review of Neurexins, see Missler M. and Sudhof, T., 1998, Trends in Genetics, 14: 20-25).

Alternative splicing is likely to represent a regulatory switch that governs different functions of CLASPs in the immune response. Alternative splice variants affecting the untranslated regions of an RNA can be a way of regulating RNA stability.

As noted supra, CLASP gene expression is characterized by alternative exon usage. Intron/exon structure can be predicted by computer analysis of genomic DNA, however, splice junctions and alternative splicing can only be elucidated by comparison of genomic clones to cDNA clones. Alternative splicing and RNA editing are mechanisms generate a variety of proteins from the same gene that are closely related to each other but that can differ in structure and thus can exert distinct functions. An example for how alternative splicing is used to generate thousands of different proteins from only a few genes is represented by the Neurexin gene family (for review of Neurexins, see Missler M. and Sudhof, T., 1998, Trends in Genetics, 14: 20-25). Comparative analysis of CLASP genomic clones and cDNA clones revealed that CLASPs are composed of numerous exons and that distinct CLASP-transcripts are generated by alternative splicing.

Numerous diseases are caused or are thought to be caused by splice site mutations that can cause exon skipping or otherwise result in a truncated protein product Some of these diseases include, e.g., Marfan Syndrome (Liu W, et al., 1997, Nat. Genet. 16: 328-9), Hunter disease (Bonucelli G, et al., 2000, Hum. Mutat. (Online) 2000 15(4): 389, Duchenne muscular dystrophy (Wibawa T, et al., 2000, Brain Dev. 22(2): 107-112), Myelomonocytic leukemia (Wutz D, et al., 1999, Leuk. Lymphoma 35: 491-9.), and Isovaleric acidemia (Vockley J, et al., 2000, Am. J. Hum. Genet. 66: 356-67). This is especially true for genes composed of many exons (such as the CLASPs). The genomic sequence around CLASP exon/intron boundaries is useful for diagnostic approaches towards the identification of diseases caused by splice site mutations. The abundance or presence of CLASP-isoforms in cell populations (e.g., hematopoietic cells, lymphocytes) is correlated with a disease state by comparing the abundance of a given CLASP in cells from subjects suffering from the disease with the level of CLASP in cells from healthy subjects. This can be accomplished by utilizing any number of assays (e.g., PCR). This can be accomplished by utilizing any number of assays (e.g., PCR).

Alternative splicing and RNA editing are mechanisms generate a variety of proteins from the same gene that are closely related to each other but that can differ in structure and thus can exert distinct functions. An example for how alternative splicing is used to generate thousands of different proteins from only a few genes is represented by the Neurexin gene family (for review of Neurexins, see Missler M. and Sudhof, T., 1998, Trends in Genetics, 14: 20-25).

5.2.3. CLASP Superfamily Members

As is illustrated in FIG. 5 and FIG. 8, the CLASPs are a superfamily of immune-cell associated proteins with similar motifs. CLASP-1 was described in PCT/US99/22996, published as WO 00/20434 on Apr. 13, 2000. CLASP-1 was also described in copending U.S Provisional Application No. 60/240,545, filed Oct. 13, 2000 (which is incorporated by reference herein in its entirety for all purposes). CLASP-1 uniquely among the known CLASPs contains SH3 binding domain motifs. The other CLASP polypeptides seem to lack adaptor binding sites or SH3 binding domains as found in CLASP-1.

CLASP-2, CLASP-3, CLASP-5 and CLASP-7 are described in copending U.S. application Ser. Nos. 09/547,276, 09/737,246, 09/736,969, 09/736,960, 09/736,968 (all filed Dec. 13, 2000); and 09/687,837, filed Oct. 13, 2000, and WO 00/61747 (published on Oct. 19, 2000), WO 01/42297, WO 01//42294, WO 01/42296, and WO 01/42295 (all published on Jun. 14, 2001), and which are incorporated by reference herein in their entirety for all purposes.

5.3.1 CLASP-mRNA Expression

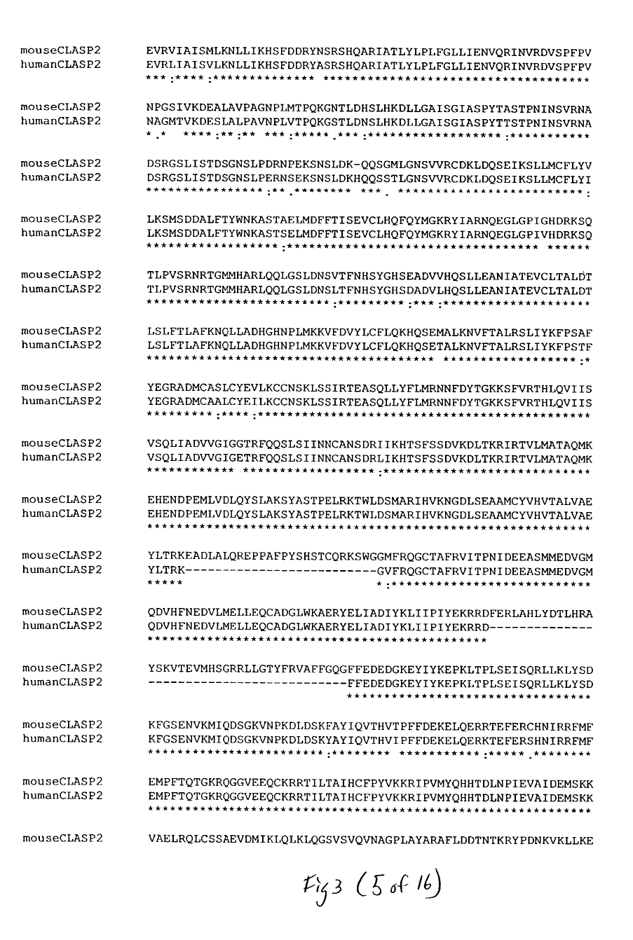

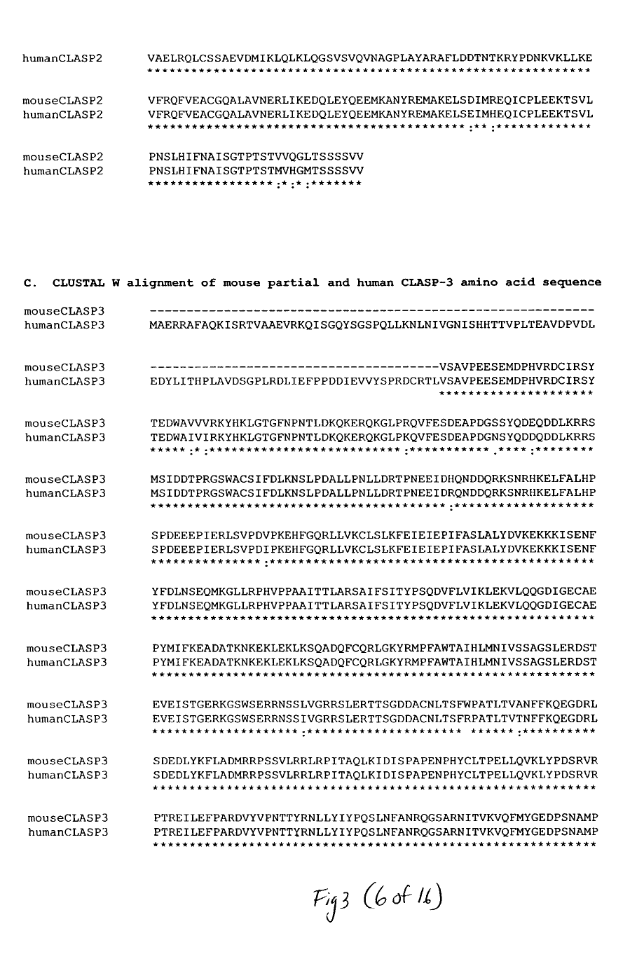

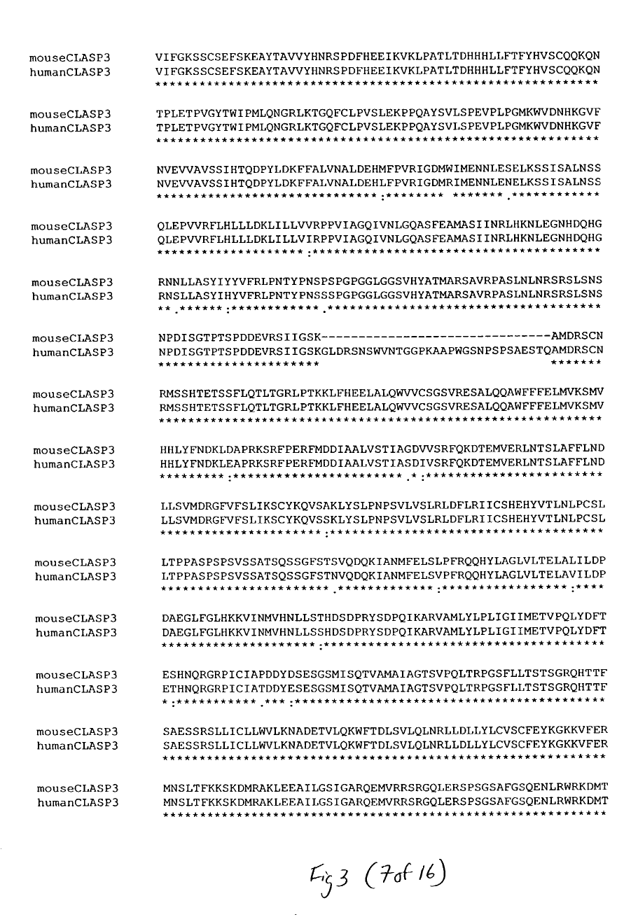

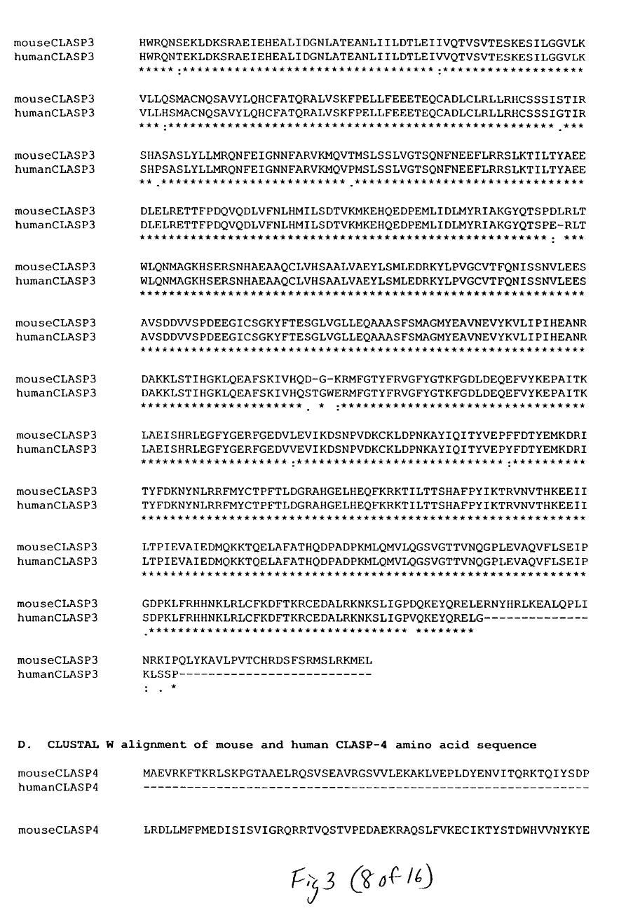

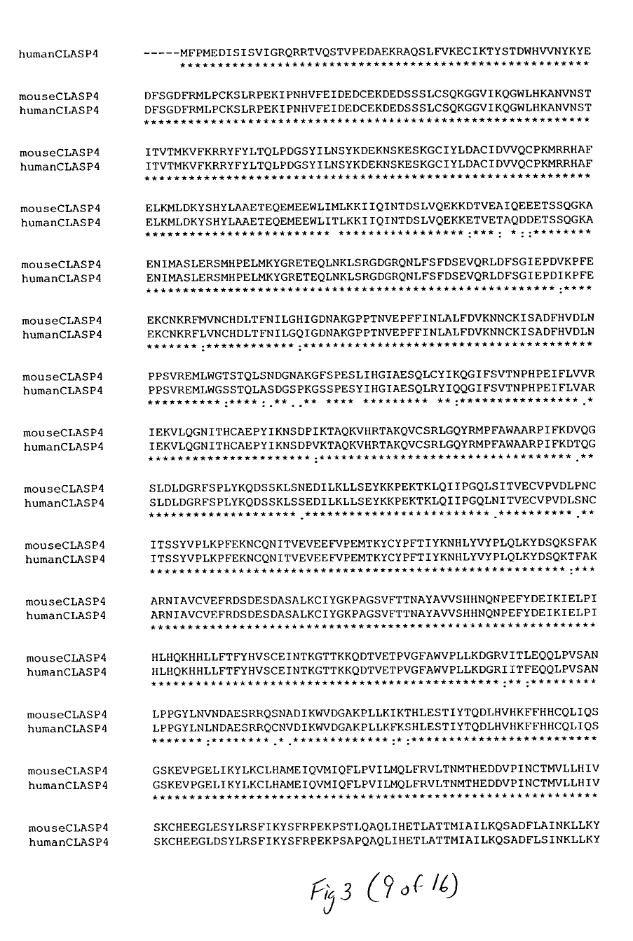

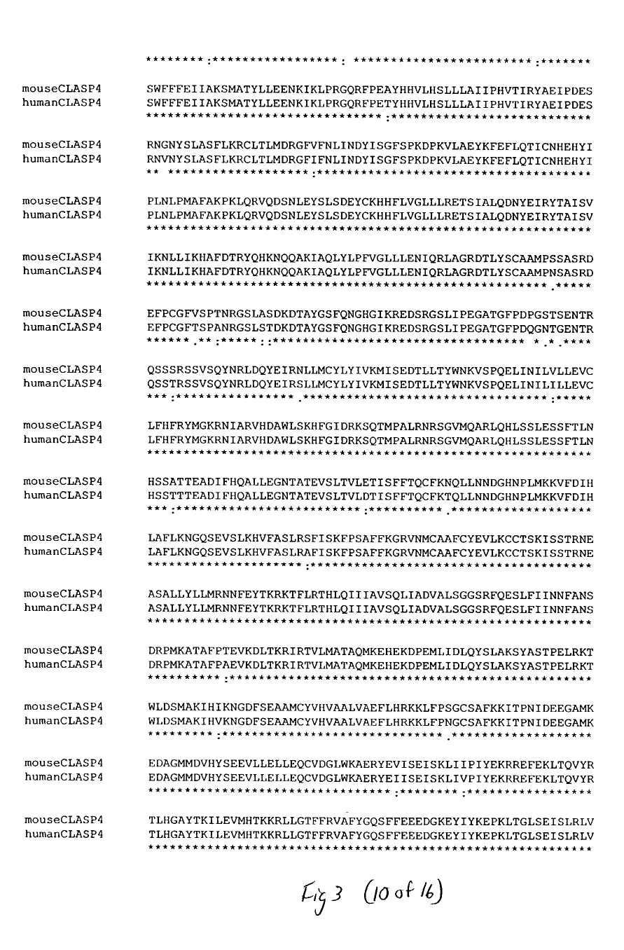

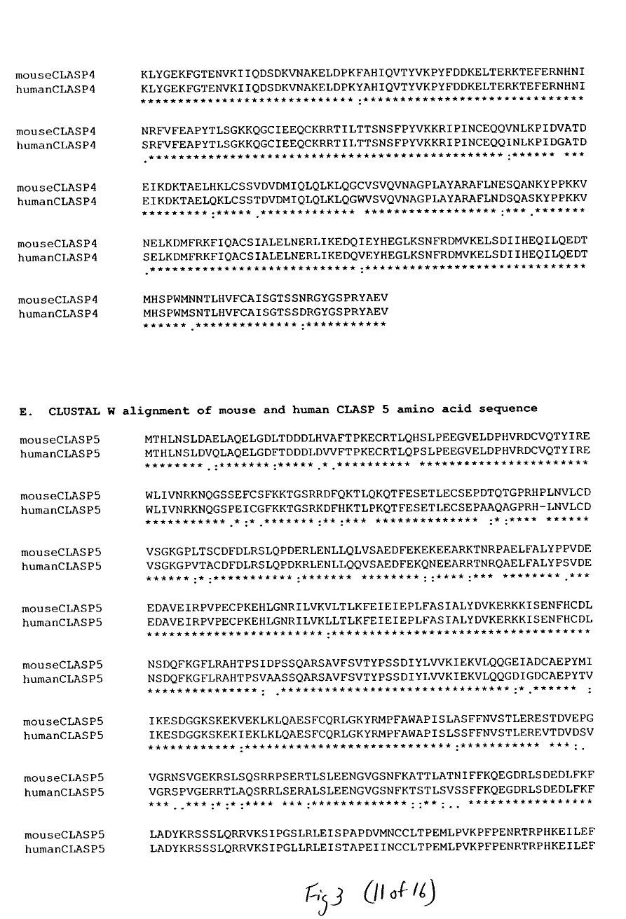

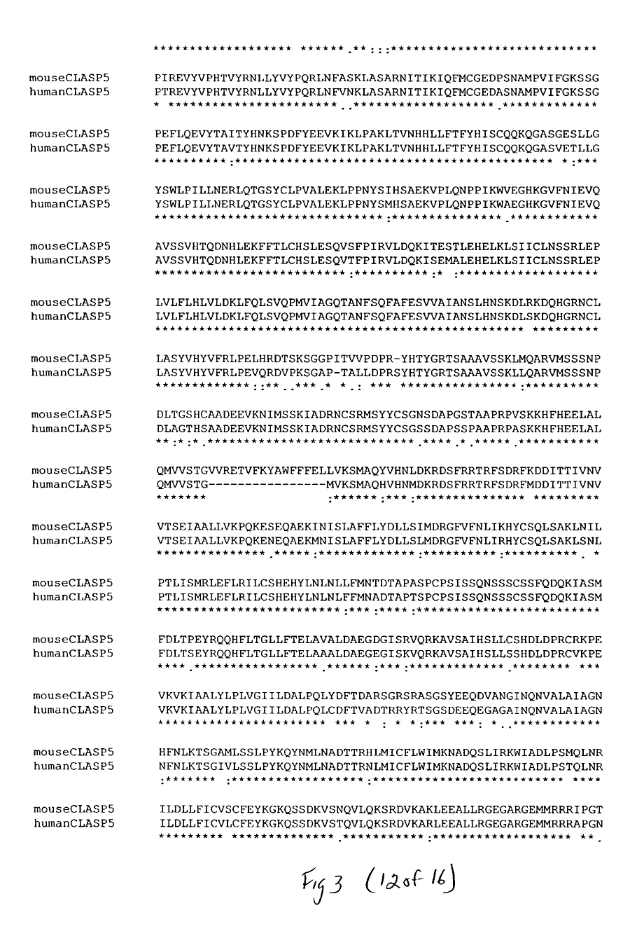

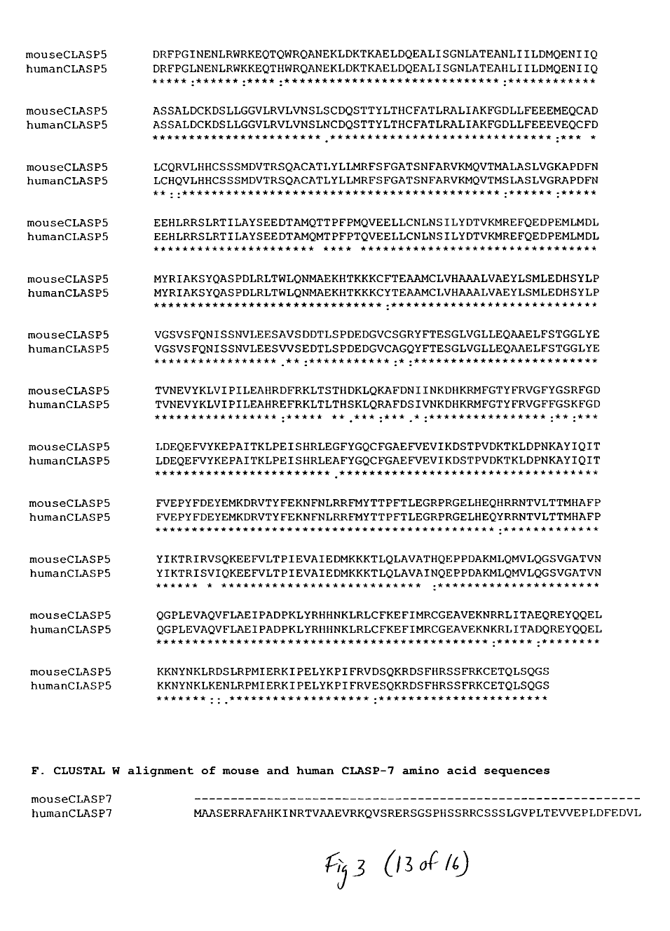

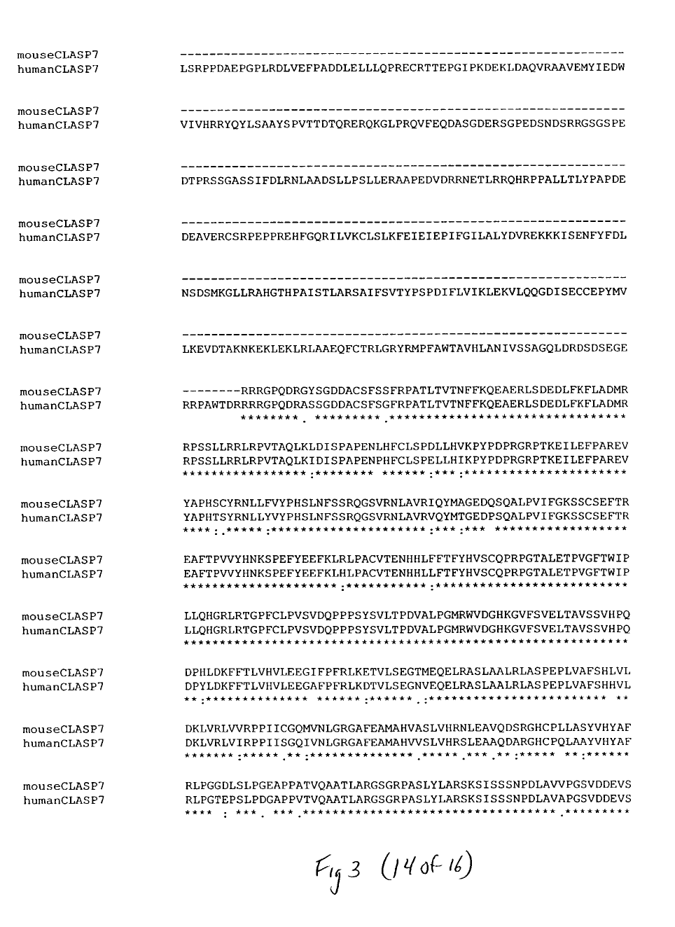

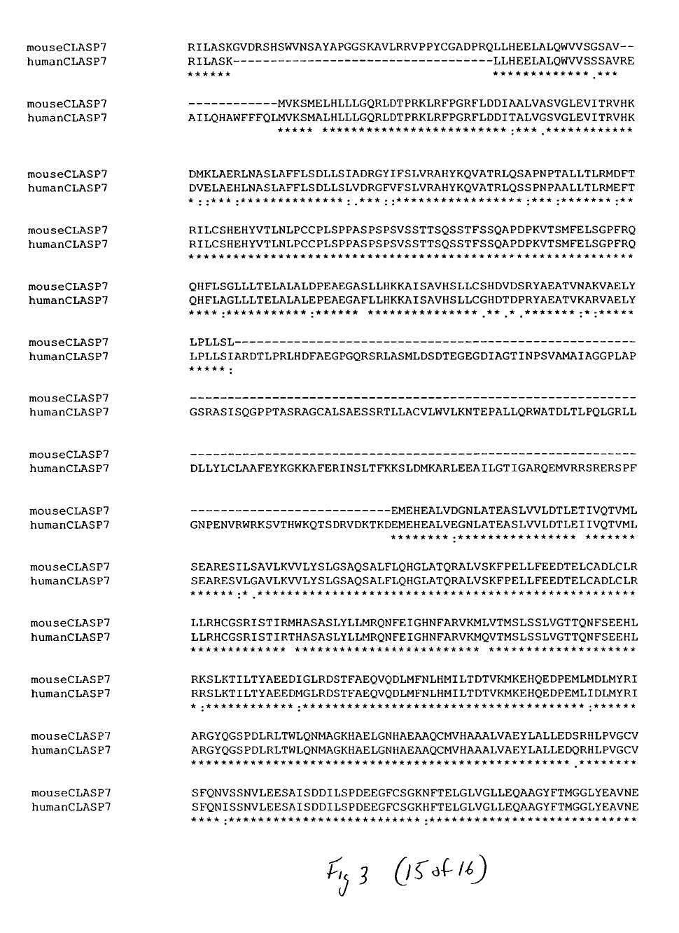

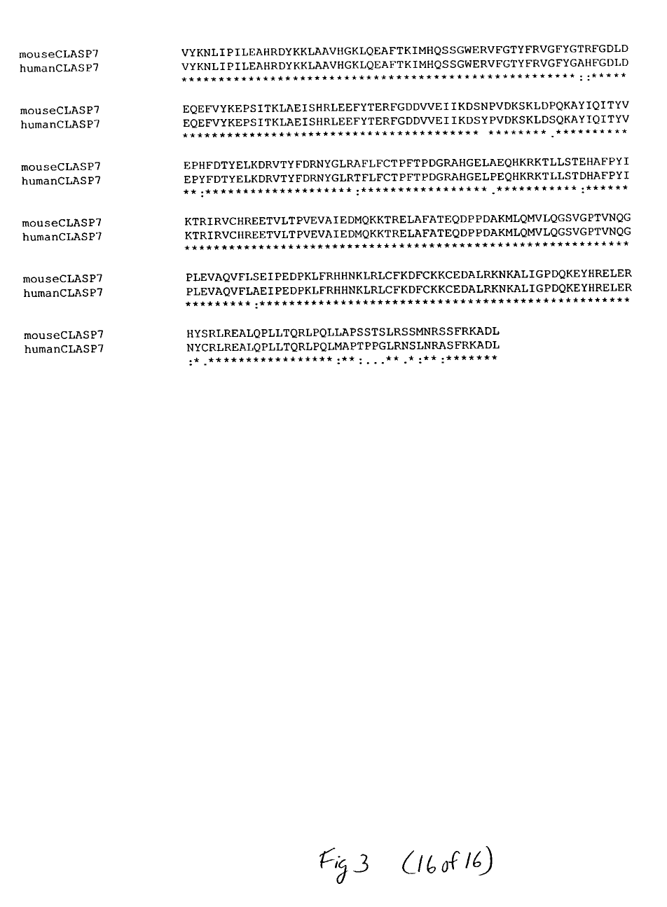

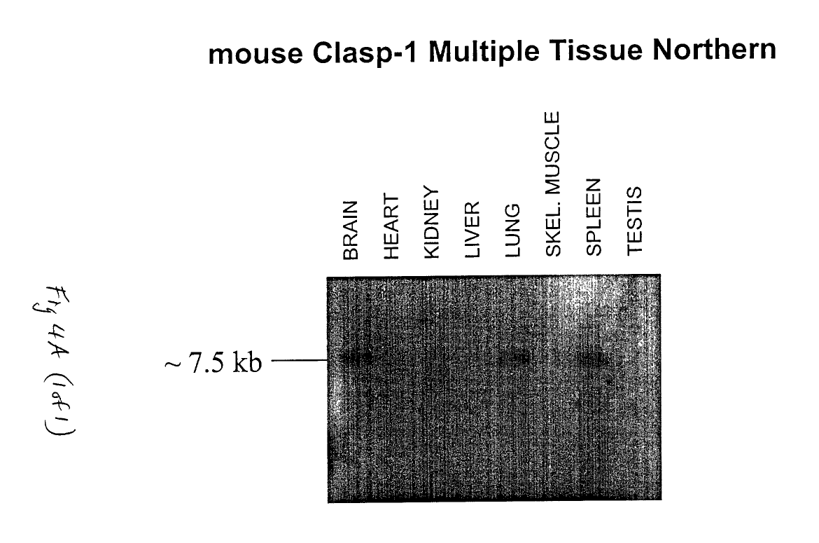

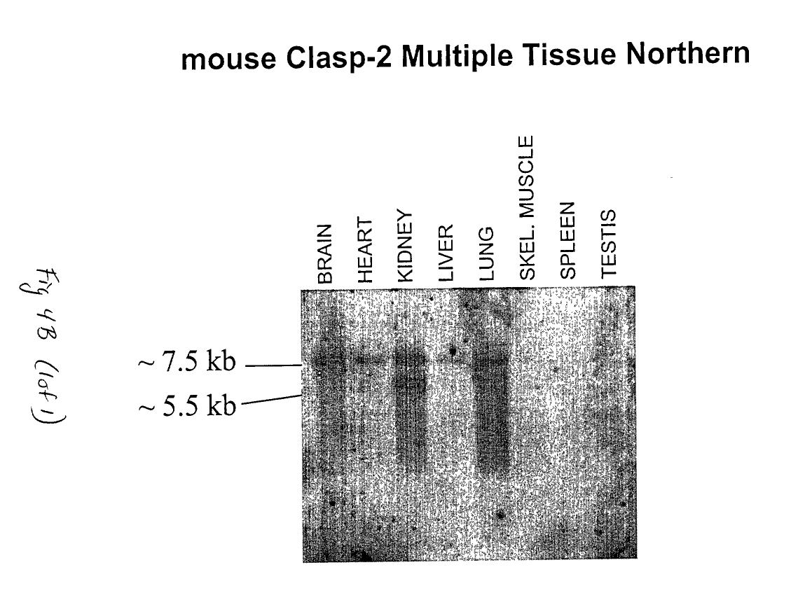

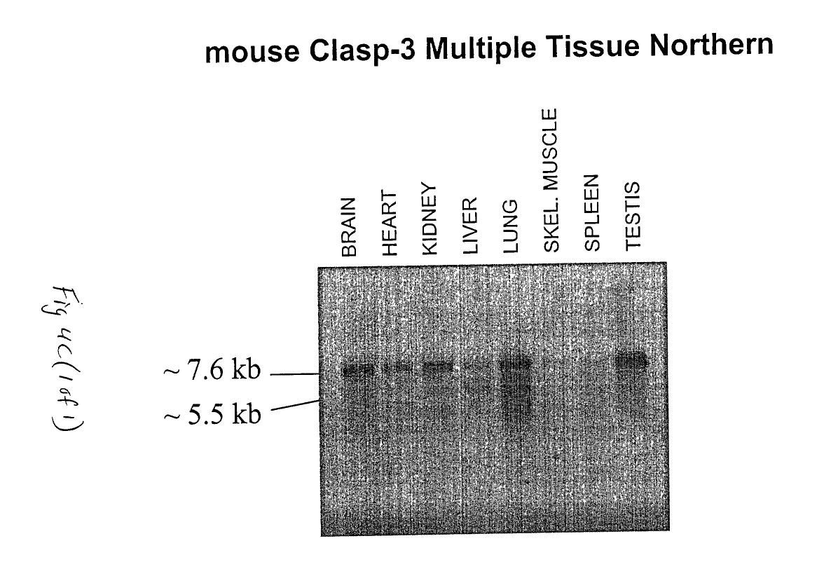

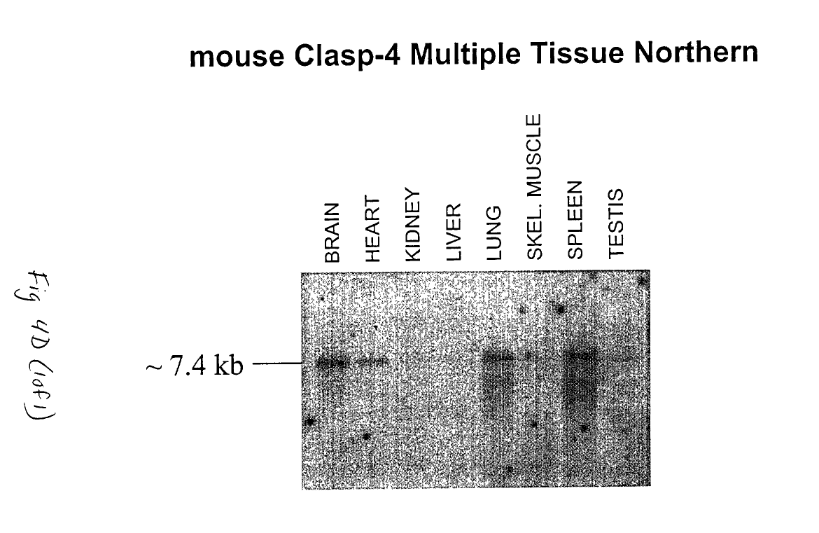

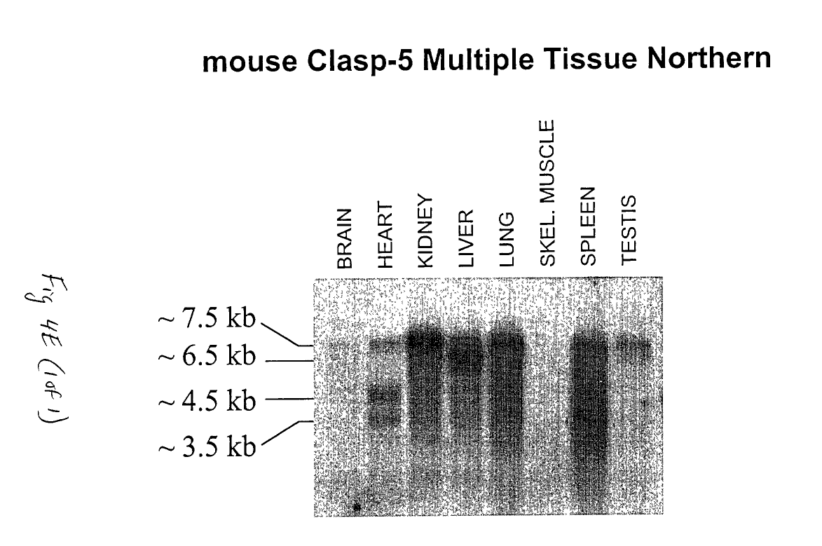

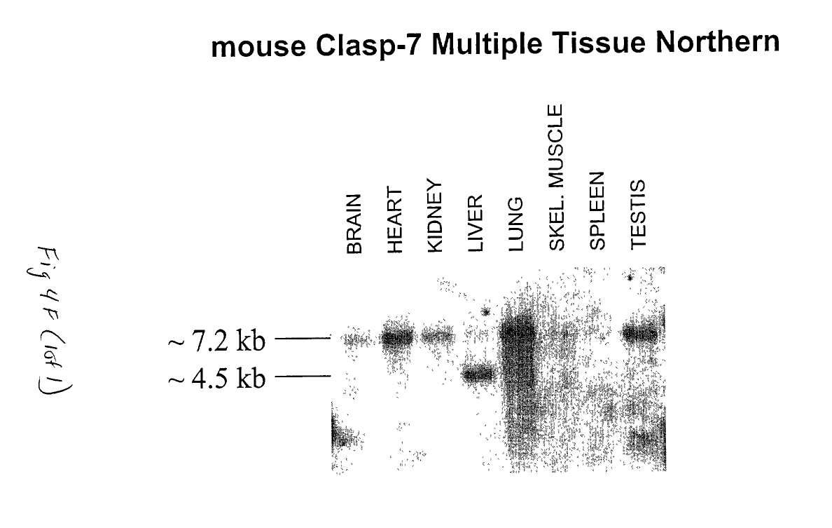

As described in Example 2, CLASP-mRNA expression was assayed in tissues and cell lines by Northern analysis. The results are shown in FIG. 4. The results of Northern Analysis of CLASP-expression and expression of other members of the CLASP family are summarized in Table 2.

TABLE 2

| | |
|---|---|
| Tissue/ | CLASP |

| Cell Line¹ | 1 | 2³ | 3 | 4 | 5 | 7 |
|---|---|---|---|---|---|---|
| PBL | +² | − | − | +++ | ++ | − |
| Lung | − | + | − | − | −/+ | +++ |
| Placenta | −/+ | +++ | + | −/+ | + | + |
| Sm Intestine | −/+ | − | − | − | −/+ | + |
| Liver | −/+ | −/+ | −/+ | − | −/+ | + |
| Kidney | −/+ | + | +++ | −/+ | + | ++ |
| Spleen | ++ | − | − | −/+ | + | −/+ |
| Thymus | ++ | − | − | −/+ | + | − |
| Colon | − | − | − | − | − | − |
| Skel Muscle | − | −/+ | ++ | − | − | −/+ |
| Heart | −/+ | ++ | +++ | −/+ | − | +++ |
| Brain | +++ | −/+ | −/+ | − | − | − |
| Jurkat | ++ | ++ | ++ | + | − | − |
| MV411 | ++ | − | ++ | + | + | + |
| THP1 | ++ | − | − | − | − | −/+ |
| HL60 | − | − | − | − | −/+ | − |
| 9D10 | ++ | ++⁵ | + | + | + | + |
| 3A9 | + | −/+ | − | − | − | − |
| CH27 | + | −/+ | − | − | − | − |
| 293 | − | ++ | +++ | + | − | + |

¹Jurkat = human T cell line; MV4-11 = B myelomonocyte; 9D10 = B cell line; THP-1 = monocyte; 3A9 = mouse T cell; CH27 = mouse B cell line; HL60 = human promyelocyte; 293 = embryonic kidney epithelial cells (293)
²Table Legend (based on Northern blot results): − = no expression; −/+ = low expression; + = medium expression; ++ medium high expression; +++ high expression.
³A CLASP-2 EST (EST 815795) was identified from a bone marrow cDNA library.

As indicated in Table 2, human CLASP-2 is expressed most strongly in placenta followed by lung, kidney and heart; human CLASP-3 is expressed strongly in kidney and heart, and less strongly in placenta and skeletal muscle; human CLASP-4 is expressed exclusively in peripheral blood lymphocytes; human CLASP-5 is expressed strongly in peripheral blood leukocytes, present in placenta, kidney, spleen and thymus, and weakly in lung, small intestine and liver. It is not expressed in brain, heart, skeletal muscle and large intestine; human CLASP-7 is expressed strongly in lung, heart, liver and kidney, but not in PBL, brain or thymus.

Differences in tissue expression patterns for different CLASP proteins indicate different CLASPs have differential roles in immune function and, accordingly, can be separately targeted to achieve different functions. For example, since CLASP proteins are necessary for proper function or signaling by the T cell receptor (TCR), the tissue specific distribution of different CLASPs permits differential modulation of the immune response in different tissues. Since CLASP-2 is present in heart, blocking CLASP-2 function or expression can be useful to selectively block immune response in the heart (for example, to selectively stop immune response in the heart compartment, e.g., following cardiac transplant rejection or post-MI inflammation, without compromising immunity elsewhere. Similarly, blocking CLASP-3 can block rejection of the kidney following kidney transplant. Furthermore, by adjusting the level of inhibition, the degree of immune blockage versus response can be modulated in the compartments represented by each CLASP.

5.3.2 Mouse CLASP mRNA Expression

As described in Example 2, mouse CLASP mRNA expression was assayed in tissues by Northern analysis. The results are shown in FIG. 4. The results of Northern Analysis of the mouse CLASP family are summarized in Table 3.

TABLE 3

Mouse CLASP

| Tissue | 1 | 2 | 3 | 4 | 5 | 7 |
|---|---|---|---|---|---|---|
| Brain | +++ | +++ | +++ | +++ | + | + |
| Heart | ++ | ++ | ++ | ++ | ++ | ++ |
| Kidney | ++ | ++++ | +++ | + | ++++ | + |
| Liver | + | + | ++ | + | ++++ | ++ |
| Lung | +++ | ++++ | +++ | +++ | ++++ | ++++ |
| Skel. Muscle | + | + | + | + | + | + |
| Spleen | +++ | | + | ++++ | ++++ | + |
| Testis | | + | ++++ | + | ++ | +++ |

Relative levels of mouse CLASP expression in multiple mouse tissues

5.3.2.1 Mouse CLASP Expression in Lymphocyte Subpopulations

TABLE 4

| | | | | monocyte |
|---|---|---|---|---|
| CLASP | CD4 | CD8 | B cell | granulocyte |
| 1 | + | + | + | + |
| 2 | − | − | − | − |
| 3 | + | + | − | + |
| 4 | + | + | − | + |
| 5 | + | + | + | + |
| 7 | − | − | + | + |

Differences in tissue expression patterns for different CLASPs indicate different CLASPs have differential roles in immune function and, accordingly, can be separately targeted to achieve different functions. For example, since CLASP proteins are necessary for proper function or signaling by the T cell receptor (TCR), the tissue specific distribution of different CLASPs permits differential modulation of the immune response in different tissues. Since mouse CLASPs are present in heart, blocking mouse CLASP function or expression is useful to selectively block immune response in the heart (for example, to selectively stop immune response in the heart compartment, e.g., following cardiac transplant rejection or post-MI inflammation, without compromising immunity elsewhere. Furthermore, by adjusting the level of inhibition, the degree of immune blockage versus response can be modulated in the compartments represented by each CLASP.

5.4. CLASP Polynucleotides and Methods of Use

The present invention provides a variety of CLASP polynucleotides and methods for using them. In one aspect, the polynucleotide of the invention encodes a polypeptide comprising at least a fragment (e.g., an immunogenic fragment) of a CLASP protein or variant thereof. In another aspect, the molecules that comprise a CLASP polynucleotide that, while not necessarily encoding a CLASP protein or fragment, is useful as a probe or primer for detecting CLASP expression, for inhibition of CLASP expression (e.g., antisense or ribozyme-mediated inhibition), for gene knockout, transgenics, other genetically modifications and the like.

5.4.1. CLASP Polynucleotides

The invention also provides isolated or purified nucleic acids having at least 8 nucleotides (i.e., a hybridizable portion) of a CLASP sequence or its complement; in other embodiments, the nucleic acids consist of at least about 25 (continuous) nucleotides, about 50 nucleotides, about 100 nucleotides, about 150 nucleotides, about 200 nucleotides, about 250 nucleotides, about 500 nucleotides, about 550 nucleotides, about 600 nucleotides, or about 650 nucleotides or more of a CLASP sequence, or a full-length CLASP coding sequence. In another embodiment, the nucleic acids are smaller than about 35, about 200 or about 500 nucleotides in length. Polynucleotides can be single or double stranded, and may be DNA, RNA, PNA or a hybrid molecule.

In specific aspects, nucleic acids are provided which comprise a sequence complementary to at least about 10, 25, 50, 100, 150, 200, 250, 500, 550, 600, or 650 nucleotides or the entire coding region of a CLASP coding sequence. Usually, the isolated polynucleotide is less than about 100 kbp, generally less than about 50 kbp, and often less than about 20 kbp, less than about 10 kbp, less than about 5 kbp, or less than about 1000 nucleotides in length.

In a specific embodiment, a nucleic acid that is hybridizable to a CLASP nucleic acid or its complement, or to a nucleic acid encoding a CLASP derivative, under conditions of low stringency is provided. Derivatives of CLASPs contemplated include, but are not limited to, splice variants of a gene encoding a CLASP, other members of a CLASP gene family which differ from one of the CLASP nucleotide or amino acid sequences disclosed herein by the insertion or deletion of one or several domains, and the like.

In one embodiment, a CLASP polynucleotide is identical or exactly complementary to its respective sequence shown in FIG. 1 or FIG. 3 or selectively hybridizes to an aforementioned sequences. In various embodiments, the polynucleotide is identical or exactly complementary to, or selectively hybridizes to, the nucleotide sequence encoding a particular protein domain or region, or a particular gene exon of the CLASP mRNA or genomic sequence. Such polynucleotides are particularly useful as probes, because they can be selected to identify a defined species of CLASP.

In addition to the polypeptide and polynucleotide sequences specifically exemplified herein, the invention contemplates CLASP homologues from other species, allelic and splice variants, and other variants disclosed herein. The CLASP gene exhibits evidence of alternative splicing of transcripts.

5.4.1.1. Substantial Identity

In some embodiments, the CLASP polynucleotides of the invention are substantially identical to the sequences shown in FIG. 1, FIG. 3, FIG. 6A or to fragments thereof.

An indication that two nucleic acid sequences are substantially identical is that the two polynucleotides have a specified percentage sequence identity e.g., usually at least about 75%, at least about 80%, at least about 85%, at least about 90%, at least about 95%, or at least about 98 identity over a specified region when optimally aligned.

Another indication that two nucleic acid sequences are substantially identical is that a polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

Yet another indication that two nucleic acid sequences are substantially identical (e.g., a naturally occurring allele of the CLASP sequence of SEQ ID NO: ______) is that the same primers can be used to amplify the sequence. For example, CLASP polynucleotides can be PCR amplified from cDNA derived from mouse lymphocytes using the primer pairs shown in Example 2.

5.4.1.2. Selective Hybridization

The invention also relates to nucleic acids that selectively hybridize to exemplified CLASP sequences (including hybridizing to the exact complements of these sequences). Selective hybridization can occur under conditions of high stringency (also called “stringent hybridization conditions”), moderate stringency, or low stringency.

5.4.1.2.1. High Stringency

“Stringent hybridization conditions” are conditions under which a probe will hybridize to its target subsequence, typically in a complex mixture of nucleic acid, but not to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. In a specific embodiment, a nucleic acid which is hybridizable to a CLASP nucleic acid under the following conditions of high stringency is provided: Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 8-16 h at 65° C. in prehybridization mixture containing 100 μg/ml denatured salmon sperm DNA and 5-20×10⁶ cpm of ³²P-labeled probe. Washing of filters is done at 65° C. for 15-30 h in a solution containing 2×SSC, 0.1% SDS. This is followed by a wash in 0.2×SSC and 0.1% at 50° C. for 15-30 min before autoradiography.

5.4.1.2.2. Moderate Stringency

In another specific embodiment, a nucleic acid, which is hybridizable to a CLASP nucleic acid under conditions of moderate stringency is provided. Examples of procedures using such conditions of moderate stringency are as follows: Filters containing DNA are pretreated for 6 h at 55° C. in a solution containing 6×SSC, 5×Denhart's solution, 0.5% SDS and 100 μg/ml denatured salmon sperm DNA. Hybridizations are carried out in the same solution and 5-20×10⁶ cpm ³²P-labeled probe is used. Filters are incubated in hybridization mixture for 12-16 h at 55° C., and then washed twice for 30 minutes at 50° C. in a solution containing 1×SSC and 0.1% SDS. Filters are blotted dry and exposed for autoradiography. Other conditions of moderate stringency which can be used are well-known in the art. Washing of filters is done at 45° C. for 1 h in a solution containing 0.2×SSC and 0.1% SDS.

5.4.1.2.3. Low Stringency

By way of example and not limitation, procedures using such conditions of low stringency are as follows (see also Shilo and Weinberg, 1981, Proc. Natl. Acad. Sci. U.S.A. 78: 6789-6792): Filters containing DNA are pretreated for 6 h at 40 C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 g/ml salmon sperm DNA, 10% (wt/vol) dextran sulfate, and 5-20×10⁶ cpm ³²P-labeled probe is used. Filters are incubated in hybridization mixture for 18-20 h at 40 C., and then washed for 1.5 h at 55 C. in a solution containing 2×SSC and 0.1% SDS. The wash solution is replaced with fresh solution and incubated an additional 30 minutes at 50-55° C. Filters are blotted dry and exposed for autoradiography. If necessary, filters are washed for a third time at 60-65° C. and reexposed to film. Other conditions of low stringency that can be used are well known in the art (e.g., as employed for cross-species hybridizations).

5.4.1.3. CLASP Variants and Fragments

The CLASP variants of the invention can contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. CLASP polynucleotide variants can be produced for a variety of reasons, e.g., to optimize codon expression for a particular host (change codons in the human mRNA to those preferred by a bacterial host such as E. coli).

Exemplary CLASP polynucleotide fragments are preferably at least about 15 nucleotides, and more preferably at least about 20 nucleotides, still more preferably at least about 30 nucleotides, and even more preferably, at least about 40 nucleotides in length, or larger 50, 150, 200, 250, 300, 350, 400, 450, 500, 550, 600, 650 nucleotides. Exemplary fragments include fragments having at least a sequence from about nucleotide number 1-50, 51-100, 101-150, 151-200, 201-250, 251-300, 301-350, 351-400, 401-450, 451-500, 501-550, 551-600 to the end of the sequences shown in FIG. 1 or FIG. 3 or comprising the cDNA coding sequence in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

Using known methods of protein engineering and recombinant DNA technology, variants can be generated to improve or alter the characteristics of the CLASP polypeptides. For instance, one or more amino acids can be deleted from the N-terminus or C-terminus of a CLASP protein without substantial loss of biological function.

Furthermore, even if deleting one or more amino acids from the N-terminus or C-terminus of a polypeptide results in modification or loss of one or more biological functions, other biological activities can still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking Nor C-terminal residues of a protein retains such immunogenic activities can readily be determined by routine methods described herein and otherwise known in the art.

Thus, the invention further includes CLASP polypeptide variants which show biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U. et al., Science 247: 1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at 30 specific positions of a cloned gene to identify regions critical for protein function. For example., site directed mutagenesis or alanine-scanning mutagenesis (introduction of single alanine mutations at every residue in the molecule) can be used. (Cunningham and Wells, 1989, Science 244: 1081-1085) The resulting mutant molecules can then be tested for biological activity.

In various embodiments, CLASP polynucleotide fragments include coding regions for, or regions hybridizable to, the CLASP structural or functional domains described supra. As set out in the Figures, such preferred regions include the following domains/motifs: DOCK, COILED/COILED, and PBM. Thus, for example, polypeptide fragments of a CLASP polynucleotide fragment falling within conserved domains are specifically contemplated by the present invention. Moreover, polynucleotide fragments encoding these domains are also contemplated. Such polypeptide fragments find use, for example, as inhibitors of CLASP function in CLASP-expressing cells.

5.4.2. Uses of CLASP Polynucleotides

The CLASP polynucleotides of the invention are useful in a variety of applications. In one aspect of the invention, the polypeptide-encoding CLASP polynucleotides of the invention are used to express CLASP polypeptides (e.g., as described herein) for example to produce anti-CLASP-antibodies or for use as therapeutic polypeptides. In another aspect, the CLASP polynucleotide or fragments thereof can be used for diagnostic purposes (e.g., as probes for CLASP expression). In particular, since CLASPs can be expressed in lymphocytes, a CLASP polynucleotide can be used to detect the expression of CLASP as a lymphocyte marker. For diagnostic purposes, a CLASP polynucleotide can be used to detect CLASP gene expression or aberrant CLASP gene expression in disease states. In another aspect, the CLASP polynucleotidse or fragments are used for therapeutic purposes. For example, included in the scope of the invention are methods for inhibiting CLASP expression, e.g., using oligonucleotide sequences, such as antisense RNA and DNA molecules and ribozymes, that function to inhibit expression of CLASPs. In another aspect, CLASP polynucleotides can be used to construct transgenic and knockout animals, e.g., for screening of CLASP agonists and antagonists. In another aspect, CLASP polynucleotides can be used for screening of CLASP agonists and antagonists.

5.4.2.1. Use of CLASP Polynucleotides for Detection, Diagnosis, and Treatment

The CLASP polynucleotides of the invention are useful for detection of CLASP expression in cells and in the diagnosis of diseases or disorders (e.g., immunodeficient states) resulting from aberrant expression of CLASPs in wild type, mutant or genetically modified animals. Aberrant expression of CLASP mRNA or protein means expression in lymphocytes (e.g., T lymphocytes or B lymphocytes) or other CLASP expressing cells of at least 2-fold, preferably at least 5-fold greater or less than expression in control lymphocytes obtained from a healthy subject or animal. CLASP polypeptide expression is easily measured by ELISA using anti-CLASP antibodies of the invention. CLASP mRNA expression (including expression of specific species or splice variants of CLASPs) can be measured by quantitative Northern analysis or quantitative PCR, LCR, or other methods, using the probes and primers of the invention.

In one embodiment, the assays of the present invention are amplification-based assays for detection of a CLASP gene product. In an amplification based assay, all or part of a CLASP mRNA or cDNA (hereinafter also referred to as “target”) is amplified, and the amplification product is then detected directly or indirectly. When there is no underlying gene product to act as a template, no amplification product is produced (e.g., of the expected size), or amplification is non-specific and typically there is no single amplification product. In contrast, when the underlying gene or gene product is present, the target sequence is amplified, providing an indication of the presence and/or quantity of the underlying gene or mRNA. Target amplification-based assays are well known to those of skill in the art.

The present invention provides a wide variety of primers and probes for detecting CLASP genes and gene products. Such primers and probes are sufficiently complementary to the CLASP gene or gene product to hybridize to the target nucleic acid. Primers are typically at least 6 bases in length, usually between about 10 and about 100 bases, typically between about 12 and about 50 bases, and often between about 14 and about 25 bases in length, often PCR primers of 15-30 (e.g., 18-22 nucleotides) are used. However, the length of primers can be adjusted by one skilled in the art. One of skill, having reviewed the present disclosure, will be able, using routine methods, to select primers to amplify all, or any portion, of the CLASP gene or gene product, or to distinguish between variant gene products, CLASP alleles, and the like. Single oligomers (e.g., U.S. Pat. No. 5,545,522), nested sets of oligomers, or even a degenerate pool of oligomers can be employed for amplification.

It will be appreciated that probes and primers can be selected to distinguish between species and splice variants based on the guidance of this disclosure, by targeting primers or probes to differentially used exons (or exon-exon junctions that differ between variants).

Methods can include the steps of collecting a sample of cells from a patient or animal, isolating nucleic acid (e.g., genomic, mRNA or both) from the cells of the sample, contacting the nucleic acid sample with one or more primers which specifically hybridize to a CLASP gene under conditions such that hybridization and amplification of the CLASP-gene (if present) occurs, and detecting the presence or absence of an amplification product, or detecting the size of the amplification product and comparing the length to a control sample. See U.S. Pat. Nos. 4,683,195 and 4,683,202, Landegran et al., 1988, Science 241: 1077-1080; Nakazawa et al., 1994, Proc. Natl. Acad. Sci. U.S.A. 91: 360-364, Abravaya et al., 1995, Nucleic Acids Res. 23: 675-682).

Because CLASP gene products are expressed in the immune system (e.g., T lymphocytes, B lymphocytes and macrophages), expression will be typically assayed in these cells. Methods which are well known to those skilled in the art can be used to isolate lymphocytes, macrophages, and alike (See, e.g., Coligan, J. E., et al. (eds.), 1991, Current Protocols in Immunology, John Wiley & Sons, NY; this reference is incorporated by reference for all purposes). In one embodiment, assays are carried out on biopsy or autopsy-derived tissue.

In various embodiments, CLASP gene expression is detected by hybridization of a detectable probe to mRNA or cDNA obtained from cells (e.g., lymphocytes). A variety of methods for specific DNA and RNA measurement using nucleic acid hybridization techniques are known to those of skill in the art (see Sambrook et al., supra). Hybridization based assays refer to assays in which a probe nucleic acid is hybridized to a target nucleic acid, forming a hybridization complex. Usually the nucleic acid hybridization probes of the invention are entirely or substantially identical to a contiguous sequence of the CLASP gene or RNA sequence. Preferably, nucleic acid probes are at least about 50 bases, often at least about 20 bases, and sometimes at least about 200 bases, at least about 300-500 nucleotides or more in length. Various hybridization techniques are well known in the art, and are in fact the basis of many commercially available diagnostic kits.

Methods of selecting nucleic acid probe sequences for use in nucleic acid hybridization are discussed in Sambrook et al., supra. In some formats, at least one of the target and probe is immobilized. The immobilized nucleic acid can be DNA, RNA, or another oligo- or poly-nucleotide, and can comprise natural or non-naturally occurring nucleotides, nucleotide analogs, or backbones. Such assays can be in any of several formats including: Southern, Northern, dot and slot blots, high-density polynucleotide or oligonucleotide arrays (e.g., GeneChips™ Affymetrix), dip sticks, pins, chips, or beads. All of these techniques are well known in the art and are the basis of many commercially available diagnostic kits. Hybridization techniques are generally described in Hames et al., ed., 1985, Nucleic Acid Hybridization, A Practical Approach IRL Press; Gall and Pardue, 1969, Proc. Natl. Acad. Sci. U.S.A., 63: 378-383; and John et al., 1969, Nature, 223: 582-587.

A variety of nucleic acid hybridization formats are known to those skilled in the art. For example, one common format is direct hybridization, in which a target nucleic acid is hybridized to a labeled, complementary probe. Typically, labeled nucleic acids are used for hybridization, with the label providing the detectable signal. One method for evaluating the presence, absence, or quantity of CLASP mRNA is carrying out a Northern transfer of RNA from a sample and hybridization of a labeled CLASP specific nucleic acid probe. A useful method for evaluating the presence, absence, or quantity of DNA encoding CLASP proteins in a sample involves a Southern blot of DNA from a sample and hybridization of a labeled specific CLASP nucleic acid probe.

Other common hybridization formats include sandwich assays and competition or displacement assays. Sandwich assays are commercially useful hybridization assays for detecting or isolating nucleic acid sequences. Such assays utilize a “capture” nucleic acid covalently immobilized to a solid support and a labeled “signal” nucleic acid in solution. The biological or clinical sample will provide the target nucleic acid. The “capture” nucleic acid and “signal” nucleic acid probe hybridize with the target nucleic acid to form a “sandwich” hybridization complex. To be effective, the signal nucleic acid cannot hybridize with the capture nucleic acid.

In one embodiment, CLASP polypeptides or polynucleotides are useful in treating deficiencies or disorders of the immune system in animals and animal model systems, by activating or inhibiting the activation, differentiation of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders can be genetic, somatic, such as cancer or some autoimmune disorders, acquired (e.g., by chemotherapy or toxins), or infectious.

In another embodiment, CLASP polynucleotides or polypeptides are useful in treating or detecting deficiencies or disorders of hematopoietic cells in animals and animal model systems. CLASP polypeptides or polynucleotides could be used to increase differentiation and proliferation of hematopoietic cells, including the pluripotent stem cells, in an effort to treat those disorders associated with a decrease in certain (or many) types hematopoietic cells in animals and animal model systems. Examples of immunologic deficiency syndromes include, but are not limited to: blood protein disorders (e.g., agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

In one embodiment, CLASP polynucleotides or polypeptides are useful in treating or detecting autoimmune diseases. The term “autoimmune disease” as used herein has the normal meaning in the art and refers to a spontaneous or induced malfunction of the immune system of mammals in which the immune system fails to distinguish between foreign immunogenic substances within the mammal and/or autologous (“self”) substances and, as a result, treats autologous (“self”) tissues and substances as if they were foreign and mounts an immune response against them. Autoimmune disease is characterized by production of either antibodies that react with self tissue, and/or the activation of immune effector T cells that are autoreactive to endogenous self antigens. Three main immunopathologic mechanisms act to mediate autoimmune diseases: 1) autoantibodies are directed against functional cellular receptors or other cell surface molecules, and either stimulate or inhibit specialized cellular function with or without destruction of cells or tissues; 2) autoantigen—autoantibody immune complexes form in intercellular fluids or in the general circulation and ultimately mediate tissue damage; and 3) lymphocytes produce tissue lesions by release of cytokines or by attracting other destructive inflammatory cell types to the lesions. These inflammatory cells in turn lead to production of lipid mediators and cytokines with associated inflammatory disease.

Since many autoimmune disorders result from inappropriate recognition of self as foreign material by immune cells. This inappropriate recognition results in an immune response leading to the destruction of the host tissue. Therefore, the administration of CLASP polypeptides or polynucleotides that can inhibit an immune response, particularly the proliferation, or differentiation of T-cells, can be an effective therapy in preventing autoimmune disorders.

Examples of autoimmune disorders that can be treated or detected by CLASPs include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems in animal and animal model systems, can also be treated by CLASP polypeptides or polynucleotides. Moreover, CLASPs can be used to treat anaphylaxis or hypersensitivity to an antigenic molecules.

In one embodiment CLASP polynucleotides or polypeptides are used to treat and/or prevent organ rejection or graft-versus-host disease (GVHD) in animals and animal model systems. Organ rejection occurs by host immune cell destruction of the transplanted tissue through an immune response. Similarly, an immune response is also involved in GVHD, but, in this case, the foreign transplanted immune cells destroy the host tissues. The administration of CLASP polypeptides or polynucleotides that inhibits an immune response, particularly the proliferation, differentiation of T-cells, can be an effective therapy in preventing organ rejection or GVHD.

Similarly, in another embodiment, CLASP polypeptides or polynucleotides are used to modulate inflammation. The term “inflammation” refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils.

For example, CLASP polypeptides or polynucleotides can inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.). Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

In another embodiment CLASP polypeptides or polynucleotides are used to treat or detect infectious agents. For example, by increasing the immune response, particularly increasing the proliferation and differentiation of B and/or T cells, infectious diseases can be treated. The immune response can be increased by either enhancing an existing immune response, or by initiating a new immune response. CLASPs polypeptides or polynucleotides can be used to treat or detect any of these symptoms or diseases.

5.4.2.2. Use of CLASP Polynucleotides in Screening

The presence or absence of CLASP nucleotide and amino acid sequences in a biological sample can be used in screening assays as medical diagnostics to aid in clinical decision-making.

In one embodiment, CLASP-based diagnostics involve screening assays for vaginal bleeding of unknown cause. In several examples discussed below, the cause of the bleeding can be in part differentiated by knowledge of whether the vaginal bleeding contains placental components (Hart F D, Ed., 1985, French's Index of Differential Diagnosis, 12th Ed. John Wright & Sons, pp. 561-63). In these cases, the high expression of hCLASP-1 nucleotide sequences in placenta relative to its low expression in blood (Table 2) will allow the detection of the presence of placenta based on the presence of the hCLASP-1 nucleotide or protein. Such detection can be achieved by quantitative RT-PCR, Northern analysis, Western analysis, ELISAs, and fluorescence activated cell sorting (FACS) by using labeled anti-hCLASP-1 antibodies (Sambrook et al., 1989, Molecular Cloning, 2nd Ed., Cold Spring Harbor Lab. Press; Harlow et. al., 1988, Antibodies, a laboratory manual, Cold Spring Harbor Lab. Press).

For example, hCLASP-1 can be used in the following screening assays:

(1) A woman gives birth and presents with post-partum bleeding. In this case the presence of placental tissue indicates a condition called “retained products of conception” that requires surgical evacuation of the uterus (Decherney and Pernol, Eds., 1996, Current Obstetric & Gynecologic Diagnosis & Treatment, 8th Ed. McGraw Hill).

(2) A pregnant woman suffers from vaginal bleeding of unknown origin. In this case the presence of placental tissue indicates a condition called “threatened abortion” that implies a poor prognosis for carrying the fetus to term (Decherney and Pernol, Eds., 1996, Current Obstetric & Gynecologic Diagnosis & Treatment, 8th Ed. McGraw Hill).

(3) A woman of child bearing age presents with vaginal bleeding and is found to have a positive pregnancy test without evidence of an intra-uterine pregnancy. In this case, the most serious of the differential diagnoses is ectopic pregnancy, a medical emergency. However, another common diagnosis is a completed abortion or miscarriage. The presence of products of conception (i.e., placenta) in the vaginal bleeding strongly favors the diagnosis of completed abortion over that of ectopic pregnancy (Decherney and Pernol, Eds., 1996, Current Obstetric & Gynecologic Diagnosis & Treatment, 8th Ed. McGraw Hill).

In another embodiment, hCLASP-1-based diagnostics involve screening assays to determine injury to vital tissues that express hCLASP-1 at high levels. Such tissues include heart, lung, kidney, liver, placenta and small intestine (Table2). Injury to these tissues can result in leakage of cells and cellular constituents including hCLASP-1 into surrounding fluids or the blood stream (specified below). Detection of high levels of hCLASP-1 protein in blood or these surrounding fluids by Western analysis or ELISA, or detection of abnormally high levels of hCLASP-1 RNA in these fluids by RT-PCR or Northern analysis is expected to aid in the diagnosis of tissue injury. The absence of hCLASP-1 in skeletal muscle and its ubiquitous presence in many internal organs makes hCLASP-1 a good serological marker for determination of general internal organ damage. Minor damage to skeletal muscle occures commonly (such as contusions) while internal organs rarely get damaged. Internal organ damage should be assessed before deciding on surgery. Typically this requires peritoneal lavage and an extensive battery of biochemical tests. hCLASP-1 can replace this invasive procedure with using an ELISA for hCLASP-1.

In another embodiment, CLASP-based diagnostics involve screening assays in animals and animal model systems to determine injury to vital tissues that express CLASPs at high levels. Such tissues include kidney, heart, and lung (FIG. 4). Injury to these tissues can result in leakage of cells and cellular constituents including CLASPs into surrounding fluids (specified below). Detection of abnormally high levels of CLASP proteins in these surrounding fluids by Western analysis or ELISA, or detection of abnormally high levels of CLASP RNAs in these fluids by RT-PCR or Northern analysis is expected to aid in the diagnosis of tissue injury.

In the case of renal injury, the CLASP nucleotide or amino acid sequences or fragments thereof would be expected to appear in the urine. Detection of abnormally high levels of CLASPs can aid in the diagnosis of both nephritis and tubular necrosis, and differentiate from non-renal causes of proteinuria. Early diagnosis of nephritis is of particular value in patients with clinical signs and symptoms suggestive of systemic lupus erythematosis in whom early diagnosis and treatment of lupus nephritis can prevent irreversible kidney damage (Cameron J. S., 1999, J Nephrol 12 Suppl 2: S29-41). While tubular necrosis currently cannot be reversed by pharmacotherapy, differentiation of tubular necrosis from pre-renal failure is critical in formulating a treatment plan for oligouric hospitalized patients (Bidani A. and Churchill P. C., 1989, Dis Mon 35: 57-132).

In the case of myocardial injury, the CLASP nucleic or amino acid sequences or fragments thereof are expected to appear in the blood. This is analogous to current standard practice of monitoring for other elevated levels myocardial proteins (e.g., creatine kinase, troponin) in the blood following myocardial infarction and ischemia by standard ELISA or electrophoretic methodologies (Fauci et al., (eds.), 1998, Harrison's Principles of Internal Medicine, 14th Ed., McGraw Hill, pp. 1352-1375). The presence of CLASPs in cardiac muscle and its absence in skeletal muscle and blood makes CLASPs an ideal marker to diagnose and monitor myocardial injury.

Unlike myocardial injury, pulmonary injury is not routinely diagnosed by assaying serum for lung-specific proteins. By analogy to myocardial infarction, pulmonary infarction also releases lung-specific proteins and cells into systemic circulation. Pulmonary infarction due to pulmonary embolism (PE) or pneumonia is expected to release CLASP-bearing cells or protein/peptides into systemic circulation. Detection of CLASP proteins in serum or RNA in blood can aid in the diagnosis of pulmonary infarction in the appropriate clinical setting. Current methods to diagnose PE are not only expensive but lack specificity and sensitivity, and the misdiagnosis of this condition is a leading cause of preventable death in hospitalized patients (Raskob G. E. and Hull R. D., 1999, Curr Opin Hematol. 6(5): 280-4).

In another embodiment, CLASP-based diagnostics involve screening assays in animal and animal model systems for identifying disorders of cells of hematopoietic lineage. Different isoforms in T and B cells permit further discrimination between malignancies of T and B lineage (FIG. 4). Precise identification of hematopoietic cell types is vital to guide chemotherapy and radiation therapy of patients with leukemia and lymphoma (Fauci et al Eds., 1998, Harrison's Principles of Internal Medicine, 14th Ed. McGraw Hill, pp. 695-712). CLASP expression differences can be detected by using FACS, immunofluorescence, immunoperoxidase staining, RT-PCR, in situ hybridization or RNA blot analysis (Sambrook, Fritsch and Maniatas, Molecular Cloning, 2nd Ed. Cold Spring Harbor Lab. Press, 1989; Ward M S, Pathology 1999 November; 31(4): 382-92).

In another embodiment, CLASP-based diagnostics involve screening assays for identifying activated immune system cells. It is known that the surface expression of CLASP-1 protein is altered during the process of lymphocyte activation. Subtyping lymphocytes specific for a particular antigen, for example, using MHC-based multimeric staining reagents (Altman et. al., 1996, Science 274: 94-6), for separating cell populations into CLASP high and CLASP low populations, can aid in determining the nature of the immune response against that antigen. Such understanding is critical, for example, in predicting the course of chronic viral infections such as hepatitis B, hepatitis C, and HIV, and to designing appropriate treatment regimens for patients suffering from these infections.

5.4.2.2.1. CLASP Antisense, Ribozyme and Triplex Polynucleotides and Methods of Use

Oligonucleotide sequences, that include anti-sense RNA and DNA molecules and ribozymes that function to inhibit the translation of a CLASP mRNA are within the scope of the invention. Such molecules are useful in cases where downregulation of CLASP expression is desired. Anti-sense RNA and DNA molecules act to directly block the translation of mRNA by binding to targeted mRNA and preventing protein translation. The invention provides methods and antisense oligonucleotide or polynucleotide reagents which can be used to reduce expression of CLASP gene products in vitro or in vivo. Administration of the antisense reagents of the invention to a target cell results in reduced CLASP activity. As will be apparent to one of skill and as discussed supra (FIG. 2 and FIG. 6B), specific CLASP splice variants can be specifically targeted for inhibition. Alternatively, by designing an, e.g, antisense molecule that recognizes a sequence found in several or all CLASP species, a general inhibition can be achieved.

A. Antisense

Without intending to be limited to any particular mechanism, it is believed that antisense oligonucleotides bind to, and interfere with the translation of, the sense CLASP mRNAs. Alternatively, the antisense molecule can render the CLASP mRNAs susceptible to nuclease digestion, interfere with transcription, interfere with processing, localization or otherwise with RNA precursors (“pre-mRNA”), repress transcription of mRNA from the CLASP genes, or act through some other mechanism. However, the particular mechanism by which the antisense molecule reduces CLASP expression is not critical.

The antisense polynucleotides of the invention comprise an antisense sequence of at least 7 to 10 to typically 20 or more nucleotides that specifically hybridize to a sequence from mRNA encoding CLASPs or mRNA transcribed from the CLASP genes. More often, the antisense polynucleotide of the invention is from about 10 to about 50 nucleotides in length or from about 14 to about 35 nucleotides in length. In other embodiments, antisense polynucleotides are polynucleotides of less than about 100 nucleotides or less than about 200 nucleotides. In general, the antisense polynucleotide should be long enough to form a stable duplex but short enough, depending on the mode of delivery, to administer in vivo, if desired. The minimum length of a polynucleotide required for specific hybridization to a target sequence depends on several factors, such as G/C content, positioning of mismatched bases (if any), degree of uniqueness of the sequence as compared to the population of target polynucleotides, and chemical nature of the polynucleotide (e.g., methylphosphonate backbone, peptide nucleic acid, phosphorothioate), among other factors. Generally, to assure specific hybridization, the antisense sequence is substantially complementary to the target CLASP mRNA sequence. In certain embodiments, the antisense sequence is exactly complementary to the target sequence. The antisense polynucleotides can also include, however, nucleotide substitutions, additions, deletions, transitions, transpositions, or modifications, or other nucleic acid sequences or non-nucleic acid moieties so long as specific binding to the relevant target sequence corresponding to CLASP RNA or its gene is retained as a functional property of the polynucleotide.

It will be appreciated that the CLASP polynucleotides and oligonucleotides of the invention can be made using nonstandard bases (e.g., other than adenine, cytidine, guanine, thymine, and uridine) or nonstandard backbone structures to provides desirable properties (e.g., increased nuclease-resistance, tighter-binding, stability or a desired TM). Techniques for rendering oligonucleotides nuclease-resistant include those described in PCT publication WO 94/12633. A wide variety of useful modified oligonucleotides may be produced, including oligonucleotides having a peptide-nucleic acid (PNA) backbone (Nielsen et al., 1991, Science 254: 1497) or incorporating 2′-O-methyl ribonucleotides, phosphorothioate nucleotides, methyl phosphonate nucleotides, phosphotriester nucleotides, phosphorothioate nucleotides, phosphoramidates. Still other useful oligonucleotides may contain alkyl and halogen-substituted sugar moieties comprising one of the following at the 2′ position: OH, SH, SCH3, F, OCN, OCH3OCH3, OCH3O(CH2)nCH3, O(CH2)nNH2 or O(CH2)nCH3, where n is from 1 to about 10; C1 to C10 lower alkyl, substituted lower alkyl, alkaryl or aralkyl; Cl; Br; CN; CF3; OCF3; O—, S—, or N-alkyl; O—, S—, or N-alkenyl; SOCH3; SO2CH3; ONO2; NO2; N3; NH2; heterocycloalkyl; heterocycloalkaryl; aminoalkylamino; polyalkylamino; substituted silyl; an RNA cleaving group; a cholesteryl group; a folate group; a reporter group; an intercalator; a group for improving the pharmacokinetic properties of an oligonucleotide; or a group for improving the pharmacodynamic properties of an oligonucleotide and other substituents having similar properties. Folate, cholesterol or other groups that facilitate oligonucleotide uptake, such as lipid analogs, may be conjugated directly or via a linker at the 2′ position of any nucleoside or at the 3′ or 5′ position of the 3′-terminal or 5′-terminal nucleoside, respectively. One or more such conjugates may be used. Oligonucleotides may also have sugar mimetics such as cyclobutyls in place of the pentofuranosyl group. Other embodiments may include at least one modified base form or “universal base” such as inosine, or inclusion of other nonstandard bases such as queosine and wybutosine as well as acetyl-, methyl-, thio- and similarly modified forms of adenine, cytidine, guanine, thymine, and uridine which are not as easily recognized by endogenous endonucleases. The antisense oligonucleotide can comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The invention further provides oligonucleotides having backbone analogues such as phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, chiral-methyl phosphonates, nucleotides with short chain alkyl or cycloalkyl intersugar linkages, short chain heteroatomic or heterocyclic intersugar (“backbone”) linkages, or CH2—NH—O—CH2, CH2—N(CH3)—OCH2, CH2—O—N(CH3)—CH2, CH2—N(CH3)—N(CH3)—CH2 and O—N(CH3)—CH2—CH2 backbones (where phosphodiester is O—P—O—CH2), or mixtures of the same. Also useful are oligonucleotides having morpholino backbone structures (U.S. Pat. No. 5,034,506).

Useful references include Oligonucleotides and Analogues, A Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan et al., Jul. 9 1993, J. Med. Chem. 36(14): 1923-1937; Antisense Research and Applications (1993, CRC Press), in its entirety and specifically Chapter 15, by Sanghvi, entitled “Heterocyclic base modifications in nucleic acids and their applications in antisense oligonucleotides;” and Antisense Therapeutics, ed. Sudhir Agrawal (Humana Press, Totowa, N.J., 1996).

In one embodiment, the antisense sequence is complementary to relatively accessible sequences of the CLASP mRNA (e.g., relatively devoid of secondary structure). This can be determined by analyzing predicted RNA secondary structures using, for example, the MFOLD program (Genetics Computer Group, Madison Wis.) and testing in vitro or in vivo as is known in the art. Another useful method for identifying effective antisense compositions uses combinatorial arrays of oligonucleotides (see, e.g., Milner et al., 1997, Nature Biotechnology 15:537). Examples of oligonucleotides that can be tested in cells for antisense suppression of CLASPs function are those capable of hybridizing to (i.e., substantially complementary to) the following.

Samples of the cells can also be harvested for analysis to determine the effects of CLASP antisense ODNs. Samples are harvested for RNA and analyzed by either Northern analysis or RT-PCR for the presence of CLASP mRNA. Functional consequences of CLASP antisense ODNs can be analyzed by measuring the ability of tissue culture cell lines (e.g. 2B4, CH27) to be activated. 2B4 cell lines are activated by exposure to anti-CD3 and anti-CD28 crosslinking antibodies, and CH27 cells are activated by exposure to anti-IgM crosslinking antibody or P. aeruginosa lipopolysaccharide. A hallmark of activation, calcium influx, can be measured by flow cytometry. Additionally, ELISA assays can be used to measure Interleukin-2 production from 2B4 cells and secreted IgM can be measured using standard assays from CH27.

Table 5 below shows exemplary mouse CLASP oligonucleotides for this assay:

TABLE 5

| | | | |
|---|---|---|---|
| mouse | | | |
| CLASP | Sequence 5′-3′ | length | notes/comments |

| | | | | |
|---|---|---|---|---|
| 1 | 1 | TTTCCTCGGAAGCT | 30 | Spans nucleotides −9 |
| | | CATCATATGCCTTG | | to 20 of FIG. 1A |
| | | A | | |
| 2 | 1 | AAGCTCATCATATG | 30 | Spans nucleotides − |
| | | CCTTGAAATGGAGT | | 19 to 21 of FIG. 1A |
| | | CA | | |
| 3 | 1 | CGCCTCCTCTTCCA | 30 | Spans nucleotides − |
| | | GAATTCCTTTCCTC | | 12 to 41 of FIG. 1A |
| | | GG | | |
| 4 | 2 | GGGAAGAGGAGCAT | 30 | Spans nucleotides − |
| | | CTCCCGCAGGCAGT | | 16 to 14 of FIG. 1B |
| | | CG | | |
| 5 | 2 | GTCATCATAAGGGA | 30 | Spans nucleotides −6 |
| | | AGAGGAGCATCTCC | | to 24 of FIG. 1B |
| | | CG | | |
| 6 | 2 | TTCCAGACGGCCAT | 30 | Spans nucleotides 25 |
| | | CCTGAGGCGGCAGG | | to 54 of FIG. 1B |
| | | GG | | |
| 7 | 3 | CCATTTCACTTTCT | 30 | Spans nucleotides 2 |
| | | TCAGGCACAGCTGA | | to 31 of FIG. 1C |
| | | AA | | |
| 8 | 3 | GTCTTCTGTGTAAC | 30 | Spans nucleotides 43 |
| | | TTCTTATACAATCT | | to 72 of FIG. 1C |
| | | CT | | |
| 9 | 3 | GATCATCTTGCTCA | 32 | Spans nucleotides |
| | | TCCTGGTAGCTGCT | | 198 to 229 of FIG. |
| | | GCCA | | 1C |
| 10 | 4 | GAATTTGCGCACTT | 31 | Spans nucleotides − |
| | | CGGCCATGGCAGAG | | 10 to 21 of FIG. 1D |
| | | GTG | | |
| 11 | 4 | CACTTCGGCCATGG | 32 | Spans nucleotides − |
| | | CAGAGGTGGCGGGC | | 20 to 12 of FIG. 1D |
| | | AGGT | | |
| 12 | 4 | CTGCCGTGCCTGGC | 31 | Spans nucleotides 22 |
| | | TTGCTCAGCCGCTT | | to 52 of FIG. 1D |
| | | GGT | | |
| 13 | 5 | CAAGCTGTTTAAGT | 33 | Spans nucleotides − |
| | | GTGTCATCACGAGT | | 12 to 21 of FIG. 1E |
| | | CCTTC | | |
| 14 | 5 | GTGTGTCATCACGA | 31 | Spans nucleotides − |
| | | GTCCTTCAAAGTCC | | 22 to 9 of FIG. 1E |
| | | ACT | | |
| 15 | 5 | GACCTCACCGATGA | 30 | Spans nucleotides 49 |
| | | CGACCTGCATGTGG | | to 78 of FIG. 1E |
| | | CC | | |
| 16 | 7 | CACTATAGCCTCGA | 30 | Spans nucleotides 5 |
| | | TCCTGGGGCCCCCG | | ot 34 of FIG. 1F, |
| | | AC | | Fragment 1 |
| 17 | 7 | GTCACAGGGCGCAA | 31 | Spans nucleotides |
| | | TCGCCTCAGCAGGG | | 164 to 194 of FIG. |
| | | ATG | | 1F, Fragment 1 |
| 18 | 7 | CAGGTTCTCGGGGG | 30 | Spans nucleotides |
| | | CCGGGGAGATGTCA | | 208 to 237 of FIG. |
| | | AG | | 1F, Fragment 1 |

Oligonucleotides presented in Table 5 are the anti-sense (i.e. reverse-complement) of the nucleotides they span as indicated in the last column.

Table 6 below shows exemplary human CLASP-1 oligonucleotides for this assay:

TABLE 6

| Oligo | Sequence 5′-3′ | length | notes/comments |
|---|---|---|---|
| 1 | AAACCTTCCCTCGAAAAC | 30-mer | reverse complement |
| | TCCATCGTATTGC¹ | | (i.e., antisense) |
| | | | of nucleotides −8 |
| | | | to +22 shown in |
| 2 | AGGTCTTTTCAAGTTCTT | 31-mer | reverse complement |
| | CAATGACAGTCTC | | (i.e., antisense) |
| | | | of nucleotides 42 |
| | | | to 172 shown in |
| 3 | TCTGCATCTTCAGGTACT | 33-mer | reverse complement |
| | GTTGAGTACAACGTG | | (i.e., antisense) |
| | | | of nucleotides 252 |
| | | | to 284 shown in |

1. Note that oligo #1 spans nucleotides encoding the initiator methionine, which is underlined

In some embodiments, administration of antisense oligonucleotides will result in reduction of mCLASP-mRNA expression by at least about 50%, as assessed by Northern analysis after administration of an antisense phosphorothioate oligonucleotide at a concentration of 1 μM, 5 μM, 10 μM or 20 μM.

The invention also provides an antisense polynucleotide that has sequences in addition to the antisense sequence (i e., in addition to anti-CLASP-sense sequence). In this case, the antisense sequence is contained within a polynucleotide of longer sequence. In another embodiment, the sequence of the polynucleotide consists essentially of, or is, the antisense sequence.

The antisense nucleic acids (DNA, RNA, modified, analogues, and the like) can be made using any suitable method for producing a nucleic acid, such as the chemical synthesis and recombinant methods disclosed herein. In one embodiment, for example, antisense RNA molecules of the invention can be prepared by de novo chemical synthesis or by cloning. For example, an antisense RNA that hybridizes to CLASP mRNAs can be made by inserting (ligating) a CLASP DNA sequence (or fragments thereof) in reverse orientation operably linked to a promoter in a vector (e.g., plasmid). Provided that the promoter and, preferably termination and polyadenylation signals, are properly positioned, the strand of the inserted sequence corresponding to the noncoding strand will be transcribed and act as an antisense oligonucleotide of the invention. The term “operably linked” refers to a functional linkage between a nucleic acid expression control sequence (such as a promoter or enhancer) and a second nucleic acid sequence, wherein the expression control sequence directs transcription of the nucleic acid corresponding to the second sequence.

In one embodiment, antisense DNA oligodeoxyribonucleotides derived from the translation initiation site, e.g., between −10 and +10 regions of a CLASP nucleotide sequence, are used. For general methods relating to antisense polynucleotides, see ANTISENSE RNA AND DNA, 1988, D.A. Melton, Ed., Cold Spring Harbor Laboratory, Cold Spring Harbor, N.Y.). See also, Dagle et al., 1991, Nucleic Acids Research, 19: 1805. For a review of antisense therapy, see, e.g., Uhlmann et al., 1990, Chem. Reviews, 90: 543-584.

B. Ribozyme

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Within the scope of the invention are engineered hammerhead motif ribozyme molecules that specifically and efficiently catalyze endonucleolytic cleavage of CLASP RNA sequences.

Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site can be evaluated for predicted structural features such as secondary structure that can render the oligonucleotide sequence unsuitable. The suitability of candidate targets can also be evaluated by testing their accessibility to hybridization with complementary oligonucleotides, using ribonuclease protection assays.

C. Triplex

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6): 569-584; Helene et al., 1992, Ann. N.Y. Acad. Sci., 660: 27-36; and Maher, 1992, Bioassays 14(12): 807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizable stretches of either purines or pyrimidines to be present on one strand of a duplex. Nucleotide sequences can be pyrimidine-based, which will result in TAT and CGC+ triplets across the three associated strands of the resulting triple helix. The pyrimidine-rich molecules provide base complementarily to a purine-rich region of a single strand of the duplex in a parallel orientation to that strand. In addition, nucleic acid molecules can be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

D. General

The anti-sense RNA and DNA molecules, ribozymes and triple helix molecules of the invention can be prepared by any method known in the art for the synthesis of RNA molecules. These include techniques for chemically synthesizing oligodeoxyribonucleotides well known in the art such as for example solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors which contain suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various modifications to the DNA molecules can be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

Methods for introducing polynucleotides into such cells or tissue include methods for in vitro introduction of polynucleotides such as the insertion of naked polynucleotide, i.e., by injection into tissue, the introduction of a CLASP polynucleotide in a cell ex vivo, the use of a vector such as a virus, (e.g., a retrovirus, adenovirus, adeno-associated virus, and the like), phage or plasmid, and the like or techniques such as electroporation or calcium phosphate precipitation.

5.4.2.2.2. Gene Therapy

By introducing gene sequences into cells, gene therapy can be used to treat conditions in animals and animal model systems in which the cells do not express normal CLASPs or express abnormal/inactive CLASPs. In some instances, the polynucleotide encoding a CLASP is intended to replace or act in the place of a functionally deficient endogenous gene. Alternatively, abnormal conditions characterized by overexpression can be treated using the gene therapy techniques described below.

In a specific embodiment, nucleic acids comprising a sequence encoding a CLASP protein or functional derivative thereof, are administered to promote CLASP function, by way of gene therapy. Gene therapy refers to therapy performed by the administration of a nucleic acid to a subject. In this embodiment of the invention, the nucleic acid produces its encoded protein that mediates a therapeutic effect by promoting CLASP function.

Any of the methods for gene therapy available in the art can be used according to the present invention. Exemplary methods are described below.

For general reviews of the methods of gene therapy, see, Goldspiel et al., 1993, Clinical Pharmacy 12: 488-505; Wu and Wu, 1991, Biotherapy 3: 87-95; Tolstoshev, 1993, Ann. Rev. Pharmacol. Toxicol. 32: 573-596; Mulligan, 1993, Science 260: 926-932; and Morgan and Anderson, 1993, Ann. Rev. Biochem. 62: 191-217; Can, 1993, TIBTECH 11(5): 155-215). Methods commonly known in the art of recombinant DNA technology which can be used are described in Ausubel et al., supra; and Kriegler, 1990, Gene Transfer and Expression, A Laboratory Manual, Stockton Press, NY.

In one aspect, the therapeutic composition comprises a CLASP nucleic acid that is part of an expression vector that encodes a CLASP protein or fragment or chimeric protein thereof in a suitable host. In particular, such a nucleic acid has a promoter operably linked to the CLASP coding region, said promoter being inducible or constitutive, and, optionally, tissue-specific. In another particular embodiment, a nucleic acid molecule is used in which the CLASP coding sequences and any other desired sequences are flanked by regions that promote homologous recombination at a desired site in the genome, thus providing for intrachromosomal expression of the CLASP nucleic acid (Koller and Smithies, 1989, Proc. Natl. Acad. Sci. U.S.A. 86: 8932-8935; Zijlstra et al., 1989, Nature 342: 435-438).

Delivery of the nucleic acid into a patient, animal or animal model can be either direct, in which case the patient is directly exposed to the nucleic acid or nucleic acid-carrying vector, or indirect, in which case, cells are first transformed with the nucleic acid in vitro, then transplanted into the patient. These two approaches are known, respectively, as in vivo or ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see, e.g., Wu and Wu, 1987, J. Biol. Chem. 262: 4429-4432) (which can be used to target cell types specifically expressing the receptors), and the like. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO 92/20316 dated Nov. 26, 1992; WO 93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. U.S.A. 86: 8932-8935; Zijlstra et al., 1989, Nature 342: 435-438).

In a specific embodiment, a viral vector that contains the CLASP nucleic acid is used. For example, a retroviral vector can be used (see, Miller et al., 1993, Meth. Enzymol. 217: 581-599). These retroviral vectors have been modified to delete retroviral sequences that are not necessary for packaging of the viral genome and integration into host cell DNA. The CLASP nucleic acid to be used in gene therapy is cloned into the vector, which facilitates delivery of the gene into a patient. More detail about retroviral vectors can be found in Boesen et al., 1994, Biotherapy 6: 291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., 1994, J. Clin. Invest. 93: 644-651; Kiem et al., 1994, Blood 83: 1467-1473; Salmons and Gunzberg, 1993, Human Gene Therapy 4: 129-141; and Grossman and Wilson, 1993, Curr. Opin. in Genetics and Devel. 3: 110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson 1993, Current Opinion in Genetics and Development 3: 499-503) present a review of adenovirus-based gene therapy. Bout et al., 1994, Human Gene Therapy 5: 3-10, demonstrated the use of adenovirus vectors to transfer genes to the respiratory epithelia of rhesus monkeys. Other instances of the use of adenoviruses in gene therapy can be found in Rosenfeld et al., 1991, Science 252: 431-434; Rosenfeld et al., 1992, Cell 68: 143-155; and Mastrangeli et al., 1993, J. Clin. Invest. 91: 225-234. Adeno-associated virus (AAV) has also been proposed for use in gene therapy (Walsh et al., 1993, Proc. Soc. Exp. Biol. Med. 204: 289-300.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, and the like. Numerous techniques are known in the art for the introduction of foreign genes into cells (see, e.g., Loeffler and Behr, 1993, Meth. Enzymol. 217: 599-618; Cohen et al., 1993, Meth. Enzymol. 217: 618-644; Cline, 1985, Pharmac. Ther. 29: 69-92) and can be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. In a preferred embodiment, epithelial cells are injected, e.g., subcutaneously. In another embodiment, recombinant skin cells can be applied as a skin graft onto the patient. Recombinant blood cells (e.g., hematopoietic stem or progenitor cells) are preferably administered intravenously. The amount of cells envisioned for use depends on the desired effect, patient state, and the like., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, and the like. In a preferred embodiment, the cell used for gene therapy is autologous to the patient.

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

5.4.2.3. Knockout Cells

In one aspect of the invention, endogenous target gene expression can also be reduced by inactivating or “knocking out” the target gene or its promoter using targeted homologous recombination (see, e.g., Smithies et al., 1985, Nature 317: 230-234; Thomas and Capecchi, 1987, Cell 51: 503-512; Thompson et al., 1989, Cell 5: 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (see, e.g., Thomas and Capecchi, 1987 and Thompson, 1989, supra). However, this approach can be adapted for use in humans provided the recombinant DNA constructs are directly administered or targeted to the required site in vivo using appropriate viral vectors.

5.4.2.4. Transgenic and Knockout Animals

The CLASP gene products can also be expressed in transgenic animals. Animals of any species, including, but not limited to, rats, rabbits, guinea pigs, pigs, micro-pigs, goats, sheep, and non-human primates, e.g., baboons, monkeys, and chimpanzees can be used to generate CLASP transgenic animals. The term “transgenic,” as used herein, refers to animals expressing CLASP genes sequences from a different species (e.g., rats expressing CLASPs gene sequences), as well as animals that have been genetically engineered to overexpress endogenous (i.e., same species) CLASP sequences or animals that have been genetically engineered to no longer express endogenous CLASPs gene sequences (i.e., “knock-out” animals), and their progeny.

Any technique known in the art can be used to introduce a CLASPs transgene into animals to produce the founder lines of transgenic animals. Such techniques include, but are not limited to pronuclear microinjection (Hoppe and Wagner, 1989, U.S. Pat. No. 4,873,191); retrovirus mediated gene transfer into germ lines (Van der Putten et al., 1985, Proc. Natl. Acad. Sci., U.S.A. 82: 6148-6152); gene targeting in embryonic stem cells (Thompson et al., 1989, Cell 56: 313-321); electroporation of embryos (Lo, 1983, Mol. Cell. Biol. 3: 1803-1814); and sperm-mediated gene transfer (Lavitrano et al., 1989, Cell 57: 717-723) (For a review of such techniques, see Gordon, 1989, Transgenic Animals, Intl. Rev. Cytol. 115, 171-229)

Any technique known in the art can be used to produce transgenic animal clones containing a CLASP transgene, for example, nuclear transfer into enucleated oocytes of nuclei from cultured embryonic, fetal or adult cells induced to quiescence (Campbell et al., 1996, Nature 380: 64-66; Wilmut et al., Nature 385: 810-813).

The present invention provides for transgenic animals that carry a CLASP transgene in all their cells, as well as animals that carry the transgene in some, but not all their cells, i.e., mosaic animals. The transgene can be integrated as a single transgene or in concatamers, e.g. head-to-head tandems or head-to-tail tandems. The transgene can also be selectively introduced into and activated in a particular cell type by following, for example, the teaching of Lasko et al (1992, Proc. Natl. Acad. Sci. U.S.A. 89: 6232-6236). The regulatory sequences required for such a cell-type specific activation will depend upon the particular cell type of interest, and will be apparent to those of skill in the art. When it is desired that the CLASP transgene be integrated into the chromosomal site of the endogenous CLASP gene, gene targeting is preferred. Briefly, when such a technique is to be utilized, vectors containing some nucleotide sequences homologous to the endogenous CLASP gene are designed for the purpose of integrating, via homologous recombination with chromosomal sequences, into and disrupting the function of the nucleotide sequence of the endogenous CLASP gene. The transgene can also be selectively introduced into a particular cell type, thus inactivating the endogenous CLASP gene in only that cell type, by following, for example, the teaching of Gu et al. (1994, Science 265: 103-106). The regulatory sequences required for such a cell-type specific inactivation will depend upon the particular cell type of interest, and will be apparent to those of skill in the art.

Once transgenic animals have been generated, the expression of the recombinant CLASP gene can be assayed utilizing standard techniques. Initial screening can be accomplished by Southern blot analysis or PCR techniques to analyze animal tissues to assay whether integration of the transgene has taken place. The level of mRNA expression of the transgene in the tissues of the transgenic animals can also be assessed using techniques that include, but are not limited to, Northern blot analysis of tissue samples obtained from the animal, in situ hybridization analysis, and RT-PCR (reverse transcriptase PCR). Samples of CLASP gene-expressing tissue, can also be evaluated immunocytochemically using antibodies specific for the CLASP transgene product.

5.4.2.5. Other Uses of CLASP Polynucleotides

There exists an ongoing need to identify new chromosome marking reagents. Sequences can be mapped to chromosomes by preparing PCR primers from the sequences shown in FIG. 1, FIG. 3 or FIG. 6A. These primers can be can be less than 50 nucleotides in length, generally less than 46 nucleotides, more generally less than 41 nucleotides, most generally less than 36 nucleotides, preferably less than 31 nucleotides, more preferably less than 26 nucleotides, and most preferably less than 21 nucleotides in length. The probes can also be less than 16 nucleotides, less than 13 nucleotides in length, less than 9 nucleotides in length and less than 7 nucleotides in length. Primers can be selected so that the primers do not span more than one predicted exon in the genomic DNA. These primers are then used for PCR screening of somatic cell hybrids containing individual chromosomes (i.e., chromosome 13). Only those hybrids containing the CLASP gene corresponding to the sequences shown in FIG. 1, FIG. 3 or FIG. 6A will yield an amplified fragment.

Similarly, somatic hybrids provide a rapid method of PCR mapping the polynucleotides to particular chromosomes. Precise chromosomal location of the CLASP polynucleotides can also be achieved using fluorescence in situ hybridization (FISH) of a metaphase chromosomal spread. See Verma, et al, Human Chromosomes: A Manual of Basic Techniques, Pergamon Press. NY., 1988. Once a polynucleotide has been mapped to a precise chromosomal location, the physical position of the polynucleotide can be used in linkage analysis. Linkage analysis establishes coinheritance between a chromosomal location and presentation of a particular disease. See McKusick, V., 1998, Mendelian Inheritance in Man: A Catalog of Human Genes and Genetic Disorders, 12th Ed, Johns Hopkins University Press.

The CLASP polynucleotides can be used for identifying animals from minute biological samples as DNA markers for restriction fragment length polymorphism (RFLP). An animal's genomic DNA is digested with one or more restriction enzymes, and probed on a Southern blot with a CLASP DNA markers to yield unique bands for identifying the animal.

As described above, it has demonstrated that upon sequencing of numerous independent cDNA products, single nucleotide polymorphisms (SNPs) have been discovered within human CLASP-1. These alterations and differences are presented in FIG. 5B. They represent mis-sense alterations.

If it is determined that certain SNPs are deleterious or advantageous, SNPs can be used as a diagnostic tool through SNP mapping or direct sequencing of the SNP region to determine which isoform is expressed. Additionally, the SNPs can be used as a general SNP marker for chromosomal defects such as rearrangement and translocations.

Human CLASP polynucleotides can be also be used as polymorphic markers for forensic analysis. See generally National Research Council, The Evaluation of Forensic DNA Evidence (Eds). 1996, Pollard et al., National Academy Press, Washington D.C.). The capacity to identify a distinguishing or unique set of forensic markers in an individual is useful for forensic analysis. For example, one can determine whether a blood sample from a suspect matches a blood or other tissue sample from a crime scene by determining whether the set of polymorphic forms occupying selected polymorphic sites is the same in the suspect and the sample. If the set of polymorphic markers does not match between a suspect and a sample, it can be concluded (barring experimental error) that the suspect was not the source of the sample. If the set of markers does match, one can conclude that the DNA from the suspect is consistent with that found at the crime scene. If frequencies of the polymorphic forms at the loci tested have been determined (e.g., by analysis of a suitable population of individuals), one can perform a statistical analysis to determine the probability that a match of suspect and crime scene sample would occur by chance.

To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample. The human CLASP polynucleotide sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 1 are particularly appropriate for this use as greater numbers of polymorphisms occur in the noncoding regions, making it easier to differentiate individuals using this technique. Examples of polynucleotide reagents include the human CLASP nucleotide sequences or portions thereof, e.g., fragments derived from the noncoding regions of SEQ ID NO: 1 having a length of at least 20 bases, preferably at least 25 bases, and more preferably at least 30 bases.

Human CLASP polynucleotides can also be used as reagents for paternity testing. The object of paternity testing is usually to determine whether a male is the father of a child. In most cases, the mother of the child is known and thus, the mother's contribution to the child's genotype can be traced. Paternity testing investigates whether the part of the child's genotype not attributable to the mother is consistent with that of the putative father. Paternity testing can be performed by analyzing sets of polymorphisms in the putative father and the child. Of course, the present invention can be expanded to the use of this procedure to determine if one individual is related to another. Even more broadly, the present invention can be employed to determine how related one individual is to another, for example, between races or species.

Bacterial infections are a major cause of health-related problems. However, the emergence of drug resistant bacteria is compromising the therapeutic value of the present spectrum of antibiotics. All the currently used antibiotics are small organic molecules, with certain level of structural similarity. This provides an advantage for bacteria to develop drug resistance, since they need to modify a limited number of genes in order to become resistant to a wide variety of antibiotics. The development of antibiotics with different chemical structure and targets can overcome antibiotic resistance, and provide therapeutic superiority in preventing infection by bacterial pathogens. Additionally, most antibiotics are not naturally occurring compounds and cause minor or sometimes serious side effects. For example, antibiotics used to treat TB can cause hearing loss.

The present invention provides new antibacterial agents. Certain CLASP-DNA sequences were difficult to clone and subclone (see Example 1). Bacteria harboring certain pieces of CLASP cDNA products were unable to be isolated, indicating that introduction of CLASP sequences compromised bacterial viability. There can be at least two possible reasons why the CLASP cDNA were unable to be cloned, which can reflect a variation of the well-established Modification and Restriction systems found in bacteria (reviewed in Wilson and Murray. (1991) Annu. Rev. Genet. 25:585-627; Bickle and Kruger (1993) Microbiol. Rev. 57:29-67). This well-described system is used by bacteria to prevent deleterious effects caused by the introduction of foreign DNA. Bacteria can recognize foreign DNA since it does not have the same modifications (e.g. methylation) as the native DNA. After recognition, the bacteria then digest and eliminate the foreign DNA (restriction). In the first scenario, the CLASP cDNA can be recognized as foreign DNA, and digested and eliminated as in the Modification and Restriction system. However, this would be unique for CLASP cDNA since the bacteria used for cloning cDNA are compromised in the Modification and Restriction system, which makes cloning of cDNA into bacteria a practice common in the art. If this is the case, the bacterial apparatus that specifically recognizes or eliminates CLASP cDNA can provide a novel target to develop antimicrobial agents. The CLASP DNA sequence would be useful in targeting the apparatus as well as an entry point for designing screens to identify potential targets. The second possibility is that CLASP cDNA behaves as an antimicrobial agent (i.e., antibiotic), and prevents bacterial growth. This, in effect, would create a new type of antibiotic mediated by the presence of foreign DNA (i.e. CLASP cDNA). In the case for the CLASP cDNA, the bacteria can recognize the DNA but instead of digesting and eliminating the DNA, the CLASP cDNA can cause a variation of the restriction and prevent the bacteria from growing, imposing a bacteriacidal effect upon the bacteria.

DNA as an antimicrobial agent has significant advantages over currently available agents. First, it is structurally unrelated to any existing antibiotics, and can overcome the present growing drug-resistance problem to structurally common agents. Second, since DNA antimicrobials composed of naturally-occurring human DNA, are expected to have minimal side effects and immune rejection. Third, DNA sequences can be tailored with sequence variation and numerous chemical modifications to circumvent the problem of resistance. Fourth, the antimicrobial DNA can be delivered specifically to bacterial cells through the use of bacteriophages (i.e., bacterial virus) which specifically infect bacteria and do not infect human cells. Further specificity can be generated to infect certain bacteria and bacterial subpopulations. Finally, this system can be economically robust since the generation of DNA and delivery vehicles are inexpensive.

5.5. Polypeptides Encoded by the CLASP Gene Coding Sequences

In accordance with the invention, a CLASP polynucleotide which encodes a CLASP polypeptide, mutant polypeptide, peptide fragment, CLASP fusion protein or functional equivalent thereof, can be used to express CLASP protein in appropriate host cells. In various embodiments, the CLASP polypeptides expressed will be identical or substantially similar to the sequences shown in FIG. 1, FIG. 3, FIG. 6A or fragments thereof.

In some embodiments, altered DNA sequences which can be used in accordance with the invention include deletions, additions or substitutions of different nucleotide residues resulting in a sequence that encodes the same or a functionally equivalent gene product. For example, due to the inherent degeneracy of the genetic code, other DNA sequences which encode substantially the same or a functionally equivalent amino acid sequence, can be used in the practice of the invention for the expression of the CLASP protein. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. One of skill will recognize that each codon in a nucleic acid sequence such as SEQ ID NO: ______ (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Thus, for example, due to the degeneracy of the genetic code, a polypeptide having the sequence of SEQ ID NO: ______ or a fragment thereof, can be encoded by numerous polynucleotides other than SEQ ID NO: ______. Typically, the degenerate sequence will hybridize with SEQ ID NO: ______ under high or moderate stringency conditions, but this is not strictly required (e.g., when a copy of a nucleic acid is created using the maximum codon degeneracy permitted by the genetic code. In such cased, the nucleic acids typically hybridize under moderately stringent hybridization conditions.)

The gene product itself can contain deletions, additions or substitutions of amino acid residues within a CLASP sequence, which result in a silent change thus producing a functionally equivalent CLASP protein. Such conservative amino acid substitutions can be made on the basis of similarity in polarity, charge, solubility, hydrophobicity, hydrophilicity, and/or the amphipathic nature of the residues involved. For example, negatively charged amino acids include aspartic acid and glutamic acid; positively charged amino acids include lysine, histidine and arginine; amino acids with uncharged polar head groups having similar hydrophilicity values include the following: glycine, asparagine, glutamine, serine, threonine, tyrosine; and amino acids with nonpolar head groups include alanine, valine, isoleucine, leucine, phenylalanine, proline, methionine, tryptophan. Creighton, 1984, PROTEINS, has grouped amino acids that are conservative substitutions for one another as follows: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M).

The DNA sequences of the invention can be engineered in order to alter a CLASP coding sequence for a variety of ends, including but not limited to, alterations which modify processing and expression of the gene product. For example, mutations can be introduced using techniques which are well known in the art, e.g., site-directed mutagenesis, to insert new restriction sites, to alter glycosylation patterns, phosphorylation, and the like. Based on the domain organization of the CLASP proteins, a large number of CLASP mutant polypeptides can be constructed by modifying or rearranging the nucleotide sequences that encode the CLASP extracellular, and cytoplasmic domains.

In various embodiments, the present invention provides homologues of the CLASP polypeptides which function as either a CLASP agonists or a CLASP antagonist. In a preferred embodiment, the CLASP agonists and antagonists stimulate or inhibit, respectively, a subset of the biological activities of the naturally occurring form of the CLASP polypeptide. Thus, specific biological effects can be elicited by treatment with a homologue of limited function. In one embodiment, treatment of a subject with a homologue having a subset of the biological activities of the naturally occurring form of the polypeptide has fewer side effects in a subject relative to treatment with the naturally occurring form of the CLASP polypeptide.

The invention contemplates both full-length CLASP polypeptides and fragments, e.g., fragments having a length of at least about 10, often 20, frequently 50 or 100 residues substantially identical to the exemplified CLASP polypeptide sequences of the invention. Protein fragments can be “free-standing,” or comprised within a larger polypeptide of which the fragment forms a part or region, most preferably as a single continuous region. Representative examples of polypeptide fragments of the invention, include, for example, fragments from about amino acid number 1-20, 2 1-40, 4 1-60, 61-80, 81-100, 102-120, 121-140, 141-160, 161-180, 181-200, or 201 to the end of the coding region. Moreover, polypeptide fragments can be about 20, 30, 40, 50, 60, 70, 80, 90, 100, 110, 120, 130, 140, 150, 200 amino acids in length. In this context “about” includes the particularly recited ranges, larger or smaller by several (5, 4, 3, 2, or 1) amino acids, at either extreme or at both extremes.

Preferred polypeptide fragments include the CLASP protein. Further preferred polypeptide fragments include the CLASP protein having a continuous series of deleted residues from the amino or the carboxy terminus, or both. For example, any number of amino acids, ranging from 1-X, can be deleted from the amino terminus of either the CLASP polypeptide. Furthermore, any combination of the above amino and carboxy terminus deletions are preferred. Similarly, polynucleotide fragments encoding these CLASP polypeptide fragments are also preferred.

Even if deletion of one or more amino acids from the N-terminus of a protein results in modification of loss of one or more biological functions of the protein, other biological activities can still be retained. Thus, the ability of shortened CLASP muteins to induce and/or bind to antibodies which recognize the complete or mature forms of the polypeptides generally will be retained when less than the majority of the residues of the complete or mature polypeptide are removed from the N-terminus. Whether a particular polypeptide lacking N-terminal residues of a complete polypeptide retains such immunologic activities can readily be determined by routine methods described herein and otherwise known in the art. It is not unlikely that a CLASP mutein with a large number of deleted N-terminal amino acid residues can retain some biological or immunogenic activities. In fact, peptides composed of as few as four CLASP amino acid residues can often evoke an immune response.

Homologues of the CLASP polypeptides can be generated by mutagenesis, e.g., discrete point mutation or truncation of the CLASP polypeptide. As used herein, the term “homologue” refers to a variant form of the CLASP polypeptide which acts as an agonist or antagonist of the activity of the CLASP polypeptide. An agonist of the CLASP polypeptide can retain substantially the same, or a subset, of the biological activities of the CLASPs polypeptide. An antagonist of the CLASP polypeptide can inhibit one or more of the activities of the naturally occurring form of the CLASP polypeptide, by, for example, competitively binding to a downstream or upstream member of the CLASP molecular pathway which includes the CLASP polypeptides.

Modulation can be assayed by determining any parameter that is indirectly or directly affected by the expression of the target gene. Such parameters include, e.g., changes in RNA or protein levels, changes in protein activity, changes in product levels, changes in downstream gene expression, changes in reporter gene transcription (luciferase, CAT, β-galactosidase, β-glucuronidase, GFP (see, e.g., Mistili & Spector, 1997, Nature Biotechnology 15: 961-964); changes in signal transduction, phosphorylation and dephosphorylation, receptor-ligand interactions, second messenger concentrations (e.g., cGMP, cAMP, IP₃, and Ca²⁺), and cell growth. These assays can be in vitro, in vivo, and ex vivo. Such functional effects can be measured by any means known to those skilled in the art, e.g., measurement of RNA or protein levels, measurement of RNA stability, identification of downstream or reporter gene expression, e.g., via chemiluminescence, fluorescence, colorimetric reactions, antibody binding, inducible markers, ligand binding assays; changes in intracellular second messengers such as cGMP and inositol triphosphate (IP₃); changes in intracellular calcium levels; cytokine release, and the like.

5.5.1. Synthesis or Expression of CLASPs: Polypeptide Expression Systems

In order to express a biologically active CLASPs, the nucleotide sequence coding for CLASPs, or a functional equivalent, is inserted into an appropriate expression vector. The CLASP gene products as well as host cells or cell lines transfected or transformed with recombinant CLASP expression vectors can be used for a variety of purposes. These include, but are not limited to, generating antibodies (i.e., monoclonal or polyclonal) that competitively inhibit activity of CLASP proteins and neutralize their activity; antibodies that activate CLASP function and antibodies that detect its presence on the cell surface or in solution. Anti-CLASP antibodies can be used in detecting and quantifying expression of CLASP levels in cells and tissues such as lymphocytes and macrophages, as well as isolating CLASP-positive cells from a cell mixture.

Methods which are well known to those skilled in the art can be used to construct recombinant expression vectors containing the CLASP coding sequences and appropriate transcriptional/translational control signals. These methods include in vitro recombinant DNA techniques, synthetic techniques and in vivo recombination/genetic recombination. (See, e.g., the techniques described in Sambrook et al., 1989, Molecular Cloning A Laboratory Manual, Cold Spring Harbor Laboratory, N.Y. and Ausubel et al., supra). The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. It will be appreciated by those skilled in the art that the design of the expression vector can depend on such factors as the choice of the host cell to be transformed, the level of expression of polypeptide desired, and the like. The expression vectors of the invention can be introduced into host cells to thereby produce polypeptides or peptides, including fusion polypeptides or peptides, encoded by nucleic acids as described herein (e.g., CLASP polypeptides, mutant forms of CLASP, fusion polypeptides, and the like).

A variety of host-expression vector systems can be utilized to express a CLASP coding sequences. These include, but are not limited to, microorganisms such as bacteria transformed with recombinant bacteriophage DNA, plasmid DNA, or cosmid DNA expression vectors containing the CLASP coding sequences; yeast transformed with recombinant yeast expression vectors containing the CLASP coding sequences; insect cell systems infected with recombinant virus expression vectors (e.g., baculovirus) containing the CLASP coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the CLASP coding sequences; or animal cell systems. The expression elements of these systems vary in their strength and specificities. Depending on the host/vector system utilized, any of a number of suitable transcription and translation elements, including constitutive and inducible promoters, can be used in the expression vector. For example, when cloning in bacterial systems, inducible promoters such as pL of bacteriophage λ, plac, ptrp, ptac (ptrp-lac hybrid promoter; cytomegalovirus promoter) and the like can be used; when cloning in insect cell systems, promoters such as the baculovirus polyhedron promoter can be used; when cloning in plant cell systems, promoters derived from the genome of plant cells (e.g., heat shock promoters; the promoter for the small subunit of RUBISCO; the promoter for the chlorophyll α/β binding protein) or from plant viruses (e.g., the ³⁵S RNA promoter of CaMV; the coat protein promoter of TMV) can be used; when cloning in mammalian cell systems, promoters derived from the genome of mammalian cells (e.g., metallothionein promoter) or from mammalian viruses (e.g., the adenovirus late promoter; the vaccinia virus 7.5K promoter) can be used; when generating cell lines that contain multiple copies of the CLASP DNA, SV40-, BPV- and EBV-based vectors can be used with an appropriate selectable marker.

In bacterial systems a number of expression vectors can be advantageously selected depending upon the use intended for the expressed CLASP products. For example, when large quantities of CLASP proteins are to be produced for the generation of antibodies or to screen peptide libraries, vectors which direct the expression of high levels of fusion protein products that are readily purified can be desirable. Such vectors include, but are not limited to, the E. coli expression vector pUR278 (Ruther et al., 1983, EMBO J. 2: 1791), in which the CLASP coding sequences can be ligated into the vector in frame with the lacZ coding region so that a hybrid protein is produced; pIN vectors (Inouye & Inouye, 1985, Nucleic acids Res. 13: 3101-3109; Van Heeke & Schuster, 1989, J. Biol. Chem. 264: 5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In yeast, a number of vectors containing constitutive or inducible promoters can be used. (Current Protocols in Molecular Biology, Vol. 2, 1988 (Suppl. 1999), Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II.)

In cases where plant expression vectors are used, the expression of the CLASP coding sequences can be driven by any of a number of promoters. For example, viral promoters such as the 35S RNA and 19S RNA promoters of CaMV (Brisson et al., 1984, Nature 310: 511-514), or the coat protein promoter of TMV (Takamatsu et al., 1987, EMBO J. 6: 307-311) can be used; alternatively, plant promoters such as the small subunit of RUBISCO (Coruzzi et al., 1984, EMBO J. 3: 1671-1680; Broglie et al., 1984, Science 224: 838-843); or heat shock promoters, e.g., soybean hsp17.5-E or hsp17.3-B (Gurley et al., 1986, Mol. Cell. Biol. 6: 559-565) can be used. These constructs can be introduced into plant cells using Ti plasmids, Ri plasmids, plant virus vectors, direct DNA transformation, microinjection, electroporation, and the like. (Weissbach & Weissbach, 1988, Methods for Plant Molecular Biology, Academic Press, NY., Section VIII, pp. 421-463; and Grierson & Corey, 1988, Plant Molecular Biology, 2d Ed., Blackie, London, Ch. 7-9.)

An alternative expression system which could be used to express CLASPs is an insect system. In one such system, Autographa californica nuclear polyhedrosis virus (AcNPV) is used as a vector to express foreign genes. The virus grows in Spodoptera frugiperda cells. The CLASP coding sequences can be cloned into non-essential regions (e.g., the polyhedron gene) of the virus and placed under control of an AcNPV promoter (e.g., the polyhedron promoter). Successful insertion of the CLASP coding sequences will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (see, e.g., Smith et al., 1983, J. Viol. 46: 584; Smith, U.S. Pat. No. 4,215,051).

In mammalian host cells, a number of viral based expression systems can be utilized. In cases where an adenovirus is used as an expression vector, the CLASP coding sequences can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing CLASP in infected hosts. (See, e.g., Logan & Shenk, 1984, Proc. Natl. Acad. Sci. U.S.A. 81: 3655-3659). Alternatively, the vaccinia 7.5K promoter can be used. (See, e.g., Mackett et al., 1982, Proc. Natl. Acad. Sci. U.S.A. 79: 7415-7419; Mackett et al., 1984, J. Virol. 49: 857-864; Panicali et al., 1982, Proc. Natl. Acad. Sci. U.S.A. 79: 4927-4931). Regulatable expression vectors such as the tetracycline repressible vectors can also be used to express a coding sequence in a controlled fashion.

Specific initiation signals can also be required for efficient translation of inserted CLASP coding sequences. These signals include the ATG initiation codon and adjacent sequences. In cases where the entire CLASPs gene, including its own initiation codon and adjacent sequences, is inserted into the appropriate expression vector, no additional translational control signals can be needed. However, in cases where only a portion of the CLASP coding sequences is inserted, exogenous translational control signals, including the ATG initiation codon, must be provided. Furthermore, the initiation codon must be in phase with the reading frame of the CLASP coding sequences to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, and the like. (see Bittner et al., 1987, Methods in Enzymol. 153: 516-544).

In addition, a host cell strain can be chosen which modulates the expression of the inserted sequences, or modifies and processes the gene product in a specific fashion desired. Such modifications (e.g., glycosylation) and processing (e.g., cleavage) of protein products can be important for the function of the protein. The presence of several consensus N-glycosylation sites in CLASP extracellular domains support the possibility that proper modification can play a role in CLASP function. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, WI38, and the like.

Host cells transformed with nucleotide sequences encoding CLASPs may be cultured under conditions suitable for the expression and recovery of the soluble protein from cell culture. The protein produced by a transformed cell may be secreted or contained intracellularly depending on the sequence and/or the vector used. As will be understood by those of skill in the art, expression vectors containing polynucleotides which encode CLASPs may be designed to contain signal sequences which direct secretion of CLASPs through a prokaryotic or eukaryotic cell membrane. Other constructions may be used to join sequences encoding CLASPs to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin,

For long-term, high-yield production of recombinant proteins, stable expression is preferred. For example, cell lines which stably express CLASP proteins can be engineered. Rather than using expression vectors which contain viral origins of replication, host cells can be transformed with the CLASP DNAs controlled by appropriate expression control elements (e.g., promoter, enhancer, sequences, transcription terminators, polyadenylation sites, and the like.), and a selectable marker. Following the introduction of foreign DNA, engineered cells can be allowed to grow for 1-2 days in an enriched medium, and then switched to a selective medium. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the CLASP proteins on the cell surface. Such engineered cell lines are particularly useful in screening for molecules or drugs that affect CLASP function.

A number of selection systems can be used, including but not limited to, the herpes simplex virus thymidine kinase (Wigler et al., 1977, Cell 11: 223), hypoxanthine-guanine phosphoribosyltransferase (Szybalska & Szybalski, 1962, Proc. Natl. Acad. Sci. U.S.A. 48: 2026), and adenine phosphoribosyltransferase (Lowy et al., 1980, Cell 22: 817) genes which can be employed in tk^{—}, hgprt^{—} or aprt^{—} cells, respectively. Also, antimetabolite resistance can be used as the basis of selection for dhfr, which confers resistance to methotrexate (Wigler et al., 1980, Natl. Acad. Sci. U.S.A. 77: 3567; O'Hare et al., 1981, Proc. Natl. Acad. Sci. U.S.A. 78: 1527); gpt, which confers resistance to mycophenolic acid (Mulligan & Berg, 1981), Proc. Natl. Acad. Sci. U.S.A. 78: 2072); neo, which confers resistance to the aminoglycoside G-418 (Colberre-Garapin et al., 1981, J. Mol. Biol. 150: 1); and hygro, which confers resistance to hygromycin (Santerre et al., 1984, Gene 30: 147). Additional selectable genes have been described, namely trpB, which allows cells to utilize indole in place of tryptophan; hisD, which allows cells to utilize histinol in place of histidine (Hartman & Mulligan, 1988, Proc. Natl. Acad. Sci. U.S.A. 85: 8047); ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.) and glutamine synthetase (Bebbington et al., 1992, Biotech 10: 169).

In an alternate embodiment of the invention, the coding sequence of CLASPs could be synthesized in whole or in part, using chemical methods well known in the art. (See, e.g., Caruthers et al., 1980, Nuc. Acids Res. Symp. Ser. 7: 215-233; Crea and Horn, 180, Nuc. Acids Res. 9(10): 2331; Matteucci and Caruthers, 1980, Tetrahedron Letter 21: 719; and Chow and Kempe, 1981, Nuc. Acids Res. 9(12): 2807-2817.) Alternatively, the protein itself could be produced using chemical methods to synthesize a CLASP amino acid sequence in whole or in part. For example, peptides can be synthesized by solid phase techniques, cleaved from the resin, and purified by preparative high performance liquid chromatography. (See Creighton, 1983, Proteins Structures And Molecular Principles, W. H. Freeman and Co., N.Y. pp. 50-60). The composition of the synthetic polypeptides can be confirmed by amino acid analysis or sequencing (e.g., the Edman degradation procedure; see Creighton, 1983, Proteins, Structures and Molecular Principles, W. H. Freeman and Co., N.Y., pp. 34-49).

In some embodiments, the CLASP polypeptide contains non-naturally occurring amino acids or amino acid analogs (i.e., compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium).

5.5.2. Identification of Cells that Express CLASPs

The recombinant host cells which contain the coding sequence and which express a mouse CLASP gene product or fragments thereof can be identified by at least four general approaches; (a) DNA-DNA or DNA-RNA hybridization; (b) the presence or absence of “marker” gene functions; (c) assessing the level of transcription as measured by the expression of CLASP mRNA transcripts in the host cell; and (d) detection of the gene product as measured by immunoassay or by its biological activity. Prior to the identification of gene expression, the host cells can be first mutagenized in an effort to increase the level of expression of CLASPs, especially in cell lines that produce low amounts of CLASPs.

In the first approach, the presence of the CLASP coding sequences inserted in the expression vector can be detected by DNA-DNA or DNA-RNA hybridization using probes comprising nucleotide sequences that are homologous to the CLASP coding sequences, respectively, or portions or derivatives thereof.

In the second approach, the recombinant expression vector/host system can be identified and selected based upon the presence or absence of certain “marker” gene functions (e.g., thymidine kinase activity, resistance to antibiotics, resistance to methotrexate, transformation phenotype, occlusion body formation in baculovirus, and the like). For example, if the CLASP coding sequences are inserted within a marker gene sequence of the vector, recombinants containing the CLASP coding sequences can be identified by the absence of the marker gene function. Alternatively, a marker gene can be placed in tandem with the CLASP sequences under the control of the same or different promoter used to control the expression of the CLASP coding sequences. Expression of the marker in response to induction or selection indicates expression of the CLASP coding sequences.

In the third approach, transcriptional activity for the CLASP coding regions can be assessed by hybridization assays. For example, RNA can be isolated and analyzed by Northern blot using a probe homologous to the CLASP coding sequences or particular portions thereof Alternatively, total nucleic acids of the host cell can be extracted and assayed for hybridization to such probes. Additionally, reverse transcription-polymerase chain reactions can be used to detect low levels of gene expression.

In the fourth approach, the expression of the CLASP protein products can be assessed immunologically, for example by Western blots, immunoassays such as radioimmuno-precipitation, enzyme-linked immunoassays, fluorescent activated cell sorting (“FACS”), and the like. This can be achieved by using an anti-CLASP antibodies. Alternatively, CLASP proteins can be expressed as a fusion protein with green-fluorescent protein to facilitate its detection in cells (U.S. Pat. Nos. 5,491,084; 5,804,387; 5,777,079).

5.5.3. Uses of CLASP Engineered Host Cells

In one embodiment of the invention, the CLASP proteins and/or cell lines that express CLASPs can be used to screen for antibodies, peptides, small molecules, natural and synthetic compounds or other cell bound or soluble molecules that bind to the CLASP proteins resulting in stimulation or inhibition of CLASP function. For example, anti-CLASP antibodies can be used to inhibit or stimulate CLASP function and to detect its presence. Alternatively, screening of peptide libraries with recombinantly expressed soluble CLASP proteins or cell lines expressing CLASP proteins can be useful for identification of therapeutic molecules that function by inhibiting or stimulating the biological activity of CLASPs in mammalian cells. The uses of CLASP proteins and engineered cell lines, described in the subsections below, can be employed equally well for homologous CLASP genes in various species.

In a specific embodiment of the invention, cell lines may be engineered to express the extracellular or intracellular domain of CLASP fused to another molecule such as GST. In addition, CLASP, its extracellular domain or its intracellular domain may be fused to an immunoglobulin constant region (Hollenbaugh and Aruffo, 1992, Current Protocols in Immunology, Unit 10. 19; Aruffo et al., 1990, Cell 61: 1303) to produce a soluble molecule with increased half life. The soluble protein or fusion protein can be used in binding assays, affinity chromatography, immunoprecipitation, Western blot, and the like. Synthetic compounds, natural products, and other sources of potentially biologically active materials can be screened in assays that are well known in the art.

Random peptide libraries consisting of all possible combinations of amino acids attached to a solid phase support can be used to identify peptides that are able to bind to a specific domain of the CLASPs (Lam, K. S. et al, 1991, Nature 354: 82-84). The screening of peptide libraries can have therapeutic value in the discovery of pharmaceutical agents that stimulate or inhibit the biological activity of the CLASPs.

Identification of molecules that are able to bind to the CLASP proteins can be accomplished by screening a peptide library with recombinant soluble CLASP protein. Methods for expression and purification of CLASP are described in Section 5.7, supra, and can be used to express recombinant full length CLASPs or fragments of CLASPs depending on the functional domains of interest. Such domains include CLASP extracellular domains, CLASP intracellular domains, tyrosine phosphorylation site-containing domains, cysteine cluster containing domains, and coil/coil domains

To identify and isolate the peptide/solid phase support that interacts and forms a complex with CLASPs, it is necessary to label or “tag” the CLASP molecules. The CLASP proteins can be conjugated to enzymes such as alkaline phosphatase or horseradish peroxidase or to other reagents such as fluorescent labels which can include fluorescein isothiocyanate (FITC), phycoerythrin (PE) or rhodamine. Conjugation of any given label to CLASPs can be performed using techniques that are well known in the art. Alternatively, CLASP expression vectors can be engineered to express chimeric CLASP proteins containing an epitope for which a commercially available antibody exists. The epitope-specific antibody can be tagged with a detectable label using methods well known in the art including an enzyme, a fluorescent dye or colored or magnetic beads.

The “tagged” CLASP conjugates are incubated with the random peptide library for 30 minutes to one hour at 22° C. to allow complex formation between CLASPs and peptide species within the library. The library is then washed to remove any unbound protein. If CLASPs have been conjugated to alkaline phosphatase or horseradish peroxidase the whole library is poured into a petri dish containing substrates for either alkaline phosphatase or peroxidase, for example, 5-bromo-4-chloro-3-indoyl phosphate (BCIP) or 3,3′,4,4″-diaminobenzidine (DAB), respectively. After incubating for several minutes, the peptide/solid phase-CLASP complex changes color, and can be easily identified and isolated physically under a dissecting microscope with a micromanipulator. If a fluorescent tagged CLASP molecule has been used, complexes can be isolated by fluorescence activated sorting. If a chimeric CLASP proteins expressing a heterologous epitope has been used, detection of the peptide/CLASP complexes can be accomplished by using a labeled epitope-specific antibody. Once isolated, the identity of the peptide attached to the solid phase support can be determined by peptide sequencing.

In addition to using soluble mouse CLASP molecules, in another embodiment, it is possible to detect peptides that bind to cell-associated CLASPs using intact cells. The use of intact cells is preferred for use with cell surface molecules. Methods for generating cell lines expressing CLASPs are described in Section 5.8. The cells used in this technique can be either live or fixed cells. The cells can be incubated with the random peptide library and bind to certain peptides in the library to form a “rosette” between the target cells and the relevant solid phase support/peptide. The rosette can thereafter be isolated by differential centrifugation or removed physically under a dissecting microscope. Techniques for screening combinatorial libraries are known in the art (Gallop et al, 1994, J. Med. Chem., 37: 1233; Gordon, 1994, J. Med. Chem., 37: 1385).

As an alternative to whole cell assays for membrane bound receptors or receptors that require the lipid domain of the cell membrane to be functional, CLASP molecules can be reconstituted into liposomes where label or “tag” can be attached.

5.5.4. CLASP Fusion Proteins

In another embodiment of the invention, a CLASP or a modified CLASP sequence can be ligated to a heterologous sequence to encode a fusion protein. For example, for screening of peptide libraries for molecules that bind CLASPs, it can be useful to produce a chimeric CLASPs proteins expressing a heterologous epitope that is recognized by a commercially available antibody. A fusion protein can also be engineered to contain a cleavage site located between a CLASP sequence and the heterologous protein sequence, so that the CLASP can be cleaved away from the heterologous moiety. In one embodiment, fusion proteins of the invention can contain the CLASP extracellular domain.

5.6. Cloning Alleles, Variants, and Species Homologs of CLASPs

In order to clone the full length cDNA sequence from any species encoding a CLASP cDNA, or to clone variant forms of the molecule, labeled DNA probes made from nucleic acid fragments corresponding to any partial cDNA disclosed herein can be used to screen a cDNA library derived from lymphoid cells or brain cells. More specifically, oligonucleotides corresponding to either the 5′ or 3′ terminus of the cDNA sequence can be used to obtain longer nucleotide sequences. Briefly, the library can be plated out to yield a maximum of 30,000 pfu for each 150 mm plate. Approximately 40 plates can be screened. The plates are incubated at 37° C. until the plaques reach a diameter of 0.25 mm or are just beginning to make contact with one another (3-8 hours). Nylon filters are placed onto the soft top agarose and after 60 seconds, the filters are peeled off and floated on a DNA denaturing solution consisting of 0.4N sodium hydroxide. The filters are then immersed in neutralizing solution consisting of 1M Tris-HCl, pH 7.5, before being allowed to air dry. The filters are prehybridized in hybridization buffer such as casein buffer containing 10% dextran sulfate, 0.5M NaCl, 50 mM Tris-HCl, pH 7.5, 0.1% sodium pyrophosphate, 1% casein, 1% SDS, and denatured salmon sperm DNA at 0.5 mg/ml for 6 hours at 60° C. The radiolabeled probe is then denatured by heating to 95° C. for 2 minutes and then added to the prehybridization solution containing the filters. The filters are hybridized at 60° C. for 16 hours. The filters are then washed in 1× wash mix (10× wash mix contains 3M NaCl, 0.6M Tris base, and 0.02M EDTA) twice for 5 minutes each at room temperature, then in 1× wash mix containing 1% SDS at 60° C. for 30 minutes, and finally in 0.3× wash mix containing 0.1% SDS at 60° C. for 30 minutes. The filters are then air dried and exposed to x-ray film for autoradiography. After developing, the film is aligned with the filters to select a positive plaque. If a single, isolated positive plaque cannot be obtained, the agar plug containing the plaques will be removed and placed in lambda dilution buffer containing 0.1M NaCl, 0.01M magnesium sulfate, 0.035M Tris HCl, pH 7.5, 0.01% gelatin. The phage can then be replated and rescreened to obtain single, well isolated positive plaques. Positive plaques can be isolated and the cDNA clones sequenced using primers based on the known cDNA sequence. This step can be repeated until a full length cDNA is obtained.

It can be necessary to screen multiple cDNA libraries from different tissues to obtain a full length cDNA. In the event that it is difficult to identify cDNA clones encoding the complete 5′ terminal coding region, an often encountered situation in cDNA cloning, the RACE (Rapid Amplification of cDNA Ends) technique can be used. RACE is a proven PCR-based strategy for amplifying the 5′ end of incomplete cDNAs. 5′-RACE-Ready RNA synthesized from human tissues containing a unique anchor sequence is commercially available (Clontech). To obtain the 5′ end of the cDNA, PCR is carried out on 5′-RACE-Ready cDNA using the provided anchor primer and the 3′primer. A secondary PCR reaction is then carried out using the anchored primer and a nested 3′ primer according to the manufacturer's instructions. Once obtained, the full length cDNA sequence can be translated into amino acid sequence and examined for certain landmarks such as a continuous open reading frame flanked by translation initiation and termination sites, a tyrosine phosphorylation site, a cysteine cluster, and finally overall structural similarity to the mouse CLASP genes disclosed herein. See, Ponassi et al., 1999, Mech. Dev. 80: 207-212; Isakov, 1998, Receptor Channels 5: 243-253; Borroto et al., 1997, Biopolymers 42: 75-88; Dimitratos et al., 1997, Mech. Dev. 63: 127-130; Apperson et al., 1996, J. Neurosci. 16: 6839-6852; Ozawa et al., 1990, Mech. Dev. 33: 49-56, which discuss protein domains and are incorporated herein by reference.

5.7. Modulating Expression of Endogenous CLASP Genes

Alternatively, the expression characteristics of an endogenous CLASP gene within a cell population can be modified by inserting a heterologous DNA regulatory element into the genome of the cell line such that the inserted regulatory element is operatively linked with the endogenous CLASP genes. For example, an endogenous CLASP gene which is normally “transcriptionally silent”, i.e., a CLASP gene which is normally not expressed, or is expressed only at very low levels in a cell population, can be activated by inserting a regulatory element which is capable of promoting the expression of a normally expressed gene product in the cells. Alternatively, a transcriptionally silent, endogenous CLASP gene can be activated by insertion of a promiscuous regulatory element that works across cell types.

A heterologous regulatory element can be inserted into a cell line population, such that it is operatively linked with an endogenous CLASP gene, using techniques, such as targeted homologous recombination, which are well known to those of skill in the art, (see e.g., in Chappel, U.S. Pat. No. 5,272,071; PCT publication No. WO 91/06667, published Jan 16, 1991).

5.8. Anti-CLASP Antibodies

Various procedures known in the art can be used for the production of antibodies to epitopes of the natural and recombinantly produced CLASP protein. Such antibodies include, but are not limited to, polyclonal, monoclonal, chimeric, single chain, human or humanized, IgG, IgM, IgA, IgD or IgE, a complementarity determining region, Fab fragments, F(ab′)₂ and fragments produced by an Fab expression library as well as anti-idiotypic antibodies. Antibodies which compete for CLASP binding are especially preferred for diagnostics and therapeutics.

Monoclonal antibodies that bind CLASPs can be radioactively labeled allowing one to follow their location and distribution in the body after injection. Radioisotope tagged antibodies can be used as a non-invasive diagnostic tool for imaging de novo lymphoid tumors and metastases that express CLASPs.

Immunotoxins can also be designed which target cytotoxic agents to specific sites in the body. For example, high affinity CLASP specific monoclonal antibodies can be covalently complexed to bacterial or plant toxins, such as diphtheria toxin or ricin. A general method of preparation of antibody/hybrid molecules can involve use of thiol-crosslinking reagents such as SPDP, which attack the primary amino groups on the antibody and by disulfide exchange, attach the toxin to the antibody. The hybrid antibodies can be used to specifically eliminate CLASP expressing lymphocytes.

For the production of antibodies, various host animals can be immunized by injection with the recombinant or naturally purified CLASP proteins, fusion proteins or peptides, including but not limited to goats, rabbits, mice, rats, hamsters, and the like Various adjuvants can be used to increase the immunological response, depending on the host species, including but not limited to Freund's (complete and incomplete), mineral gels such as aluminum hydroxide, surface active substances such as Iysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to CLASPs can be prepared by using any technique which provides for the production of antibody molecules by continuous cell lines in culture. These include, but are not limited to, the hybridoma technique originally described by Kohler and Milstein, (Nature, 1975, 256: 495-497), the human B-cell hybridoma technique (Kosbor et al., 1983, Immunology Today, 4: 72; Cote et al., 1983, Proc. Natl. Acad. Sci. U.S.A., 80: 2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci. U.S.A., 81: 6851-6855; Neuberger et al., 1984, Nature, 312: 604-608; Takeda et al., 1985, Nature, 314: 452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce CLASP-specific single chain antibodies. In some embodiments, phage display technology is used to identify antibodies and heteromeric Fab fragments that specifically bind to selected antigens (see, e.g., McCafferty et al., Nature 348: 552-554 (1990); Marks et al., Biotechnology 10: 779-783 (1992)).

Hybridomas can be screened using enzyme-linked immunosorbent assays (ELISA) in order to detect cultures secreting antibodies specific for refolded recombinant mouse CLASPs. Cultures can also be screened by ELISA to identify those cultures secreting antibodies specific for mammalian-produced CLASPs. Confirmation of antibody specificity can be obtained by western blot using the same antigens. Subsequent ELISA testing can use recombinant CLASP fragments to identify the specific portion of the CLASP molecule with which a monoclonal antibody binds. Additional testing can be used to identify monoclonal antibodies with desired functional characteristics such as staining of histological sections, immunoprecipitation of CLASPs, inhibition of CLASP binding or stimulation of CLASPs to transmit an intracellular signal. Determination of the monoclonal antibody isotype can be accomplished by ELISA, thus providing additional information concerning purification or function.

Some anti-CLASP monoclonal antibodies of the present invention are humanized, human or chimeric, in order to reduce their potential antigenicity, without reducing their affinity for their target. Humanized antibodies have been described in the art. See, e.g., Queen, et al., 1989, Proc. Natl Acad. Sci. U.S.A. 86: 10029; U.S. Pat. Nos. 5,563,762; 5,693,761; 5,585,089 and 5,530,101. The human antibody sequences used for humanization can be the sequences of naturally occurring human antibodies or can be consensus sequences of several human antibodies. See Kettleborough et al., 1991, Protein Engineering 4: 773; Kolbinger et al., 1993, Protein Engineering 6: 971. Humanized monoclonal antibodies against CLASP peptides can also be produced using transgenic animals having elements of a human immune system (see, e.g., U.S. Pat. Nos. 5,569,825; 5,545,806; 5,693,762; 5,693,761; and 5,7124,350).

In some embodiments, an anti-CLASP polypeptide monoclonal or polyclonal antiserum is produced that is specifically immunoreactive with a particular CLASP polypeptide and is selected to have low cross-reactivity against other molecules (e.g., other CLASP polypeptides) and any such cross-reactivity is removed by immunoabsorbtion prior to use in the immunoassay. Methods for screening and characterizing monoclonal antibodies for specificity are well known in the art and are described generally in Harlow and Lane, supra. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background. Alternatively, antibodies that cross-react with a selected set of polypeptides may be prepared.

Antibody fragments which contain specific binding sites of V can be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries can be constructed (Huse et al., 1989, Science, 246: 1275-1281) to allow rapid and easy identification of monoclonal Fab fragments with the desired specificity to CLASPs.

Anti-CLASPs antibodies can also be used to identify, isolate, inhibit or eliminate CLASP-expressing cells. In one embodiment, the present invention includes a method of identifying an abnormal T cell profile of an immunocompromised subject relative to the T cell profile of a non-immunocompromised subject. The method includes (i) sorting a sample of peripheral blood mononuclear cells (PBMC) isolated from the immunocompromised subject into sets of T cell types, (ii) determining the ratio of mouse CLASP⁺ cells relative to the total number of cells (mouse CLASP⁺: total) in each set, and identifying an abnormal T cell profile in the immunocompromised subject by comparing the CLASP⁺: total ratios of sets from the immunocompromised subject with the CLASP⁺: total ratios of analogous sets from a non-immunocompromised subject or animal.

In other embodiments, anti-CLASP antibodies can be used for detection of CLASP proteins in assays such as fluorescent activated cell sorting (FACS), ELISA, fluorescent or electron immunomicroscopy, Western blots, gel shift analyses, CLASP expression in various cells, localization within cells, interactions with other proteins, and differentiation between CLASP isoform expression can be determined by use of the techniques listed herein.

5.9. Screening Assays

The invention provides methods for identifying compounds or agents that modulate (i.e., inhibit or enhance) CLASP expression or activity. CLASP expression or activity modulators are useful for treatment of disorders characterized by (or associated with) aberrant or abnormal CLASP expression or activity. Aberrant expression of CLASP mRNAs or proteins means expression in lymphocytes (e.g., T lymphocytes or B lymphocytes) or other CLASP expressing cells of at least 2-fold, preferably at least 5-fold greater than expression in control lymphocytes obtained from a healthy subject or animal or animal model.

The CLASP expression assays can include the steps of contacting a cell expressing CLASPs with a compound or agent and assaying CLASP expression. CLASP polypeptide expression is easily measured by ELISA using anti-CLASP antibodies of the invention. CLASP mRNA expression (including expression of specific species or splice variants of CLASPs) can be measured by quantitative Northern analysis or quantitative PCR.

CLASP activities include, for example, the CLASP polypeptide binding to PDZ-domain containing molecules and CLASP polypeptides involvement in signal transduction (e.g., leading to T cell activation). Compounds or agents that modulate the interaction of a CLASP polypeptide and a target molecule, modulate CLASP nucleic acid expression, or modulate CLASP polypeptide activity are all contemplated by the methods of the present invention.

Test compounds include, for example, 1) peptides (e.g., soluble peptides, including Ig-tailed fusion peptides and members of random peptide libraries (see, e.g., Lam, K. S. et al., 1991, Nature 354: 82-84; Houghten, R. et al., 1991, Nature 354: 84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al., 1993, Cell 72: 767-778); 3) CLASP antibodies (as described above); 4) small organic and inorganic molecules (e.g., molecules obtained from combinatorial and natural product libraries); 5) antisense RNA and DNA molecules and ribozymes (described above).

The CLASP modulators can be any of a large variety of compounds, both naturally occurring and synthetic, organic and inorganic, and including polymers (e.g., oligopeptides, polypeptides, oligonucleotides, and polynucleotides), small molecules, antibodies, sugars, fatty acids, nucleotides and nucleotide analogs, analogs of naturally occurring structures (e.g., peptide mimetics, nucleic acid analogs, and the like), and numerous other compounds.

In one embodiment, the invention provides assays for screening test compounds which bind to CLASP polypeptides. The assays can be recombinant cell based or cell-free assays. These assays can include the steps of combining a cell expressing a CLASP polypeptide or a binding fragment thereof, and a compound or agent under conditions which allow binding of the compound or agent to the CLASP polypeptide to form a complex. Complex formation can then be determined. The ability of the candidate compound or agent to bind to the CLASP polypeptide or fragment thereof is indicated by the presence of the candidate compound in the complex. Formation of complexes between the CLASP polypeptide and the candidate compound can be quantitated, for example, using standard immunoassays.

In another embodiment, the invention provides screening assays to identify test compounds which modulate the interaction (and most likely CLASP activity as well) between a CLASP polypeptide and a molecule (target molecule with which the CLASP polypeptide normally interacts.

In one embodiment, these CLASP target molecules can be tyrosine kinases (e.g., lyn, lck, fyn, ZAP-70 m SyK, and CSK). In another embodiment, these CLASP target molecules can be tyrosine phosphatases (e.g., EZRIN, SHP-1, SHP-2 and PTP36). In another embodiment, these CLASP target molecules can be adaptor proteins (e.g., NCK, CBL, SHC, LNK, SLP-76, HS1, SIT, VAV, GrB2, and BRDG1). In another embodiment, these CLASP target molecules can be cytoskeletal associated proteins such as ankyrin, spectrin, talin, ezrin, tropomyosin, myosin, plectin, syndecans, paralemmin, Band 3 protein, cytoskeletal protein 4.1, and PTP36. In a further embodiment, CLASP target molecules can be members of the integrin family.

Typically, the assays are recombinant cell based or cell-free assay. These assays can include the steps of combining a cell expressing a CLASP polypeptide or a binding fragment thereof, a CLASP target molecule (e.g., a CLASP ligand) and a test compound, under conditions where but for the presence of the candidate compound, the CLASP polypeptide or biologically active portion thereof binds to the target molecule. Detecting complex formation between the CLASP polypeptide or the binding fragment thereof, the mouse CLASP target molecule and a test compound detecting the formation of a complex which includes the CLASP polypeptide and the target molecule can be accomplished. Detection of complex formation can include direct quantitation of the complex by, for example, measuring inductive effects, such as T cell activation, of the CLASP polypeptide. A significant change, such as a decrease, in the interaction of the CLASP and target molecule (e.g., in the formation of a complex between the CLASP and the target molecule) in the presence of a candidate compound (relative to what is detected in the absence of the candidate compound) is indicative of a modulation of the interaction between the CLASP polypeptide and the target molecule. Modulation of the formation of complexes between the CLASP polypeptide and the target molecule can be quantitated using, for example, an immunoassay. To perform cell free drug screening assays, it is desirable to immobilize either CLASP or its target molecule to facilitate separation of complexes from uncomplexed forms of one or both of the polypeptides, as well as to accommodate automation of the assay. CLASP binding to a target molecule, in the presence and absence of a candidate compound, can be accomplished in any vessel suitable for containing the reactants. Examples of such vessels include microtiter plates, test tubes, and microcentrifuge tubes.

In one embodiment, a fusion polypeptide can be provided which adds a domain that allows the polypeptide to be bound to a matrix. Alternatively, the complexes can be dissociated from the matrix, separated by SDS-PAGE, and the level of CLASP-binding polypeptide found in the bead fraction quantitated from the gel using standard electrophoretic techniques.

Other techniques for immobilizing polypeptides on matrices can also be used in the drug screening assays of the invention. For example, either CLASPs or its target molecule can be immobilized utilizing conjugation of biotin and streptavidin. Biotinylated CLASP molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with CLASPs but which do not interfere with binding of the polypeptide to its target molecule can be derivatized to the wells of the plate, and CLASPs trapped in the wells by antibody conjugation. As described above, preparations of a CLASP-binding polypeptide and a candidate compound are incubated in the CLASP-presenting wells of the plate, and the amount of complex trapped in the well can be quantitated. Methods for detecting such complexes include immunodetection of complexes using antibodies reactive with the CLASP target molecule, or which are reactive with CLASP polypeptide and compete with the target molecule; as well as enzyme-linked assays which rely on detecting an enzymatic activity associated with the target molecule.

One method of drug screening utilizes eukaryotic or prokaryotic host cells which are stably transformed with recombinant DNA molecules expressing CLASPs. Such cells, either in viable or fixed form, can be used for standard ligand/receptor binding assays (see, e.g., Parce et al. (1989) Science 246: 243-247; and Owicki et al. (1990) Proc. Natl Acad. Sci. U.S.A. 87: 4007-4011, which describe sensitive methods to detect cellular responses. A test compound, often labeled, can be assayed for binding or for competition with another ligand for binding. Viable cells could also be used to screen for the effects of drugs on CLASP-mediated functions, e.g., T cell activation, second messenger levels, and others).

In another embodiment, the invention provides a method for identifying a compound (e.g., a screening assay) capable of use in the treatment of a disorder characterized by (or associated with) aberrant or abnormal CLASP nucleic acid expression or CLASP polypeptide activity. This method typically includes the step of assaying the ability of the compound or agent to modulate the expression of the CLASP nucleic acid or the activity of the CLASP polypeptides thereby identifying a compound for treating a disorder characterized by aberrant or abnormal CLASP nucleic acid expression or CLASP polypeptide activity.

Methods for assaying the ability of the compound or agent to modulate the expression of the CLASP nucleic acid or activity of the CLASP polypeptide are typically cell-based assays. For example, cells which are sensitive to ligands which transduce signals via a pathway involving a CLASP or CLASPs can be induced to overexpress a CLASP polypeptide in the presence and absence of a candidate compound. Candidate compounds which produce a change in CLASP-dependent responses can be identified. In one embodiment, expression of the CLASP nucleic acid or activity of a CLASP polypeptide is modulated in cells and the effects of candidate compounds on the readout of interest (such as T cell activation) are measured. For example, the expression of genes which are up- or down-regulated in response to a CLASP-dependent signal cascade can be assayed.

Alternatively, modulators of CLASP expression can be identified in a method where a cell is contacted with a candidate compound and the expression of CLASP mRNAs or polypeptides in the cell is determined. The level of expression of CLASPs mRNA or polypeptide in the presence of the candidate compound is compared to the level of expression of CLASP mRNA or polypeptide in the absence of the candidate compound. The candidate compound can then be identified as a modulator of CLASP nucleic acid expression based on this comparison. For example, when expression of CLASP mRNA or polypeptide is greater in the presence of the candidate compound than in its absence, the candidate compound is identified as a stimulator of CLASP nucleic acid expression. Alternatively, when CLASP nucleic acid expression is less in the presence of the candidate compound than in its absence, the candidate compound is identified as an inhibitor of CLASP nucleic acid expression. The level of CLASP nucleic acid expression in the cells can be determined by methods described herein for detecting CLASP mRNA or polypeptide.

Modulators of CLASP polypeptide activity and CLASP nucleic acid expression identified according to these drug screening assays can be used to treat, for example, immune disorders. These methods of treatment include the steps of administering the modulators of CLASP polypeptide activity or nucleic acid expression, e.g., in a pharmaceutical composition as described in §5.10.1 below, to a subject in need of such treatment, e.g., a subject with a disorder described herein.

5.10. Therapeutic Administration of CLASP Modulators

CLASP proteins are expressed in lymphocytes and, as noted supra, play a role in regulating T cell and B cell interactions, thus making CLASP activity (e.g., CLASP-1 binding of regulatory proteins) a target for diagnostic and treatment of immune disorders and for modulation of immune function (e.g., T cell activation). Additionally, since CLASPs contain domains capable of transducing an intracellular signal, cell surface CLASP can be triggered by an anti-CLASP antibodies, soluble CLASPs or a fragment thereof in order to enhance the activation state of a lymphocyte.

5.10.1. Formulation and Route of Administration

A CLASP polypeptide, a fragment thereof, anti-CLASP antibody, CLASP polynucleotide (e.g., antisense or ribozyme), or small molecule CLASP-agonist or antagonist can be administered to a subject, animal, or animal model per se or in the form of a pharmaceutical or therapeutic composition. Pharmaceutical compositions comprising the proteins of the invention can be manufactured by means of conventional mixing, dissolving, granulating, dragee-making, levigating, emulsifying, encapsulating, entrapping or lyophilizing processes. Pharmaceutical compositions can be formulated in conventional manner using one or more physiologically acceptable carriers, diluents, excipients or auxiliaries which facilitate processing of the protein or active peptides into preparations which can be used pharmaceutically. Proper formulation is dependent upon the route of administration chosen.

Currently, there are three major classes of protein-derived cell-penetrating peptides that have been used for delivering of proteins into cells and animals (Lindgren, M.; et al., 2000, Trends Pharmacol Sci. 21: 99-103). In one embodiment, a CLASP protein or fragment (encoding a functional domain of a CLASP) can be introduced into the cell as a fusion protein tied to a transporter protein derived from homeoprotein transcription factors such as ANTP. In another embodiment, a CLASP protein or fragment (encoding a functional domain of a CLASP) can be introduced into the cell as a fusion protein tied to other transcription factors such as the HIV Tat protein and the herpes simplex virus type 1 (HSV-1) VP22 protein. Members in this family have been widely used in different cellular and animal systems (Schwarze, S.; et al.; 2000, Trends Pharmacol Sci. 21: 45-48). In another embodiment, a CLASP protein or fragment (encoding a functional domain of a CLASP) can be introduced into the cell as a fusion protein tied to peptides derived from signal-sequences present in several proteins such as HIV-1 gp41. In other embodiments, there are several synthetic and/or chemeric cell-penetrating peptides such as transportan and Amphiphiloc model peptide (Lindgren, M.; et al., 2000, Trends Pharmacol Sci. 21: 99-103) that can be used. In another embodiment, a mouse CLASP protein or fragment can be introduced by using anti-DNA antibodies (see, e.g., Zack, D. J., et al., 1996, J. Immunol. 157: 2082-8

For topical administration the proteins of the invention can be formulated as solutions, gels, ointments, creams, suspensions, and the like. as are well-known in the art.

Systemic formulations include those designed for administration by injection, e.g., subcutaneous, intravenous, intramuscular, intrathecal or intraperitoneal injection, as well as those designed for transdermal, transmucosal, oral or pulmonary administration.

For injection, the proteins of the invention can be formulated in aqueous solutions, preferably in physiologically compatible buffers such as Hanks's solution, Ringer's solution, or physiological saline buffer. The solution can contain formulatory agents such as suspending, stabilizing and/or dispersing agents. Alternatively, the proteins can be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, a composition can be readily formulated by combining the proteins with pharmaceutically acceptable carriers well known in the art. Such carriers enable the proteins to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents can be added, such as the cross-linked polyvinylpyrrolidone, agar, or alginic acid or a salt thereof such as sodium alginate.

If desired, solid dosage forms can be sugar-coated or enteric-coated using standard techniques.

For oral liquid preparations such as, for example, suspensions, elixirs and solutions, suitable carriers, excipients or diluents include water, glycols, oils, alcohols, and the like. Additionally, flavoring agents, preservatives, coloring agents and the like can be added.

For buccal administration, the proteins can take the form of tablets, lozenges, and the like. formulated in conventional manner.

For administration by inhalation, the proteins for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The proteins can also be formulated in rectal or vaginal compositions such as suppositories or retention enemas, e.g., containing conventional suppository bases such as cocoa butter or other glycerides.

In addition to the formulations described previously, the proteins can also be formulated as a depot preparation. Such long acting formulations can be administered by implantation (for example subcutaneously or intramuscularly) or by intramuscular injection. Thus, for example, the proteins can be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver the proteins or peptides of the invention. Certain organic solvents such as dimethylsulfoxide also can be employed, although usually at the cost of greater toxicity. Additionally, the proteins can be delivered using a sustained-release system, such as semipermeable matrices of solid polymers containing the therapeutic agent. Various sustained-release materials have been established and are well known by those skilled in the art. Sustained-release capsules can, depending on their chemical nature, release the proteins for a few weeks up to over 100 days. Depending on the chemical nature and the biological stability of the therapeutic reagent, additional strategies for protein stabilization can be employed.

As the proteins and peptides of the invention can contain charged side chains or termini, they can be included in any of the above-described formulations as the free acids or bases or as pharmaceutically acceptable salts. Pharmaceutically acceptable salts are those salts which substantially retain the biologic activity of the free bases and which are prepared by reaction with inorganic acids. Pharmaceutical salts tend to be more soluble in aqueous and other protic solvents than are the corresponding free base forms.

5.10.2. Effective Dosages

CLASP polypeptides, CLASP fragments and anti-CLASP antibodies will generally be used in an amount effective to achieve the intended purpose in animals and animal models. For use to inhibit an immune response, the proteins of the invention, or pharmaceutical compositions thereof, are administered or applied in a therapeutically effective amount. By therapeutically effective amount is meant an amount effective ameliorate or prevent the symptoms, or prolong the survival of, the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture (i.e., the concentration of test compound that inhibits 50% of CLASP binding interactions). Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval can be adjusted individually to provide plasma levels of the proteins which are sufficient to maintain therapeutic effect. Usual patient dosages for administration by injection range from about 0.1 to 5 mg/kg/day, preferably from about 0.5 to 1 mg/kg/day. Therapeutically effective serum levels can be achieved by administering multiple doses each day.

In cases of local administration or selective uptake, the effective local concentration of the proteins can not be related to plasma concentration. One having skill in the art will be able to optimize therapeutically effective local dosages without undue experimentation.

The amount of CLASP administered will, of course, be dependent on the subject being treated, on the subject's weight, the severity of the affliction, the manner of administration and the judgment of the prescribing physician.

The therapy can be repeated intermittently while symptoms detectable or even when they are not detectable. The therapy can be provided alone or in combination with other drugs. In the case of autoimmune disorders in mammals, animals or animal models, the drugs that can be used in combination with CLASPs or fragments thereof include, but are not limited to, steroid and non-steroid immunosuppressive agents.

5.10.3. Toxicity

Preferably, a therapeutically effective dose of the proteins described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch.1, p.1).

5.11. Binding Assays

CLASP polypeptides can be used to screen for molecules that bind to CLASPs or for molecules to which CLASPs bind. The binding of CLASPs by the molecule can activate (agonist), increase, inhibit (antagonist), or decrease activity of the CLASPs or the molecule bound. Examples of such molecules include antibodies, oligonucleotides, proteins (e.g., receptors), or small molecules. Preferably, the molecule is closely related to the natural ligand of a CLASP, e.g., a fragment of the ligand, or a natural substrate, a ligand, a structural or functional mimetic. (See, Coligan et al., Current Protocols in Immunology 1(2): Chapter 5 (1991).) Similarly, the molecule can be closely-related to the natural receptor to which CLASP may bind, or at least, a fragment of the receptor capable of being bound by a CLASP (e.g., active site). In either case, the molecule can be rationally designed using known techniques.

Preferably, the screening for these molecules involves producing appropriate cells which express a CLASP polypeptide or CLASPs, either as a secreted protein or on the cell membrane. Preferred cells include cells from mammals, yeast, Drosophila, or E. coli. Cells expressing CLASPs (or cell membrane containing the expressed polypeptide) are then preferably contacted with a test compound potentially containing the molecule to observe binding, stimulation, or inhibition of activity of either CLASPs or the molecule.

The assay can simply test binding of a candidate compound to CLASPs, where binding is detected by a label, or in an assay involving competition with a labeled competitor. Further, the assay can test whether the candidate compound results in a signal generated by binding to CLASPs.

Alternatively, the assay can be carried out using cell-free preparations, polypeptide affixed to a solid support, chemical libraries, or natural product mixtures. The assay can also simply comprise the steps of mixing a candidate compound with a solution containing a CLASP or CLASPs, measuring CLASP activity or binding, and comparing the CLASP activity or binding to a standard. Preferably, an ELISA assay can measure CLASP level or activity in a sample (e.g., biological sample) using a monoclonal or polyclonal antibody. The antibody can measure CLASP level or activity by either binding, directly or indirectly, to CLASPs or by competing with CLASPs for a substrate.

In another aspect of the invention, the CLASP polypeptides, or fragments thereof, can be used as “bait proteins” in a two-hybrid assay (see, e.g, U.S. Pat. No. 5,283,317; Zervos et al., 1993, Cell 72: 223-232; Madura et al., 1993, J. Biol. Chem. 268: 12046-12054; Bartel et al., 1993, Biotechniques 14: 920-924; Iwabuchi et al., 1993, Oncogene 8: 1693-1696; and Brent WO 94/10300), to identify other proteins, which bind to or interact with CLASPs (“CLASP-binding proteins” or “CLASP-bp”) and modulate CLASP polypeptide activity. Such CLASP-binding proteins are also likely to be involved in the propagation of signals by the CLASP polypeptides as, for example, upstream or downstream elements of the CLASP pathway.

All of these above assays can be used as diagnostic or prognostic markers. The molecules discovered using these assays can be used to treat disease or to bring about a particular result in a patient by activating or inhibiting the CLASP molecules. Moreover, the assays can discover agents which can inhibit or enhance the production of CLASPs from suitably manipulated cells or tissues.

Therefore, the invention includes a method of identifying compounds or agents that bind to CLASP polypeptides comprising the steps of: (a) contacting a CLASP polypeptide with a compound or agent under conditions which allow binding of the compound to the CLASP polypeptide to form a complex and (b) determining if binding has occurred. Moreover, the invention includes a method of identifying agonists or antagonists comprising the steps of: (a) incubating a candidate compound with a CLASP, (b) assaying a biological activity, and (b) determining if a biological activity of a CLASP has been altered.

Several methods of automating assays have been developed in recent years so as to permit screening of tens of thousands of compounds in a short period. See, e.g., Fodor et al., 1991, Science 251: 767-773, and other descriptions of chemical diversity libraries, which describe means for testing of binding affinity by a plurality of compounds.

5.12. Other Uses of CLASP Polynucleotides and Polypeptides

The polynucleotides, polypeptides, polypeptide homologues, modulators, and antibodies described herein can be used in one or more of the following methods: a) drug screening assays; b) diagnostic assays particularly in disease identification, allelic screening and pharmocogenetic testing; and c) pharmacogenomics. CLASP polypeptides of the invention have one or more of the activities described herein and can thus be used to, for example, modulate an immune response in an immune cell, for example by binding to a CLASP binding partner making it unavailable for binding to a naturally present CLASP polypeptide.

In one embodiment, these CLASP binding partners can be tyrosine kinases (e.g., lyn, lck, fyn, ZAP-70 m SyK, and CSK). In another embodiment, these CLASP binding partners can be tyrosine phosphatases (e.g., EZRIN, SHP-1, SHP-2 and PTP36). In another embodiment, these CLASP target molecules can be adaptor proteins (e.g., NCK, CBL, SHC, LNK, SLP-76, HS1, SIT, VAV, GrB2, and BRDG1. In another embodiment, these CLASP binding partners can be cytoskeletal associated proteins such as ankyrin, spectrin, talin, ezrin, tropomyosin, myosin, plectin, syndecans, paralemmin, Band 3 protein, cytoskeletal protein 4.1, and PTP36. In a further embodiment, CLASP binding partners can be members of the integrin family.

The isolated nucleic acid molecules of the invention can be used to express CLASP polypeptides (e.g., via a recombinant expression vector in a host cell or in gene therapy applications), to detect CLASP mRNAs (e.g., in a biological sample) or a naturally occurring or recombinantly generated genetic mutation in a CLASP gene, and to modulate CLASP activity, as described further below. In addition, the CLASP polypeptides can be used to screen drugs or compounds which modulate CLASP polypeptide activity as well as to treat disorders characterized by insufficient production of CLASP polypeptide or production of CLASP polypeptide forms which have decreased activity compared to wild type CLASPs. Moreover, the anti-CLASP antibodies of the invention can be used to detect and isolate a CLASP polypeptide, particularly fragments of CLASPs present in a biological sample, and to modulate CLASP polypeptide activity.

5.13. Diagnostic Assays

The invention further provides a method for detecting the presence of CLASPs, or fragments thereof, in a biological sample. Usually the biological sample contains lymphocytes (e.g., from blood). The method involves contacting the biological sample with a compound or an agent capable of detecting CLASP polypeptides or mRNAs such that the presence of CLASPs are detected in the biological sample.

A preferred agent for detecting CLASP mRNA is a directly or indirectly labeled nucleic acid probe capable of hybridizing to CLASP mRNA. The nucleic acid probe can be, for example, the full-length CLASP cDNA of the sequences shown in FIG. 1, FIG. 3, FIG. 6A or a portion thereof, such as an oligonucleotide of at least 15, 30, 50, 100, 250 or 500 nucleotides in length and sufficient to specifically hybridize under stringent conditions to CLASP mRNAs.

A preferred agent for detecting CLASP polypeptides is a directly or indirectly labeled antibody capable of binding to a CLASP polypeptide. Antibodies can be polyclonal, or more preferably, monoclonal. An intact antibody, or a fragment thereof (e.g., Fab or F(ab)₂) can be used. The term “directly or indirectly”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The detection method of the invention can be used to detect CLASP mRNAs or polypeptide in a biological sample in vitro as well as in vivo. For example, in vitro techniques for detection of CLASP mRNAs include Northern hybridizations and in situ hybridizations. In vitro techniques for detection of CLASP polypeptides include enzyme linked immunosorbent assays (ELISAs), Western blots, immunoprecipitations and immunofluorescence. Alternatively, CLASP polypeptides can be detected in vivo in a subject by introducing into the subject a labeled anti-CLASPs antibodies. For example, the antibodies can be labeled with a radioactive marker whose presence and location in a subject can be detected by standard imaging techniques. Particularly useful are methods which detect the allelic variants of CLASPs expressed in a subject and methods which detect fragments of a CLASP polypeptide in a sample.

The invention also encompasses kits for detecting the presence of a CLASP polypeptide or CLASPs in a biological samples. For example, the kit can comprise a directly or indirectly labeled compound or agent capable of detecting CLASP polypeptides or mRNAs in a biological sample; means for determining the amount of CLASPs in the sample; and means for comparing the amount of CLASPs in the sample with a standard. The compound or agent can be packaged in a suitable container. The kit can further comprise instructions for using the kit to detect CLASP mRNAs or polypeptides.

The methods of the invention can also be used to detect naturally occurring genetic mutations in a CLASP gene, thereby determining if a subject, animal or animal model with the mutated gene is at risk for a disorder characterized by aberrant or abnormal CLASP nucleic acid expression or CLASP polypeptide activity as described herein. In preferred embodiments, the methods include detecting, in a sample of cells from the subject, the presence or absence of a genetic mutation characterized by at least one of an alteration affecting the integrity of a gene encoding a CLASP polypeptide, or the misexpression of CLASP genes.

5.14. Biological Activities of CLASPs

As described herein, CLASPs mediate a variety of cell functions in lymphocytes and other cells. As described herein, a variety of assays are useful for detecting or quantitating CLASP activity, or for identifying agents (including polynucleotides, polypeptides, and antibodies of the invention) that modulate CLASP activity (i.e., biological activity, e.g., binding) or expression. Such agents are useful for treatment of diseases and conditions associated with aberrant CLASP expression or activity. Further, following the guidance provided herein, other CLASP-mediated activities can be identified by those of skill using routine assays, such as those described below.

Exemplary assays for CLASP function (or modulation of function) include assays for modulation of an in vitro or in vivo cell response (e.g., an immune response such as lymphocyte activation, antibody production, inflammation) by detecting a change in an activity (e.g., cytokine production, calcium flux, tyrosine phosphorylation, regulation of early activation markers, cell metabolism, proliferation, and the like, as described below) of cells in vitro or in vivo. In one embodiment, the cells are lymphocytes.

In one assay, for example, recombinant CLASP proteins, peptides, or antibodies corresponding to CLASP extracellular domains can be mixed directly with T and B cells. Cytokine production by these cells can then be measured and the degree of modulation of the immune response quantitated. Alternatively, antigen-presenting B cells are mixed with untransfected T cells or T cells that have been transfected with CLASP isoforms. Cytokine production (or calcium flux or other assays in §5.14.3) is be measured at the appropriate time to determine the effect of CLASPs on such an immune response. In a similar assay, B cells transfected with CLASP constructs are tested for their ability to stimulate a T cell to generate an immune response. Transfected constructs in any of these cases could encode, for example, full or partial length CLASP sequences, or antisense constructs to inhibit translation of endogenous CLASP genes. Any of the examples described herein can be used to stimulate an immune response in the presence or absence of CLASP isoforms or antibodies and assay the resulting effects on immune response by the methods listed in §5.14.3.

5.14.1 Methods for Generating an Immune Response in vitro

In various assays, an effect of an agent on immune cells is detected using an in vitro assay. The degree of an immune response can be measured or quantitated by a number of standard assays including those described below.

In one assay, human peripheral blood mononuclear cells (PBMC), human T cell clones (e.g., Jurkat E6, ATCC TIB-152), EBV-transformed B cell clones (e.g., 9D10, ATCC CRL-8752), antigen-specific T cell clones or lines can be used to examine immune responses in vitro. Activation, enhanced activation or inhibition of activation of these cells or cell lines can be used for the evaluation of potential CLASP therapeutics. Standard methods by which hematopoietic cells are stimulated to undergo activation characteristic of an immune response are, for example:

A) Antigen specific stimulation of immune responses. Either pre-immunized or naive mouse splenocytes can be generated by standard procedures. In addition, antigen-specific T cell clones and hybridomas (e.g., MBP-specific), and numerous B cell lymphoma cell lines (e.g., CH27), have been previously characterized are available for the assays discussed below. Antigen specific splenocytes or B-cells can be mixed with specific T-cells in the presence of antigen to generate an immune response. This can be performed in the presence or absence of CLASPs to assay whether CLASPs modulate the immune response as measured by any of the assays in section 5.14.2.

B) Non-specific T cell activation. The following methods can be used to activate T cells in the absence of antigen: 1) cross-linking T cell receptor (TCR) by addition of antibodies against receptor activation molecules (e.g., TCR, CD3, or CD2) together with antibodies against co-stimulator molecules, for example anti-CD28; 2) activating cell surface receptors in a non-specific fashion using lectins such as concanavalin A (con A) and phytohemagglutinin (PHA); 3) mimicking cell surface receptor-mediated activation using pharmacological agents that activate protein kinase C (e.g., phorbol esters) and increase cytoplasmic Ca²⁺ (e.g., ionomycin).

C) Non-specific B cell activation: 1) application of antibodies against cell surface molecules such as IgM, CD20, or CD21. 2) Lipopolysaccharide (LPS), phorbol esters, calcium ionophores and ionomycin can also be used to by-pass receptor triggering.

D) Mixed lymphocyte reaction (MLR). Mix donor PBMC with recipient PBMC to activate lymphocytes by presentation of mismatched tissue antigens, which occurs in all cases except identical twins.

E) Generation of a specific T cell clone or line that recognizes a particular antigen. A standard approach is to generate tetanus toxin-specific T cells from a donor that has recently been boosted with tetanus toxin. Major histocompatability complex-(MHC-) matched antigen presenting cells and a source of tetanus toxin are used to maintain antigen specificity of the cell line or T cell clone (Lanzavecchia, A., et al., 1983, Eur. J. Immun. 13: 733-738).

The anticipated mechanism of action of a CLASP polypeptide or polynucleotide should define the appropriate assay to use to investigate its potential enhancement or inhibition of lymphocyte activation. For example, soluble proteins containing a CLASP extracellular domain may interfere with the interaction between T cells and antigen presenting cells. Such interaction plays a role in the MLR and in antigen-specific T cell activation, but not in non-specific T or B cell activation. The assays described above have the advantage of several possible detection methods for quantitation.

5.14.2. Methods for Generating an Immune Response in vivo

In various assays, an effect of an agent on immune cells is detected using an in vivo assay. The degree of an immune response can be measured or quantitated by a number of standard assays including those described below.

(A) Animal Model for Transplantation Rejection: Ectopic Heart Transplantation

In one embodiment, a standard animal model for graft versus host rejection is ectopic heart transplantation (Fulmer et al, 1963, Am. J. Anat. 113: 273-281). This method involves using BALB/C mice (either sex, and range from 1-9 months) for transplanting cardiac tissue into a surgically-created pocket on the dorsum for both ears made by slitting the skin over the auricular artery at the base of the ear. Small curved forceps are forced into the slit, bluntly dissecting between the skin and the cartilage plate. Donor tissue is eased into the base of the pocket near the distal edge of the ear. The auricular artery is used to seal off the opening of the pocket. Within 10 to 14 days pulsatile activity of the transplant should be observed. Gross appearance of the graft, patterns of vacuolar supply to the graft area and pulsatile activity can be easily observed utilizing transilluminated light during the first three weeks post-transplantation. Follow-up can continue for for several months.

(B) Animal model for Autoimmune Disease: Induction of Collagen Induced Arthritis (CIA)

Collagen Induced Arthritis (CIA) is a standard model for studying progression and immune (Courtenay et al., 1980, Nature 283: 666 and Wooley et al., 1981, J. Exp. Med. 154: 688). DBA/a mice can be used as an assay for the in vivo relevance of mouse CLASPs in vitro testing potential immune therapeutics. In vivo experiments will be performed to examine the ability of potential therapeutics to prevent CIA. We will use 3-5 mice per group to statistically justify our results.

Once a titer of the potency of collagen type II (CII) is obtained therapeutics can be tested. In one embodiment, three mice will be immunized with three different concentrations of CII 50, 200, and 400 μg per animal (Nabozny et al., 1996, J. Exp. Med., 183: 27-37). To induce CIA, animals can be immunized with an appropriate concentration of CII, determined as described above. One half of a 1:1 ratio of antigen:CFA can be injected at the base of the tail and the remainder equally divided in each hind footpad. Mice can be carefully monitored every day for the onset and progression of CIA thoughout the experiment until its termination 12 weeks post-immunization with CII. The pieces of heart transplanted can be approximately 3×3 mm in size. The severity of arthritis can be assessed following standard procedures known to one of skill in the art.

5.14.3 Assay Quantitation

(A) Tyrosine Phosphorylation

Tyrosine phosphorylation of early response proteins such as HS 1, PLC-r, ZAP-76, and Vav is an early biochemical event following T cell activation. The tyrosine phosphorylated proteins can be detected by Western blot using antibodies against phosphorylated tyrosine residues. Tyrosine phosphorylation of these early response proteins can be used as a standard assay for T cell activation (J. Biol. Chem., 1997, 272(23): 14562-14570). Any change in the phosphorylation pattern of these or related proteins when immune responses are generated in the presence of a CLASP or CLASPs is indicative of a CLASP modulation of this response.

(B) Intracellular Calcium Flux

The kinetics of intracellular Ca²⁺ concentrations are measured over time after stimulation of cells preloaded with a calcium sensitive dye. Upon binding the Ca²⁺ indicator dye, Fluor-4 (Molecular Probes), exhibits an increase in fluorescence level using flow cytometry, solution fluorometry, and confocal microscopy. Any change in the level or timing of calcium flux when immune responses are generated in the presence of a CLASP or CLASPs is indicative of a CLASP modulation of this response.

(C) Regulation of Early Activation Markers

Increased and diminished expression/regulation of early lymphocyte activation marker levels such as CD69, IL-2R, MHC class II, B7, and TCR are commonly measured with fluorescently labeled antibodies using flow cytometry. All antibodies are commercially available. Any change in the level or timing of calcium flux when immune responses are generated in the presence of a CLASP or CLASPs is indicative of a CLASP modulation of this response.

(D) Increased metabolic activity/acid release

Activation of most known signal transduction pathways trigger increases in acidic metabolites. This reproducible biological event is measured as the rate of acid release using a microphysiometer (Molecular Devices), can be used as an early activation marker when comparing the treatment of cells with potential biological therapeutics (McConnell, H. M. et al., 1992, Science 257: 1906-1912 and McConnell, H. M., 1995, Proc. Natl. Acad. Sci. 92: 2750-2754). Any statistically significant increase or decrease in acid release from a CLASP-treated sample, as compared to control sample (no treatment), suggest and effect of CLASP on biological function.

(E) Cell Proliferation/cell Viability Assays

(1) ³H-thimidine Incorporation

Exposure of lymphocytes to antigen or mitogen in vitro induces DNA synthesis and cellular proliferation. The measurement of mitotic activity by ³H-thimidine incorporation into newly synthesized DNA is one of the most frequently used assays to quantitative T cell activation. Depending on the cell population and form of stimulation used to activate the T cells, mitotic activity can be measured within 24-72 hrs. in vitro, post ³H-thimidine pulse (Mishell, B. B. and S. M. Shiigi, 1980, Selected Methods in Cellular Immunology, W. H. Freeman and Company and Dutton, R. W. and Pearce, J. D., 1962, Nature 194: 93). Any statistically significant increase or decrease in CPM of aCLASP-treated sample, as compared to control sample (no treatment), suggest an effect of CLASP on biological function.

(2) MTS [5-(3-carboxymethoxyphenyl)-2-(4,5-dimethylthiazolyl)-3(4-sulfophenyl)tetrazolium, inner salt] is a colorimetric method for determining the number of viable cells in proliferation or cytotoxicity assays (Barltrop, J. A. et al., 1991, Bioorg. & Med. Chem. Lett. 1: 611). 1-5 days after lymphocyte activation, MTS tetrazolium compound, Owen's reagent, is bioreduced by cells into a colored formazan product that is soluble in tissue culture media. Color intensity is read at 490 nm minus 650 nm using a microplate reader. Any statistically significant increase or decrease in color intensity of a CLASP-treated sample, as compared to control sample (no treatment), can suggest an effect of CLASP on biological function (Mosmann, T., 1983, J. Immunol. Methods 65: 55 and Barltrop, J. A. et al. (1991)).

(3) Bromodeoxyuridine (BrdU), a thymidine analogue, readily incorporates into cells undergoing DNA synthesis. BrdU-pulsed cells are labeled with an enzyme-conjugated anti-BrdU antibody (Gratzner, H. G., 1982, Science 218: 474-475.). A calorimetric, soluble substrate is used to visualize proliferating cells that have incorporated BrdU. Reaction is stopped with sulfuric acid and plate is read at 450 nm using a microplate reader. Any statistically significant increase or decrease in color intensity of a CLASP-treated sample, as compared to control sample (no treatment), suggest an effect of CLASP on biological function.

(F) Apoptosis by Annexin V

Programmed cell death or apoptosis is an early event in a cascade of catabolic reactions leading to cell death. A lose in the integrity of the cell membrane allows for the binding of fluorescently conjugated phosphatidylserine. Stained cells can be measured by fluorescence microscopy and flow cytometry (Vermes, I., 1995, J. Immunol. Methods. 180: 39-52). In one embodiment, any statistically significant increase or decrease in apoptotic cell number of a CLASP-treated sample, as compared to control sample (no treatment), suggests an effect of CLASP on biological function. For evaluating apoptosis in situ, assays for evaluating cell death in tissue samples can also be used in vivo studies.

(G) Quantitation of Cytokine Production

Cell supernatants harvested after cell stimulation for 16-48 hrs are stored at −80° C. until assayed or directly tested for cytokine production. Multiple cytokine assays can be performed on each sample. IL-2, IL-3, IFN-gamma and other cytokine ELISA Assays are available for mouse, rat, and human (Endogen, Inc. and BioSource). Cytokine production is measured using a standard two-antibody sandwich ELISA protocol as described by the manufacturer. The presence of horseradish peroxidase is detected with 3, 3′5, 5′ tertamethyl benzidine (TMB) substrate and the reaction is stopped with sulfuric acid. The absorbance at 450 nm is measured using a microplate reader. Any statistically significant increase or decrease in color intensity of a CLASP-treated sample, as compared to control sample (no treatment), suggests an effect of CLASP on biological function.

(H) NF-AT can be visualized by Immunostaining

T cell activation requires the import of nuclear factor of activated T cells (NFAT) to the nucleus. This translocation of NF-AT can be visualized by immunostaining with anti-NF-AT antibody (Cell 1998, 93: 851-861). Therefore, NF-AT nuclear translocation has been used to assay T cell activation. Similarly, NF-AT/luciferase reporter assays have been used as a standard measurement of T cell activation (MCB 1996, 12: 7151-7160).

(I) ELISA for collagen type II (CII)-specific antibodies (see above for related in vivo assay)

C(II) titers from serum of animals immunized with CLASPs can be measured and compared. Both TH1-dependent IgG2a and TH2-dependent IgG1 and IgE CII-specific antibody isotypes will be measured by ELISA. Mouse blood will be obtained by orbital bleed one and two months post-immunization with CII. Samples will be allowed to coagulate and centrifuge to obtain sera, and stored at −80° C. until assayed by ELISA. Coat ELISA plates with CII and dilute sera. HRP conjugated goat, isotype specific antibody. Plates are then expose to TMB substrate and read at 450 nm using a microplate reader (Nabozny et al., 1996, J. Exp. Med. 183: 27-37). Any change in the levels of Collagen specific antibodies by colorimetric test when immune responses are generated in the presence of CLASPs is indicative of a CLASP modulation of this response.

(J) Antibody Production by ELISPOT Assay

A solid-phase enzyme-linked immunospot (ELISPOT) assay for the quantification of isotype-specific antibody secreting cells (Czerkinsky et al., 1983, J Immunol. Methods. 65: 109-121). Both human and mouse B cells can be tested for isotype and antigen specific antibody production. Although based on a standard ELISA, this technique becomes more sensitive by detecting antibody secretion from single cells. Any change in ELISPOT levels when immune responses are generated in the presence ofa CLASP is indicative of a CLASP modulation of this response.

(K) Cellular Degranulation Following IgE Cross-linking.

Two cell lines have been obtained from ATCC (MEG01 and HEL-17.92), both of which express the human FCεFR1 receptor. FCεR1 is the high affinity receptor for IgE complexes, which when coupled to biotin can be cross-linked with avidin to induce degranulation and histamine release of lymphocytes. Following acylatation of the sample, histamine is quantified with an enzyme immunoassay competition assay (Immunotech). Histamine release. A statistically significant increase or decrease in histamine concentration of a CLASP treated sample, as compared to control sample (no treatment), suggest an effect of CLASP on biological function. Any change in frequency of degranulation or histamine levels when immune responses are generated in the presence of a CLASP is indicative of a CLASP modulation of this response.

(L) Cellular phenotyping of lymphocytes by flow cytometry and Immunocytochemistry

Determining the tissue distribution of lymphocytes following a pathological disorder can aid in identifying specific organ, tissue and lymphocyte involved in an immune response. Cellular phenotyping of lymphocyte trafficking is generally performed with by flow cytometry and Immunocytochemistry. There are several cluster determination (CD) molecules that are routinely used to identify phenotype, activation kinetics, and regulation events of cells. Any change in levels or distribution of CD molecules when immune responses are generated in the presence of a CLASP is indicative of a CLASP modulation of this response.

(M) Structure/Function Assays: Homotypic and/or Heterotypic, Calcium-dependant Cell Adhesion

L929 cells can be transfected with a CLASP or CLASPs and Neomycin. G418-resistant clones can be screened for CLASP-expression with anti-CLASP peptide-specific antibodies. These CLASP-expressing clones can then be used to test for homotypic and/or heterotypic calcium dependent cell adhesion using the “cell aggregation assay” described for cadherin molecules (Murphy-Erdosh, C. et al., 1995, J. Cell Biol. 129: 1379-1390). Any change in the levels of cellular aggregation when immune responses are generated in the presence of CLASPs is indicative of a CLASP modulation of this response.

The following cDNA clones described in the Specification and further described in the Examples below have been deposited with the American Type Culture Collection, 10801 University Boulevard, Manassas, Va. 20110-2209 under the Budapest Treaty on ______, 200_ and given the Accession Nos. indicated: ______

### 6. EXAMPLES

### Example 1

### Cloning of CLASP cDNAs

Cloning of Human CLASP-1

Human CLASP-1 was cloned in the following manner: an expressed sequence tag or EST clone (IMAGE clone 712567, derived from human germinal B cells) was identified based on a BLAST search of human GenBank human EST database using mouse CLASP-1 sequences. IMAGE clone 712567 was sequenced completely. A polynucleotide probe prepared from 712567 sequence was labeled with ³²P-dCTP and used to screen human cDNA libraries including Jurkat (Stratagene) and Ramos B cell cDNA library (James Boulter, UCLA). The screening methods employed were as described in Maniatis et al., 1989, Molecular Cloning A Laboratory Manual, Cold Spring Harbor Laboratory, New York. Several clones were identified, subcloned, sequenced (ABI dye-sequencing system, PE Applied Biosystems; Perkin-Elmer Corporation, 761 Main Avenue, Norwalk, Conn., U.S.A.), and were aligned to generate a contiguous cDNA sequence encoding the 3′ terminal ˜1.8 kb of hCLASP-1. Probes for bacteriophage library screening were then produced by PCR corresponding to the 5′ portions of these clones and used to re-screen these and other libraries (heart, brain and testis—Stratagene). Clones with extended 5′ sequence were identified by library screening and characterization of clones using anchored PCR (antisense primer directed against hCLASP-1 and a vector primer). Although some bacteriophage clones contained additional 5′ sequence, no clones extending between the region approximately 1800 nucleotides to ˜4200 nucleotides from the 3′ end of the hCLASP-1 sequence presented in FIG. 1A were ever able to be genetically excised (Zap-out, Stratagene) out of phage into bacterial vectors after repeated attempts with independent clones from different bacteriophage libraries. Attempts were made to amplify the inserts in these clones by polymerase chain reaction or by large scale bacteriophae preps (Maniatis) and clone them directly into PCR product cloning vectors (TA cloning vector, Invitrogen; pGEM T-Easy, Promega) or cloning vectors (Bluescript, Stratagene). Transformed bacteria carrying these vectors were identified at an extremely low frequency and when the inserts were analyzed it was noted that these contained mutations or rearrangements in the hCLASP-1 inserts. Using primers designed from partial sequencing of these clones and degenerate primers designed against homologous regions in the partial mouse CLASP-1 and human CLASP-2 genes, smaller portions of the hCLASP-1 gene were able to be isolated and sequenced. Regions between these smaller clones were confirmed by direct sequencing of RT-PCR products. These techniques resulted in the identification of clones encompassing the C-terminal 5.55 kb of hCLASP-1. Further bacteriophage library screening from both oligo-dT and random primed libraries failed to produce clones containing additional 5′ sequence (over 100 genome equivalents screened). Commercial libraries from multiple tissue sources including human placenta, B cell, T cell and peripheral blood were exhaustively screened and re-screened resulting in the acquisition of only partial cDNAs. Generation of cDNA libraries using oligo dT or CLASP-specific primers also resulted in the acquisition of partial cDNAs. Genomic libraries were screened to obtain a portion of the genomic locus for each of the CLASP genes, and a genomic walk was initiated to obtain 5′ exons and extend the cDNA sequence.

To obtain additional 5′ CLASP-1 sequence, portions of the cDNA and genomic sequence from a BAC (Bacterial Artificial Chromosome) genomic library were compared to the NCBI database by BLAST. A genomic clone (Genbank identifier: gi8705162) comprising random, shotgun genomic sequence was identified. Using TFASTX (Pearson and Lipman, PNAS (1988) 85:2444-2448), the amino-terminal sequence of human CLASP-4 was compared to 6 frame translation of gi8705162. Areas of gi8705162 that encoded amino acids with high similarity to CLASP-4 amino acid sequence were used to design CLASP-1-specific oligonucleotides for RTPCR (reverse transcriptase polymerase chain reaction according to manufacturers instructions: Reverse transcriptase Gibco/BRL, Taq Polymerase from Sigma). Using oligonucleotides hC1gS7 (nucleotides 124-144 of FIG. 1) and C1AS2 (reverse complement of nucleotides 1571-1590 of FIG. 1) an RTPCR product of approximately 1.3 kb was generated, sequenced (dideoxynucleotide sequencing, Beckman Coulter CEQ2000) and shown to be additional human CLASP-1 5′ sequence. Further complicating the cloning full-length CLASP cDNA products was the difficulty to clone (and subclone) certain CLASP cDNA products. Standard isolation of some of the CLASP cDNAs from a pure phage population following screening of commercially available cDNA libraries (“ZAP-out” procedure, Stratagene) resulted in no bacterial colonies. Similarly, certain RT-PCR products could not be cloned into standard plasmid vectors. No colonies were isolated by cloning these fragments into vectors lacking promoters, reverse orientations, low copy vectors, or by growth at altered temperatures or levels of antibiotic for plasmid selection (examples: CLASP-7—HC7gS6 to HC7gAS1 and HC7gS3 to HC7AS14; CLASP-4—C4P2 to hC4ASTM and C4P2 to HC4AS3′; CLASP-1—hC1S5′ to hC1AS3′ Kpn and C1S7 to hC1AS3′Kpn; see Primer Table below). One possibility is that sequences contained within certain regions of CLASP cDNAs are bacteriacidal and therefore not amenable to cloning. To circumvent these problems direct sequencing of RT-PCR products was performed.

Primer Table

| CLASP | Sense | Sense | Antisense | Antisense |
|---|---|---|---|---|
| gene | Primer | sequence | Primer | sequence |
| CLASP-7 | HC7gS5 | AGGCCTTGTCTCT | HC7gAS1 | TGTCATGTACTGC |
| | | GTTTACCTG | | ACTCGCACAGC |
| CLASP-7 | HC7gS3 | ACAGGAACCTGCT | HC7AS14 | TCGTGGCTGCACA |
| | | GTACGTGTAC | | GGATGCGGGTG |
| CLASP-4 | C4P2 | GACCCATTAGGAG | HC4AS3′ | CGGGATCCATrGT |
| | | GTCTAC | | CACCGTACATCTG |
| | | | | C |
| CLASP-4 | C4P2 | GACCCATTAGGAG | HC4AS3′ | CGGGATCCATTGT |
| | | GTCTAC | | CACCGTACATCTG |
| | | | | C |
| CLASP-1 | hC1S5′ | TATGTCTCAGTCA | HC1AS3′Kpn | CTTGGTACCACTT |
| | | CCTACCTG | | CAGCACTAGATGA |
| | | | | GATG |
| CLASP-1 | C1S7 | TCAAGACCAGGOC | HC1AS3′Kpn | CTTGGTACCACTT |
| | | ATGCAAG | | CAGCACTAGATGA |
| | | | | GATG |

In-frame stop codons were not present suggesting that the cDNA was not full length. To obtain the 5′ terminus of CLASP-1 5′ RACE was employed. Antisense oigonucleotides directed against the 5′ end of the longest CLASP-1 sequence were generated:

Primers Used for Human CLASP-1 5′ RACE

| | | |
|---|---|---|
| Primer | seguence(5′ TO 3′) | nucleotide position |

| | | |
|---|---|---|
| HClRACE2 | TGTTGAGTACAACGTGCGGATATCC | 243 to 267 |
| HClRACE3 | TCATATTTGTAGTTTACCACATGCCACTG | 337 to 365 |
| HClRACE4 | TCAAAGGAATGTGAAGGAAGCTTCTCTGG | 412 to 440 |

RACE was carried out using Invitrogen's Generacer kit according to manufacturers specifications using polyA selected mRNA from 9D10 B cell tissue culture line. The sequence of the oligonucleotides presented is the reverse complement (i.e. antisense) of the the CLASP-1 cDNA at the indicated position based upon numbering in FIG. 1.

The full length cDNA (presented in FIG. 1) is therefore a compilation of cDNA from cDNA libraries, RTPCR products and 5′ RACE products. The sequence of the CLASP-1 cDNA is shown in FIG. 1.

Cloning of Mouse CLASP-1

Degenerate oligonucleotides encoding the highly conserved cadherin domains TAPPYD and FKKLAD (5′ sense primer GGAATTCCACNGCNCCNCCNTA(CT)GA, and 3′ antisense primer GCTCTAGATCNGCNA(AG)(CT)TT(CT)TT(AG)AA, where N is any nucleotide) were used in a reverse-transcriptase polymerase chain reaction (RT-PCR) to obtain a representative clone that was used to screen a mouse neonatal thymus library. This yielded parital mouse CLASP-1 cDNAs represented by nucleotides 2261 and greater in the sequence presented in FIG. 1A. To obtain the full length mouse CLASP-1 cDNA sequence, the human CLASP-1 amino acid sequence was searched against the mouse expressed sequence tag (EST) database (National Center for Biotechnology Information, NCBI) and multiple ESTs corresponding to mouse CLASP-1 were identified. Oligonucleotides were designed against these ESTs and used in polymerase chain reactions (PCR) upon cDNA obtained from reverse transcribed (RT) mRNA from various mouse tissue culture cell lines according to manufacturers instructions (Reverse transcriptase Gibco/BRL, Herculase Polymerase from Stratagene). These experiments yielded 5 kb of mouse CLASP-1 cDNA sequence that was missing the 5′ terminus. To obtain the complete cDNA sequence of mouse CLASP-1, 5′ RACE was employed using Invitrogen's Generace Kit according to manufacturers specifications and the following primers:

| | | |
|---|---|---|
| Primer | Sequence | Nucleotide Position |

| | | |
|---|---|---|
| mC1GR1 | AGCCTGTTATTCTGTACTACACCT | 696-722 |

GTG

| | | |
|---|---|---|
| mC1GR2 | TTCGTCCATATCTGACTCTGTTTC | 779-804 |

AG

| | | |
|---|---|---|
| mC1GR3 | TCACGCAGTTATCCAGTGGATCTA | 892-916 |

G

The sequence of the oligonucleotides presented is the reverse complement (i.e. antisense) of the mouse CLASP-1 cDNA at the indicated positions based upon numbering in FIG. 1A. RACE was carried out using polyA-selected mRNA from 3A9 T cell hybridoma mouse tissue culture cell line.

The full length cDNA is therefore a compilation of cDNA from RTPCR products and 5′ RACE products. Multiple cDNA products from RTPCR and 5′ RACE were double-stranded sequenced (Beckman-Coulter CEQ) to obtain accurate sequence information. The sequence of the mouse CLASP-1 cDNA is shown in FIG. 1.

Cloning of Mouse CLASP-2 cDNA

The human CLASP-2 amino acid sequence was searched against the mouse expressed sequence tag (EST) database (National Center for Biotechnology Information, NCBI) and multiple ESTs corresponding to mouse CLASP-2 were identified. Oligonucleotides were designed against these ESTs and used in polymerase chain reactions (PCR) upon cDNA obtained from reverse transcribed (RT) mRNA from various mouse tissue culture cell lines (inlcuing 2B4, 3A9, L929, BW and CH27) according to manufacturers instructions (Reverse transcriptase Gibco/BRL, Herculase Polymerase from Stratagene). These experiments yielded 6454 nucleotides of mouse CLASP-2 cDNA that is presented in FIG. 1A. This partital cDNA is therefore a compilation of cDNA from RTPCR products. Multiple cDNA products from RTPCR were double-stranded sequenced (Beckman-Coulter CEQ) to obtain accurate sequence information. The sequence of the partial mouse CLASP-2 cDNA is shown in FIG. 1.

Alignment of human and mouse CLASP-2 amino acid sequences indicates that the full coding region may be present in the sequence shown in FIG. 1A since a methionine designated as the start of open reading frame for human CLASP-2 is present in a similar location in the amino acid sequence for mouse CLASP-2 (see FIG. 1B). However, since there are no stop codons upstream of the putative initiator methionine in mouse CLASP-2, it is possible that the open reading frame of mouse CLASP-2 continues. Until the full length cDNA is obtained we cannot conclusively assign the amino terminus of the mouse CLASP-2 open reading frame. However, this does not detract from the fact that we have cloned greater than 90% of the mouse CLASP-2 cDNA.

Cloning of Mouse CLASP-3 cDNA

The human CLASP-3 amino acid sequence was searched against the mouse expressed sequence tag (EST) database (National Center for Biotechnology Information, NCBI) and multiple ESTs corresponding to mouse CLASP-3 were identified. Oligonucleotides were designed against these ESTs and used in polymerase chain reactions (PCR) upon cDNA obtained from reverse transcribed (RT) mRNA from various mouse tissue culture cell lines including (L929, 2B4 and BW) according to manufacturers instructions (Reverse transcriptase Gibco/BRL, Herculase Polymerase from Stratagene). These experiments yielded 4712 nucleotides of mouse CLASP-3 cDNA that is presented in FIG. 1A from nucleotides 1 to 4712. The rest of the cDNA from nucleotides 4713 to 6226 is represented by the following expressed sequenced tag (EST) entries from the National Center for Biotechnology Information (NCBI) Genbank database: genbank identfier (gi) 11039719, gi12756923, gi14551922, gi10377137, gi8931971, gi1811208, gi1715789, gi1715773, gi1426790, gi1680900, gi12563379, gi9815330, gi10712472, gi4315485, gi1904433, gi12757011, and gi8185153. This partital cDNA is therefore a compilation of cDNA from RTPCR products and ESTs form NCBI Genbank database. cDNA products from RTPCR were double-stranded sequenced (Beckman-Coulter CEQ) to obtain accurate sequence information. The sequence of the partial mouse CLASP-3 cDNA is shown in FIG. 1.

Cloning of Mouse CLASP-4 cDNA

To obtain the full length mouse CLASP-4 cDNA sequence, the human CLASP-4 amino acid sequence was searched against the mouse expressed sequence tag (EST) database (National Center for Biotechnology Information, NCBI) and multiple ESTs corresponding to mouse CLASP-4 were identified. Oligonucleotides were designed against these ESTs and used in polymerase chain reactions (PCR) upon cDNA obtained from reverse transcribed (RT) mRNA from various mouse tissue culture cell lines (including 3A9, CH27, 2B4 and BW) according to manufacturers instructions (Reverse transcriptase Gibco/BRL, Herculase Polymerase from Stratagene). These experiments yielded approximately 6.2 kb of mouse CLASP-4 cDNA sequence represented by nucleotides 261 to 6408 in FIG. 1A. Nulceotides 6409 to 6579 were derived from an EST (Genbank identifier (gi) 1275784) from NCBI Genbank database. To obtain the 5′ terminus and complete the cDNA sequence of mouse CLASP4, 5′ RACE was employed using Invitrogen's Generace Kit according to manufacturers specifications and the following primers:

| | | |
|---|---|---|
| Primer | Sequence | Nucleotide Position |

| | | |
|---|---|---|
| mC4GR1 | TCTCAAAGACTTGCATGGCAACAT | 381-405 |

C

| | | |
|---|---|---|
| mC4GR2 | TTGAACACCTTCATGGTTACTGTG | 540-566 |

ATG

| | | |
|---|---|---|
| mC4GR3 | TGCTCCGTTTCAGCTGCCAGATAA | 748-773 |

TG

The sequence of the oligonucleotides presented is the reverse complement (i.e. antisense) of the mouse CLASP-4 cDNA at the indicated positions based upon numbering in FIG. 1A. RACE was carried out using polyA-selected mRNA from BW thymoma and 3A9 T cell hybridoma mouse tissue culture cell lines.

The full length cDNA is therefore a compilation of cDNA from RTPCR products and 5′ RACE products and a single EST. Multiple cDNA products from RTPCR and 5′ RACE were double-stranded sequenced (Beckman-Coulter CEQ) to obtain accurate sequence information. The sequence of the mouse CLASP-4 cDNA is shown in FIG. 1A.

Cloning of mouse CLASP-5 cDNA

To obtain the full length mouse CLASP-5 cDNA sequence, the human CLASP-5 amino acid sequence was searched against the mouse expressed sequence tag (EST) database (National Center for Biotechnology Information, NCBI) and multiple ESTs corresponding to mouse CLASP-5 were identified. Oligonucleotides were designed against these ESTs and used in polymerase chain reactions (PCR) upon cDNA obtained from reverse transcribed (RT) mRNA from various mouse tissue culture cell lines (including 3A9, CH27, L929, 2B4 and BW) according to manufacturers instructions (Reverse transcriptase Gibco/BRL, Herculase Polymerase from Stratagene). These experiments yielded approximately 6.0 kb of mouse CLASP-5 cDNA sequence represented by nucleotides 169 to 6156 in FIG. 1A. To obtain the 5′ terminus and complete the cDNA sequence of mouse CLASP-5, 5′ RACE was employed using Invitrogen's Generace Kit according to manufacturers specifications and the following primers:

| Primer | | |
|---|---|---|
| Position | Sequence | Nucleotide |
| mC5GR2 | GTTCACTGCACTCCAAGGTTTCTGAC | 288-313 |
| mC5GR3 | TGGAGCAGGTTTTCCAGCCGCTCATC | 418-443 |

The sequence of the oligonucleotides presented is the reverse complement (i.e. antisense) of the mouse CLASP-5 cDNA at the indicated positions based upon numbering in FIG. 1A. RACE was carried out using polyA-selected mRNA from BW thymoma and 3A9 T cell hybridoma mouse tissue culture cell lines.

The full length cDNA is therefore a compilation of cDNA from RTPCR products and 5′ RACE products. Multiple cDNA products from RTPCR and 5′ RACE were double-stranded sequenced (Beckman-Coulter CEQ) to obtain accurate sequence information. The sequence of the mouse CLASP-5 cDNA is shown in FIG. 1A.

Cloning of Mouse CLASP-7 cDNA

The human CLASP-7 amino acid sequence was searched against the mouse expressed sequence tag (EST) database (National Center for Biotechnology Information, NCBI) and multiple ESTs corresponding to mouse CLASP-7 were identified. Oligonucleotides were designed against these ESTs and used in polymerase chain reactions (PCR) upon cDNA obtained from reverse transcribed (RT) mRNA from various mouse tissue culture cell lines including (L929 and CH27) according to manufacturers instructions (Reverse transcriptase Gibco/BRL, Herculase Polymerase from Stratagene). These experiments yielded three fragments of mouse CLASP-7 sequence presented in FIG. 1. In addition to cDNA sequence derived from RT-PCR, the three cDNA fragments presented in FIG. 1 contain sequence from ESTs culled from NCBI Genbank database. These include: for Fragment 1—gi9973144, gi14295340, and gi1702072; and for Fragment 3—gi14619196, gi12681060, gi2943152, gi2916671, gi6516809, gi4301714, gi14497304 and gi2200647.

The sequence of the partial mouse CLASP-7 cDNA is therefore a compilation of RTPCR products and ESTs and is shown in FIG. 1.

### Example2

Expression Profile of Mouse CLASPs in Multiple Mouse Tissues

Results

CLASP-Mouse CLASP-1

To determine mouse CLASP-1 expression profile Northern blot analysis was carried out. A probe representing a portion of the mouse CLASP-1 cDNA was hybridized against RNA from multiple mouse tissues. As FIG. 4A shows, this probe reveals a specific transcript of approximately 7.5 kb, which correlates with the length of the mouse CLASP-1 cDNA sequence presented in FIG. 1A (˜7.3 kb). Expression of mouse CLASP-1 is clearly detected in brain, lung and spleen with slight expression detected in heart and kidney, and very weak expression in liver and skeletal muscle. Signal intensities correlate to the relative abundance of mouse CLASP-1-specific RNA in the various tissues.

Mouse CLASP-2

To determine mouse CLASP-2 expression profile Northern blot analysis was carried out. A probe representing a portion of the mouse CLASP-2 cDNA was hybridized against RNA from multiple mouse tissues. As FIG. 4B shows, this probe reveals a specific transcript of approximately 7.5 kb, which is consistent with the human CLASP-2 cDNA. An additional specific transcript is detected at 5.5 kb, which could represent an alternative splice variant of mouse CLASP-2 mRNA. Expression of mouse CLASP-2 is clearly detected in brain, heart, kidney, liver and lung with slight expression detected in skeletal muscle. Signal intensities correlate to the relative abundance of mouse CLASP-2-specific RNA in the various tissues.

Mouse CLASP-3

To determine mouse CLASP-3 expression profile Northern blot analysis was carried out. A probe representing a portion of the mouse CLASP-3 cDNA was hybridized against RNA from multiple mouse tissues. As FIG. 4C shows, this probe reveals a specific transcript of approximately 7.6 kb, which is consistent with the human CLASP-3 cDNA. An additional specific transcript is detected at 5.5 kb, which could represent an alternative splice variant of mouse CLASP-3 mRNA. Expression of mouse CLASP-3 is clearly detected in brain, heart, kidney, liver and lung and testis with slight expression detected in skeletal muscle and spleen. Signal intensities correlate to the relative abundance of mouse CLASP-3-specific RNA in the various tissues.

Mouse CLASP-4

To determine mouse CLASP-4 expression profile Northern blot analysis was carried out. A probe representing a portion of the mouse CLASP-4 cDNA was hybridized against RNA from multiple mouse tissues. As FIG. 4D shows, this probe reveals a specific transcript of approximately 7.5 kb, which is consistent with the human CLASP-4 cDNA. Expression of mouse CLASP-4 is clearly detected in brain, heart, lung, and spleen with slight expression detected in kidney, liver, skeletal muscle, and testis. Signal intensities correlate to the relative abundance of mouse CLASP-4-specific RNA in the various tissues.

Mouse CLASP-5

To determine expression of mouse CLASP-5-specific RNA in various tissues, Northern analysis was performed. A radioactively labeled (DNA) probe representing the DNA portion that encodes mouse CLASP-5 sequence was hybridized against membrane-immobilized RNA isolated from various human tissues. As FIG. 4E shows, hybridization using this probe revealed the predominant expression of mouse CLASP-5 in kidney, liver, lung and spleen. Trancript size is approximately 7.5 kb, with the exception of liver, where the predominantly expressed transcript is only ˜6.5 kb in length. To a significantly lower extent, the 7.5 kb transcripts was testis and heart, and very faint expression of that transcript was detected in brain. In heart, additional transcripts of 4.5 kb and 3.5 kb length are seen to a slightly higher extend than the 7.5 kb transcript. The size of the 7.5 kb transcript correlates to the length of mouse CLASP-5 RNA as predicted from mouse CLASP-5 cDNA sequence, as well as to the size of human CLASP-5 transcripts.

Mouse CLASP-7

To determine mouse CLASP-7 expression profile Northern blot analysis was carried out. A probe representing a portion of the mouse CLASP-7 cDNA was hybridized against RNA from multiple mouse tissues. As FIG. 4F shows, this probe revealed a predominant transcript of 7.2 kb, which is consistent with the human CLASP-7 cDNA. Another transcript of approximately 4.5 kb was also detected, which could represent alternative splice variants of mouse CLASP-7 mRNA. Expression of mouse CLASP-7 is clearly detected in heart, liver (4.5 kb transcript), lung and testis with weaker expression in brain, kidney, and skeletal muscle, and slight expression detected in liver (7.2 kb transcript) and spleen. Signal intensities correlate to the relative abundance of mouse CLASP-7-specific RNA in the various tissues.

Methods

Northern Membranes

Messagemap Multiple Tissue Northern blot membranes carrying poly A+RNA from various mouse tissues was purchased from Stratagene

Probes

Mouse CLASP-1

A mouse CLASP-1 5′ RACE product representing nucleotides-169 to 804 presented in FIG. 1 was used as a probe. The DNA fragment was isolated by digesting the 5′ RACE subclone with NotI and gel purifying (Sephaglass, Amersham Pharmacia) the fragment away from the vector.

Mouse CLASP-2

A DNA probe representing a portion of mouse CLASP-2 cDNA was generated by PCR.

Primer mC2S3 (5′-AGCTGCTGGAGCAGTGTGCGGATG-3′) and primer mC2AS12 (5′-GATCCGCTTCTTCACGTAG-3′) were used to generate a 616 bp fragment representing nucleotides 509-5685 of the mouse CLASP-2 cDNA presented in FIG. 1A.

Mouse CLASP-3

A DNA probe representing a portion of mouse CLASP-2 was generated by PCR.

Primer mC3S17 (5′-TCAGAAGGATACAGAAATG-3′) and primer mC3AS15 (5′-ACCGTCTGGCTTATCATG-3′) were used to generate a 686 bp fragment representing nucleotides 2790-3476 of the CLASP-3 cDNA presented in FIG. 1A.

Mouse CLASP-4

A DNA probe representing a portion of mouse CLASP-4 cDNA was generated by PCR.

Primer mC4S16 (5′-ATTTGCTTGGGCAGCCAGAC-3′) and primer mC4AS9 (5′-TGGCTGAGACTGGCAGCTG-3′) were used to generate a 760 bp fragment representing nucleotides 1578-2338 of the mouse CLASP-4 cDNA sequence presented in FIG. 1A.

Mouse CLASP-5

A DNA probe representing a portion of mouse CLASP-5 was generated by PCR.

Primer mC5S8 (5′-ATAACATCATCAACAAGGAC-3′) and mC5AS18 (5′-TGGCTCCTACAGAGCCCTGCAGTA-3′) were used to generate a 611 bp fragment representing nucleotides 5138 to 5749 of the mouse CLASP-5 cDNA sequence presented in FIG. 1A.

Mouse CLASP-7

A DNA probe representing a portion of mouse CLASP-7 cDNA was generated by PCR.

Primer mC7S2 (5′-TCACGCTGGTACATGTTCTAGAG-3′) and primer mC7AS2 (5′-AGCTGGAAGAAGAACCAGGCATG-3′) were used L929 cDNA to generate a probe encompassing sequences in Fragment 1 presented in FIG. 1.

Probe Labelling

For Northern hybridizations, 20 ng of mouse CLASP specific agarose gel-purified (Sephaglass, Amersham Pharmacia) DNA fragment was radioactively labeled using using the Ready-to-Go Kit from Amersham-Pharmacia and ³²P-dCTP (Amersham). The specific activity for each labeled fragment was approximately 2×10⁹ cpm/ug DNA.

Hybridization Conditions

Hybridizations of ³²P-dCTP labeled mouse CLASP-2, 3, 4, 5 and 7 DNA probes to the membrane immobilized RNA were carried out over night in Church hybridization solution (0.5 M Na-phosphate pH 7.2, 1 mM EDTA, 7% SDS, 1% BSA), or in Quikhyb hybridization solution (Stratagene) for mouse CLASP-1 at 65° C. Blots were washed 2 times in 2×SSC 0.1% SDS for 10′ each @ 50 C. and then 3 times in 0.2×SSC 0.1% SDS for 10′ each at 65° C., followed by a 5′ wash in 2×SSC at room temperature. Exposure to Kodak BioMax MS film was carried out at −75° C. for 36 hours using intensifying screens.

### Example 3

### Antisense Inhibition of Mouse CLASP Expression

### Example 3A

Inhibition of Mouse CLASP Expression in vitro

In this example, inhibition of mouse CLASP expression is examined using an in vitro cell-free expression system. To identify the useful antisense oligonucleotides, a series of antisense phosphorothioate oligonucleotides (PS-ODNs), which span portions of mouse CLASP sequences, can be systematically assayed for the ability to block mouse CLASP expression in vitro.

For inhibition of mouse CLASP expression in vitro, a mouse CLASP transcription/expression plasmid can be used according to standard methodology for in vitro transcription and translation of sense mouse CLASP RNAs. Coupled transcription-translation reactions can be performed with a reticulocyte lysate system (Promega TNTTM) according to standard conditions. Each coupled transcription/translation reaction can include amouse CLASP RNA transcribed from the expression plasmid, and a test antisense polynucleotide at a range of standard test concentrations, as well as the luciferase transcription/translation internal control to normalize each reaction (see, e.g., Sambrook et al., supra, Ausubel et al., supra). The translation reaction can also be performed with a sense mouse CLASP RNA that is synthesized in vitro in a separate reaction and then added to the translation reaction. ³⁵S-Met is included in the reaction to label the translation products. The negative control is performed without added PS-ODN or a sense PS-ODN.

The labeled translation products can be separated by gel electrophoresis and quantitated after exposing the gel to a phosphorimager screen. The amount of mouse CLASP protein expressed in the presence of amouse CLASP specific PS-ODNs can be normalized to the co-expressed luciferase control.

### Example 3B

Inhibition of Mouse CLASPs Expression ex vivo

A. Reagents

Cells: Jurkat, Clone E6-1 ATCC TIB-152; 9D10 ATCC CRL8752; additional cells from the ATCC or NCI.

Media and solutions: RPMI 1640 medium, BioWhitaker; DMEM/M199 medium, BioWhitaker; EMEM, BioWhitaker; Fetal Bovine Serum, Summit (stored frozen at −20° C., stored thawed at 4° C.); Trypsin-EDTA, GIBCO (catalogue #25300-054) (stored frozen at −20° C., stored thawed 4° C.; Isoton II (stored at RT); DMSO (stored at RT); oligonucleotides (see Table 1 and FIG. 3, stored in solution at −20° C.); PBS (Ca²⁺/Mg²⁺ free); TE; 10 mM Tris-HCL, pH 8.0; 1 mM EDTA.

To prepare oligonucleotide stocks: Oligonucleotide nucleotides (PS-ODNs) can dissolved in the appropriate amount of TE to make a concentrated stock solution (1-20 mM).

B. Treatment of Cells ex vivo with Antisense Mouse CLASP Oligonucleotides

Stock cultures of cells in log-phase growth (in T75 flask) can be used. Jurkat, and 9D 10 cells are used in this assay. Jurkat and 9D10 are suspension cultures and are passed through dilutions in media. Cell density is measured using a Coulter counter or hemacytometer.

For 6-well dishes, 1.1×10⁵ cells total per well, 2 ml/well is added. The amount of cells can be scaled up or down proportionally for 12-well, 100 mm, or 150 mm dishes. For example, for 12-well dishes, use 4.6×10⁴ cells in 2 ml media; for 100 mm dishes use 6×10⁵ cells in 10 ml media; for 150 mm dishes use 1.7×10⁶ cells in 35 ml media.

An appropriate number of cells (as described in step 2 above) are collected, centrifuged and resuspended in media containing a range of ODN concentrations. The cells are treated in single, duplicate, or triplicate wells. Control wells are treated with TE or sense ODNs diluted in media.

The suspension cultures are washed and resuspended daily with PS-ODN media.

Suspension cultures are grown for 2-4 days. Cells are washed with PBS and density measured using a Coulter counter or a hemocytometer. If necessary, the cells are replated at 1.1×10⁵ cells per well, 2 ml media per well, and fed with PS-ODN as described above.

Samples of the cells can also be harvested for analysis to determine the effects of mouse CLASP antisense ODNs. Samples are harvested for RNA and analyzed by either Northern analysis or RT-PCR for the presence of mouse CLASP mRNA. Funtional consequences of mouse CLASP antisense ODNs can be analyzed by measuring the ability of tissue culture cell lines (e.g. 2B4, CH27, Jurkat and 9D10) to be activated. 2B4 and Jurkat cells lines are activated by exposure to anti-CD3 and anti-CD28 crosslinking antibodies, and 9D10 and CH27 cells are activated by exposure to anti-IgM crosslinking antibody or P. aeruginosa lipopolysaccharide. A hallmark of activation, calcium influx, can be measured by flow cytometry. Additionally, ELISA assays can be used to measure Interleukin-2 production from Jurkat and 2B4 cells and secreted IgM can be measured using standard assays from 9D10 and CH27. Table 7 below shows exemplary oligonucleotides for this assay:

TABLE 7

| mouse | Sequence | | |
|---|---|---|---|
| CLASP | 5′-3′ | length | notes/comments |

| | | | | |
|---|---|---|---|---|
| 1 | 1 | TTTCCTCG | 30 | Spans nucleotides −9 to 20 |
| | | GAAGCTCA | | of FIG. 1A |
| | | TCATATGC | | |
| | | CTTGA | | |
| 2 | 1 | AAGCTCAT | 30 | Spans nucleotides −19 to 21 |
| | | CATATGCC | | of FIG. 1A |
| | | TTGAAATG | | |
| | | GAGTCA | | |
| 3 | 1 | CGCCTCCT | 30 | Spans nucleotides −12 to 41 |
| | | CTTCCAGA | | of FIG. 1A |
| | | ATTCCTTT | | |
| | | CCTCGG | | |
| 4 | 2 | GGGAAGAG | 30 | Spans nucleotides −16 to 14 |
| | | GAGCATCT | | of FIG. 1B |
| | | CCCGCAGG | | |
| | | CAGTCG | | |
| 5 | 2 | GTCATCAT | 30 | Spans nucleotides −6 to 24 |
| | | AAGGGAAG | | of FIG. 1B |
| | | AGGAGCAT | | |
| | | CTCCCG | | |
| 6 | 2 | TTCCAGAC | 30 | Spans nucleotides 25 to 54 |
| | | GGCCATCC | | of FIG. 1B |
| | | TGAGGCGG | | |
| | | CAGGGG | | |
| 7 | 3 | CCATTTCA | 30 | Spans nucleotides 2 to 31 of |
| | | CTTTCTTC | | |
| | | AGGCACAG | | |
| | | CTGAAA | | |
| 8 | 3 | GTCTTCTG | 30 | Spans nucleotides 43 to 72 |
| | | TGTAACTT | | of FIG. 1C |
| | | CTTATACA | | |
| | | ATCTCT | | |
| 9 | 3 | GATCATCT | 32 | Spans nucleotides 198 to 229 |
| | | TGCTCATC | | of FIG. 1C |
| | | CTGGTAGC | | |
| | | TGCTGCCA | | |
| 10 | 4 | GAATTTGC | 31 | Spans nucleotides −10 to 21 |
| | | GCACTTCG | | of FIG. 1D |
| | | GCCATGGC | | |
| | | AGAGGTG | | |
| 11 | 4 | CACTTCGG | 32 | Spans nucleotides −20 to 12 |
| | | CCATGGCA | | of FIG. 1D |
| | | GAGGTGGC | | |
| | | GGGCAGGT | | |
| 12 | 4 | CTGCCGTG | 31 | Spans nucleotides 22 to 52 |
| | | CCTGGCTT | | of FIG. 1D |
| | | GCTCAGCC | | |
| | | GCTTGGT | | |
| 13 | 5 | CAAGCTGT | 33 | Spans nucleotides −12 to 21 |
| | | TTAAGTGT | | of FIG. 1E |
| | | GTCATCAC | | |
| | | GAGTCCTT | | |
| | | C | | |
| 14 | 5 | GTGTGTCA | 31 | Spans nucleotides −22 to 9 |
| | | TCACGAGT | | of FIG. 1E |
| | | CCTTCAAA | | |
| | | GTCCACT | | |
| 15 | 5 | GACCTCAC | 30 | Spans nucleotides 49 to 78 of |
| | | CGATGACG | | |
| | | ACCTGCAT | | |
| | | GTGGCC | | |
| 16 | 7 | CACTATAG | 30 | Spans nucleotides 5 ot 34 of |
| | | CCTCGATC | | FIG. 1F, Fragment 1 |
| | | CTGGGGCC | | |
| | | CCCGAC | | |
| 17 | 7 | GTCACAGG | 31 | Spans nucleotides 164 to 194 |
| | | GCGCAATC | | of FIG. 1F, Fragment 1 |
| | | GCCTCAGC | | |
| | | AGGGATG | | |
| 18 | 7 | CAGGTTCT | 30 | Spans nucleotides 208 to 237 |
| | | CGGGGGCC | | of FIG. 1F, Fragment 1 |
| | | GGGGAGAT | | |
| | | GTCAAG | | |

Oligonucleotides presented in Table 7 are the anti-sense (i.e., reverse-complement) of the nucleotides they span as indicated in the last column.

### Example 4

### Example 4A

Synthesis of Carboxyl-termini PDZ-ligand peptides

The GST-PDZ fusion proteins are made following standard procedures. An exemplary GST-PDZ fusion protein was constructed as follows: A 572 bp fragment encoding two PDZ domains of the human neDLG gene (Genbank Accession No. U49089.1) was amplified from total Jurkat RNA by RT-PCR according to standard protocols (Sambrook, Fritsch, and Maniatis, 1989, Molecular Cloning—A Laboratory Manual. Cold Spring Harbor Press.) using primers flanked by restriction endonuclease sites for cloning. Fragments were purified by Sephaglas (Pharmacia), digested with the appropriate enzymes, and ligated into the GST expression vector pGEX-3X (Pharmacia) cut with similar enzymes. Recombinant constructs were confirmed by sequencing. Fusion proteins were expressed by IPTG induction in DH5α and purified using glutathione-Sepharose (Pharmacia) according to instructions from Pharmacia. Excess glutathione was removed using a PD10 desalting column (Pharmacia) and samples were diaconcentrated by placing the protein in dialysis tubing (14,000 MW cutoff) and laying the tubing on polyethylene glycol (3350; Sigma) until volume had been reduced by approximately 50%. Glycerol was then added to 35% final concentration and samples were stored at −20° C. These recombinant proteins have been used to generate antibodies (Josman laboratories) by standard protocols and for biochemical studies describe herein.

Synthetic peptides corresponding to the carboxyl-terminus of a protein of interest are synthesized by standard resin-based chemistry (e.g., FMOC), labeled with biotin at the amino-terminus when indicated, and cleaved from the resin using a halide containing acid (e.g, trifluoroacetic acid). The synthetic peptides are then purified by reverse phase high performance liquid chromatography (HPLC) and the identity of the peptides are confirmed by mass spectrometry.

### Example 4B

Measurement of Mouse CLASP Peptide Binding to PDZ Domain-containing Proteins

The binding of a biotinylated carboxyl-terminal peptide to a GST-PDZ fusion protein is measured as follows:

(1) GST fusion protein containing one or more PDZ domain(s) is coated onto a protein-binding surface. The protein-binding surface is the surface of a polystyrene plate, which in some cases has been pre-treated by coating with 5 μg/ml of goat-anti-GST polyclonal antibody followed by blocking with excess bovine serum albumin (BSA). The concentration of GST fusion protein used is 5-10 μg/ml and the reaction of the GST fusion protein with the plate is carried out in PBS for 1-16 hours at 4° C. If not already blocked, the plate is then blocked with BSA (2% in PBS, 2 hours, 4° C.)

(2) The plate is washed with PBS.

(3) The biotinylated peptide (generally 0.2-20 μM) is then added to the plate and allowed to react in PBS/2% BSA buffer with the GST fusion protein for 10 minutes at 4° C. followed by 20 minutes at 25° C. In cases where competition between a labeled (biotinylated) and unlabeled (non-biotinylated) peptide is performed, the unlabeled peptide is added immediately prior to adding the labeled peptide.

(4) The plate is washed with PBS.

(1) 0.5 μg/ml steptavidin-HRP conjugate is added to the plate in PBS/2% BSA buffer and allowed to react for 20 minutes at 4° C.

(6) The plate is washed 5× with detergent (tween 20) containing solution.

(7) The plate is developed by addition of HRP-substrate solution for 20 minutes at room temperature.

(8) The reaction of the HRP and its substrate is terminated by addition of 1 M sulfuric acid.

(9) The optical density of each well of the plate is read at 450 nm.

In cases where measurement of the apparent affinity of PDZ-ligand interaction is desired, the above procedure is carried out with multiple concentrations of the labeled peptide being used in a single experiment. A plot of binding versus peptide concentration added is then fit to the equation:

Binding [peptide]=Saturation Binding×([peptide]/([peptide]+Kd))

where “Binding [peptide]” is the binding of a given concentration of peptide to the GST-PDZ fusion protein minus binding to the GST alone control, “Kd” is the apparent affinity of the binding reaction, and “Saturation Binding” is computed to allow the best fit of the data to the above equation. The term apparent affinity is used because the reaction may not reach equilibrium during the duration of the binding reaction in which case the apparent affinity would underestimate the actual affinity (i.e., actual Kd <observed Kd).

### Example 5

Expression of HC1 and MC1 in Mammalian Cells

Expression of mouse and human CLASP1 (MC1, HC1) molecules was evaluated in different cell lines using DNA transfection of tagged fusion protein constructs. Two standard methods for DNA transfection, DNA precipitation by calcium phosphate (Graham, F. and van der Eb, A. and Gorman, C. et al.,) and electroporation (Potter, H.) were used. Two different methods of monitoring expression of human and mouse CLASP-1 were used in mouse and human lymphocytic cells and in human 293 cells: human IgG (Fc specific fragment) and FLAG expression tags. Human CLASP-1 was tested using the hIgG expression system only. The expression systems were designed to control for proper protein folding of MC1 and HC1 by expressing the tagged protein c-terminal to the CLASP1 molecules.

Material and Methods

Cell Lines Used for Transfection of CLASP-tagged Constructs

Jurkat E6 human T cells, (ATCC TIB-152), were maintained and tested in complete IMDM (IMDM medium supplemented with 2 mM glutamine, 100 U/mL penicillin, 100 μg/mL streptomycin, 0.1 mM nonessential amino acids, 1 mM sodium pyruvate (Gibco BRL), 50 μM beta mercaptoethanol (Sigma), and 10% fetal calf serum (Gemini Bio-Products)). Human embryonic kidney cells, 293, were maintained and tested in complete DMEM (DMEM medium supplemented with 2 mM glutamine, 10 mM HEPES, 100 U/mL penicillin, 100 μg/mL streptomycin, and 10% fetal calf serum). CH27 mouse B cell lymphoma and 2B4 mouse T cell hybrid were maintained and tested in complete RPMI (RPMI 1640 medium supplemented with 2 mM glutamine, 100 U/mL penicillin, 100 μg/mL streptomycin, 0.1 mM nonessential amino acids, 1 mM sodium pyruvate, 10 mM HEPES, 50 μM beta mercaptoethanol, and 10% fetal calf serum).

Transfection Methods

DNA precipitation by calcium phosphate was used to transfect 293 cells. At least 2 hrs before transfection, ˜5×10⁵ cells, in 5 ml of complete DMEM, were plated onto one 60 mm TC-treated dish for each transfection. For each plate, 1-10 μg of DNA was brought to a volume of 110 μl with deionized H₂O. Fifteen μl of 2M CaCl₂ was added to the DNA solution, which was then slowly added to 125 μl of 2×HBS pH 7.0, (2×HBS=1.64% NaCl 1.188% and Hepes 0.04% Na₂HPO₄). A fine precipitate formed, which was then added dropwise to the plate of cells and incubated at 37° C., 5% CO₂. Cells were analyzed for expression on day 1, 2 and 3 post-transfection (see Analysis of CLASP-tagged constructs).

The lymphocytic cell lines (Jurkat E6, CH27, and 2B4) cells were transfected by electroporation and tested for protein expression on day 1 and 2 post-transfection. The BTX ECM830 generator was used to transfect the Jurkat E6 cells as follows: two cuvettes, with a 4 mm electrode gap, containing 5×10⁶ cells in 0.5 ml serum free-IMDM and 5-30 μg of DNA, were electroporated with a single pulse at 260 volts for 50 msec. Cuvettes were immediately placed on ice for 10-15 minutes before being transferred to 10 ml of complete medium. CH27, and 2B4 cells were transfected in cuvettes with a 4 mm electrode gap, using the BioRad Gene pulser. The protocol using the BioRad Gene pulser is the same as for the BTX ECM830 generator except that they were electroporated in serum free-RPMI @ 0.45 kV, 960 μF, and unlimited resistance. The time constant using the BioRad electroporator ranged from 38-44.

Analysis of CLASP-tagged Fusion Constructs

Analysis of MC1- and HC1-hIgG (Fc specific fragment)-tagged constructs peak10-CLASP1-IgG fusion constructs: HC1-EC12-Ig, MC1-EC12-IgG, MC1-ECM-IgG, MC1-ECC-IgG, and CD4-IgG and IgG only controls. A second vector was also tested, CD5-IgG, for HC1-EC12-IgG expression. These fusions extend from the putative cadherin cleavage homology (nt 3036) to various points up to the predicted transmembrane domain following nucleotide 5100 of FIG. 1A.

293 cells transfected with the CLASP1-hIgG (Fc sp.) fusion constructs were analyzed using flow cytometry, fluorescent microscopy and human IgG ELISA. On day two and seven post-transfection, cells were fixed and permeabilized and tested for intracellular fusion protein expression using FITC conjugated-goat anti-hIgG (Fc specific) antibody (Caltag). A control FITC conjugated-goat antibody control was also tested. Day two expression of MC1-ECM-IgG, and CD4-IgG were shown to be between 30-40% positive for expression of hIgG using flow cytometry. By day seven, expression levels were still visible by fluorescent microscopy and flow cytometry levels had dropped to 5-10%.

IgG (Fc fragment)-tagged Expression by Human IgG ELISA

We have developed a highly sensitive anti-IgG sandwich ELISA in which goat anti-human antibody (Jackson ImmunoResearch) is used to capture the CLASP1-tagged-IgG(Fc) proteins, and Protein A-conjugated-HRP (Pierce) is used to detect any captured IgG-fusion protein. Capture antibody is coated (0.5 μg/ml) onto Nunc maxisorp 96-w ELISA plates overnight at 4° C. 0. 1% fish skin gelatin (Sigma) in PBS was used as the blocking buffer for one hour at RT. All samples were diluted in PBS with 2% BSA and after each step the plate was washed five times with 50 mM Tris pH 7.5, 0.2% tween 20. Following incubation of samples for 30 min., the plate was incubated with 100 μl of Protein A-HRP at 1:10,0000 for 30 min. Assay development begins with 100 μl of TMB solution (Dako) incubated in the dark for 15-30 min. The reaction is stopped by adding 100 μl of 1M H₂SO₄. The absorbency is taken at 450 nm using a Molecular Devices ThermoMax microplate reader. A normal plasma standard control was serially diluted beginning at 1:50,000. Unknown sample results are expressed as either A450 nm or as ng/ml of human IgG calculated from plasma standard curve.

Supernatant from all 293 CLASP1-hIgG-fusion transfectants did show soluble IgG-immunoreactivity in transient and stable cells but cell lysates and intracellular staining from the same transfectants revealed that most of the CLASP-hIgG-fusion products were intracellularly localized.

MC1-EC12-IgG, MC1-ECM-IgG, MC1-ECC-IgG, and IgG control plasmids were transfected into Jurkat E6 cells, but no IgG-immunoreactivity was found in the cell lysate on day one post-transfection (detection level sensitivity is estimated to be 1-2 ng/ml IgG). This suggests that although these proteins are able to be expressed in human 293 cells, they are either detrimental to Jurkat cells or a mechanism exists for limiting expression of these constructs such prevention of translation or rapid post-translational degradation.

RT-PCR positive Transcription in CH27 Cells

To distinguish between transcriptional and translational mechanisms for preventing mCLASP-1 expression in lymphocytes, it was determined that the vectors encoding the mCLASP-1 protein fusions were being transcribed after a month of selection in G418. RNA was extracted from stable mouse B cell lines (CH27) carrying the three mouse CLASP-1 expression constructs by Trizol extraction (Gibco-BRL). After extensive DNAseI treatment, RT-PCR was performed using primers that could distinguish between vector and endogenous copies of mCLASP-1. It was found that the cells contained stably expressed RNA from the vectors, suggesting that the lack of mCLASP-1 protein expression is not due to transcriptional downregulation.

Analysis of MC1-FLAG-tagged Constructs

The expression of mouse CLASP-1 fusion proteins was analyzed in several lymphocytic cell lines. There was no stable protein expression although similar regions had been successfully expressed in human 293 cells. Mouse CH27 (B-cell), 2B4 (T-cell) or Jurkat (human T-cell) lines were transiently transfected by electroporation with pBJ-mCLASP-FLAG fusion constructs. On day 1 or 2 post transfection, 0.5 to 1 million cells were pelleted and resuspended in SDS-PAGE denaturing sample buffer. These were analyzed by western blot using antibodies against the carboxy-terminal FLAG tag (Sigma) and standard secondary antibodies for detection (Sheep anti-mouse IgG-HRP; Amersham). This western was able to detect a control bacterial alkaline phosphatase-FLAG fusion (49 kD, Sigma) down to 12.5 ng but failed to recognize mCLASP1-FLAG fusions in any lymphocytic cell line.

Discussion

Given the lack of expression of truncated forms of both mouse and human CLASP-1 using different expression systems, it was concluded that the level of CLASP-1 expression is highly regulated in mouse and human lymphocytes, but not in human 293 cells. This regulation is at the level of translation or post-translation, possibly affecting a lymphocyte process necessary for cell survival, protein expression, or sequestering proteins away from where they are needed for these processes.

References

Graham, F. and van der Eb, A., (1973). Virology 52:456.

Gorman, C., Science, (1983). 221, 551-553.

Potter, H. (1995). Recombinant DNA Methodology II. Academic Press, Inc. Chapter 31, pg. 467-484. Applications of Electroporation in Recombinant DNA Technology.

### Example 6

Expression of Human CLASP-1 in Activated T-cells

General Experimental Design

The expression profiles of human CLASP-1 in T cells upon T cell activation was determined by Northern analysis. Jurkat E6 lymphoblasts were activated by treatment with anti-CD28, PMA, and Ionomycin. Subsequently, total RNA was extracted from cell aliquots harvested at 0, 1, 2, 4, 8, and 14 hours post activation. The RNA concentration of each preparation was determined by the absorption at 260 nm using a spectrophotometer and concentrations of the different RNA preparations were adjusted such that equal quantities of each RNA preparation could be subjected to Northern analysis. Even gel loading was monitored by ethidium bromide staining of the formaldehyde-agarose gel. Northern membranes were hybridized to radioactively labeled probes corresponding to portions of human CLASP-4 and human beta-actin. Expression levels of CLASP-1 at different time points post T-cell activation are proportional to the radioactive signal generated by hybridization by the CLASP-1 specific radioactively labeled probe that remained bound to the Northern membrane under stringent washing conditions. The entire experiment was done in duplicate.

Jurkat E6 Cell Activation

Jurkat E6 cells were maintained and tested in complete IMDM medium supplemented with 2 mM glutamine, 10 mM HEPES, 100 u/mL penicillin, 100 μg/mL streptomycin, 0.1 mM nonessential amino acids, 1 mM sodium pyruvate (Gibco/BRL), 50 μM beta mercaptoethanol (Sigma), and 10% fetal calf serum (Gemini). T cells were activated as described per Fraser et. al., using 0.1 μg/mL mouse anti-human CD28 monoclonal antibody (PharMingen International catalog number 33741A), 50 ng/mL PMA (Sigma), and 1 μM ionomycin (Calbiochem). Following incubation at 37° C. and 5.0% v/v CO₂, 0.5×10⁶ cells were harvested by centrifugation at 500×g for 10 minutes (min) at room temperature at 0, 1, 2, 4, 8 and 14 hours post activation and subjected to RNA extraction.

RNA Preparation

Jurkat cell pellets corresponding to 0.5×10⁶ cells were resuspended in 100 μl PBS (phosphat buffered saline) and 8 mL of TRIZOL (GIBCO/BRL #15596-026)was added to cell suspensions, followed by 5 min incubation at room temperature (RT). RNA was extracted from TRIZOL with 1.6 mL chloroform (Kodac#IB05040) by shaking for ˜15 seconds, followed by a 3 min incubation at RT. The mixture TRIZOL/chloroform mixture was centrifuged (in Sarstedt tubes #60.540) in a SA-600 rotor (Sorvall) at 9000 RPM for 10 min at 4° C. using the Sorvall RC5B centrifuge. The upper aqueous layer containing the RNA was transferred to another 13 mL test tube and the RNA was preciptated by addition of 4 mL isopropyl alcohol. After a 10 min incubation at RT, samples were centrifuged as above. The supernatant was removed by decanting and the pellet was washed with 8 mL of 70% ethanol, vortexed, and centrifuged at 7000 RPM for 10 min at 4° C. in a Sorvall RC5B+ centrifuge, SA600 rotor. Again the supernatant was removed by decanting and the RNA pellet was air dried. The pellet was then dissolved into RNAse-free, DEPC-treated dH₂O and incubated for 10 min at 65° C. All samples were quantified and tested for purity (1.5±0.1, expected ratio) by measuring the absorbancy at 260 and 280 nm on a Beckman DU 640B spectrophotometer. Until Northern blot analysis, samples were stored at −80° C.

Probe Labeling

PCR generated DNA fragments were 32P-dCTP radioactively labeled using the “ready to go” system from Amersham-Pharmacia (# 27-9240-01). All procedures were as recommended in the suppliers recommendation. Reaction volumes were 50 μl with 20-40 ng of DNA to be labeled. For labeling, samples were incubated for 15 min at 37° C. Unincorporated nucleotides were separated from the labeled substrate by column gel chromatography (Amersham-Pharmacia Nick-Columns, # 17-0855-22)

Northern Analysis

RNA concentrations were determined by the 260nm/280nm light absorption of the RNA solution. An aliquot corresponding to 20 μg of RNA was ethanol precipitated, the pellet was resuspended in formamide/formaldehyde buffer and the RNA was incubated for 15 min at 65° C. to eliminate RNA secondary structure formation. RNAs were then run over night on a 1.1% agarose gel containing 3% formaldehyde. Gel-and running buffer was 20 mM sodium phosphat, pH 7.5. To visualize RNA after gel migration, approx. 0.5 μg ethidiumbromide was added to each sample prior to the run together with RNA loading buffer. RNA in the gel was then visualized by 260 nm wavelenght UV light and a polaroid picture of the gel was taken for documentation.

After soaking the gel for 15 min in deionized water to reduce the concentration of ethidium bromide and formaldehyde in the gel, the RNA was transfered onto AMERSHAM Hybond-N plus membrane by capillary blotting in 20×SSC buffer for 5 hours. Subsequent to blotting, the membrane was washed in 5×SSC for 1 min and RNA was crosslinked to the membrane by UV light (STRATAGENE STRATALINKER). Hybridizations of ³²PdCTP labeled human CLASP4-specific DNA probe to the membrane bound RNAs was carried out in “Church” hybridization solution (7% w/v sodium dodecyl sulfate, 0.5 M sodium phosphate pH 7.2, 1 mM EDTA) at 65° C. and for 8-12 hours. The CLASP-1-specific probe was generated by PCR from a plasmid containing portions of CLASP-1 cDNA. Primers were 114CF(S) and 115CR(AS), the probe covers N5704 to N 6549 of CLASP-1.

Blots were washed twice in 2×SSC 0.1% SDS for 10 min each at 60° C. and then twice in 0.2×SSC 0.1% SDS for 10 min each at 60° C., followed by a 5′ wash in 2×SSC at 60° C. Exposure to KODAK BIOMAX MS film was carried out at minus 80° C. using amplifying screens. Typically, exposure times were 10 to 36 hours.

Signal intensities on Northern membranes were quantified by the use of a phosphor imager system (STORM, Molecular Dynamics). Signals were counted in the “volume report” mode.

Results

CLASP-1 expression as determined by Northern analysis does not change throughout 14 hours post activation (FIG. 4). Intensities of CLASP-1-specific signals were quantified by phosphor imager analysis. Rectangles were drawn around the areas of CLASP-1-specific signal and total quantity of signal was determined by the “volume report” mode. As determined by ethidium bromide staining, RNA sampes were loaded in equal quantities onto the corresponding Northern analysis gel.

### Example 7

Chromosomal Location of CLASP-1 and Possible Disease Associations

CLASP-1 cDNA sequences have been mapped to the genomic clone (GI:8705162) by use of sequence homology bioinformatics tools BLAST.

Clone (GI:8705162) has previously been mapped to the chromosomal location 2q36.2. The literature research reports that the mutations, deletions, rearrangements, disomies and/or breakpoints (in general: chromosomal aberations) in below listed genes make the genes strong candidates for the onset of the listed diseases/disorders. Because the CLASP-1 gene is localized in the chromosome location 2q36.2, abnormal CLASP-1 gene regulation or deletion, rearrangement and/or mutations in CLASP-1 locus might be directly or indirectly associated with the onset of the listed diseases. Further, CLASP-1 gene can be used as a genetic probe to detect the abnormality in regions of these below listed genes and as a diagnostic marker for the related disease/disorders.

| CANDIDATE | | RELATED |
|---|---|---|
| GENE | LOCUS | DISEASE/DISORDERS |
| PAX3: PAIRED BOX | 2q36.1-q36.2 | CDHS: facial cranial and hand |
| (DNA BINDING) | | defect/malformation. |
| CONTAINING | | |
| PROTEIN 3 | | |
| IRS1: INSULIN | 2q36 | NIDDM3: non insulin-dependent |
| RECEPTOR | | Diabetes type 3 with insulin |
| SUBSTRATE 1 | | resistance. |

The present invention is not to be limited in scope by the exemplified embodiments which are intended as illustrations of single aspects of the invention, and any clones, DNA or amino acid sequences which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims. It is also to be understood that all base pair sizes given for nucleotides are approximate and are used for purposes of description.

All publications and patent documents cited above are hereby incorporated by reference in their entirety for all purposes to the same extent as if each were so individually denoted.

What is claimed is:

1. An isolated CLASP-1 polynucleotide, wherein said polynucleotide is
(a) a polynucleotide that has the sequence of SEQ ID NO: 1 or
(b) a polynucleotide that hybridizes under stringent hybridization conditions to (a) and encodes a polypeptide having the sequence of SEQ ID NO: 2 or an allelic variant or homologue of a polypeptide having the sequence of SEQ ID NO: 2; or
(c) a polynucleotide that hybridizes under stringent hybridization conditions to (a) and encodes a polypeptide with at 25 contiguous residues of the polypeptide of SEQ ID NO: 2; or
(d) a polynucleotide that hybridizes under stringent hybridization conditions to (a) and has at least 12 contiguous bases identical to or exactly complementary to SEQ ID NO: 1.

2. The polynucleotide of claim 1 that encodes a polypeptide having the full-length sequence of SEQ ID NO: 2.

3. The isolated polynucleotide of claim 1, comprising the cDNA coding sequence of ATCC accession number ______.

4. An isolated CLASP-1 polynucleotide comprising a nucleotide sequence that has at least 90% percent identity to SEQ ID NO: 1.

5. An isolated polypeptide comprising a nucleotide sequence that has at least 90% sequence identity to SEQ ID NO: 2 and is immunologically crossreactive with SEQ ID NO: 2 or shares a biological function with native CLASP-1.

6. A vector comprising the polynucleotide of claim 1.

7. An expression vector comprising the polynucleotide of claim 1 in which the nucleotide sequence of the polynucleotide is operatively linked with a regulatory sequence that controls expression of the polynucleotide in a host cell.

8. A host cell comprising the polynucleotide of claim 1, or progeny of the cell.

9. A host cell comprising the polynucleotide of claim 1, wherein the nucleotide sequence of the polynucleotide is operatively linked with a regulatory sequence that controls expression of the polynucleotide in a host cell, or progeny of the cell.

10. The host cell of claim 8 which is a eukaryote.

11. The polynucleotide of claim 1 that is an antisense polynucleotide less than about 200 bases in length.

12. An antisense oligonucleotide complementary to a messenger RNA comprising SEQ ID NO: 1 and encoding CLASP-1, wherein the oligonucleotide inhibits the expression of CLASP-1.

13. An isolated DNA that encodes a CLASP-1 protein as shown in SEQ ID NO: 2.

14. The polynucleotide of claim 1 that is RNA.

15. A method for producing a polypeptide comprising:
(a) culturing the host cell of claim 8 under conditions such that the polypeptide is expressed; and
(b) recovering the polypeptide from the cultured host cell or its cultured medium.

16. An isolated polypeptide encoded by a polynucleotide of claim 1.

17. The polypeptide of claim 16 that has the amino acid sequence of SEQ ID NO: 2 or a fragment thereof.

18. The isolated polypeptide of claim 16, wherein the polypeptide is cell-membrane associated.

19. The isolated polypeptide of claim 16, wherein the polypeptide is soluble.

20. The polypeptide of claim 17, wherein the polypeptide is fused with a heterologous polypeptide.

21. An isolated CLASP-1 protein having the sequence as shown in SEQ ID NO: 2.

22. A protein comprising the sequence as shown in SEQ. ID. NO: 1 and variants thereof that are at least 95% identical to SEQ ID. NO: 2 and specifically binds spectrin.

23. An isolated antibody that specifically binds to a polypeptide having the amino acid sequence as shown in SEQ ID NO: 2, or a binding fragment thereof.

24. The antibody of claim 23, that is monoclonal.

25. A hybridoma capable of secreting the antibody of claim 24.

26. A method for identifying a compound or agent that binds a CLASP-1 polypeptide comprising:
i) contacting a CLASP-1 polypeptide of claim 17 with the compound or agent under conditions which allow binding of the compound to the CLASP-1 polypeptide to form a complex and
ii) detecting the presence of the complex.

27. A method of detecting a CLASP-1 polypeptide in a sample, comprising:
(a) contacting the sample with an antibody or binding fragment of claim 24 and (b) determining whether a complex has been formed between the antibody and with CLASP-1 polypeptide.

28. A method of detecting a CLASP-1 polypeptide in a sample, comprising:
(a) contacting the sample with a polynucleotide of claim 1 or a polynucleotide that comprises a sequence of at least 12 nucleotides and is complementary to a contiguous sequence of the polynucleotide of section (a) of claim 1, and (b) determining whether a hybridization complex has been formed.

29. A method of detecting a CLASP-1 nucleotide in a sample, comprising:
(a) using a polynucleotide that comprises a sequence of at least 12 nucleotides and is complementary to a contiguous sequence of the polynucleotide of section (a) of claim 1, in an amplification process; and
(b) determining whether a specific amplification product has been formed.

30. A pharmaceutical composition comprising a polynucleotide of claim 1, a polypeptide of claim 16, or an antibody of claim 23 and a pharmaceutically acceptable carrier.

31. A method of inhibiting an immune response in a cell comprising:
(a) interfering with the expression of a CLASP-1 gene in the cell;
(b) interfering with the ability of a CLASP-1 protein to bind to another cell;
(c) interfering with the ability of a CLASP-1 protein to bind to another protein.

32. The method of claim 31, wherein the cell is a T cell or a B cell.

33. The method of claim 31 comprising contacting the cell with an effective amount of a polypeptide which comprises the amino acid sequence of SEQ ID NO: 2 or a fragment thereof.

34. A method of inhibiting an immune response in a subject, comprising administering to the subject a therapeutically effective amount of an antibody which specifically binds a polypeptide having the sequence of SEQ ID NO: 2.

35. A method of preventing or treating a CLASP-1-mediated disease comprising administering to a subject in need thereof a therapeutically effective amount of a pharmaceutical composition of claim 30.

36. The method claim 35, wherein the CLASP-1-mediated disease is an autoimmune disease.

37. A method of treating an autoimmune disease in a subject caused or exacerbated by increased activity of T_{H}1 cells consisting of administering a therapeutically effective amount of a pharmaceutical composition of claim 30 to the subject.
